(19) United States
(12) Patent Application Publication
(10) Pub. No.: US 2012/0129820 A1
(43) Pub. Date: May 24, 2012

(54) NEW PHARMACEUTICAL COMPOSITIONS FOR TREATMENT OF RESPIRATORY AND GASTROINTESTINAL DISORDERS

(75) Inventors: Peter Seither, Biberach an der Riss (DE); Abhya Gupta, Biberach an der Riss (DE)

(73) Assignee: BOEHRINGER INGELHEIM INTERNATIONAL GMBH, Ingelheim am Rhein (DE)

(21) Appl. No.: 13/147,395

(22) PCT Filed: Feb. 8, 2010
(86) PCT No.: PCT/EP2010/051499

§ 371 Date: Oct. 5, 2011

(30) Foreign Application Priority Data
Feb. 9, 2009 (EP) 09152407.4

(51) Int. Cl. A61K 31/56; A61K 31/506; A61K 31/5377; A61P 11/00; A61P 11/06; A61P 1/06; A61P 29/00; A61K 31/522; A61P 37/00; A61P 37/06; A61P 1/10; A61K 31/505; A61P 11/02

(52) U.S. Cl. 514/171; 514/256; 514/235.8; 514/263.34

(57) ABSTRACT

The present invention provides pharmaceutical compositions comprising one or more CRTH2 antagonists 1 and one or more further active compounds 2.

The present invention relates to novel pharmaceutical compositions comprising one or more, preferably one, selected CRTH2 antagonist 1, and at least one further active compound 2, processes for preparing them and their use as medicament in the treatment of respiratory or gastrointestinal complaints, as well as inflammatory diseases of the joints and allergic diseases of the nasopharynx, eyes, and skin.

### DETAILED DESCRIPTION OF THE INVENTION

The present invention relates to pharmaceutical compositions comprising one or more CRTH2 antagonists 1 and one or more further active compounds 2.

### The CRTH2 Antagonists 1

The CRTH2 antagonists 1 herein are as disclosed in WO 2004/096777 and selected from the group consisting of the compounds of general formula (I), their tautomeric and stereoisomeric form, or salts, ester or prodrug thereof:

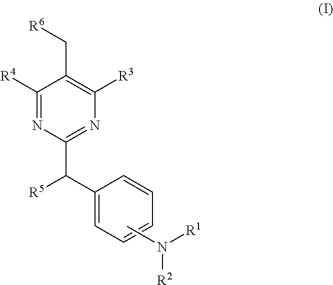

wherein
R¹ represents hydrogen,

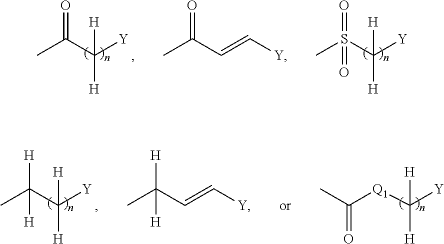

in which
n represents an integer of 0 to 6;
-Q₁- represents —NH—, —N(C₁₋₆alkyl)-, or —O—;
Y represents hydrogen, C₃₋₈ cycloalkyl optionally substituted by C₁₋₆ alkyl, C₃₋₈ cycloalkyl fused by benzene, aryl or heteroaryl, wherein said aryl and heteroaryl are optionally substituted at a substitutable position with one or more substituents selected from the group consisting of cyano, halogen, nitro, guanidino, pyrrolyl, sulfamoyl, C₁₋₆ alkylaminosulfonyl, di(C₁₋₆ alkyl)aminosulfonyl, phenyloxy, phenyl, amino, C₁₋₆ alkylamino, di(C₁₋₆)alkylamino, di(C₁₋₆)alkylamino, C₁₋₆ alkoxycarbonyl, C₁₋₆ alkanoyl, C₁₋₆ alkanoylamino, carbamoyl, C₁₋₆ alkylcarbamoyl, di-(C₁₋₆alkyl)carbamoyl, C₁₋₆ alkylsulfonyl, C₁₋₆ alkyl optionally mono-, di-, or tri-substituted by halogen, C₁₋₆ alkoxy optionally mono-, di-, or tri-substituted by halogen and C₁₋₆ alkylthio optionally mono-, di-, or tri-substituted by halogen,
   or aryl fused by 1,3-dioxolane;
R² represents hydrogen or C₁₋₆ alkyl;
R³ represents halogen, C₁₋₆ alkoxy optionally mono-, di-, or tri-substituted by halogen,

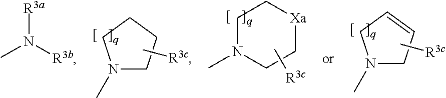

in which
R^{3a} and R^{3b} independently represent C₃₋₈ cycloalkyl, or C₁₋₆ alkyl, which C₁₋₆ alkyl is optionally substituted by hydroxy, carboxy, C₃₋₈ cycloalkyl, carbamoyl, C₁₋₆ alkylcarbamoyl, aryl-substituted C₁₋₆ alkylcarbamoyl, C₁₋₆ alkylcarbamoyl, di(C₁₋₆alkyl)carbamoyl, C₃₋₈ cycloalkylcarbamoyl, C₃₋₈ heterocyclocarbonyl, (C₁₋₆)alkylamino, di(C₁₋₆)alkylamino or C₁₋₆ alkoxy,
q represents an integer of 1 to 3;
R^{3c} represents hydrogen, hydroxy, carboxy, or C₁₋₆ alkyl optionally substituted by hydroxy, carboxy or (phenyl-substituted C₁₋₆ alkyl)carbamoyl;
Xa represents —O—, —S— or —N(R^{3d})—
   in which
R^{3d} represents C₁₋₆ alkyl;
R⁴ represents hydrogen, halogen, C₁₋₆ alkoxy, di(C₁₋₆ alkyl)amino or C₁₋₆ alkyl optionally substituted by C₁₋₆ alkoxy, or mono-, di-, or tri-halogen;
R⁵ represents hydrogen, or C₁₋₆ alkyl; and
R⁶ represents carboxy, carboxamide, nitrile or tetrazolyl.

In one embodiment, compounds of the formula (I) are those wherein:
R¹ represents

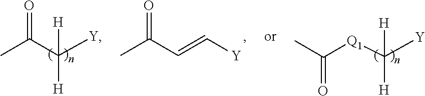

in which
n represents an integer of 0 to 2;
-Q₁- represents —NH—, —N(C₁₋₆alkyl)-, or —O—;
Y represents C₁₋₆ alkyl, C₃₋₈ cycloalkyl optionally substituted by C₁₋₆ alkyl, C₃₋₈ cycloalkyl fused by benzene selected from the group consisting of indenyl, and tetrahydronaphthyl, aryl selected from the group consisting of phenyl and naphthyl, or heteroaryl selected from the group consisting of indolyl, quinolyl, benzofuranyl, furanyl, chromanyl, and pyridyl, wherein said aryl and heteroaryl are optionally substituted at a substitutable position with one or more substituents selected from the group consisting of cyano, halogen, nitro, pyrrolyl, sulfamoyl, C₁₋₆ alkylaminosulfonyl, di(C₁₋₆ alkyl)aminosulfonyl, phenyloxy, phenyl, C₁₋₆alkylamino, di(C₁₋₆)alkylamino, C₁₋₆ alkoxycarbonyl, C₁₋₆ alkanoylamino, carbamoyl, C₁₋₆ alkylcarbamoyl, di-(C₁₋₆ alkyl)carbamoyl, C₁₋₆ alkylsulfonyl, C₁₋₆ alkyl optionally mono-, di-, or tri-substituted by halogen, C₁₋₆ alkoxy optionally mono-, di-, or tri-substituted by halogen and C₁₋₆ alkylthio optionally mono-, di-, or tri-halogen; and
R² represents hydrogen.

In another embodiment, compounds of the formula (I) are those wherein:
R³ represents C₁₋₆ alkoxy optionally mono-, di-, or tri-substituted by halogen,

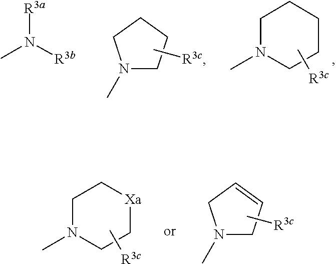

in which
R^{3a} and R^{3b} independently represent C₁₋₆ alkyl optionally substituted by hydroxy, carboxy, C₃₋₈ cycloalkyl, carbamoyl, C₁₋₆ alkylcarbamoyl, di(C₁₋₆ alkyl)carbamoyl, C₃₋₈ cycloalkylcarbamoyl, C₃₋₈ heterocyclocarbonyl, (C₁₋₆)alkylamino, di(C₁₋₆)alkylamino or C₁₋₆ alkoxy,
R^{3c} represents hydrogen, hydroxy, carboxy, or C₁₋₆ alkyl optionally substituted by hydroxy, carboxy or (phenyl-substituted C₁₋₆ alkyl)carbamoyl; and
Xa represents —O—, —S— or —N(R^{3d})—,
   in which
   R^{3d} represents C₁₋₆ alkyl.

In another embodiment, compounds of the formula (I-i) are those wherein:

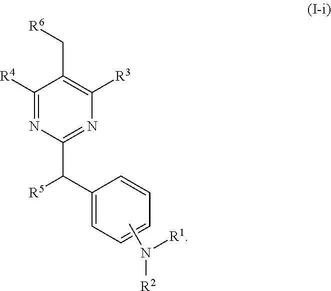

R¹ represents

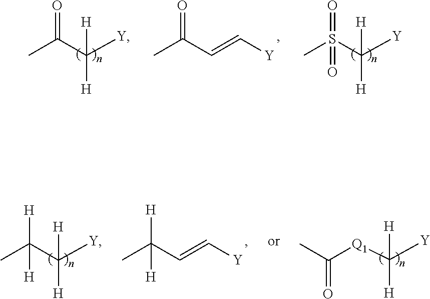

in which
n represents an integer of 0 to 2;
-Q₁- represents —NH—, —N(C₁₋₆ alkyl)-, or —O—;
Y represents phenyl, naphthyl, indolyl, quinolyl, benzofuranyl, furanyl or pyridyl, wherein said phenyl, naphthyl, indolyl, quinolyl, benzofuranyl, furanyl and pyridyl are optionally substituted at a substitutable position with one or two substituents selected from the group consisting of cyano, halogen, nitro, phenyloxy, phenyl, C₁₋₆ alkyl optionally mono-, di-, or tri-substituted by halogen, C₁₋₆ alkoxy optionally mono-, di-, or tri-substituted by halogen and C₁₋₆ alkylthio optionally mono-, di-, or tri-substituted by halogen;
R² represents hydrogen or C₁₋₆ alkyl;
R³ represents

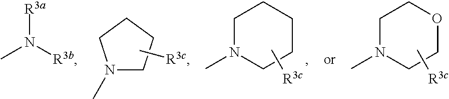

in which
R^{3a} and R^{3b} independently represent C₃₋₈ cycloalkyl, or C₁₋₆ alkyl optionally substituted by C₃₋₈ cycloalkyl, carbamoyl, C₁₋₆ alkylcarbamoyl, phenyl-substituted C₁₋₆ alkylcarbamoyl, C₁₋₆ alkylcarbamoyl, di(C₁₋₆alkyl)carbamoyl, C₃₋₈ cycloalkylcarbamoyl, C₃₋₈heterocyclocarbonyl, (C₁₋₆)alkylamino, di(C₁₋₆)alkylamino or C₁₋₆ alkoxy,
R^{3c} represents hydrogen, hydroxy, carboxy, or C₁₋₆ alkyl optionally substituted by hydroxy, carboxy or (phenyl-substituted C₁₋₆ alkyl)carbamoyl;
R⁴ represents hydrogen, chloro, bromo, C₁₋₆ alkoxy, di(C₁₋₆ alkyl)amino or C₁₋₆ alkyl optionally substituted by C₁₋₆ alkoxy;
R⁵ represents hydrogen, or methyl; and

R⁶ represents carboxy or tetrazolyl,

optionally in the form of their esters, prodrugs, enantiomers, racemates, diastereomers, tautomers, solvates, hydrates and their addition salts with pharmacologically acceptable acids or bases. Also included are the isotope equivalents of the compounds under formula I, wherein e.g. ¹²C is partially or fully exchanged by ¹³C or ¹H is partially or fully exchanged by ²H.

Preferred compounds are (1.1) to (1.187), which are disclosed in WO 2004/096777:

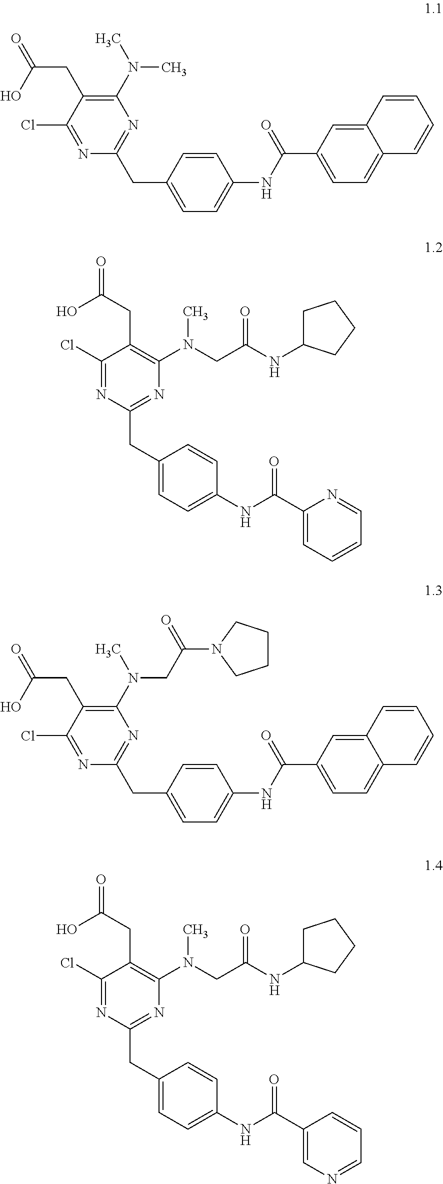

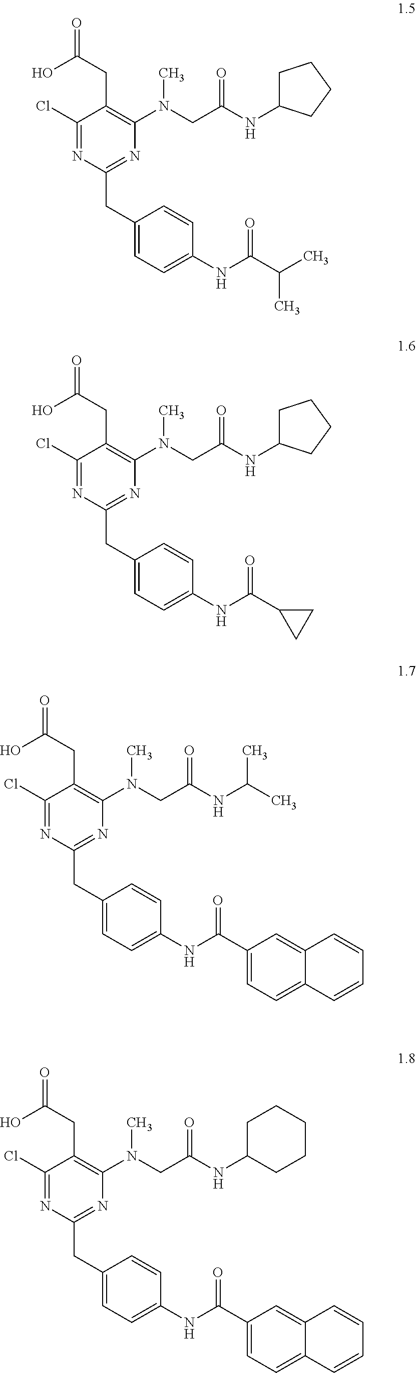

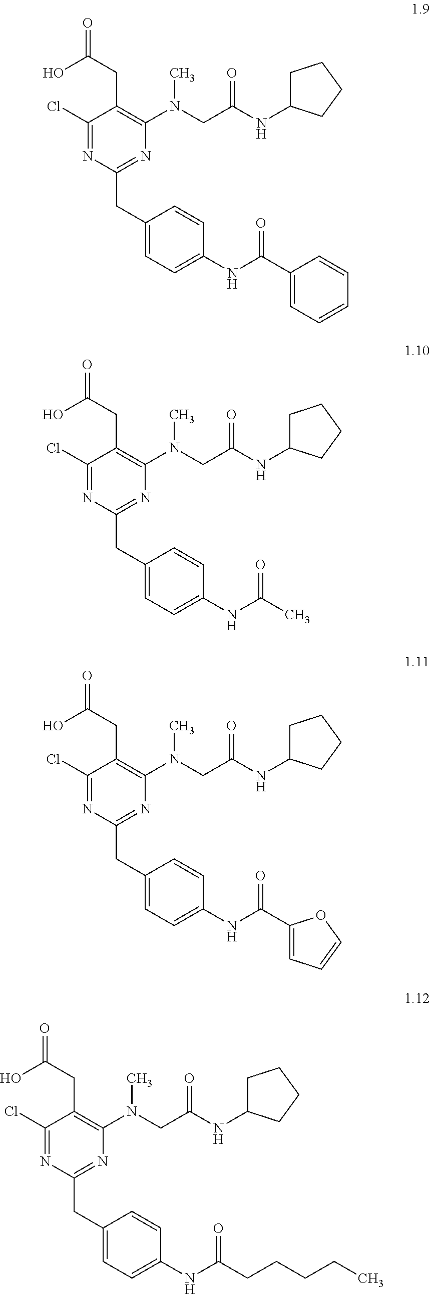

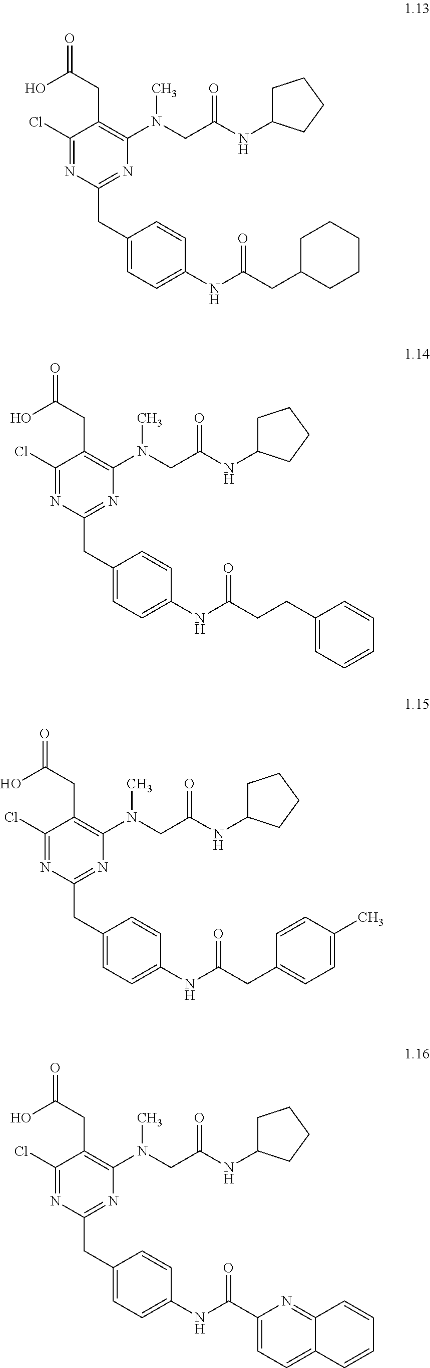

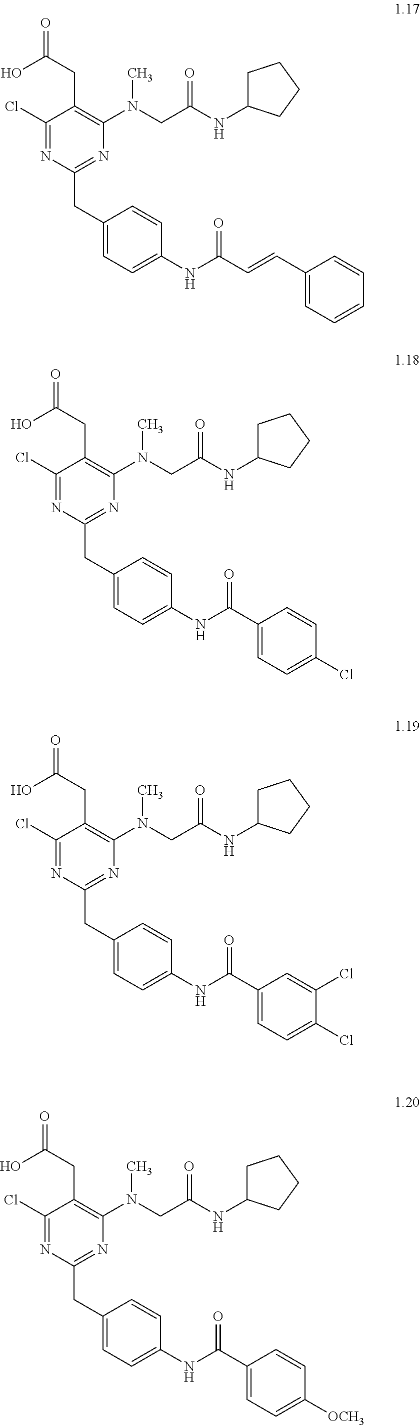

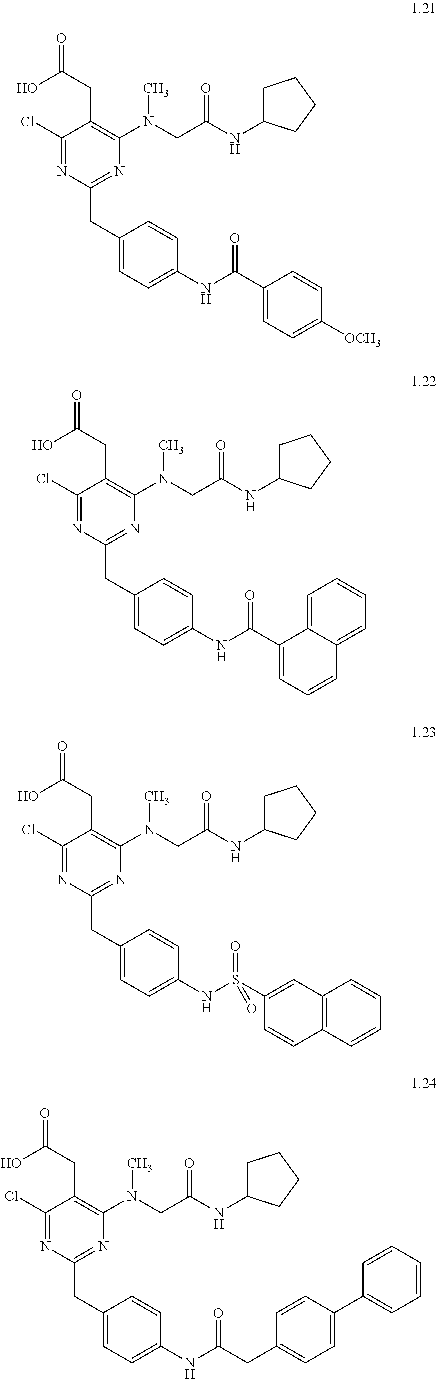

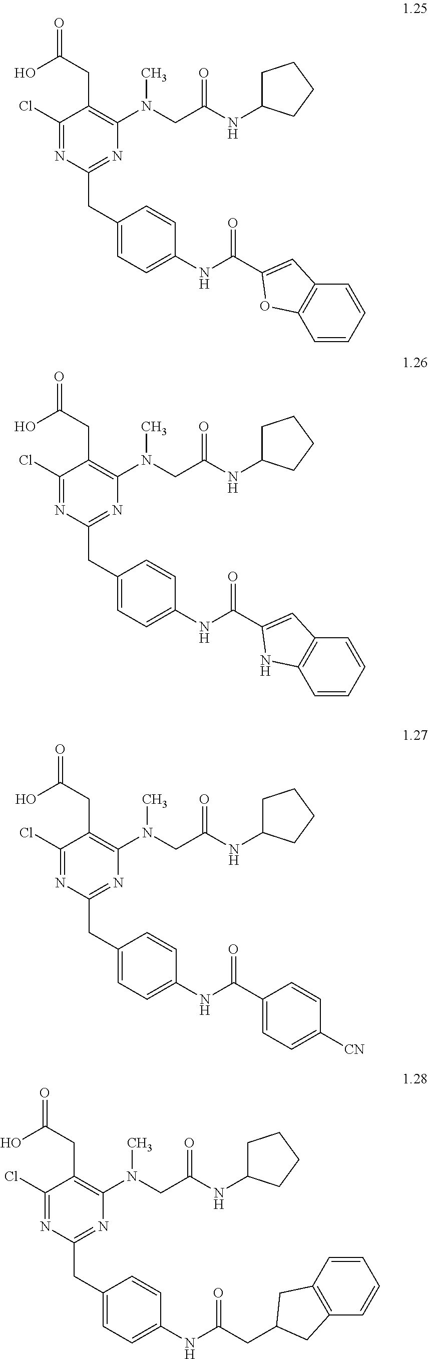

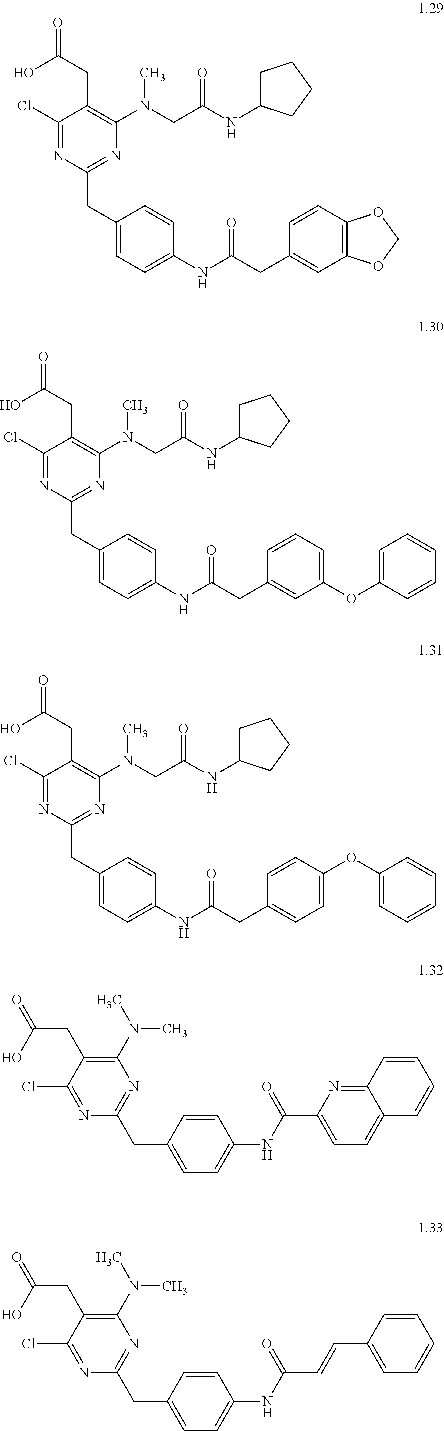

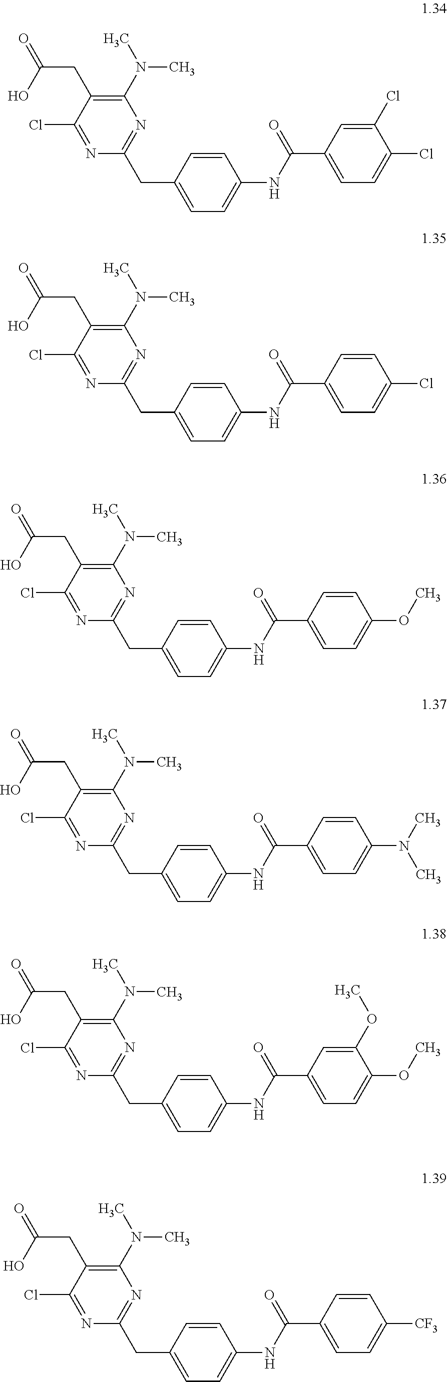

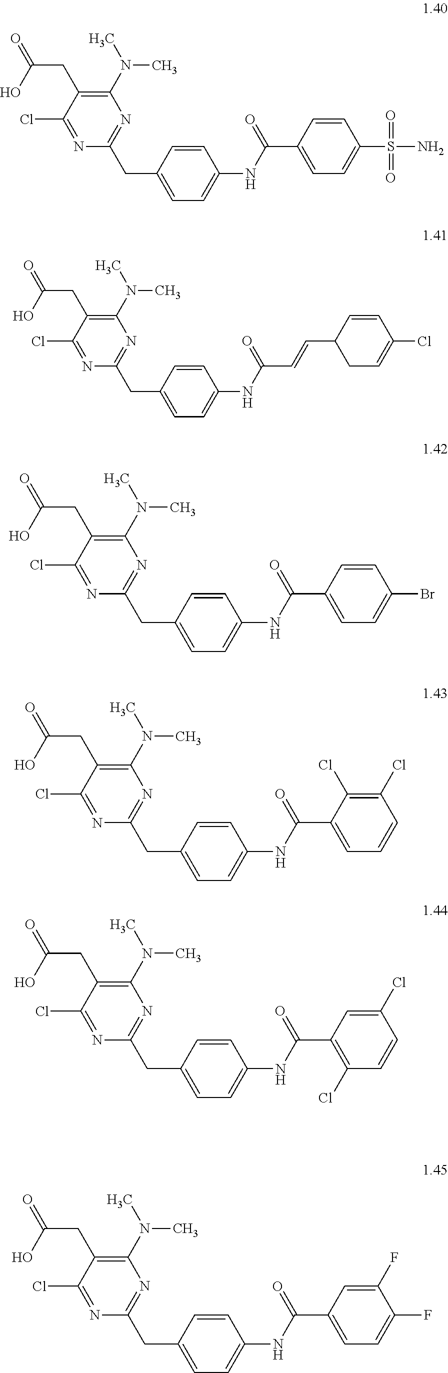

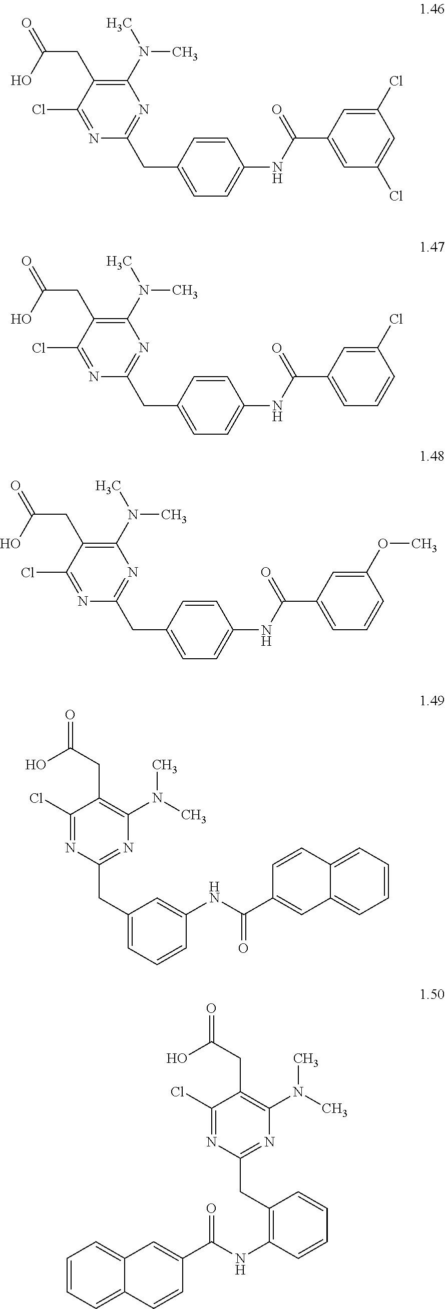

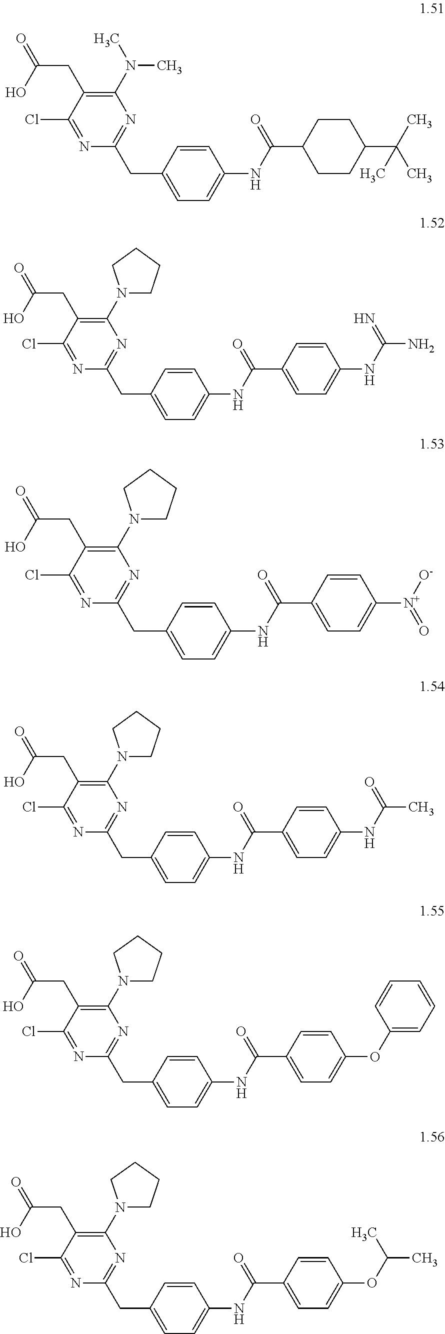

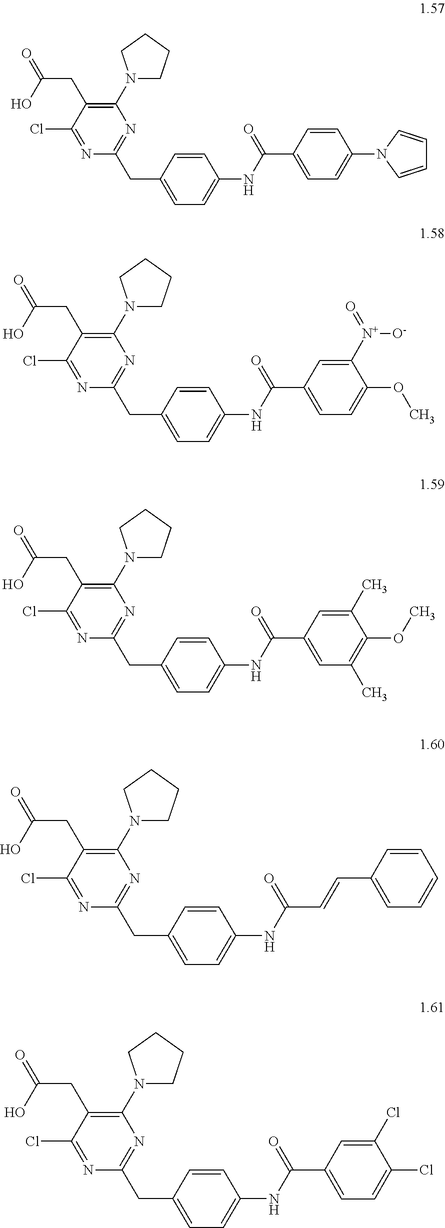

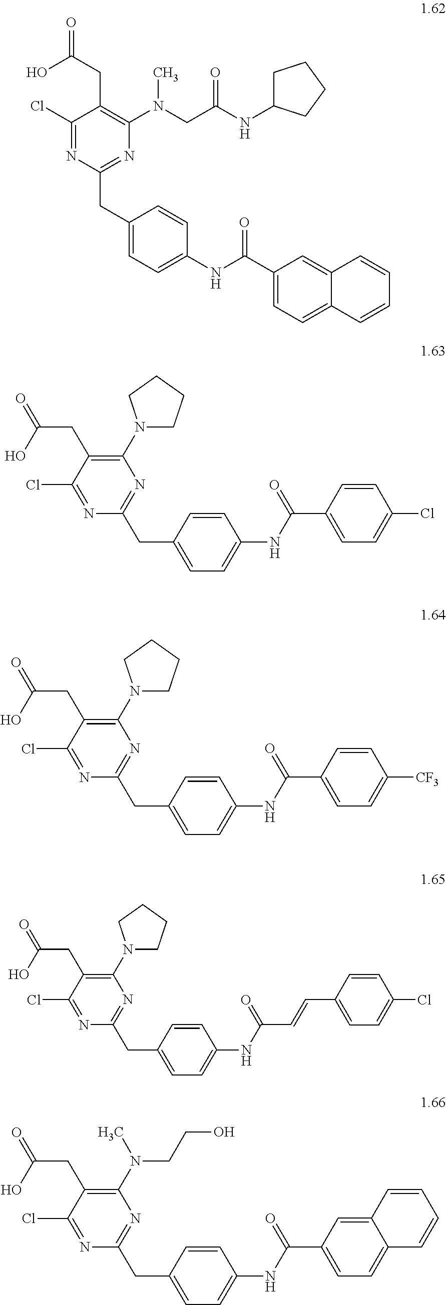

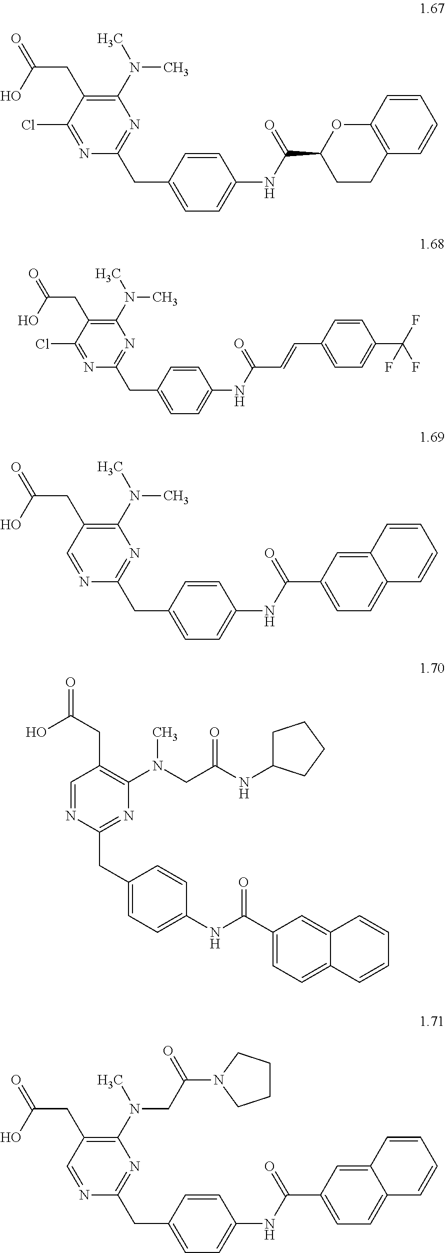

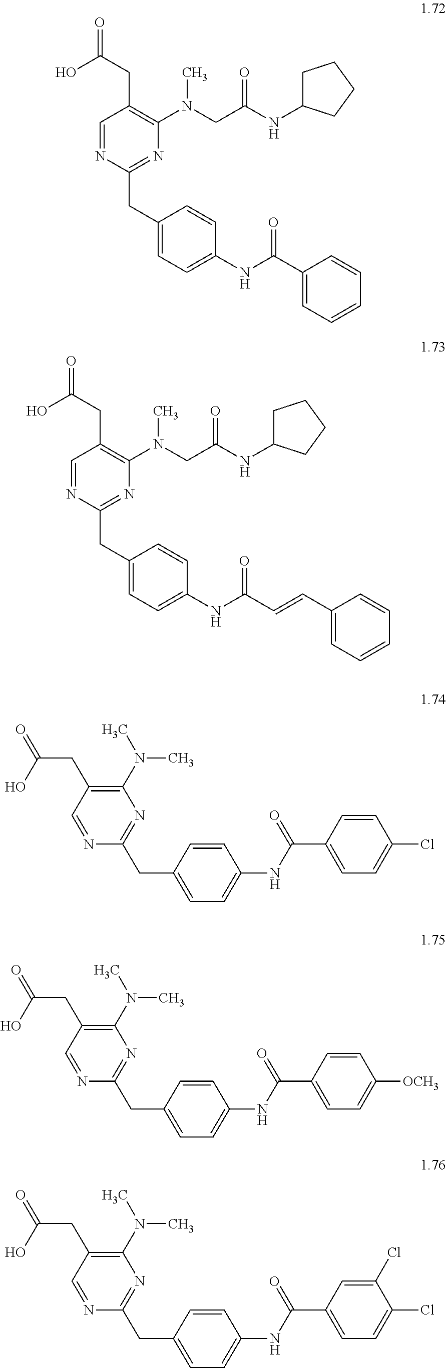

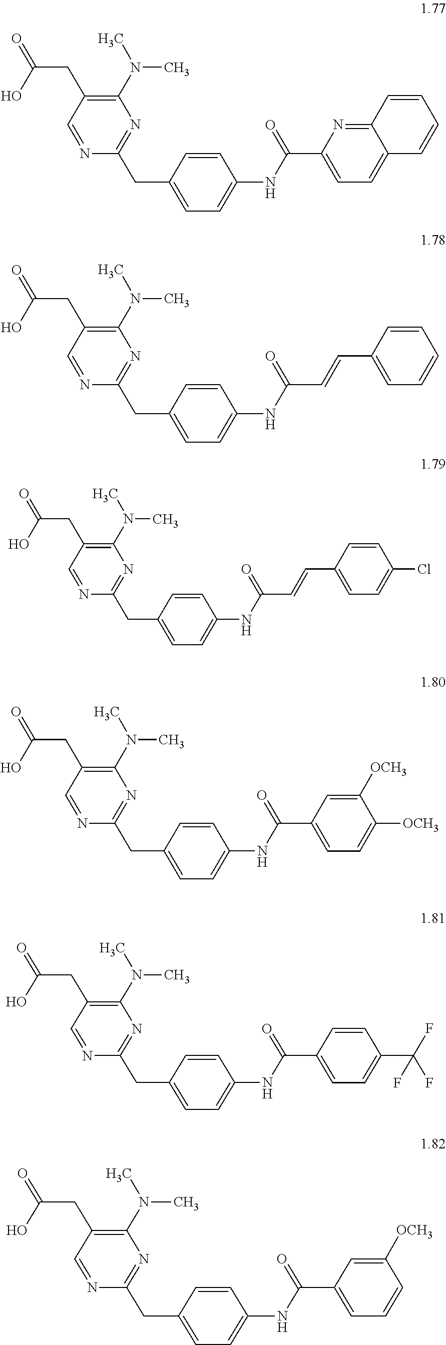

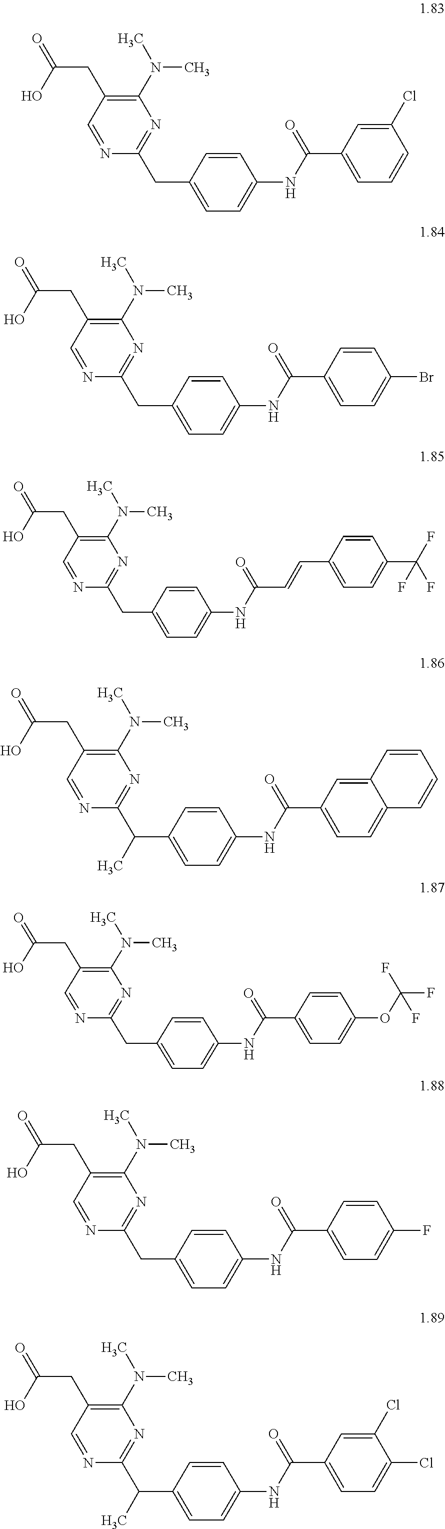

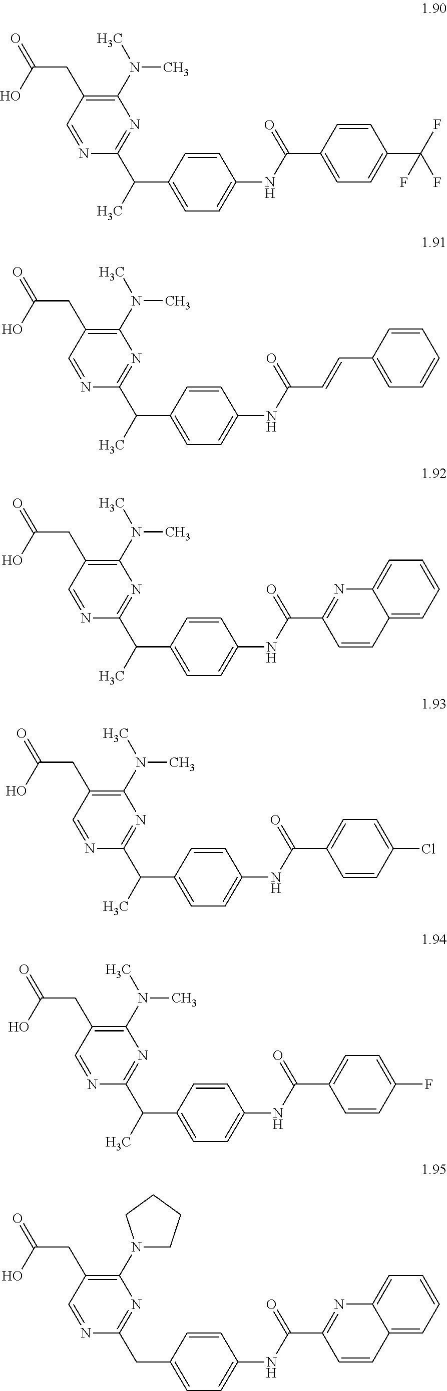

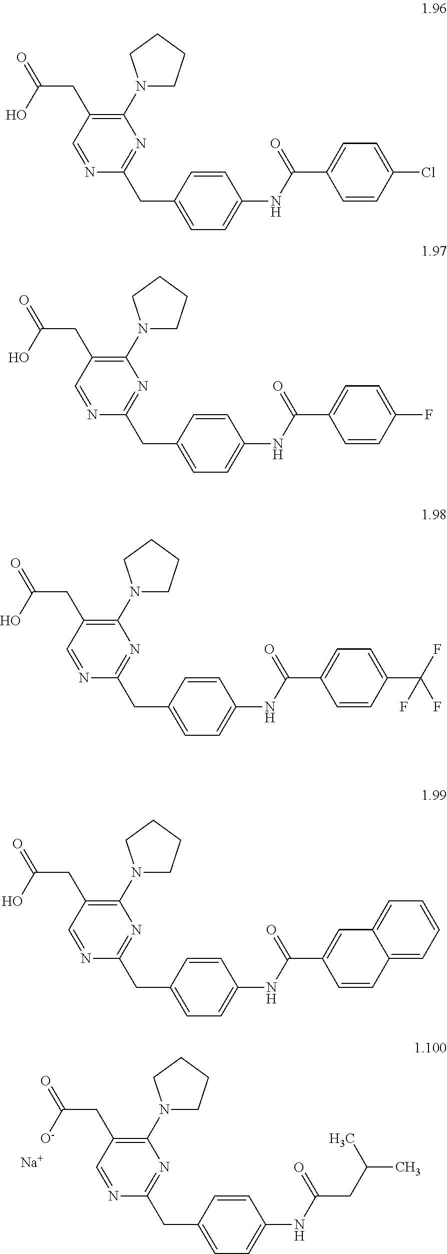

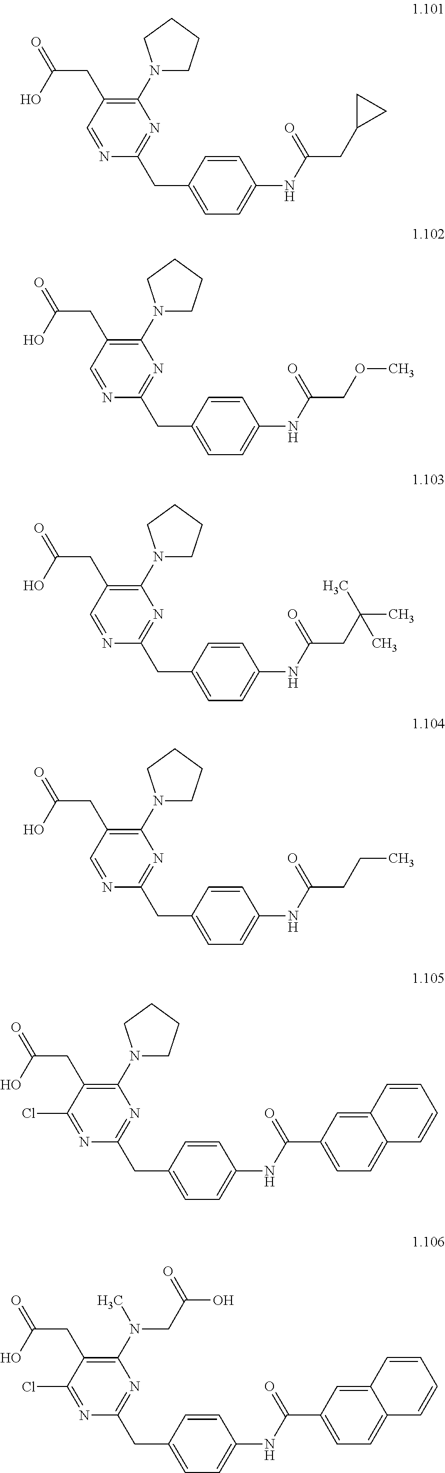

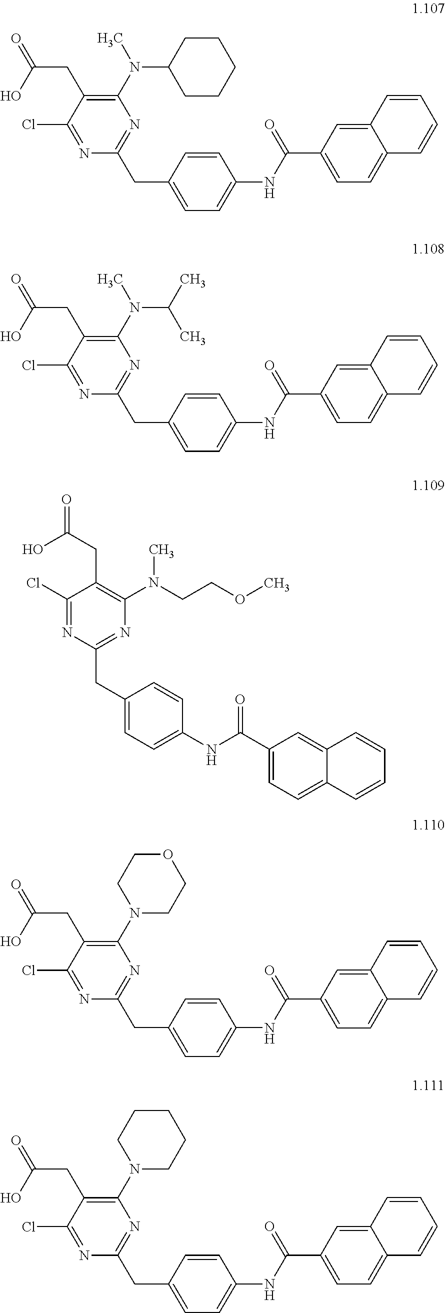

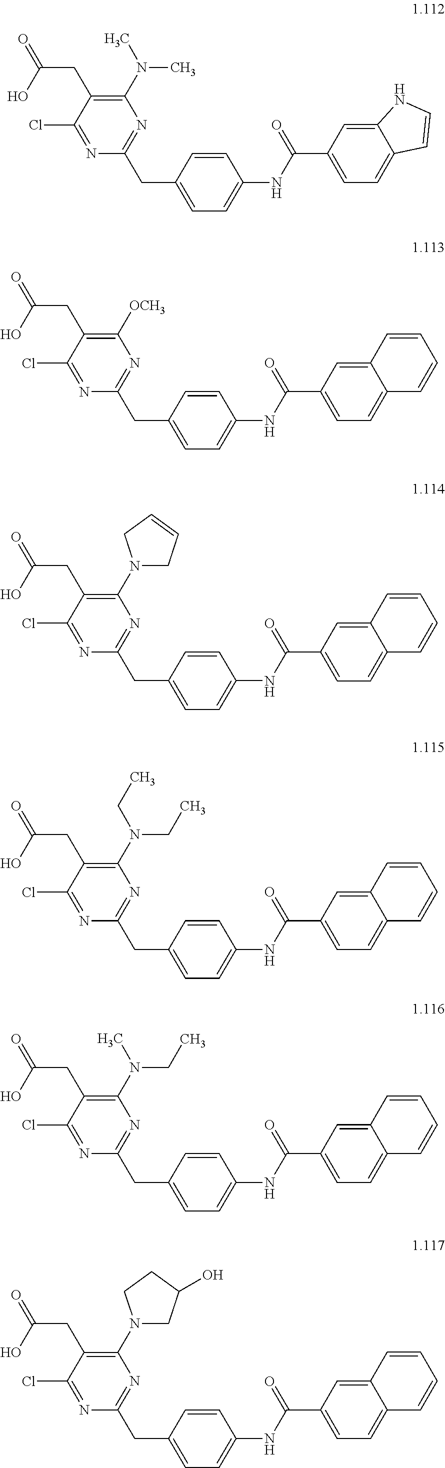

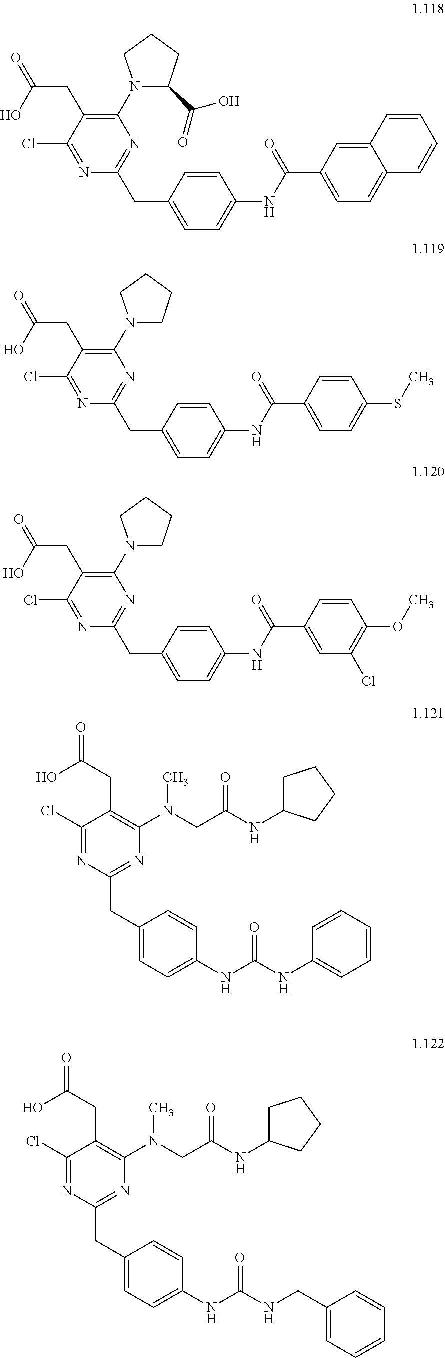

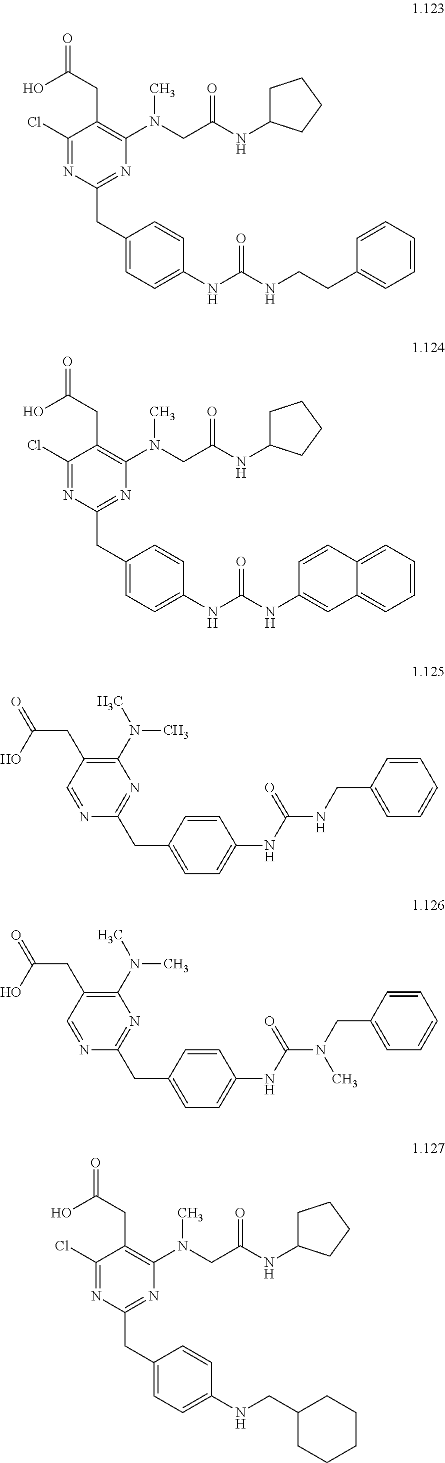

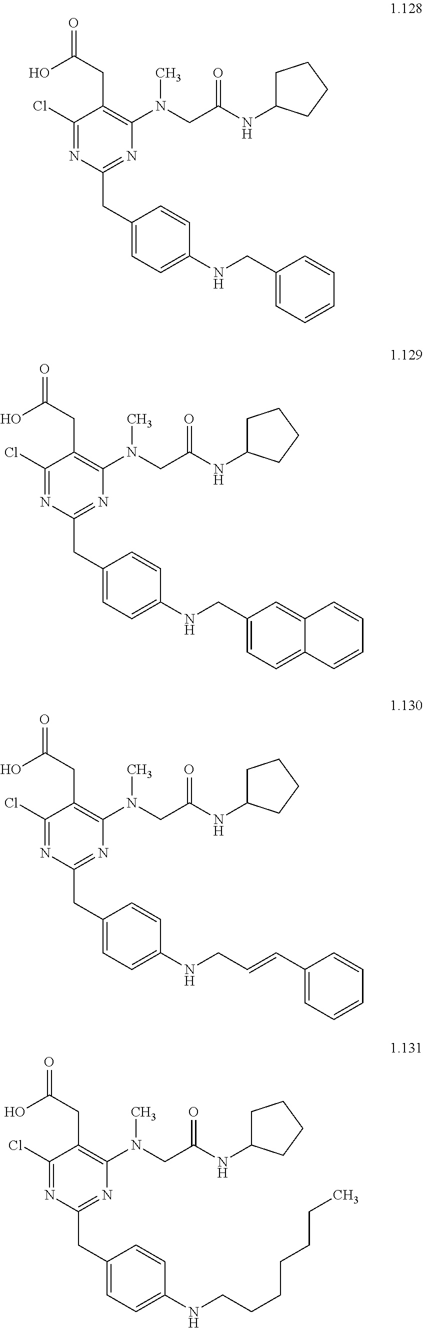

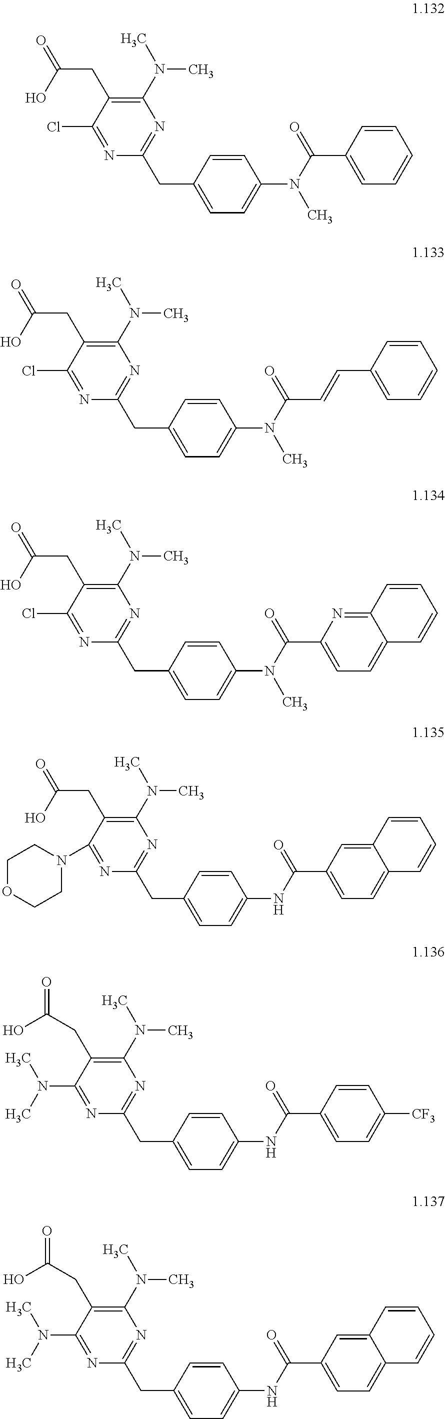

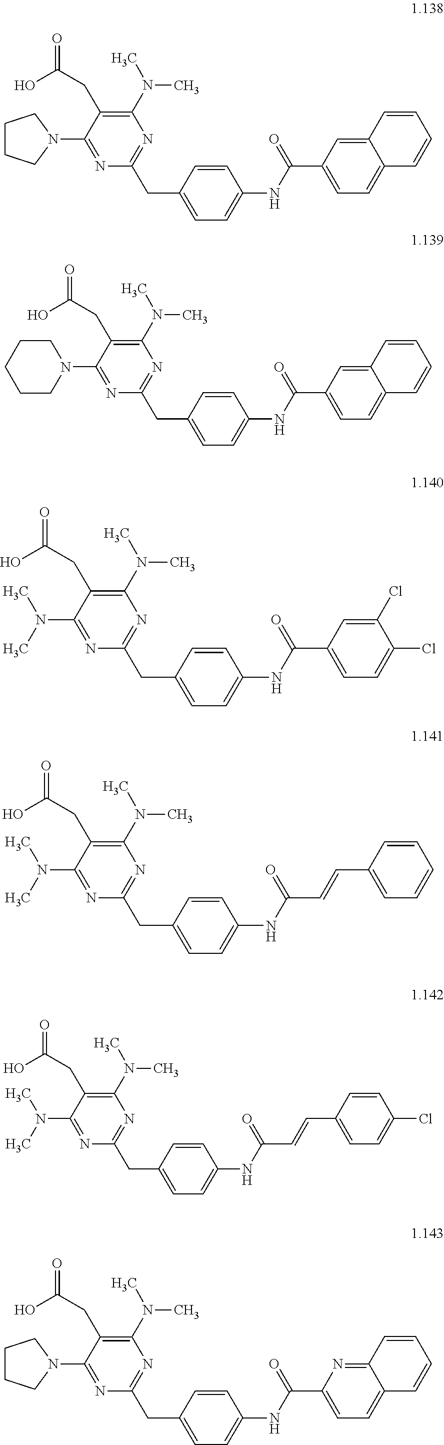

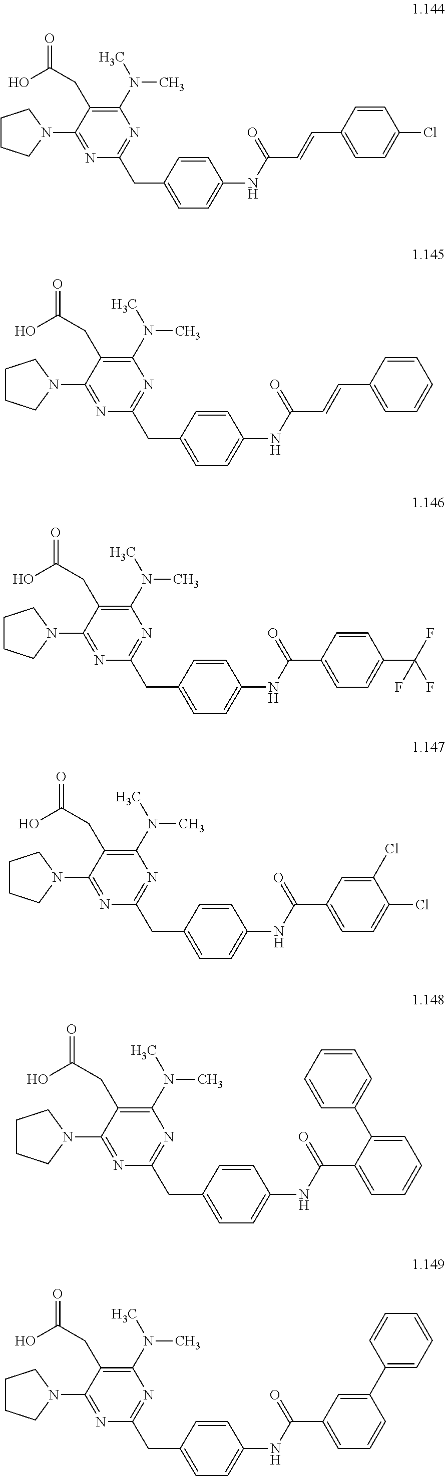

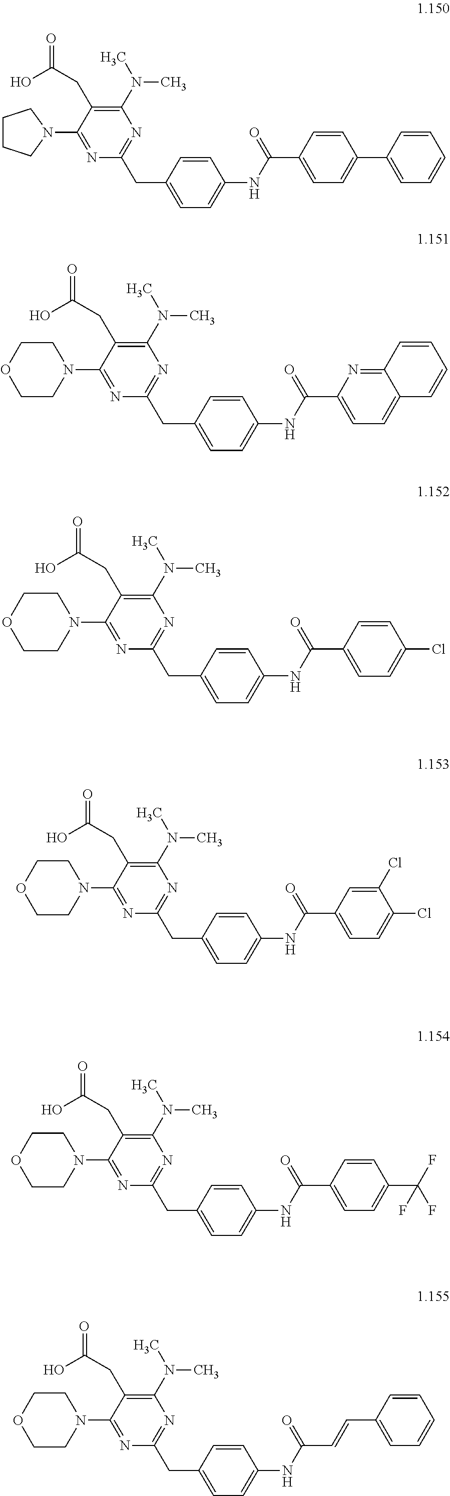

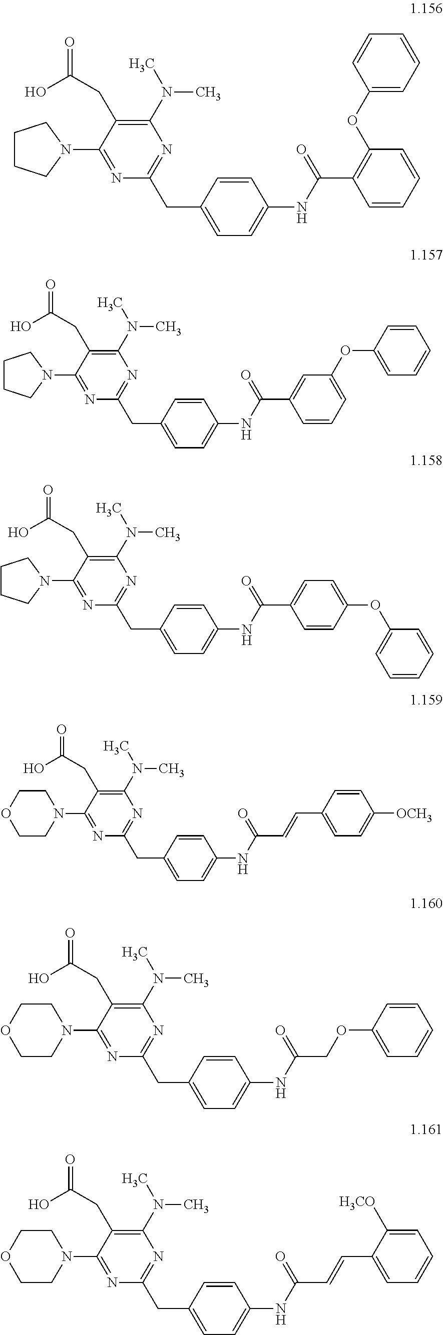

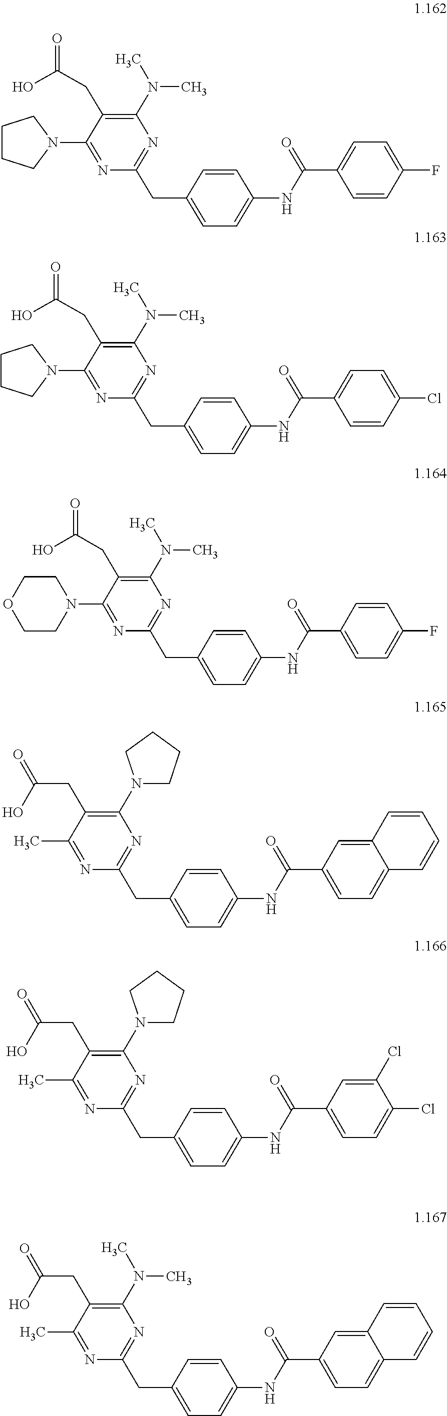

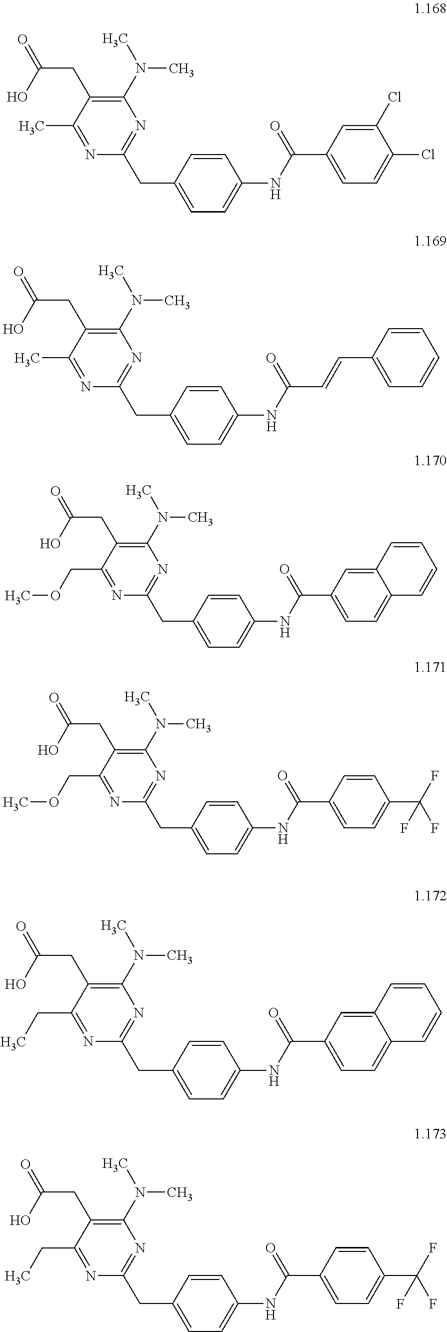

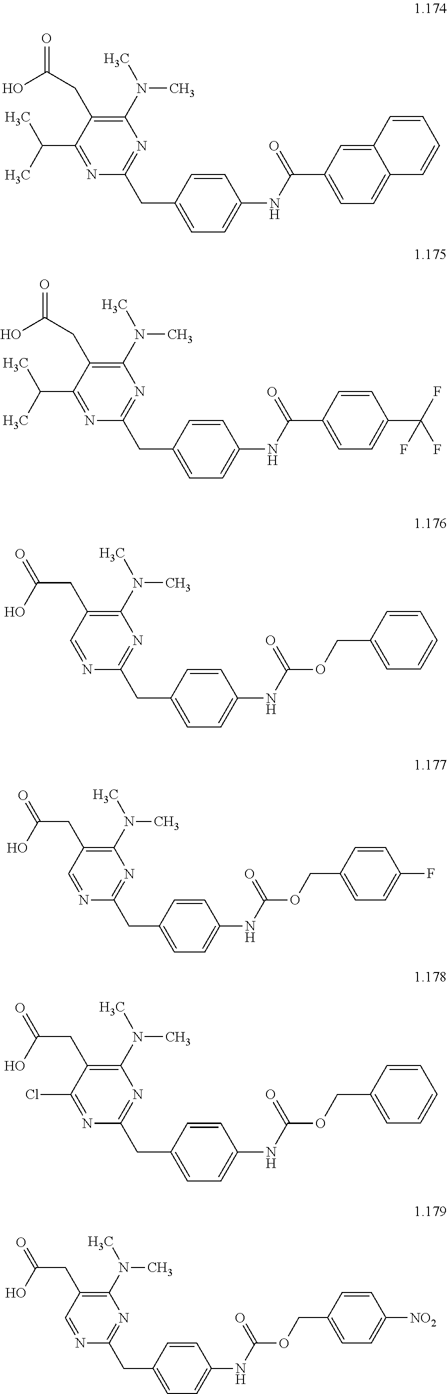

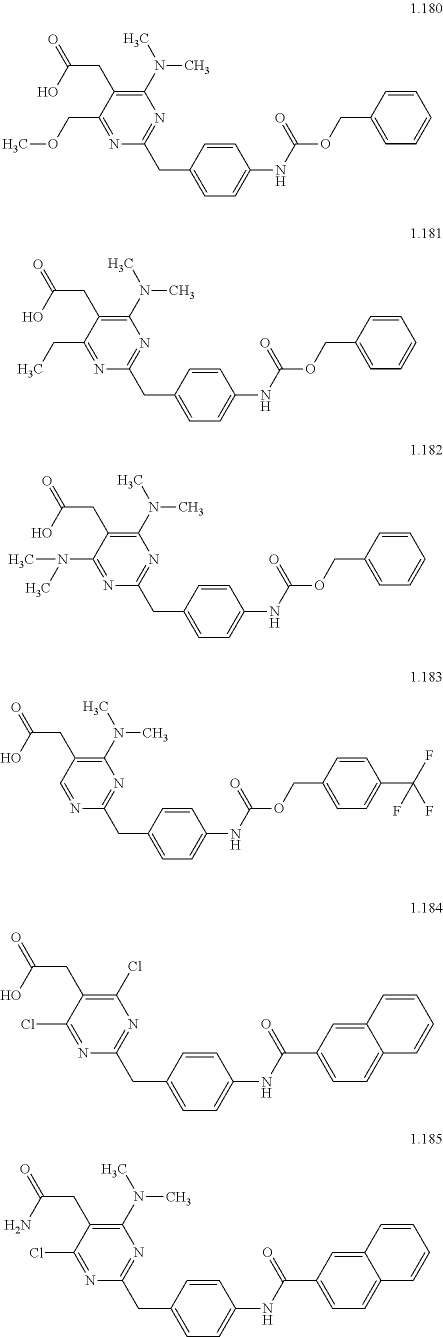

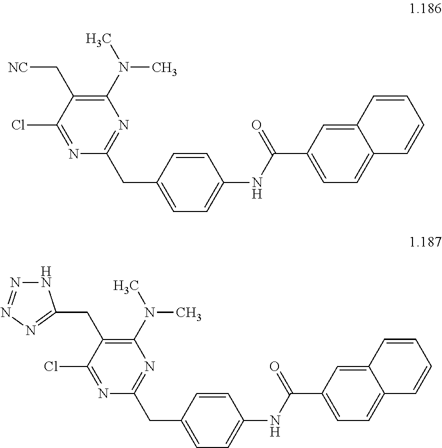

optionally in the form of their esters, prodrugs, enantiomers, racemates, diastereomers, tautomers, solvates, hydrates and their addition salts with pharmacologically acceptable acids or bases. Also included are the isotope equivalents of the compounds under formula I, wherein e.g. ¹²C is partially or fully exchanged by ¹³C and/or ¹H is partially or fully exchanged by ²H.

The Alkyl per se and “alk” and “alkyl” in alkoxy, alkanoyl, alkylcarbamoyl, alkylthio, alkylamino, alkylaminocarbonyl, alkylaminosulphonyl, alkylsulphonylamino, alkoxycarbonyl, alkoxycarbonyl-amino and alkanoylamino represent a linear or branched alkyl radical having generally 1 to 6, preferably 1 to 4 and particularly preferably 1 to 3 carbon atoms, representing illustratively and preferably methyl, ethyl, n-propyl, isopropyl, tert-butyl, n-pentyl and n-hexyl.

Alkoxy illustratively and preferably represents methoxy, ethoxy, n-propoxy, isopropoxy, tert-butoxy, n-pentoxy and n-hexoxy.

Alkanoyl illustratively and preferably represents acetyl and propanoyl.

Alkylamino represents an alkylamino radical having one or two (independently selected) alkyl substituents, illustratively and preferably representing methylamino, ethylamino, n-propylamino, isopropylamino, tert-butylamino, n-pentylamino, n-hexyl-amino, N,N-dimethylamino, N,N-diethylamino, N-ethyl-N-methylamino, N-methyl-N-n-propylamino, N-isopropyl-N-n-propyl-amino, N-t-butyl-N-methylamino, N-ethyl-N-n-pentylamino and N-n-hexyl-N-methylamino.

Alkylaminocarbonyl or alkylcarbamoyl represents an alkylaminocarbonyl radical having one or two (independently selected) alkyl substituents, illustratively and preferably representing methylaminocarbonyl, ethylaminocarbonyl, n-propylaminocarbonyl, isopropylamino-carbonyl, tert-butylaminocarbonyl, n-pentylaminocarbonyl, n-hexylaminocarbonyl, N,N-dimethyl-aminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-t-butyl-N-methylaminocarbonyl, N-ethyl-N-n-pentylamino-carbonyl and N-n-hexyl-N-methylaminocarbonyl.

Alkylaminosulphonyl represents an alkylaminosulphonyl radical having one or two (independently selected) alkyl substituents, illustratively and preferably representing methylaminosulphonyl, ethylaminosulphonyl, n-propylaminosulphonyl, isopropylaminosulphonyl, tert-butylamino-sulphonyl, n-pentylaminosulphonyl, n-hexyl-aminosulphonyl, N,N-dimethylaminosulphonyl, N,N-diethylaminosulphonyl, N-ethyl-N-methylaminosulphonyl, N-methyl-N-n-propylaminosulphonyl, N-isopropyl-N-n-propylaminosulphonyl, N-t-butyl-N-methylaminosulphonyl, N-ethyl-N-n-pentyl-aminosulphonyl and N-n-hexyl-N-methylaminosulphonyl.

Alkylsulphonylamino illustratively and preferably represents methylsulphonylamino, ethyl-sulphonylamino, n-propylsulphonylamino, isopropylsulphonylamino, tert-butyl-sulphonylamino, n-pentylsulphonylamino and n-hexylsulphonylamino.

Alkoxycarbonyl illustratively and preferably represents methoxycarbonyl, ethoxycarbonyl, n-propoxycarbonyl, isopropoxycarbonyl, tert-butoxycarbonyl, n-pentoxycarbonyl and n-hexoxycarbonyl. Alkoxycarbonylamino illustratively and preferably represents methoxy-carbonylamino, ethoxycarbonylamino, n-propoxycarbonylamino, isopropoxycarbonylamino, tert-butoxycarbonylamino, n-pentoxycarbonylamino and n-hexoxycarbonylamino.

Alkanoylamino illustratively and preferably represents acetylamino and ethylcarbonylamino. Cycloalkyl per se and in cycloalkylamino and in cycloalkylcarbonyl represents a cycloalkyl group having generally 3 to 8 and preferably 5 to 7 carbon atoms, illustratively and preferably representing cyclopropyl, cyclobutyl, cyclopentyl, cyclohexyl and cycloheptyl.

Cycloalkylamino represents a cycloalkylamino radical having one or two (independently selected) cycloalkyl substituents, illustratively and preferably representing cyclopropylamino, cyclo-butylamino, cyclopentylamino, cyclohexylamino and cycloheptylamino.

Cycloalkylcarbonyl illustratively and preferably represents cyclopropylcarbonyl, cyclobutyl-carbonyl, cyclopentylcarbonyl, cyclohexylcarbonyl and cycloheptylcarbonyl.

Aryl per se and in arylamino and in arylcarbonyl represents a mono- to tricyclic aromatic carbocyclic radical having generally 6 to 14 carbon atoms, illustratively and preferably representing phenyl, naphthyl and phenanthrenyl.

Arylamino represents an arylamino radical having one or two (independently selected) aryl substituents, illustratively and preferably representing phenylamino, diphenylamino and naphthylamino.

Arylcarbonyl illustratively and preferably represents phenylcarbonyl and naphthylcarbonyl.

Heteroaryl per se and in heteroarylamino and heteroarylcarbonyl represents an aromatic mono- or bicyclic radical having generally 5 to 10 and preferably 5 or 6 ring atoms and up to 5 and preferably up to 4 hetero atoms selected from the group consisting of S, O and N, illustratively and preferably representing thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl.

Heteroarylamino represents an heteroarylamino radical having one or two (independently selected) heteroaryl substituents, illustratively and preferably representing thienylamino, furylamino, pyrrolylamino, thiazolylamino, oxazolylamino, imidazolyl-amino, pyridylamino, pyrimidylamino, pyridazinylamino, indolylamino, indazolylamino, benzofuranylamino, benzothiophenylamino, quinolinylamino, isoquinolinylamino.

Heteroarylcarbonyl illustratively and preferably represents thienylcarbonyl, furylcarbonyl, pyrrolylcarbonyl, thiazolylcarbonyl, oxazolylcarbonyl, imidazolylcarbonyl, pyridylcarbonyl, pyrimidylcarbonyl, pyridazinylcarbonyl, indolylcarbonyl, indazolylcarbonyl, benzofuranyl-carbonyl, benzothiophenylcarbonyl, quinolinylcarbonyl, isoquinolinylcarbonyl.

Heterocyclyl per se and in heterocyclylcarbonyl represents a mono- or polycyclic, preferably mono- or bicyclic, nonaromatic heterocyclic radical having generally 4 to 10 and preferably 5 to 8 ring atoms and up to 3 and preferably up to 2 hetero atoms and/or hetero groups selected from the group consisting of N, O, S, SO and SO₂. The heterocyclyl radicals can be saturated or partially unsaturated. Preference is given to 5- to 8-membered monocyclic saturated heterocyclyl radicals having up to two hetero atoms selected from the group consisting of O, N and S, such as illustratively and preferably tetrahydrofuran-2-yl, pyrrolidin-2-yl, pyrrolidin-3-yl, pyrrolinyl, piperidinyl, morpholinyl, perhydroazepinyl.

Heterocyclylcarbonyl illustratively and preferably represents tetrahydrofuran-2-carbonyl, pyrrolidine-2-carbonyl, pyrrolidine-3-carbonyl, pyrrolinecarbonyl, piperidinecarbonyl, morpholinecarbonyl, perhydroazepinecarbonyl.

### The Further Active Compound 2

The pharmaceutical composition of the present invention further comprises at least one further active compound 2 selected from the classes consisting of β2-adrenoceptor-agonists (short and long-acting beta mimetics), anti-cholinergics (short and long-acting), anti-inflammatory steroids (oral and topical corticosteroids), dissociated-glucocorticoidmimetics, PDE3 inhibitors, PDE4 inhibitors, PDE7 inhibitors, LTD4 antagonists, EGFR inhibitors, PAF antagonists, Lipoxin A4 derivatives, FPRL1 modulators, LTB4-receptor (BLT1, BLT2) antagonists, Histamine receptor antagonists, PI3-kinase inhibitors, inhibitors of non-receptor tyrosine kinases as for example LYN, LCK, SYK, ZAP-70, FYN, BTK or ITK, inhibitors of MAP kinases as for example p38, ERK1, ERK2, JNK1, JNK2, JNK3 or SAP, inhibitors of the NF-κB signalling pathway as for example IKK2 kinase inhibitors, iNOS inhibitors, MRP4 inhibitors, leukotriene biosynthese inhibitors as for example 5-Lipoxygenase (5-LO) inhibitors, cPLA2 inhibitors, Leukotriene A4 hydrolase inhibitors or FLAP inhibitors, non-steroidale anti-inflammatory agents (NSAIDs), DP1-receptor modulators, thromboxane receptor antagonists, CCR1 antagonists, CCR2 antagonists, CCR3 antagonists, CCR4 antagonists, CCR5 antagonists, CCR6 antagonists, CCR7 antagonists, CCR8 antagonists, CCR9 antagonists, CCR10 antagonists, CXCR1 antagonists, CXCR2 antagonists, CXCR3 antagonists, CXCR4 antagonists, CXCR5 antagonists, CXCR6 antagonists, CX3CR1 antagonists, Neurokinin (NK1, NK2) antagonists, sphingosine 1-phosphate receptor modulators, sphingosine 1 phosphate lyase inhibitors, adenosine receptor modulators as for example A2a-agonists, modulators of purinergic receptors as for example P2X7 inhibitors, histone deacetylase (HDAC) activators, bradykinin (BK1, BK2) antagonists, TACE inhibitors, PPAR gamma modulators, Rho-kinase inhibitors, interleukin 1-beta converting enzyme (ICE) inhibitors, Toll-like receptor (TLR) modulators, HMG-CoA reductase inhibitors, VLA-4 antagonists, ICAM-1 inhibitors, SHIP agonists, GABAa receptor antagonist, ENaC-inhibitors, Melanocortin receptor (MC1R, MC2R, MC3R, MC4R, MC5R) modulators, CGRP antagonists, Endothelin antagonists, mucoregulators, immunotherapeutic agents, compounds against swelling of the airways, compounds against cough, CB2 agonists, retinoids, immunosuppressants, mast cell stabilizers, methylxanthine, opioid receptor agonists, laxatives, anti-foaming agents, antispasmodic agents, 5-HT4 agonists but also combinations of two or three active substances.

Preferred examples of the further active compounds 2 herein are the following:

### 2-1 Beta Mimetics

Preferred betamimetics are albuterol, bambuterol, bitolterol, broxaterol, carbuterol, clenbuterol, fenoterol, formoterol, indacaterol, carmoterol, arformoterol, zinterol, hexoprenaline, ibuterol, isoetharine, isoprenaline, levosalbutamol, mabuterol, meluadrine, metaproterenol, orciprenaline, pirbuterol, procaterol, reproterol, rimiterol, ritodrine, salmeterol, salmefamol, soterenol, sulphonterol, tiaramide, terbutaline, tolubuterol, 6-hydroxy-8-{1-hydroxy-2-[2-(4-methoxy-phenyl)-1,1-dimethyl-ethylamino]-ethyl}-4H-benzo[1,4]oxazin-3-one, 8-{2-[2-(2,4-difluoro-phenyl)-1,1-dimethyl-ethylamino]-1-hydroxy-ethyl}-6-hydroxy-4H-benzo[1,4]oxazin-3-one, 8-{2-[2-(3,5-difluoro-phenyl)-1,1-dimethyl-ethylamino]-1-hydroxy-ethyl}-6-hydroxy-4H-benzo[1,4]oxazin-3-one, 8-{2-[2-(4-ethoxy-phenyl)-1,1-dimethyl-ethylamino]-1-hydroxy-ethyl}-6-hydroxy-4H-benzo[1,4]oxazin-3-one, 8-{2-[2-(4-Fluor-phenyl)-1,1-dimethyl-ethylamino]-1-hydroxy-ethyl}-6-hydroxy-4H-benzo[1,4]oxazin-3-one, N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methansulfonamid, N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methansulfonamid, N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methansulfonamid, N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methansulfonamid, 8-{2-[1,1-dimethyl-3-(2-oxo-2,3-dihydro-benzoimidazol-1-yl)-propylamino]-1-hydroxy-ethyl}-6-hydroxy-4H-benzo[1,4]oxazin-3-one, 8-{2-[1,1-dimethyl-3-(6-methyl-2-oxo-2,3-dihydro-benzoimidazol-1-yl)-propylamino]-1-hydroxy-ethyl}-6-hydroxy-4H-benzo[1,4]oxazin-3-one, 8-{2-[1,1-dimethyl-3-(2-oxo-5-trifluormethyl-2,3-dihydro-benzoimidazol-1-yl)-propylamino]-1-hydroxy-ethyl}-6-hydroxy-4H-benzo[1,4]oxazin-3-one, 8-{2-[1,1-dimethyl-3-(3-methyl-2-oxo-2,3-dihydro-benzoimidazol-1-yl)-propylamino]-1-hydroxy-ethyl}-6-hydroxy-4H-benzo[1,4]oxazin-3-one, CHF-1035, HOKU-81, KUL-1248, 3-(4-{6-[2-Hydroxy-2-(4-hydroxy-3-hydroxymethyl-phenyl)-ethylamino]-hexyloxy}-butyl)-benzyl-sulfonamid, 5-[2-(5,6-Diethyl-indan-2-ylamino)-1-hydroxy-ethyl]-8-hydroxy-1H-quinolin-2-one, 4-hydroxy-7-[2-{[2-{[3-(2-phenylethoxy)propyl]sulphonyl}ethyl]-amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-one, 1-(4-amino-3-chloro-5-trifluoromethylphenyl)-2-tert.-butylamino)ethanol, 6-hydroxy-8-{1-hydroxy-2-[2-(4-methoxy-phenyl)-1,1-dimethyl-ethylamino]-ethyl}-4H-benzo[1,4]oxazin-3-one, 6-hydroxy-8-{1-hydroxy-2-[2-(4-phenoxy-acetic acid ethylester)-1,1-dimethyl-ethylamino]-ethyl}-4H-benzo[1,4]oxazin-3-one, 6-hydroxy-8-{1-hydroxy-2-[2-(4-phenoxy-acetic acid)-1,1-dimethyl-ethylamino]-ethyl}-4H-benzo[1,4]oxazin-3-one, 8-{2-[1,1-dimethyl-2-(2,4,6-trimethylphenyl)-ethylamino]-1-hydroxy-ethyl}-6-hydroxy-4H-benzo[1,4]oxazin-3-one, 6-hydroxy-8-{1-hydroxy-2-[2-(4-hydroxy-phenyl)-1,1-dimethyl-ethylamino]-ethyl}-4H-benzo[1,4]oxazin-3-one, 6-hydroxy-8-{1-hydroxy-2-[2-(4-isopropyl-phenyl)-1,1-dimethyl-ethylamino]-ethyl}-4H-benzo[1,4]oxazin-3-one, 8-{2-[2-(4-ethyl-phenyl)-1,1-dimethyl-ethylamino]-1-hydroxy-ethyl}-6-hydroxy-4H-benzo[1,4]oxazin-3-one, 8-{2-[2-(4-ethoxy-phenyl)-1,1-dimethyl-ethylamino]-1-hydroxy-ethyl}-6-hydroxy-4H-benzo[1,4]oxazin-3-one, 4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butanoic acid, 8-{2-[2-(3,4-difluoro-phenyl)-1,1-dimethyl-ethylamino]-1-hydroxy-ethyl}-6-hydroxy-4H-benzo[1,4]oxazin-3-on and 1-(4-Ethoxy-carbonylamino-3-cyano-5-fluorophenyl)-2-(tert.-butylamino)ethanol, optionally in racemic form, as enantiomers, diastereomeres or as pharmacologically acceptable salts, solvates or hydrates.

Preferred are bambuterol, bitolterol, carbuterol, clenbuterol, fenoterol, formoterol, hexoprenaline, ibuterol, pirbuterol, procaterol, reproterol, salmeterol, sulphonterol, terbutaline, tolubuterol, 6-hydroxy-8-{1-hydroxy-2-[2-(4-methoxy-phenyl)-1,1-dimethyl-ethylamino]-ethyl}-4H-benzo[1,4]oxazin-3-one, 8-{2-[2-(2,4-difluoro-phenyl)-1,1-dimethyl-ethylamino]-1-hydroxy-ethyl}-6-hydroxy-4H-benzo[1,4]oxazin-3-one, 8-{2-[2-(3,5-difluoro-phenyl)-1,1-dimethyl-ethylamino]-1-hydroxy-ethyl}-6-hydroxy-4H-benzo[1,4]oxazin-3-one, 8-{2-[2-(4-Ethoxy-phenyl)-1,1-dimethyl-ethylamino]-1-hydroxy-ethyl}-6-hydroxy-4H-benzo[1,4]oxazin-3-one, 8-{2-[2-(4-Fluor-phenyl)-1,1-dimethyl-ethylamino]-1-hydroxy-ethyl}-6-hydroxy-4H-benzo[1,4]oxazin-3-one, N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methansulfonamid, N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methansulfonamid, N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methansulfonamid, N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methansulfonamid, 8-{2-[1,1-dimethyl-3-(2-oxo-2,3-dihydro-benzoimidazol-1-yl)-propylamino]-1-hydroxy-ethyl}-6-hydroxy-4H-benzo[1,4]oxazin-3-one, 8-{2-[1,1-dimethyl-3-(6-methyl-2-oxo-2,3-dihydro-benzoimidazol-1-yl)-propylamino]-1-hydroxy-ethyl}-6-hydroxy-4H-benzo[1,4]oxazin-3-one, 8-{2-[1,1-dimethyl-3-(2-oxo-5-trifluormethyl-2,3-dihydro-benzoimidazol-1-yl)-propylamino]-1-hydroxy-ethyl}-6-hydroxy-4H-benzo[1,4]oxazin-3-one, 8-{2-[1,1-dimethyl-3-(3-methyl-2-oxo-2,3-dihydro-benzoimidazol-1-yl)-propylamino]-1-hydroxy-ethyl}-6-hydroxy-4H-benzo[1,4]oxazin-3-one, 3-(4-{6-[2-hydroxy-2-(4-hydroxy-3-hydroxymethyl-phenyl)-ethylamino]-hexyloxy}-butyl)-benzenesulfoneamide, 5-[2-(5,6-diethyl-indan-2-ylamino)-1-hydroxy-ethyl]-8-hydroxy-1H-quinolin-2-on, 4-hydroxy-7-[2-{[2-{[3-(2-phenylethoxy)propyl]sulphonyl}ethyl]-amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-one, 1-(4-amino-3-chloro-5-trifluoromethylphenyl)-2-tert.-butylamino)ethanol, 6-Hydroxy-8-{1-hydroxy-2-[2-(4-methoxy-phenyl)-1,1-dimethyl-ethylamino]-ethyl}-4H-benzo[1,4]oxazin-3-one, 6-hydroxy-8-{1-hydroxy-2-[2-(4-phenoxy-acetic acid ethylester)-1,1-dimethyl-ethylamino]-ethyl}-4H-benzo[1,4]oxazin-3-one, 6-hydroxy-8-{1-hydroxy-2-[2-(4-phenoxy-acetic acid)-1,1-dimethyl-ethylamino]-ethyl}-4H-benzo[1,4]oxazin-3-one, 8-{2-[1,1-dimethyl-2-(2,4,6-trimethylphenyl)-ethylamino]-1-hydroxy-ethyl}-6-hydroxy-4H-benzo[1,4]oxazin-3-one, 6-hydroxy-8-{1-hydroxy-2-[2-(4-hydroxy-phenyl)-1,1-dimethyl-ethylamino]-ethyl}-4H-benzo[1,4]oxazin-3-one, 6-hydroxy-8-{1-hydroxy-2-[2-(4-isopropyl-phenyl)-1,1-dimethyl-ethylamino]-ethyl}-4H-benzo[1,4]oxazin-3-one, 8-{2-[2-(4-ethyl-phenyl)-1,1-dimethyl-ethylamino]-1-hydroxy-ethyl}-6-hydroxy-4H-benzo[1,4]oxazin-3-one, 8-{2-[2-(4-ethoxy-phenyl)-1,1-dimethyl-ethylamino]-1-hydroxy-ethyl}-6-hydroxy-4H-benzo[1,4]oxazin-3-one, 4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butanoic acid, 8-{2-[2-(3,4-difluoro-phenyl)-1,1-dimethyl-ethylamino]-1-hydroxy-ethyl}-6-hydroxy-4H-benzo[1,4]oxazin-3-on and 1-(4-ethoxycarbonylamino-3-cyano-5-fluorophenyl)-2-(tert.-butylamino)ethanol, optionally in racemic form, as enantiomers, diastereomeres or as pharmacologically acceptable salts, solvates or hydrates.

Preferred are fenoterol, formoterol, salmeterol, 6-hydroxy-8-{1-hydroxy-2-[2-(4-methoxy-phenyl)-1,1-dimethyl-ethylamino]-ethyl}-4H-benzo[1,4]oxazin-3-one, 8-{2-[2-(2,4-difluoro-phenyl)-1,1-dimethyl-ethylamino]-1-hydroxy-ethyl}-6-hydroxy-4H-benzo[1,4]oxazin-3-one, 8-{2-[2-(3,5-difluoro-phenyl)-1,1-dimethyl-ethylamino]-1-hydroxy-ethyl}-6-hydroxy-4H-benzo[1,4]oxazin-3-one, 8-{2-[2-(4-ethoxy-phenyl)-1,1-dimethyl-ethylamino]-1-hydroxy-ethyl}-6-hydroxy-4H-benzo[1,4]oxazin-3-one, 8-{2-[2-(4-fluoro-phenyl)-1,1-dimethyl-ethylamino]-1-hydroxy-ethyl}-6-hydroxy-4H-benzo[1,4]oxazin-3-one, N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methansulfonamid, N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methansulfonamid, N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methansulfonamid, N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methansulfonamid, 8-{2-[1,1-dimethyl-3-(2-oxo-2,3-dihydro-benzoimidazol-1-yl)-propylamino]-1-hydroxy-ethyl}-6-hydroxy-4H-benzo[1,4]oxazin-3-one, 8-{2-[1,1-dimethyl-3-(6-methyl-2-oxo-2,3-dihydro-benzoimidazol-1-yl)-propylamino]-1-hydroxy-ethyl}-6-hydroxy-4H-benzo[1,4]oxazin-3-one, 8-{2-[1,1-dimethyl-3-(2-oxo-5-trifluormethyl-2,3-dihydro-benzoimidazol-1-yl)-propylamino]-1-hydroxy-ethyl}-6-hydroxy-4H-benzo[1,4]oxazin-3-one, 8-{2-[1,1-dimethyl-3-(3-methyl-2-oxo-2,3-dihydro-benzoimidazol-1-yl)-propylamino]-1-hydroxy-ethyl}-6-hydroxy-4H-benzo[1,4]oxazin-3-one, 3-(4-{6-[2-Hydroxy-2-(4-hydroxy-3-hydroxymethyl-phenyl)-ethylamino]-hexyloxy}-butyl)-benzenesulfoneamid, 5-[2-(5,6-diethyl-indan-2-ylamino)-1-hydroxy-ethyl]-8-hydroxy-1H-quinolin-2-one, 1-[3-(4-Methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-Hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 6-hydroxy-8-{1-hydroxy-2-[2-(4-methoxy-phenyl)-1,1-dimethyl-ethylamino]-ethyl}-4H-benzo[1,4]oxazin-3-one, 6-hydroxy-8-{1-hydroxy-2-[2-(4-phenoxy-acetic acid ethylester)-1,1-dimethyl-ethylamino]-ethyl}-4H-benzo[1,4]oxazin-3-one, 6-Hydroxy-8-{1-hydroxy-2-[2-(4-phenoxy-acetic acid)-1,1-dimethyl-ethylamino]-ethyl}-4H-benzo[1,4]oxazin-3-one, 8-{2-[1,1-dimethyl-2-(2,4,6-trimethylphenyl)-ethylamino]-1-hydroxy-ethyl}-6-hydroxy-4H-benzo[1,4]oxazin-3-one, 6-Hydroxy-8-{1-hydroxy-2-[2-(4-hydroxy-phenyl)-1,1-dimethyl-ethylamino]-ethyl}-4H-benzo[1,4]oxazin-3-one, 6-hydroxy-8-{1-hydroxy-2-[2-(4-isopropyl-phenyl)-1,1-dimethyl-ethylamino]-ethyl}-4H-benzo[1,4]oxazin-3-one, 8-{2-[2-(4-ethyl-phenyl)-1,1-dimethyl-ethylamino]-1-hydroxy-ethyl}-6-hydroxy-4H-benzo[1,4]oxazin-3-one, 8-{2-[2-(4-ethoxy-phenyl)-1,1-dimethyl-ethylamino]-1-hydroxy-ethyl}-6-hydroxy-4H-benzo[1,4]oxazin-3-one, 4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butanoic acid, 8-{2-[2-(3,4-difluoro-phenyl)-1,1-dimethyl-ethylamino]-1-hydroxy-ethyl}-6-hydroxy-4H-benzo[1,4]oxazin-3-one and 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, optionally in racemic form, as enantiomers, diastereomeres or as pharmacologically acceptable salts, solvates or hydrates.

Preferred are formoterol, salmeterol, indacaterol, carmoterol, orciprelanine, salbutamol, metaproterol, terbutaline, 6-hydroxy-8-{1-hydroxy-2-[2-(4-methoxy-phenyl)-1,1-dimethyl-ethylamino]-ethyl}-4H-benzo[1,4]oxazin-3-one, 8-{2-[2-(2,4-difluoro-phenyl)-1,1-dimethyl-ethylamino]-1-hydroxy-ethyl}-6-hydroxy-4H-benzo[1,4]oxazin-3-one, 8-{2-[2-(3,5-difluoro-phenyl)-1,1-dimethyl-ethylamino]-1-hydroxy-ethyl}-6-hydroxy-4H-benzo[1,4]oxazin-3-one, 8-{2-[2-(4-Ethoxy-phenyl)-1,1-dimethyl-ethylamino]-1-hydroxy-ethyl}-6-hydroxy-4H-benzo[1,4]oxazin-3-one, 8-{2-[2-(4-Fluor-phenyl)-1,1-dimethyl-ethylamino]-1-hydroxy-ethyl}-6-hydroxy-4H-benzo[1,4]oxazin-3-one, N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methansulfonamid, N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methansulfonamid, N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methansulfonamid, N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methansulfonamid, 8-{2-[1,1-dimethyl-3-(2-oxo-2,3-dihydro-benzoimidazol-1-yl)-propylamino]-1-hydroxy-ethyl}-6-hydroxy-4H-benzo[1,4]oxazin-3-one, 8-{2-[1,1-dimethyl-3-(6-methyl-2-oxo-2,3-dihydro-benzoimidazol-1-yl)-propylamino]-1-hydroxy-ethyl}-6-hydroxy-4H-benzo[1,4]oxazin-3-one, 8-{2-[1,1-dimethyl-3-(2-oxo-5-trifluoromethyl-2,3-dihydro-benzoimidazol-1-yl)-propylamino]-1-hydroxy-ethyl}-6-hydroxy-4H-benzo[1,4]oxazin-3-one, 8-{2-[1,1-dimethyl-3-(3-methyl-2-oxo-2,3-dihydro-benzoimidazol-1-yl)-propylamino]-1-hydroxy-ethyl}-6-hydroxy-4H-benzo[1,4]oxazin-3-one, 3-(4-{6-[2-hydroxy-2-(4-hydroxy-3-hydroxymethyl-phenyl)-ethylamino]-hexyloxy}-butyl)-benzenesulfoneamide, 6-hydroxy-8-{1-hydroxy-2-[2-(4-methoxy-phenyl)-1,1-dimethyl-ethylamino]-ethyl}-4H-benzo[1,4]oxazin-3-one, 6-hydroxy-8-{1-hydroxy-2-[2-(4-phenoxy-acetic acid ethylester)-1,1-dimethyl-ethylamino]-ethyl}-4H-benzo[1,4]oxazin-3-one, 6-hydroxy-8-{1-hydroxy-2-[2-(4-phenoxy-acetic acid)-1,1-dimethyl-ethylamino]-ethyl}-4H-benzo[1,4]oxazin-3-one, 8-{2-[1,1-dimethyl-2-(2,4,6-trimethylphenyl)-ethylamino]-1-hydroxy-ethyl}-6-hydroxy-4H-benzo[1,4]oxazin-3-one, 6-hydroxy-8-{1-hydroxy-2-[2-(4-hydroxy-phenyl)-1,1-dimethyl-ethylamino]-ethyl}-4H-benzo[1,4]oxazin-3-one, 6-hydroxy-8-{1-hydroxy-2-[2-(4-isopropyl-phenyl)-1,1-dimethyl-ethylamino]-ethyl}-4H-benzo[1,4]oxazin-3-one, 8-{2-[2-(4-ethyl-phenyl)-1,1-dimethyl-ethylamino]-1-hydroxy-ethyl}-6-hydroxy-4H-benzo[1,4]oxazin-3-one, 8-{2-[2-(4-ethoxy-phenyl)-1,1-dimethyl-ethylamino]-1-hydroxy-ethyl}-6-hydroxy-4H-benzo[1,4]oxazin-3-one, 4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butanoic acid, 8-{2-[2-(3,4-difluoro-phenyl)-1,1-dimethyl-ethylamino]-1-hydroxy-ethyl}-6-hydroxy-4H-benzo[1,4]oxazin-3-on and 5-[2-(5,6-diethyl-indan-2-ylamino)-1-hydroxy-ethyl]-8-hydroxy-1H-quinolin-2-one, optionally in racemic form, as enantiomers, diastereomeres or as pharmacologically acceptable salts, solvates or hydrates.

Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-2 Anticholinergics

Examples of preferred anticholinergics which may be mentioned include tiotropium salts, preferred the bromide salt, oxitropium salts, preferred the bromide salt, flutropium salts, preferred the bromide salt, ipratropium salts, preferred the bromide salt, glycopyrronium salts, preferred the bromide salt, trospium salts, preferred the chloride salt, tolterodin, atropine, hyoscyamine, dicycloverine, otilonium bromide, scopolamine, scopolamine methyl hydroxide. From the above mentioned salts the pharmacologically active part is the cation, possible anions are chloride, bromide, iodide, sulfate, phosphate, methansulfonate, nitrate, maleate, acetate, citrate, fumarate, tartrate, oxalate, succinate, benzoate or p-toluenesulfonate. Furthermore
2,2-diphenylpropionic acid tropenolester-methobromide
2,2-diphenylpropionic acid scopinester-methobromide
2-fluoro-2,2-diphenylacetic acid scopinester-methobromide
2-fluoro-2,2-diphenylacetic acid tropenolester-methobromide
3,3′,4,4′-tetrafluorobenzilic acid tropenolester-methobromide
3,3′,4,4′-tetrafluorobenzilic acid scopinester-methobromide
4,4′-difluorobenzilic acid tropenolester-methobromide
4,4′-difluorobenzilic acid scopinester-methobromide
3,3′-difluorobenzilic acid tropenolester methobromide
3,3′-difluorobenzilic acid scopineste rmethobromide
9-hydroxy-fluoren-9-carboxylic acid tropenolester methobromide
9-fluoro-fluoren-9-carboxylic acid tropenolester-methobromide
9-hydroxy-fluoren-9-carboxylic acid scopinester methobromide
9-fluoro-fluoren-9-carboxylic acid scopinester methobromide
9-methyl-fluoren-9-carboxylic acid tropenolester methobromide
9-methyl-fluoren-9-carboxylic acid scopinester methobromide
benzilic acid cyclopropyltropinester methobromide
2,2-diphenylpropionic acid cyclopropyltropinester methobromide
9-hydroxy-xanthen-9-carboxylic acid cyclopropyltropinester methobromide
9-methyl-fluoren-9-carboxylic acid cyclopropyltropinester methobromide
9-methyl-xanthen-9-carboxylic acid cyclopropyltropinester methobromide
9-hydroxy-fluoren-9-carboxylic acid cyclopropyltropinester methobromide
4,4′-difluorobenzilic acid methylestercyclopropyltropinester methobromide
9-hydroxy-xanthen-9-carboxylic acid tropenolester methobromide
9-hydroxy-xanthen-9-carboxylic acid scopinester methobromide
9-methyl-xanthen-9-carboxylic acid tropenolester methobromide
9-methyl-xanthen-9-carboxylic acid scopinester methobromide
9-ethyl-xanthen-9-carboxylic acid tropenolester methobromide
9-difluoromethyl-xanthen-9-carboxylic acid tropenolester methobromide
9-hydroxymethyl-xanthen-9-carboxylic acid scopinester methobromide

### 2-3 Corticosteroids

Examples of preferred corticosteroids which may be mentioned include beclomethasone, betamethasone, budesonide, butixocorte, ciclesonide, clobetasol, deflazacorte, desoximetasone, dexamethasone, etiprednole, flunisolide, fluticasone, fluocinonide, loteprednole, hydrocortisone, cortisone, methylprednisolone, mometasone, prednicarbate, prednisolone, prednisone, rofleponide, triamcinolone, RPR-106541, NS-126 and
6,9-difluoro-17-[(2-furanylcarbonyl)oxy]-11-hydroxy-16-methyl-3-oxo-androsta-1,4-dien-17-carbothionic acid (S)-fluoromethylester
6,9-difluoro-11-hydroxy-16-methyl-3-oxo-17-propionyloxy-androsta-1,4-dien-17-carbothionic acid (S)-(2-oxo-tetrahydro-furan-3S-yl)ester,
6α,9α-difluoro-11β-hydroxy-16α-methyl-3-oxo-17α-(2,2,3,3-tertamethylcyclopropylcarbonyl)oxy-androsta-1,4-diene-17β-carboxylic acid cyanomethyl ester

Preferred are ciclesonide, dexamethasone, prednisolone, prednisone, hydrocortisone and cortisone.

optionally in racemic form, as enantiomers, diastereomeres or as pharmacologically acceptable salts, solvates or hydrates. Examples for preferred salts and derivatives are alkali salts, i.e. sodium or potassium salts, sulfobenzoates, phosphates, isonicotinates, acetates, dichloroacetates, propionates, dihydrogenphosphates, palmitates, pivalates or furoates.

### 2-4 PDE4-Inhibitors

Examples of preferred PDE4-inhibitors which may be mentioned include enprofylline, theophylline, aminophylline, oxtriphylline, apremilast, roflumilast, ariflo (cilomilast), tofimilast, pumafentrine, lirimilast, arofylline, atizorame, oglemilastum, D-4418, Bay-198004, BY343, CP-325,366, D-4396 (Sch-351591), AWD-12-281 (GW-842470), NCS-613, CDP-840, D-4418, PD-168787, T-440, T-2585, V-11294A, C1-1018, CDC-801, CDC-3052, D-22888, YM-58997, Z-15370 and
N-(3,5-dichloro-1-oxo-pyridin-4-yl)-4-difluoromethoxy-3-cyclopropylmethoxybenzamide
(−)p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamid
(R)-(+)-1-(4-bromobenzyl)-4-[(3-cyclopentyloxy)-4-methoxyphenyl]-2-pyrrolidon
3-(cyclopentyloxy-4-methoxyphenyl)-1-(4-N′-[N-2-cyano-S-methyl-isothioureido]benzyl)-2-pyrrolidone
cis[4-cyano-4-(3-cyclopentyloxy-4-methoxyphenyl)cyclohexan-1-carboxylic acid]
2-carbomethoxy-4-cyano-4-(3-cyclopropylmethoxy-4-difluoromethoxyphenyl)cyclohexan-1-one
cis[4-cyano-4-(3-cyclopropylmethoxy-4-difluoromethoxyphenyl)cyclohexan-1-ol]
(R)-(+)-ethyl[4-(3-cyclopentyloxy-4-methoxyphenyl)pyrrolidin-2-yliden]acetate
(S)-(−)-ethyl[4-(3-cyclopentyloxy-4-methoxyphenyl)pyrrolidin-2-yliden]acetate
9-cyclopentyl-5,6-dihydro-7-ethyl-3-(2-thienyl)-9H-pyrazolo[3,4-c]-1,2,4-triazolo[4,3-a]pyridine
9-cyclopentyl-5,6-dihydro-7-ethyl-3-(tert-butyl)-9H-pyrazolo[3,4-c]-1,2,4-triazolo[4,3-a]pyridine

Particularly preferred are theophylline, aminophylline, oxtriphylline, roflumilast, and apremilast, with roflumilast being particularly preferred.

optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-5 LTD4-Antagonists

Examples of preferred LTD4 antagonists which may be mentioned include Montelukast, Pranlukast, Zafirlukast, MCC-847 (ZD-3523), MN-001, MEN-91507 (LM-1507), VUF-5078, VUF-K-8707, L-733321, CR-3465, ONO-RS-531, BAY-u9773 and
1-(((R)-(3-(2-(6,7-difluoro-2-quinolinyl)ethenyl)phenyl)-3-(2-(2-hydroxy-2-propyl)phenyl)thio)methylcyclopropane-acetic acid,
1-(((1(R)-3 (3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropane acetic acid
[2-[[2-(4-tert-butyl-2-thiazolyl)-5-benzofuranyl]oxymethyl]phenyl]acetic acid
   optionally in racemic form, as enantiomers, diastereomeres or as pharmacologically acceptable salts, solvates or hydrates.

Particularly preferred are montelukast, pranlukast and zafirlukast. Most preferred is montelukast, especially in the form of montelukast sodium.

Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate. Further examples for optionally preferred salts and derivatives are alkali salts, i.e. sodium or potassium salts, sulfobenzoates, phosphates, isonicotinates, acetates, propionates, dihydrogenphosphates, palmitates, pivalates or furoates.

### 2-6 EGFR-Kinase Inhibitors

Examples of preferred EGFR-inhibitors which may be mentioned include Cetuximabe, Trastuzumabe, ABX-EGF, Mab ICR-62 and
4-[(3-Chloro-4-fluorophenyl)amino]-6-{[4-(morpholin-4-yl)-1-oxo-2-buten-1-yl]amino}-7-cyclopropylmethoxy-quinazoline
4-[(3-Chloro-4-fluorophenyl)amino]-6-{[4-(N,N-diethylamino)-1-oxo-2-buten-1-yl]amino}-7-cyclopropylmethoxy-quinazoline
4-[(3-Chloro-4-fluorophenyl)amino]-6-{[4-(N,N-dimethylamino)-1-oxo-2-buten-1-yl]amino}-7-cyclopropylmethoxy-quinazoline
4-[(R)-(1-Phenyl-ethyl)amino]-6-{[4-(morpholin-4-yl)-1-oxo-2-buten-1-yl]amino}-7-cyclopentyloxy-quinazoline
4-[(3-Chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline
4-[(3-Chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline
4-[(3-Chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline
4-[(3-Chloro-4-fluoro-phenyl)amino]-6-[2-((S)-6-methyl-2-oxo-morpholin-4-yl)-ethoxy]-7-methoxy-quinazoline
4-[(3-Chloro-4-fluorophenyl)amino]-6-({4-[N-(2-methoxy-ethyl)-N-methyl-amino]-1-oxo-2-buten-1-yl}amino)-7-cyclopropylmethoxy-quinazoline
4-[(3-Chloro-4-fluorophenyl)amino]-6-{[4-(N,N-dimethylamino)-1-oxo-2-buten-1-yl]amino}-7-cyclopentyloxy-quinazoline
4-[(R)-(1-Phenyl-ethyl)amino]-6-{[4-(N,N-bis-(2-methoxy-ethyl)-amino)-1-oxo-2-buten-1-yl]amino}-7-cyclopropylmethoxy-quinazoline
4-[(R)-(1-Phenyl-ethyl)amino]-6-({4-[N-(2-methoxy-ethyl)-N-ethyl-amino]-1-oxo-2-buten-1-yl}amino)-7-cyclopropylmethoxy-quinazoline
4-[(R)-(1-Phenyl-ethyl)amino]-6-({4-[N-(2-methoxy-ethyl)-N-methyl-amino]-1-oxo-2-buten-1-yl}amino)-7-cyclopropylmethoxy-quinazoline
4-[(R)-(1-Phenyl-ethyl)amino]-6-({4-[N-(tetrahydropyran-4-yl)-N-methyl-amino]-1-oxo-2-buten-1-yl}amino)-7-cyclopropylmethoxy-quinazoline
4-[(3-Chloro-4-fluorphenyl)amino]-6-{[4-(N,N-dimethylamino)-1-oxo-2-buten-1-yl]amino}-7-((R)-tetrahydro furan-3-yloxy)-quinazoline
4-[(3-Chloro-4-fluorophenyl)amino]-6-{[4-(N,N-dimethylamino)-1-oxo-2-buten-1-yl]amino}-7-((S)-tetrahydro furan-3-yloxy)-quinazoline
4-[(3-Chloro-4-fluorophenyl)amino]-6-({4-[N-(2-methoxy-ethyl)-N-methyl-amino]-1-oxo-2-buten-1-yl}amino)-7-cyclopentyloxy-quinazoline
4-[(3-Chloro-4-fluorophenyl)amino]-6-{[4-(N-cyclopropyl-N-methyl-amino)-1-oxo-2-buten-1-yl]amino}-7-cyclopentyloxy-quinazoline
4-[(3-Chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline
4-[(3-Chloro-4-fluorphenyl)amino]-6-{[4-(N,N-dimethylamino)-1-oxo-2-buten-1-yl]amino}-7-[(S)-(tetrahydro furan-2-yl)methoxy]-quinazoline
4-[(3-Ethinyl-phenyl)amino]-6,7-bis-(2-methoxy-ethoxy)-quinazoline
4-[(3-Chloro-4-fluorophenyl)amino]-7-[3-(morpholin-4-yl)-propyloxy]-6-[(vinylcarbonyl)amino]-quinazoline
4-[(R)-(1-Phenyl-ethyl)amino]-6-(4-hydroxy-phenyl)-7H-pyrrolo[2,3-d]pyrimidine
3-Cyano-4-[(3-chloro-4-fluorophenyl)amino]-6-{[4-(N,N-dimethylamino)-1-oxo-2-buten-1-yl]amino}-7-ethoxy-quino line
4-{[3-Chloro-4-(3-fluoro-benzyloxy)-phenyl]amino}-6-(5-{[(2-methansulfonyl-ethyl)amino]methyl}-furan-2-yl)quinazoline
4-[(R)-(1-Phenyl-ethyl)amino]-6-{[4-((R)-6-methyl-2-oxo-morpholin-4-yl)-1-oxo-2-buten-1-yl]amino}-7-methoxy-quinazoline
4-[(3-Chloro-4-fluorophenyl)amino]-6-{[4-(morpholin-4-yl)-1-oxo-2-buten-1-yl]amino}-7-[(tetrahydro furan-2-yl)methoxy]-quinazoline
4-[(3-Chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline
4-[(3-Ethinyl-phenyl)amino]-6-{[4-(5,5-dimethyl-2-oxo-morpholin-4-yl)-1-oxo-2-buten-1-yl]amino}-quinazoline
4-[(3-Chloro-4-fluoro-phenyl)amino]-6-[2-(2,2-dimethyl-6-oxo-morpholin-4-yl)-ethoxy]-7-methoxy-quinazoline
4-[(3-Chloro-4-fluoro-phenyl)amino]-6-[2-(2,2-dimethyl-6-oxo-morpholin-4-yl)-ethoxy]-7-[(R)-(tetrahydro furan-2-yl)methoxy]-quinazoline
4-[(3-Chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline
4-[(3-Chloro-4-fluoro-phenyl)amino]-6-{2-[4-(2-oxo-morpholin-4-yl)-piperidin-1-yl]-ethoxy}-7-methoxy-quinazoline
4-[(3-Chloro-4-fluoro-phenyl)amino]-6-[1-(tert.-butyloxycarbonyl)-piperidin-4-yloxy]-7-methoxy-quinazoline
4-[(3-Chloro-4-fluoro-phenyl)amino]-6-(trans-4-amino-cyclohexan-1-yloxy)-7-methoxy-quinazoline
4-[(3-Chloro-4-fluoro-phenyl)amino]-6-(trans-4-methansulfonylamino-cyclohexan-1-yloxy)-7-methoxy-quinazoline
4-[(3-Chloro-4-fluoro-phenyl)amino]-6-(tetrahydropyran-3-yloxy)-7-methoxy-quinazoline
4-[(3-Chloro-4-fluoro-phenyl)amino]-6-(1-methyl-piperidin-4-yloxy)-7-methoxy-quinazoline
4-[(3-Chloro-4-fluoro-phenyl)amino]-6-{1-[(morpholin-4-yl)carbonyl]-piperidin-4-yloxy}-7-methoxy-quinazoline
4-[(3-Chloro-4-fluoro-phenyl)amino]-6-{1-[(methoxymethyl)carbonyl]-piperidin-4-yloxy}-7-methoxy-quinazoline
4-[(3-Chloro-4-fluoro-phenyl)amino]-6-(piperidin-3-yloxy)-7-methoxy-quinazoline
4-[(3-Chloro-4-fluoro-phenyl)amino]-6-[1-(2-acetylamino-ethyl)-piperidin-4-yloxy]-7-methoxy-quinazoline
4-[(3-Chloro-4-fluoro-phenyl)amino]-6-(tetrahydropyran-4-yloxy)-7-ethoxy-quinazoline
4-[(3-Chloro-4-fluoro-phenyl)amino]-6-((S)-tetrahydrofuran-3-yloxy)-7-hydroxy-quinazoline
4-[(3-Chloro-4-fluoro-phenyl)amino]-6-(tetrahydropyran-4-yloxy)-7-(2-methoxy-ethoxy)-quinazoline
4-[(3-Chloro-4-fluoro-phenyl)amino]-6-{trans-4-[(dimethylamino)sulfonylamino]-cyclohexan-1-yloxy}-7-methoxy-quinazoline
4-[(3-Chloro-4-fluoro-phenyl)amino]-6-{trans-4-[(morpholin-4-yl)carbonylamino]-cyclohexan-1-yloxy}-7-methoxy-quinazoline
4-[(3-Chloro-4-fluoro-phenyl)amino]-6-{trans-4-[(morpholin-4-yl)sulfonylamino]-cyclohexan-1-yloxy}-7-methoxy-quinazoline
4-[(3-Chloro-4-fluoro-phenyl)amino]-6-(tetrahydropyran-4-yloxy)-7-(2-acetylamino-ethoxy)-quinazoline
4-[(3-Chloro-4-fluoro-phenyl)amino]-6-(tetrahydropyran-4-yloxy)-7-(2-methansulfonylamino-ethoxy)-quinazoline
4-[(3-Chloro-4-fluoro-phenyl)amino]-6-{1-[(piperidin-1-yl)carbonyl]-piperidin-4-yloxy}-7-methoxy-quinazoline
4-[(3-Chloro-4-fluoro-phenyl)amino]-6-(1-aminocarbonylmethyl-piperidin-4-yloxy)-7-methoxy-quinazoline
4-[(3-Chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline
4-[(3-Chloro-4-fluoro-phenyl)amino]-6-(cis-4-{N-[(morpholin-4-yl)carbonyl]-N-methyl-amino}-cyclohexan-1-yloxy)-7-methoxy-quinazoline
4-[(3-Chloro-4-fluoro-phenyl)amino]-6-(cis-4-{N-[(morpholin-4-yl)sulfonyl]-N-methyl-amino}-cyclohexan-1-yloxy)-7-methoxy-quinazoline
4-[(3-Chloro-4-fluoro-phenyl)amino]-6-(trans-4-ethansulfonylamino-cyclohexan-1-yloxy)-7-methoxy-quinazoline
4-[(3-Chloro-4-fluoro-phenyl)amino]-6-(1-methansulfonyl-piperidin-4-yloxy)-7-ethoxy-quinazoline
4-[(3-Chloro-4-fluoro-phenyl)amino]-6-(1-methansulfonyl-piperidin-4-yloxy)-7-(2-methoxy-ethoxy)-quinazoline
4-[(3-Chloro-4-fluoro-phenyl)amino]-6-[1-(2-methoxy-acetyl)-piperidin-4-yloxy]-7-(2-methoxy-ethoxy)-quinazoline
4-[(3-Chloro-4-fluoro-phenyl)amino]-6-(cis-4-acetylamino-cyclohexan-1-yloxy)-7-methoxy-quinazoline
4-[(3-Ethinyl-phenyl)amino]-6-[1-(tert.-butyloxycarbonyl)-piperidin-4-yloxy]-7-methoxy-quinazoline
4-[(3-Ethinyl-phenyl)amino]-6-(tetrahydropyran-4-yloxy]-7-methoxy-quinazoline
4-[(3-Chloro-4-fluoro-phenyl)amino]-6-(cis-4-{N-[(piperidin-1-yl)carbonyl]-N-methyl-amino}-cyclohexan-1-yloxy)-7-methoxy-quinazoline
4-[(3-Chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline
4-[(3-Chloro-4-fluoro-phenyl)amino]-6-{cis-4-[(morpholin-4-yl)carbonylamino]-cyclohexan-1-yloxy}-7-methoxy-quinazoline
4-[(3-Chloro-4-fluoro-phenyl)amino]-6-{1-[2-(2-oxopyrrolidin-1-yl)ethyl]-piperidin-4-yloxy}-7-methoxy-quinazoline
4-[(3-Chloro-4-fluoro-phenyl)amino]-6-{1-[(morpholin-4-yl)carbonyl]-piperidin-4-yloxy}-7-(2-methoxy-ethoxy)-quinazoline
4-[(3-Ethinyl-phenyl)amino]-6-(1-acetyl-piperidin-4-yloxy)-7-methoxy-quinazoline
4-[(3-Ethinyl-phenyl)amino]-6-(1-methyl-piperidin-4-yloxy)-7-methoxy-quinazoline
4-[(3-Ethinyl-phenyl)amino]-6-(1-methansulfonyl-piperidin-4-yloxy)-7-methoxy-quinazoline
4-[(3-Chloro-4-fluoro-phenyl)amino]-6-(1-methyl-piperidin-4-yloxy)-7 (2-methoxy-ethoxy)-quinazoline
4-[(3-Chloro-4-fluoro-phenyl)amino]-6-(1-isopropyloxycarbonyl-piperidin-4-yloxy)-7-methoxy-quinazoline
4-[(3-Chloro-4-fluoro-phenyl)amino]-6-(cis-4-methylamino-cyclohexan-1-yloxy)-7-methoxy-quinazoline
4-[(3-Chloro-4-fluoro-phenyl)amino]-6-{cis-4-[N-(2-methoxy-acetyl)-N-methyl-amino]-cyclohexan-1-yloxy}-7-methoxy-quinazoline
4-[(3-Ethinyl-phenyl)amino]-6-(piperidin-4-yloxy)-7-methoxy-quinazoline
4-[(3-Ethinyl-phenyl)amino]-6-[1-(2-methoxy-acetyl)-piperidin-4-yloxy]-7-methoxy-quinazoline
4-[(3-Ethinyl-phenyl)amino]-6-{1-[(morpholin-4-yl)carbonyl]-piperidin-4-yloxy}-7-methoxy-quinazoline
4-[(3-Chloro-4-fluoro-phenyl)amino]-6-{1-[(cis-2,6-dimethyl-morpholin-4-yl)carbonyl]-piperidin-4-yloxy}-7-methoxy-quinazoline
4-[(3-Chloro-4-fluoro-phenyl)amino]-6-{1-[(2-methyl-morpholin-4-yl)carbonyl]-piperidin-4-yloxy}-7-methoxy-quinazoline
4-[(3-Chloro-4-fluoro-phenyl)amino]-6-{1-[(S,S)-(2-oxa-5-aza-bicyclo[2.2.1]hept-5-yl)carbonyl]-piperidin-4-yloxy}-7-methoxy-quinazoline
4-[(3-Chloro-4-fluoro-phenyl)amino]-6-{1-[(N-methyl-N-2-methoxyethyl-amino)carbonyl]-piperidin-4-yloxy}-7-methoxy-quinazoline
4-[(3-Chloro-4-fluoro-phenyl)amino]-6-(1-ethyl-piperidin-4-yloxy)-7-methoxy-quinazoline
4-[(3-Chloro-4-fluoro-phenyl)amino]-6-{1-[(2-methoxyethyl)carbonyl]-piperidin-4-yloxy}-7-methoxy-quinazoline
4-[(3-Chloro-4-fluoro-phenyl)amino]-6-{1-[(3-methoxypropyl-amino)-carbonyl]-piperidin-4-yloxy}-7-methoxy-quinazoline
4-[(3-Chloro-4-fluoro-phenyl)amino]-6-[cis-4-(N-methansulfonyl-N-methyl-amino)-cyclohexan-1-yloxy]-7-methoxy-quinazoline
4-[(3-Chloro-4-fluoro-phenyl)amino]-6-[cis-4-(N-acetyl-N-methyl-amino)-cyclohexan-1-yloxy]-7-methoxy-quinazoline
4-[(3-Chloro-4-fluoro-phenyl)amino]-6-(trans-4-methylamino-cyclohexan-1-yloxy)-7-methoxy-quinazoline
4-[(3-Chloro-4-fluoro-phenyl)amino]-6-[trans-4-(N-methansulfonyl-N-methyl-amino)-cyclohexan-1-yloxy]-7-methoxy-quinazoline
4-[(3-Chloro-4-fluoro-phenyl)amino]-6-(trans-4-dimethylamino-cyclohexan-1-yloxy)-7-methoxy-quinazoline
4-[(3-Chloro-4-fluoro-phenyl)amino]-6-(trans-4-{N-[(morpholin-4-yl)carbonyl]-N-methyl-amino}-cyclohexan-1-yloxy)-7-methoxy-quinazoline
4-[(3-Chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline
4-[(3-Chloro-4-fluoro-phenyl)amino]-6-(1-methansulfonyl-piperidin-4-yloxy)-7-methoxy-quinazoline
4-[(3-Chloro-4-fluoro-phenyl)amino]-6-(1-cyano-piperidin-4-yloxy)-7-methoxy-quinazoline
   optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-7 PAF-Antagonisten

Examples of preferred PAF antagonists which may be mentioned include
4-(2-Chlorophenyl)-9-methyl-2-[3(4-morpholinyl)-3-propanon-1-yl]-6H-thieno-[3,2-f]-[1,2,4]triazolo[4,3-a][1,4]diazepine
6-(2-Chlorophenyl)-8,9-dihydro-1-methyl-8-[(4-morpholinyl)carbonyl]-4H,7H-cyclo-penta-[4,5]thieno-[3,2-f][1,2,4]triazolo[4,3-a][1,4]diazepine
   optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-8 Lipoxin A4 Derivatives

Examples of preferred Lipoxin A4 derivatives which may be mentioned include 15-epi-lipoxin a4, ATL-1, aspirin-triggered lipoxin A(4) and analogs, protectin D1, Resolvin E1 and benzo-lipoxin A4 analogs

optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-9 FPRL1-Modulators

Examples of preferred FPRL1-modulators which may be mentioned include

CGEN-855 and BML-111: 5(S),6(R),7-trihydroxyheptanoic acid methyl ester,
optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-10 LTB4-Receptor Antagonists

Examples of preferred LTB4-receptor antagonists which may be mentioned include BIIL260, BIIL284 optionally in racemic form, as enantiomers, diastereomeres or as pharmacologically acceptable salts, solvates or hydrates.

### 2-11 Histamine Receptor Antagonists

Examples of preferred Histamine receptor antagonists which may be mentioned include Acrivastine, Azatadine, Azelastine, Bamipine, Brompheniramine, Carbinoxamine, Cetirizine, Chlorphenoxamine, Chlorphenaramine, Clemastine, Cexchlorpheniramine, Cyproheptadine, Desloratidine, Dexbromphenarimine, Dexchlorpheniramine, Dimenhydrinate, Dimetinden, Diphenhydramine, Doxylamine, Ebastine, Emedastine, Epinastine, Fexofenadine, Hydroxyzine, Ketotifen, Levocetirizine, Levocabastine, Loratadine, Meclozine, Methdilazine, Mizolastine, Olopatadine, Phenindamine, Pheniramine, Phenyltoloxamine, Promethazine, Pyrilamine, Tecastemizole, Trimepramine, Trimethobenzamide, Triprolidine, JNJ-7777120, PF-2988403 and CZC-13788, optionally in racemic form, as enantiomers, diastereomeres or as pharmacologically acceptable salts, solvates or hydrates.

Particularly preferred are azelastine, cetirizine, desloratidine, ebastine, epinastine, fexofenadine, ketotifen, levocetirizine, loratadine and olopatadine.

### 2-12 PI3 Kinase Antagonists

Examples of preferred PI3 kinase antagonists which may be mentioned include AS-605240, CZC-19091, D-106669, D-87503, XL-147, IC-980033, IC-87114, GDC-0941 and BEZ-235 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-13 SYK-Inhibitors

Examples of preferred SYK inhibitors which may be mentioned include Rigels R-788 and prodrugs thereof, such as R-406, R-112
2-[(2-aminoethyl)amino]-4-[(3-bromophenyl)amino]-5-pyrimidinecarboxamide;
2-[[7-(3,4-dimethoxyphenyl)imidazo[1,2-c]pyrimidin-5-yl]amino]-3-pyridinecarboxamide;
6-[[5-fluoro-2-[3,4,5-trimethoxyphenyl)amino]-4-pyrimidinyl]amino]-2,2-dimethyl-2H-pyrido[3,2-b]-1,4-oxazin-3(4H)-one;
N-[3-bromo-7-(4-methoxyphenyl)-1,6-naphthyridin-5-yl]-1,3-diaminopropane
7-(4-methoxyphenyl)-N-methyl-1,6-naphthyridin-5-amine;
N-[7-(4-methoxyphenyl)-1,6-naphthyridin-5-yl]-1,3-diaminopropane;
N-[7-(2-thienyl)-1,6-naphthyridin-5-yl-1,3-propanediamine;
N-[7-[4-(dimethylamino)phenyl]-1,6-naphthyridin-5-yl]-1,2-diaminoethane;
N-[7-(4-methoxyphenyl)-2-(trifluoromethyl)-1,6-naphthyridin-5-yl]-1,3-diaminopropane;
N-[7-(4-methoxyphenyl)-3-phenyl-1,6-naphthyridin-5-yl]-1,3-diaminopropane;
N-(7-phenyl-1,6-naphthyridin-5-yl)-1,3-diaminopropane;
N-[7-(3-fluorophenyl)-1,6-naphthyridin-5-yl]-1,3-diaminopropane;
N-[7-(3-chlorophenyl)-1,6-naphthyridin-5-yl]-1,3-diaminopropane;
N-[7-[3-(trifluoromethoxy)phenyl]-1,6-naphthyridin-5-yl]-1,3-diaminopropane;
N-[7-(4-fluorophenyl)-1,6-naphthyridin-5-yl]-1,3-diaminopropane;
N-[7-(4-fluorophenyl)-1,6-naphthyridin-5-yl]-1,3-diaminopropane;
N-[7-(4-chlorophenyl)-1,6-naphthyridin-5-yl]-1,3-diaminopropane;
N-[7-(4′-methyl[1,1′-biphenyl]-4-yl)-1,6-naphthyridin-1,3-diaminopropane;
N-[7-[4-(dimethylamino)phenyl]-1,6-naphthyridin-5-yl]-1,3-diaminopropane;
N-[7-[4-(diethylamino)phenyl]-1,6-naphthyridin-5-yl]-1,3-diaminopropane;
N-[7-[4-(4-morpholinyl)phenyl]-1,6-naphthyridin-5-yl]-1,3-diaminopropane;
N-[7-[4-[[2-(dimethylamino) ethyl]methylamino]phenyl]-1,6-naphthyridin-5-yl]-1,3-diaminopropane;
N-[7-(4-bromophenyl)-1,6-naphthyridin-5-yl]-1,3-diaminopropane;
N-[7-(4-methylphenyl)-1,6-naphthyridin-5-yl]-1,3-diaminopropane;
N-[7-[4-(methylthio)phenyl]-1,6-naphthyridin-5-yl]-1,3-diaminopropane;
N-[7-[4-(1-methylethyl)phenyl]-1,6-naphthyridin-5-yl]-1,3-diaminopropane;
7-[4-(dimethylamino)phenyl]-N-methyl-1,6-naphthyridin-5-amine;
7-[4-(dimethylamino)phenyl]-N,N-dimethyl-1,6-naphthyridin-5-amine;
N-[7-[4-(dimethylamino)phenyl]-1,6-naphthyridin-5-yl]-1,4-diaminobutane;
N-[7-[4-(dimethylamino)phenyl]-1,6-naphthyridin-5-yl]-1,5-diaminopentane;
3-[[7-[4-(dimethylamino)phenyl]-1,6-naphthyridin-5-yl]oxy]-1-propanol;
4-[5-(4-aminobutoxy)-1,6-naphthyridin-7-yl]-N,N-dimethyl-benzenamine;
4-[[7-[4-(dimethylamino)phenyl]-1,6-naphthyridin-5-yl]amino]-1-butanol;
N-[7-[4-(dimethylamino)phenyl]-1,6-naphthyridin-5-yl]-N-methyl-1,3-diaminopropane;
N-[7-[4-(dimethylamino)phenyl]-1,6-naphthyridin-5-yl]-N′-methyl-1,3-diaminopropane;
N-[7-[4-(dimethylamino)phenyl]-1,6-naphthyridin-5-yl]-N,N′-dimethyl-1,3-diaminopropane;
1-amino-3-[[7-[4-(dimethylamino)phenyl]-1,6-naphthyridin-5-yl]amino]-2-propanol;
N-[7-[4-(dimethylamino)phenyl]-1,6-naphthyridin-5-yl]-2,2-dimethyl-1,3-diaminopropane;
7-[4-(dimethylamino)phenyl]-N-(3-pyridinylmethyl)-1,6-naphthyridin-5-amine;
N-[(2-aminophenyl)methyl]-7-[4-(dimethylamino)phenyl]-1,6-naphthyridin-5-amine;
N-[7-[6-(dimethylamino) [1,1′-biphenyl]-3-yl]-1,6-naphthyridin-5-yl]-1,3-diaminopropane;
N-[7-[3-chloro-4-(diethylamino)phenyl]-1,6-naphthyridin-5-yl]-1,3-diaminopropane;
N-[7-[4-(dimethylamino)-3-methoxyphenyl]-1,6-naphthyridin-5-yl]-1,3-diaminopropane;
N-[7-[4-(diethylamino)phenyl]-3-methyl-1,6-naphthyridin-5-yl]-1,3-diaminopropane;
N-[7-(3′-fluoro[1,1′-biphenyl]-3-yl)-1,6-naphthyridin-5-yl]-1,2-diamino ethane,
N-[7-(4-methoxyphenyl)-1,6-naphthyridin-5-yl]-1,6-Naphthyridine-1,3-diaminopropane;
N,N′-bis(3-aminopropyl)-7-(4-methoxyphenyl)-2,5-diamine;
N-[7-(4-methoxyphenyl)-2-(phenylmethoxy)-1,6-naphthyridin-5-yl]-1,6-naphthyridine-1,3-diaminopropane;
N5-(3-aminopropyl)-7-(4-methoxyphenyl)-N2-(phenylmethyl)-2,5-diamine;
N-[7-(2-naphthalenyl)-1,6-naphthyridin-5-yl]-1,3-diaminopropane;
N-[7-(2′-fluoro[1,1′-biphenyl]-4-yl)-1,6-naphthyridin-5-yl]-1,3-diaminopropane;
N-[7-(3,4,5-trimethoxyphenyl)-1,6-naphthyridin-5-yl]-1,3-diaminopropane;
N-[7-(3,4-dimethylphenyl)-1,6-naphthyridin-5-yl]-1,3-diaminopropane;
1-amino-3-[[7-(2-naphthalenyl)-1,6-naphthyridin-5-yl]amino]-2-propanol;
1-amino-3-[[7-(2′-fluoro[1,1′-biphenyl]-4-yl)-1,6-naphthyridin-5-yl]amino]-2-propanol;
1-amino-3-[[7-(4′-methoxy[1,1′-biphenyl]-4-yl)-1,6-naphthyridin-5-yl]amino]-2-propanol;
1-amino-3-[[7-(3,4,5-trimethoxyphenyl)-1,6-naphthyridin-5-yl]amino]-2-propanol;
1-amino-3-[[7-(4-bromophenyl)-1,6-naphthyridin-5-yl]amino]-2-propanol;
N-[7-(4′-methoxy[1,1′-biphenyl]-4-yl)-1,6-naphthyridin-5-yl]-2,2-dimethyl-1,3-diaminopropane;
1-[[7-[4-(dimethylamino)phenyl]-1,6-naphthyridin-5-yl]amino]-2-propanol;
2-[[2-[[7-[4-(dimethylamino)phenyl]-1,6-naphthyridin-5-yl]amino]ethyl]thio]-ethanol;
7-[4-(dimethylamino)phenyl]-N-(3-methyl-5-isoxazolyl)-1,6-naphthyridin-5-amine;
7-[4-(dimethylamino)phenyl]-N-4-pyrimidinyl-1,6-naphthyridin-5-amine;
N-[7-[4-(dimethylamino)phenyl]-1,6-naphthyridin-5-yl]-1,3-diaminocyclohexane;
N,N-dimethyl-4-[5-(1-piperazinyl)-1,6-naphthyridin-7-yl]-benzenamine;
4-[5-(2-methoxyethoxy)-1,6-naphthyridin-7-yl]-N,N-dimethyl-benzenamine;
1-[7-[4-(dimethylamino)phenyl]-1,6-naphthyridin-5-yl]-4-piperidinol;
1-[7-[4-(dimethylamino)phenyl]-1,6-naphthyridin-5-yl]-3-pyrrolidinol;
7-[4-(dimethylamino)phenyl]-N-(2-furanylmethyl)-1,6-naphthyridin-5-amine;
7-[4-(dimethylamino)phenyl]-N-[3-(1H-imidazol-1-yl)propyl]-1,6-naphthyridin-5-amine;
1-[7-[4-(dimethylamino)phenyl]-1,6-naphthyridin-5-yl]-4-piperidinecarboxamide;
1-[3-[[7-[4-(dimethylamino)phenyl]-1,6-naphthyridin-5-yl]amino]propyl]-2-pyrrolidinone;
N-[3′-[5-[(3-aminopropyl)amino]-1,6-naphthyridin-7-yl][1,1′-biphenyl]-3-yl]-acetamide;
N-[7-(4′-fluoro[1,1′-biphenyl]-4-yl)-1,6-naphthyridin-5-yl]-1,3-diaminopropane;
N-[4′-[5-[(3-aminopropyl)amino]-1,6-naphthyridin-7-yl][1,1′-biphenyl]-3-yl]-acetamide;
N-[7-[4-(1,3-benzodioxol-5-yl)phenyl]-1,6-naphthyridin-5-yl]-1,3-diaminopropane;
N-[7-[4-(2-thienyl)phenyl]-1,6-naphthyridin-5-yl]-1,3-diaminopropane;
N-[7-[4-fluoro-3-(trifluoromethyl)phenyl]-1,6-naphthyridin-5-yl]-1,3-diaminopropane;
N-[7-[4-(3-pyridinyl)phenyl]-1,6-naphthyridin-5-yl]-1,3-diaminopropane;
N-[7-(1,3-benzodioxol-5-yl)-1,6-naphthyridin-5-yl]-1,3-diaminopropane;
N-[7-(6-methoxy-2-naphthalenyl)-1,6-naphthyridin-5-yl]-1,3-diaminopropane;
7-[4-(dimethylamino)phenyl]-N-(4-pyridinylmethyl)-1,6-naphthyridin-5-amine;
3-[[7-[4-(dimethylamino)phenyl]-1,6-naphthyridin-5-yl]methylamino]-propanenitrile;
7-[4-(dimethylamino)phenyl]-N-[1-(phenylmethyl)-4-piperidinyl]-1,6-naphthyridin-5-amine;
N-[7-[4-(dimethylamino)phenyl]-1,6-naphthyridin-5-yl]-1,2-diaminocyclohexane,
   optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-14 MAP Kinase Inhibitors

Examples of preferred MAP kinase inhibitors, as for example p38, ERK1, ERK2, JNK1, JNK2, JNK3 or SAP, which may be mentioned include SB-681323, GW-856553, PH-797804, BMS-582949, SCIO-323, SX-011, SD-282, SD-169, NPC-037282, SX-004, VX-702, GSK-681323, GSK-856553, ARRY-614, ARRY-797, ARRY-438162, ARRY-p38-002, ARRY-371797, AS-602801, AS-601245, AS-602183, CEP-1347, KC706, TA-5493, RO-6226, Ro-1487, SC-409, CBS-3595, VGX-1027, PH-797804, BMS-582949, TA-5493 and BIRB-796 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate

### 2-15 Inhibitors of the NF-kB Signalling Pathway

Examples of preferred inhibitors of the NF-kB signalling pathway including IKK2 kinase inhibitors which may be mentioned include: MD-1041, MLN-041 and AVE-0547 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

2-16 iNOS-Inhibitors

Examples of preferred iNOS-Inhibitors which may be mentioned include S-(2-Aminoethyl)isothio-urea, Aminoguanidin, 2-Aminomethylpyridin, AMT, L-Canavanin, 2-Iminopiperidin, S-Isopropylisothioharnstoff, S-Methylisothio-urea, S-Ethylisothioharnstoff, S-Methyltiocitrullin, 5-Ethylthiocitrullin, L-NA (N^{ω}-Nitro-L-arginin), L-NAME (N^{ω}-Nitro-L-argininmethylester), L-NMMA (N^{ω}-Monomethyl-L-arginin), L-NIO (N^{ω}-Iminoethyl-L-ornithin), L-NIL (N^{ω}-Iminoethyl-lysin), (S)-6-acetimidoylamino-2-amino-hexanoic acid (1H-tetrazol-5-yl)-amide (SC-51), 1400W, (S)-4-(2-acetimidoylamino-ethylsulfanyl)-2-amino-butyric acid (GW274150), 2-[2-(4-methoxy-pyridin-2-yl)-ethyl]-3H-imidazo[4,5-b]pyridin (BYK191023), 2-((R)-3-amino-1-phenyl-propoxy)-4-chloro-5-fluorobenzonitrile, 2-((1R,3S)-3-amino-4-hydroxy-1-thiazol-5-yl-butylsulfanyl)-6-trifluoromethyl-nicotinonitrile, 2-((1R,3S)-3-amino-4-hydroxy-1-thiazol-5-yl-butylsulfanyl)-4-chloro-benzonitrile, 2-((1R,3S)-3-amino-4-hydroxy-1-thiazol-5-yl-butylsulfanyl)-5-chloro-benzonitrile, (2S,4R)-2-amino-4-(2-chloro-5-trifluoromethyl-phenylsulfanyl)-4-thiazol-5-yl-butan-1-ol, 2-((1R,3S)-3-amino-4-hydroxy-1-thiazol-5-yl-butylsulfanyl)-5-chloro-nicotinonitrile, 4-((S)-3-amino-4-hydroxy-1-phenyl-butylsulfanyl)-6-methoxy-nicotinonitrile, substituted 3-phenyl-3,4-dihydro-1-isoquinolinamines, e.g. AR-C102222, (1S,5S,6R)-7-chloro-5-methyl-2-aza-bicyclo[4.1.0]hept-2-en-3-ylamin (ONO-1714), (4R,5R)-5-ethyl-4-methyl-thiazolidin-2-ylideneamin, (4R,5R)-5-ethyl-4-methyl-selenazolidin-2-ylideneamin, 4-aminotetrahydrobiopterin, (E)-3-(4-chloro-phenyl)-N-(1-{2-oxo-2-[4-(6-trifluoromethyl-pyrimidin-4-yloxy)-piperidin-1-yl]-ethylcarbamoyl}-2-pyridin-2-yl-ethyl)-acrylamide (FR260330), 3-(2,4-difluoro-phenyl)-6-[2-(4-imidazol-1-ylmethyl-phenoxy)-ethoxy]-2-phenyl-pyridine (PPA250), 3-{[(benzo[1,3]dioxol-5-ylmethyl)-carbamoyl]-methyl}-4-(2-imidazol-1-yl-pyrimidin-4-yl)-piperazin-1-carboxylic acid methyl ester (BBS-1), (R)-1-(2-imidazol-1-yl-6-methyl-pyrimidin-4-yl)-pyrrolidin-2-carboxylic acid (2-benzo[1,3]dioxol-5-yl-ethyl)-amide (BBS-2), optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-17 MPR4 Inhibitors

Examples of preferred MRP4-Inhibitors which may be mentioned include N-acetyl-dinitrophenyl-Cysteine, cGMP, cholate, diclofenac, dehydroepiandrosterone 3-glucuronide, dehydroepiandrosterone 3-sulphate, dilazep, dinitrophenyl-5-glutathione, estradiol 17-β-glucuronide, estradiol 3,17-disulphate, estradiol 3-glucuronide, estradiol 3-sulphate, estrone 3-sulphate, flurbiprofen, folate, N5-formyl-tetrahydrofolate, glycocholate, glycolithocholic acid sulphate, ibuprofen, indomethacin, indoprofen, ketoprofen, lithocholic acid sulphate, methotrexate, MK571 ((E)-3-[[[3-[2-(7-chloro-2-quinolinyl)ethenyl]phenyl]-[[3-dimethylamino)-3-oxopropyl]thio]methyl]thio]-propanoic acid), α-naphthyl-β-D-glucuronide, nitrobenzyl mercaptopurine riboside, probenecid, PSC833, sildenafil, sulfinpyrazone, taurochenodeoxycholate, taurocholate, taurodeoxycholate, taurolithocholate, taurolithocholic acid sulphate, topotecan, trequinsin, zaprinast and dipyridamol, optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

Especially preferred are N-Acetyl-dinitrophenyl-Cysteine, Dehydroepiandrosterone 3-sulphate, Dilazep, Dinitrophenyl-S-glutathione, Estradiol 3,17-disulphate, Flurbiprofen, Glycocholate, Glycolithocholic acid sulphate, Ibuprofen, Indomethacin, Indoprofen, Lithocholic acid sulphate, MK571, PSC833, Sildenafil, Taurochenodeoxycholate, Taurocholate, Taurolithocholate, Taurolithocholic acid sulphate, Trequinsin, Zaprinast and Dipyridamol, optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-17 Leukotriene Biosynthesis Inhibitors

Examples of preferred Leukotriene biosynthesis inhibitors, as for example 5-Lipoxygenase (5-LO) inhibitors, cPLA2 inhibitors, Leukotriene A4 hydrolase inhibitors oder FLAP inhibitors, which may be mentioned include zileuton, tipelukast, licofelone, darapladib, TA-270, IDEA-033, IDEA-070, NIK-639, ABT-761, fenleuton, tepoxalin, AM-103, AM-803, Abbott-79175, Abbott-85761, PLT-3514, CMI-903, PEP-03, CMI-977, MLN-977, CMI-947, LDP-977, efipladib, PLA-695, veliflapon, MK-591, MK-886 and BAYx1005 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-19 Non-Steroidal Anti-Inflammatory Agents

Examples of preferred non-steroidal anti-inflammatory agents (NSAIDs) which may be mentioned include Piroxicam, Diclofenac, Naproxen, Flurbiprofen, Fenoprofen, Ketoprofen, Ibuprofen, Nimesulide, Indomethacin, Sulindac, Azapropazone, Phenylbutazone, Aspirin; Meloxicam, Celecoxib, Rofecoxib, Valdecoxib, Lumarocoxib, Parecoxib, Tenoxicam and Etoricoxib, optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-20 DP1 Receptor Modulators

Examples of preferred DP1 receptor modulators which may be mentioned include S-5751, laropiprant, SAR-389644, S-555739 and TS-002, optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-21 Thromboxane Receptor Antagonists

Examples of preferred thromboxane receptor antagonists which may be mentioned include Ramatroban, Seratrodast, BM-573, (+/−)-sodium[2-(4-chlorophenylsulfonylaminomethyl)-indan-5-yl]acetate monohydrate (Z-335) and KP-496, optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-22 CCR1 Antagonists

Examples of preferred CCR1 antagonists which may be mentioned include AZD-4818, CCX-354, MLN-3701, MLN-3897, optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-23 CCR2 Antagonists

Examples of preferred CCR2 antagonists which may be mentioned include CCX-140, INCB-8696, BMS-741672, SSR-150106, JNJ-17166864 and MLN-1202 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-24 CCR3 Antagonists

Examples of preferred CCR3 antagonists which may be mentioned include GW766994, AZD 1744 and BMS-639623 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-25 CCR4 Antagonists

Examples of preferred CCR4 antagonists which may be mentioned include KW-0761 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-26 CCR5 Antagonists

Examples of preferred CCR5 antagonists which may be mentioned include maraviroc, vicriviroc, nifeviroc, INCB-15050, CCR5 mAb004, GSK-706769, PRO-140, and SCH-532706, optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-27 CCR9 Antagonists

Examples of preferred CCR9 antagonists which may be mentioned include Traficet-E (CCX282) and MLN-3126 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-28 CXCR1 or CXCR2 Antagonists

Examples of preferred CXCR1 or CXCR2 antagonists which may be mentioned include SCH-527123 and SB-656933 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-29 CXCR4 Antagonists

Examples of preferred CXCR4 antagonists which may be mentioned include CTCE-0214, MSX-122, POL-2438, POL-6326, POL-3026, TG-0054 and AMD3100 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-30 Neurokinin (NK1 or NK2) Antagonists

Examples of preferred Neurokinin (NK1 or NK2) antagonists which may be mentioned include Saredutant, Figopitant, Nepadutant, Rolapitant, LY-686017, PRX-96026 and optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-31 Sphingosine 1-Phosphate Receptor Modulators or Sphingosine 1 Phosphate Lyase Inhibitors

Examples of preferred Sphingosine 1-phosphate receptor modulators or Sphingosine 1 phosphate lyase inhibitors which may be mentioned include c-6448, FTY720, LX-2931, sonepcizumab, BAF-312, ONO-4641 and R-3477 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-32 Adenosine Receptor Modulators

Examples of preferred adenosine receptor modulators, including A2a agonists, which may be mentioned include CGH-2466, CVT-6883, MRS-1754, UK-432097 and L-971 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-33 Purinergic Receptor Modulators

Examples of preferred purinergic receptor modulators, including P2X7 inhibitors, which may be mentioned include AZD-9056 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-34 Bradykinin Receptor Antagonists

Examples of preferred bradykinin receptor antagonists which may be mentioned include Icatibant, AMG-379 and MEN-16132 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-35 PPAR Gamma Modulators

Examples of preferred PPAR gamma modulators which may be mentioned include Rosiglitazone, Ciglitazone, Pioglitazone and SMP-028 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate;

Examples of preferred Rho kinase inhibitors which may be mentioned include Fasudil optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-36 Interleukin 1-Beta Converting Enzyme

Examples of preferred Interleukin 1-beta converting enzyme (ICE) inhibitors which may be mentioned include Pralnacasan, VRT-18858, RU-36384, VX-765 and VRT-43198 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-37 Toll-Like Receptor

Examples of preferred Toll-like receptor (TLR) modulators which may be mentioned include Resiquimod, PF-3512676, AVE-0675, Heplisav, IMO-2055, CpG-28, TAK-242, SAR-21609, RC-52743198 and 852A optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-38 HMG-CoA Reductase Inhibitors

Examples of preferred HMG-CoA reductase inhibitors which may be mentioned include Lovastatin, Simvastatin, Pravastatin, Fluvastatin and Avorvastatin optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-39 VLA4 Antagonists

Examples of preferred VLA4 antagonists which may be mentioned include Natalizumab, Valategrast, TBC-4746, CDP-323 and TL-1102 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-40 ICAM-1 Inhibitors

Examples of preferred ICAM-1 inhibitors which may be mentioned include BIRT-2584 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-41 SHIP Agonists

Examples of preferred SHIP agonists which may be mentioned include AQX-MN100 and MN-106 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-42 Melanocortin Receptor (MC1R, MC2R, MC3R, MC4R, MC5R) Modulators

Examples of preferred melanocortin receptor (MC1R, MC2R, MC3R, MC4R, MC5R) modulators which may be mentioned include AP-1030 and AP-214 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-43 Endothelin Antagonists

Examples of preferred endothelin antagonists which may be mentioned include Actelion-1, Ambrisentan, Sitaxsentan, TBC-3711, TBC-3214 and Bosentan

optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-44 Mucoregulators

Examples of preferred mucoregulators which may be mentioned include Acetylcysteine, Ambroxol, Bromhexine, Carbocisteine, Domiodol, Dornase alfa, Eprazinone, Erdosteine, Fluorovent, Talniflumate, MSI-2216, INO-4995, BIO-11006, VR-496 and fudosteine optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-45 Agents Against Swelling of the Airways

Examples of preferred agents against swelling of the airways which may be mentioned include Phenylephrine, Phenylpropanolamine, Pseudophedrine, Oxymetazoline, Epinephrine, Naphazoline, Xylometazoline, Propylhexedrine and Llevo-desoxyephedrine optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-46 Agents Against Cough

Examples of preferred agents against cough which may be mentioned include Hydrocodone, Caramiphen, Carbetapentane and Dextramethorphan optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-47 CB2 Agonists

Examples of preferred CB2 agonists which may be mentioned include GRC-10693, HU-308, JWH-015, JWH-133, L-759,633 and L-759,656 optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-48 Retinoids

Examples of preferred retinoids which may be mentioned include Acitretin optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-49 Immunosuppressants

Examples of preferred immunosuppressants which may be mentioned include Mycophenolate, Cyclosporine, Azathioprine, Penicillamine and Leflunomide optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-50 Mast Cell Stabilizers

Examples of preferred mast cell stabilizers which may be mentioned include cromoglycate optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate and hydro-p-toluenesulfonate.

### 2-51 5-HT4 Agonists

Examples of preferred 5-HT4 agonists which may be mentioned include tegaserod, Mosapride, Prucalopride.

### 2-52 Agents in the Treatment of Inflammatory Bowel Disease

Examples of preferred systemic glucocorticosteroids include Betamethasone, Budesonide, Cortisone, Dexamethasone, Hydrocortisone, Methylprednisolone, Prednisolone, Prednisone, Tixocortal, Triamcinolone.

### 2-53 Sulfonamides

Examples of preferred sulfonamides which may be mentioned include Sulfasalazine, and Sulfapyradine.

### 2-54 Amino-Salicylates

Examples of preferred amino-salicylates which may be mentioned include Belsalazide, Mesalazine, Olzalazine, and Sulfasalazine.

### 2-55 Immunosuppressive Agents

Examples of orally administered immunosuppressive agents which may be mentioned include Azathioprine, Cyclosporine, Mercaptopurine, Methotrexate, and Tacrolimus.

### 2-56 Opioid Receptor Agonists

Preferred are loperamide and diphenoxylate.

### 2-57 Antispasmodic Agents

Preferred are mebeverine and alverine.

Especially preferred further active compounds 2 are PDE4 inhibitors 2-4. Particularly preferred is Roflumilast.

Especially preferred further active compounds 2 are LTD4 antagonists 2-5. Particularly preferred are montelukast, pranlukast and zafirlukast.

Especially preferred further active compounds 2 are Histamine receptor antagonists 2-11. Particularly preferred are azelastine, cetirizine, desloratidine, ebastine, epinastine, fexofenadine, hydroxyzine, ketotifen, levocetirizine, loratadine and olopatadine.

Especially preferred further active compounds 2 are 5-LO inhibitors 2-17. Particularly preferred is Zileuton.

Especially preferred further active compounds 2 are CCR5 antagonists 2-26. Particularly preferred is Maraviroc.

Especially preferred further active compounds 2 are CCR9 antagonists 2-27. Particularly preferred is Trafficet.

Especially preferred further active compounds 2 are Sulfonamides 2-53. Particularly preferred is Mesalazine and Sulfasalazine.

In the pharmaceutical compositions according to the present invention the CRTH2 antagonists 1 may be contained in a form selected from tautomers, optical isomers, enantiomers, racemates, diastereomers, pharmacologically acceptable acid addition salts, solvates or hydrates, as far as such forms exist, depending on the individual compound. Pharmaceutical compositions comprising one or more, preferably one, compound 1 in form of a substantially pure enantiomer are preferred.

In the pharmaceutical compositions according to the present invention more than one CRTH2 antagonists 1 and more than one further active compound 2 can be present.

In an especially preferred embodiment herein the pharmaceutical composition of the present invention comprises the CRTH2 antagonist 1.136 in combination with any of the further active compounds 2 mentioned herein before or herein after. Particularly preferred are combinations of 1.136 with LTD4 antagonists 2-5, especially montelukast, zafirlukast and pranlukast, in particular montelukast sodium, which is the monosodium salt of montelukast.

Pharmacological acceptable salts of CRTH2 antagonists 1 comprise salts selected from the group consisting of the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrolactate, hydrooxalate, hydrosuccinate, hydrobenzoate and hydro-p-toluolsulphonate, preferably hydrochloride, hydrobromide, hydrosulphate, hydrophosphate, hydromaleate, hydrofumarate and hydromethansulphonate. Some of the compounds 1 may add more than one equivalent acid, e.g. two equivalents.

Preferred salts of the CRTH2 antagonists 1 are base addition salts, especially amine salts from primary amines, including methylamine, ethylamine, ethanolamine, tris(hydroxymethyl)aminomethane, and ethylenediamine; secondary amines, including dimethylamine, diethylamine, diisopropylamine, dibutylamine, di-sec-butylamine, dicyclohexylamine, diethanolamine, meglumine, pyrrolidine, piperidine, piperazine, and benzathine; tertiary amines, including trimethylamine, triethylamine, triethanolamine, and 1-(2-hydroxyethyl)-pyrrolidine; quaternary ammoniums, including choline, tetra-methylammonium, and tetraethylammonium. For a review on additional amines, see “Handbook of Pharmaceutical Salts Properties, Selection, and Use” by Stahl and Wermuth, Wiley-VCH, 2002.

The base addition salts of the CRTH2 antagonists 1, especially 1.136, with ethylenediamine and choline are especially preferred.

In the composition of he present invention the CRTH2 antagonist 1.136 and montelukast sodium are preferably present at a weight ratio of from (1.136:montelukast sodium) 2.5:1 to 40:1. In individual examples of the composition of the present invention the CRTH2 antagonist 1.136 and montelukast sodium are preferably present at a weight ratio of (1.136:montelukast sodium) 1:1; 1.5:1; 2:1; 2.5:1; 3:1; 3.5:1, 4:1; 4.5:1; 5:1; 5.5:1; 6:1; 6.5:1; 7:1; 7.5:1; 8:1; 8.5:1; 9:1; 9.5:1; 10:1; 10.5:1; 11:1; 11.5:1; 12:1; 12.5:1; 13:1; 13.5:1; 14:1; 14.5:1; 15:1; 15.5:1; 16:1; 16.5:1; 17:1; 17.5:1; 18:1; 18.5:1; 19:1; 19.5:1; 20:1; 20.5:1; 21:1; 21.5:1; 22:1; 22.5:1; 23:1; 23.5:1; 24:1; 24.5:1; 25:1; 25.5:1; 26:1; 26.5:1; 27:1; 27.5:1; 28:1; 28.5:1; 29:1; 29.5:1; 30:1; 30.5:1; 31:1; 31.5:1; 32:1; 32.5:1; 33:1; 33.5:1; 34:1; 34.5:1; 35:1; 35.5:1; 36:1; 36.5:1; 37:1; 37.5:1; 38:1; 38.5:1; 39:1; 39.5:1; 40:1.

In other embodiments of the present invention the CRTH2 antagonist 1.136 and zafirlukast are preferably present at a weight ratio of from (1.136:zafirlukast) 2.5:1 to 40:1. In individual examples of the composition of the present invention the CRTH2 antagonist 1.136 and zafirlukast are preferably present at a weight ratio of (1.136:zafirlukast) 1:1; 1.5:1; 2:1; 2.5:1; 3:1; 3.5:1, 4:1; 4.5:1; 5:1; 5.5:1; 6:1; 6.5:1; 7:1; 7.5:1; 8:1; 8.5:1; 9:1; 9.5:1; 10:1; 10.5:1; 11:1; 11.5:1; 12:1; 12.5:1; 13:1; 13.5:1; 14:1; 14.5:1; 15:1; 15.5:1; 16:1; 16.5:1; 17:1; 17.5:1; 18:1; 18.5:1; 19:1; 19.5:1; 20:1; 20.5:1; 21:1; 21.5:1; 22:1; 22.5:1; 23:1; 23.5:1; 24:1; 24.5:1; 25:1; 25.5:1; 26:1; 26.5:1; 27:1; 27.5:1; 28:1; 28.5:1; 29:1; 29.5:1; 30:1; 30.5:1; 31:1; 31.5:1; 32:1; 32.5:1; 33:1; 33.5:1; 34:1; 34.5:1; 35:1; 35.5:1; 36:1; 36.5:1; 37:1; 37.5:1; 38:1; 38.5:1; 39:1; 39.5:1; 40:1.

In other embodiments of the present invention the CRTH2 antagonist 1.136 and pranlukast are preferably present at a weight ratio of from (1.136:pranlukast) 2.5:1 to 40:1. In individual examples of the composition of the present invention the CRTH2 antagonist 1.136 and pranlukast are preferably present at a weight ratio of (1.136:pranlukast) 1:1; 1.5:1; 2:1; 2.5:1; 3:1; 3.5:1, 4:1; 4.5:1; 5:1; 5.5:1; 6:1; 6.5:1; 7:1; 7.5:1; 8:1; 8.5:1; 9:1; 9.5:1; 10:1; 10.5:1; 11:1; 11.5:1; 12:1; 12.5:1; 13:1; 13.5:1; 14:1; 14.5:1; 15:1; 15.5:1; 16:1; 16.5:1; 17:1; 17.5:1; 18:1; 18.5:1; 19:1; 19.5:1; 20:1; 20.5:1; 21:1; 21.5:1; 22:1; 22.5:1; 23:1; 23.5:1; 24:1; 24.5:1; 25:1; 25.5:1; 26:1; 26.5:1; 27:1; 27.5:1; 28:1; 28.5:1; 29:1; 29.5:1; 30:1; 30.5:1; 31:1; 31.5:1; 32:1; 32.5:1; 33:1; 33.5:1; 34:1; 34.5:1; 35:1; 35.5:1; 36:1; 36.5:1; 37:1; 37.5:1; 38:1; 38.5:1; 39:1; 39.5:1; 40:1.

### The Unit Dose Form

The pharmaceutical composition herein comprising the aforementioned amount of the CRTH2 antagonists 1 and of the further active compounds 2 is applied to the patient as a unit dose form. “Unit dose form” herein refers to the actual product, through which the pharmaceutical composition is administered to the patient. Non-limiting examples are tablets, lozenges, capsules, inhalation powder capsules, unit dose vials, metered doses provided by a metered dose inhaler (MDI), injection vials and others commonly known by the skilled artisan.

The pharmaceutical composition can be applied to the patient via the unit dose form in one administration or in more than one sub-administrations. Furthermore, the CRTH2 antagonists 1 and the further active compounds 2 can be applied to the patient in a combined administration or in separate administrations. It is preferred herein that the unit dose form of the present invention is administered to the patient in a combined administration, where the CRTH2 antagonists 1 and the further active compounds 2 are administered together. In one embodiment the daily dosages mentioned herein above are administered to the patient in a three-times-daily (t-d) administration scheme, in another embodiment the daily dosages mentioned herein above are administered to the patient in a twice-daily (b-i-d) administration scheme, and in another embodiment the daily dosages mentioned herein above are administered to the patient in a once-daily (q-d) administration scheme.

In the pharmaceutical composition herein the daily dosage of the CRTH2 antagonists 1 is in the range of from 1 mg to 1000 mg, preferably from 5 mg to 800 mg, preferably from 10 mg to 700 mg, preferably from 15 mg to 600 mg, preferably from 20 mg to 500 mg, preferably from 25 mg to 400 mg.

In the pharmaceutical composition herein the daily dosage of the further active compounds 2 is in the range from 1 mg to 1000 mg, preferably from 2 mg to 800 mg, preferably from 3 mg to 500 mg, preferably from 4 mg to 300 mg, preferably from 5 mg to 200 mg, preferably from 6 mg to 150 mg.

For the preferred LTD4 antagonists 2-5 the daily dosage is in the range of from 7 mg to 100 mg, preferably from 8 mg to 50 mg, preferably from 9 mg to 20 mg, preferably 9 mg to 15 mg, preferably 9 mg to 12 mg.

In a preferred embodiment the pharmaceutical composition of the present invention comprises the CRTH2 antagonist 1.136 in combination with one or more LDT4 antagonists 2-5, wherein 1.136 is present in the composition at a daily dosage of from 25 mg to 400 mg and the one or more LDT4 antagonists 2-5 is present at the composition at a total daily dosage of from 8 mg to 50 mg, preferably from 9 mg to 20 mg. In a particularly preferred embodiment herein CRTH2 antagonist 1.136 is present at a daily dosage of from 25 mg to 400 mg and the LTD4 antagonist montelukast (as montelukast sodium) is present at a daily dosage of from 9 mg to 12 mg, preferably 10 mg.

Based on the data listed herein unit dose forms containing the following amounts of 1.136 and montelukast sodium (MLS) are explicitly encompassed by the present invention:

10 mg 1.136 and 1 mg MLS; 10 mg 1.136 and 2 mg MLS; 10 mg 1.136 and 3 mg MLS; 10 mg 1.136 and 4 mg MLS; 10 mg 1.136 and 5 mg MLS; 10 mg 1.136 and 6 mg MLS; 10 mg 1.136 and 7 mg MLS; 10 mg 1.136 and 8 mg MLS; 10 mg 1.136 and 9 mg MLS; 10 mg 1.136 and 10 mg MLS; 10 mg 1.136 and 11 mg MLS; 10 mg 1.136 and 12 mg MLS; 10 mg 1.136 and 13 mg MLS; 10 mg 1.136 and 14 mg MLS; 10 mg 1.136 and 15 mg MLS; 10 mg 1.136 and 16 mg MLS; 10 mg 1.136 and 17 mg MLS; 10 mg 1.136 and 18 mg MLS; 10 mg 1.136 and 19 mg MLS; 10 mg 1.136 and 20 mg MLS;

15 mg 1.136 and 1 mg MLS; 15 mg 1.136 and 2 mg MLS; 15 mg 1.136 and 3 mg MLS; 15 mg 1.136 and 4 mg MLS; 15 mg 1.136 and 5 mg MLS; 15 mg 1.136 and 6 mg MLS; 15 mg 1.136 and 7 mg MLS; 15 mg 1.136 and 8 mg MLS; 15 mg 1.136 and 9 mg MLS; 15 mg 1.136 and 10 mg MLS; 15 mg 1.136 and 11 mg MLS; 15 mg 1.136 and 12 mg MLS; 15 mg 1.136 and 13 mg MLS; 15 mg 1.136 and 14 mg MLS; 15 mg 1.136 and 15 mg MLS; 15 mg 1.136 and 16 mg MLS; 15 mg 1.136 and 17 mg MLS; 15 mg 1.136 and 18 mg MLS; 15 mg 1.136 and 19 mg MLS; 15 mg 1.136 and 20 mg MLS;

20 mg 1.136 and 1 mg MLS; 20 mg 1.136 and 2 mg MLS; 20 mg 1.136 and 3 mg MLS; 20 mg 1.136 and 4 mg MLS; 20 mg 1.136 and 5 mg MLS; 20 mg 1.136 and 6 mg MLS; 20 mg 1.136 and 7 mg MLS; 20 mg 1.136 and 8 mg MLS; 20 mg 1.136 and 9 mg MLS; 20 mg 1.136 and 10 mg MLS; 20 mg 1.136 and 11 mg MLS; 20 mg 1.136 and 12 mg MLS; 20 mg 1.136 and 13 mg MLS; 20 mg 1.136 and 14 mg MLS; 20 mg 1.136 and 15 mg MLS; 20 mg 1.136 and 16 mg MLS; 20 mg 1.136 and 17 mg MLS; 20 mg 1.136 and 18 mg MLS; 20 mg 1.136 and 19 mg MLS; 20 mg 1.136 and 20 mg MLS;

25 mg 1.136 and 1 mg MLS; 25 mg 1.136 and 2 mg MLS; 25 mg 1.136 and 3 mg MLS; 25 mg 1.136 and 4 mg MLS; 25 mg 1.136 and 5 mg MLS; 25 mg 1.136 and 6 mg MLS; 25 mg 1.136 and 7 mg MLS; 25 mg 1.136 and 8 mg MLS; 25 mg 1.136 and 9 mg MLS; 25 mg 1.136 and 10 mg MLS; 25 mg 1.136 and 11 mg MLS; 25 mg 1.136 and 12 mg MLS; 25 mg 1.136 and 13 mg MLS; 25 mg 1.136 and 14 mg MLS; 25 mg 1.136 and 15 mg MLS; 25 mg 1.136 and 16 mg MLS; 25 mg 1.136 and 17 mg MLS; 25 mg 1.136 and 18 mg MLS; 25 mg 1.136 and 19 mg MLS; 25 mg 1.136 and 20 mg MLS;

30 mg 1.136 and 1 mg MLS; 30 mg 1.136 and 2 mg MLS; 30 mg 1.136 and 3 mg MLS; 30 mg 1.136 and 4 mg MLS; 30 mg 1.136 and 5 mg MLS; 30 mg 1.136 and 6 mg MLS; 30 mg 1.136 and 7 mg MLS; 30 mg 1.136 and 8 mg MLS; 30 mg 1.136 and 9 mg MLS; 30 mg 1.136 and 10 mg MLS; 30 mg 1.136 and 11 mg MLS; 30 mg 1.136 and 12 mg MLS; 30 mg 1.136 and 13 mg MLS; 30 mg 1.136 and 14 mg MLS; 30 mg 1.136 and 15 mg MLS; 30 mg 1.136 and 16 mg MLS; 30 mg 1.136 and 17 mg MLS; 30 mg 1.136 and 18 mg MLS; 30 mg 1.136 and 19 mg MLS; 30 mg 1.136 and 20 mg MLS;

35 mg 1.136 and 1 mg MLS; 35 mg 1.136 and 2 mg MLS; 35 mg 1.136 and 3 mg MLS; 35 mg 1.136 and 4 mg MLS; 35 mg 1.136 and 5 mg MLS; 35 mg 1.136 and 6 mg MLS; 35 mg 1.136 and 7 mg MLS; 35 mg 1.136 and 8 mg MLS; 35 mg 1.136 and 9 mg MLS; 35 mg 1.136 and 10 mg MLS; 35 mg 1.136 and 11 mg MLS; 35 mg 1.136 and 12 mg MLS; 35 mg 1.136 and 13 mg MLS; 35 mg 1.136 and 14 mg MLS; 35 mg 1.136 and 15 mg MLS; 35 mg 1.136 and 16 mg MLS; 35 mg 1.136 and 17 mg MLS; 35 mg 1.136 and 18 mg MLS; 35 mg 1.136 and 19 mg MLS; 35 mg 1.136 and 20 mg MLS;

40 mg 1.136 and 1 mg MLS; 40 mg 1.136 and 2 mg MLS; 40 mg 1.136 and 3 mg MLS; 40 mg 1.136 and 4 mg MLS; 40 mg 1.136 and 5 mg MLS; 40 mg 1.136 and 6 mg MLS; 40 mg 1.136 and 7 mg MLS; 40 mg 1.136 and 8 mg MLS; 40 mg 1.136 and 9 mg MLS; 40 mg 1.136 and 10 mg MLS; 40 mg 1.136 and 11 mg MLS; 40 mg 1.136 and 12 mg MLS; 40 mg 1.136 and 13 mg MLS; 40 mg 1.136 and 14 mg MLS; 40 mg 1.136 and 15 mg MLS; 40 mg 1.136 and 16 mg MLS; 40 mg 1.136 and 17 mg MLS; 40 mg 1.136 and 18 mg MLS; 40 mg 1.136 and 19 mg MLS; 40 mg 1.136 and 20 mg MLS;

45 mg 1.136 and 1 mg MLS; 45 mg 1.136 and 2 mg MLS; 45 mg 1.136 and 3 mg MLS; 45 mg 1.136 and 4 mg MLS; 45 mg 1.136 and 5 mg MLS; 45 mg 1.136 and 6 mg MLS; 45 mg 1.136 and 7 mg MLS; 45 mg 1.136 and 8 mg MLS; 45 mg 1.136 and 9 mg MLS; 45 mg 1.136 and 10 mg MLS; 45 mg 1.136 and 11 mg MLS; 45 mg 1.136 and 12 mg MLS; 45 mg 1.136 and 13 mg MLS; 45 mg 1.136 and 14 mg MLS; 45 mg 1.136 and 15 mg MLS; 45 mg 1.136 and 16 mg MLS; 45 mg 1.136 and 17 mg MLS; 45 mg 1.136 and 18 mg MLS; 45 mg 1.136 and 19 mg MLS; 45 mg 1.136 and 20 mg MLS;

50 mg 1.136 and 1 mg MLS; 50 mg 1.136 and 2 mg MLS; 50 mg 1.136 and 3 mg MLS; 50 mg 1.136 and 4 mg MLS; 50 mg 1.136 and 5 mg MLS; 50 mg 1.136 and 6 mg MLS; 50 mg 1.136 and 7 mg MLS; 50 mg 1.136 and 8 mg MLS; 50 mg 1.136 and 9 mg MLS; 50 mg 1.136 and 10 mg MLS; 50 mg 1.136 and 11 mg MLS; 50 mg 1.136 and 12 mg MLS; 50 mg 1.136 and 13 mg MLS; 50 mg 1.136 and 14 mg MLS; 50 mg 1.136 and 15 mg MLS; 50 mg 1.136 and 16 mg MLS; 50 mg 1.136 and 17 mg MLS; 50 mg 1.136 and 18 mg MLS; 50 mg 1.136 and 19 mg MLS; 50 mg 1.136 and 20 mg MLS;

55 mg 1.136 and 1 mg MLS; 55 mg 1.136 and 2 mg MLS; 55 mg 1.136 and 3 mg MLS; 55 mg 1.136 and 4 mg MLS; 55 mg 1.136 and 5 mg MLS; 55 mg 1.136 and 6 mg MLS; 55 mg 1.136 and 7 mg MLS; 55 mg 1.136 and 8 mg MLS; 55 mg 1.136 and 9 mg MLS; 55 mg 1.136 and 10 mg MLS; 55 mg 1.136 and 11 mg MLS; 55 mg 1.136 and 12 mg MLS; 55 mg 1.136 and 13 mg MLS; 55 mg 1.136 and 14 mg MLS; 55 mg 1.136 and 15 mg MLS; 55 mg 1.136 and 16 mg MLS; 55 mg 1.136 and 17 mg MLS; 55 mg 1.136 and 18 mg MLS; 55 mg 1.136 and 19 mg MLS; 55 mg 1.136 and 20 mg MLS;

60 mg 1.136 and 1 mg MLS; 60 mg 1.136 and 2 mg MLS; 60 mg 1.136 and 3 mg MLS; 60 mg 1.136 and 4 mg MLS; 60 mg 1.136 and 5 mg MLS; 60 mg 1.136 and 6 mg MLS; 60 mg 1.136 and 7 mg MLS; 60 mg 1.136 and 8 mg MLS; 60 mg 1.136 and 9 mg MLS; 60 mg 1.136 and 10 mg MLS; 60 mg 1.136 and 11 mg MLS; 60 mg 1.136 and 12 mg MLS; 60 mg 1.136 and 13 mg MLS; 60 mg 1.136 and 14 mg MLS; 60 mg 1.136 and 15 mg MLS; 60 mg 1.136 and 16 mg MLS; 60 mg 1.136 and 17 mg MLS; 60 mg 1.136 and 18 mg MLS; 60 mg 1.136 and 19 mg MLS; 60 mg 1.136 and 20 mg MLS;

65 mg 1.136 and 1 mg MLS; 65 mg 1.136 and 2 mg MLS; 65 mg 1.136 and 3 mg MLS; 65 mg 1.136 and 4 mg MLS; 65 mg 1.136 and 5 mg MLS; 65 mg 1.136 and 6 mg MLS; 65 mg 1.136 and 7 mg MLS; 65 mg 1.136 and 8 mg MLS; 65 mg 1.136 and 9 mg MLS; 65 mg 1.136 and 10 mg MLS; 65 mg 1.136 and 11 mg MLS; 65 mg 1.136 and 12 mg MLS; 65 mg 1.136 and 13 mg MLS; 65 mg 1.136 and 14 mg MLS; 65 mg 1.136 and 15 mg MLS; 65 mg 1.136 and 16 mg MLS; 65 mg 1.136 and 17 mg MLS; 65 mg 1.136 and 18 mg MLS; 65 mg 1.136 and 19 mg MLS; 65 mg 1.136 and 20 mg MLS;

70 mg 1.136 and 1 mg MLS; 70 mg 1.136 and 2 mg MLS; 70 mg 1.136 and 3 mg MLS; 70 mg 1.136 and 4 mg MLS; 70 mg 1.136 and 5 mg MLS; 70 mg 1.136 and 6 mg MLS; 70 mg 1.136 and 7 mg MLS; 70 mg 1.136 and 8 mg MLS; 70 mg 1.136 and 9 mg MLS; 70 mg 1.136 and 10 mg MLS; 70 mg 1.136 and 11 mg MLS; 70 mg 1.136 and 12 mg MLS; 70 mg 1.136 and 13 mg MLS; 70 mg 1.136 and 14 mg MLS; 70 mg 1.136 and 15 mg MLS; 70 mg 1.136 and 16 mg MLS; 70 mg 1.136 and 17 mg MLS; 70 mg 1.136 and 18 mg MLS; 70 mg 1.136 and 19 mg MLS; 70 mg 1.136 and 20 mg MLS;

75 mg 1.136 and 1 mg MLS; 75 mg 1.136 and 2 mg MLS; 75 mg 1.136 and 3 mg MLS; 75 mg 1.136 and 4 mg MLS; 75 mg 1.136 and 5 mg MLS; 75 mg 1.136 and 6 mg MLS; 75 mg 1.136 and 7 mg MLS; 75 mg 1.136 and 8 mg MLS; 75 mg 1.136 and 9 mg MLS; 75 mg 1.136 and 10 mg MLS; 75 mg 1.136 and 11 mg MLS; 75 mg 1.136 and 12 mg MLS; 75 mg 1.136 and 13 mg MLS; 75 mg 1.136 and 14 mg MLS; 75 mg 1.136 and 15 mg MLS; 75 mg 1.136 and 16 mg MLS; 75 mg 1.136 and 17 mg MLS; 75 mg 1.136 and 18 mg MLS; 75 mg 1.136 and 19 mg MLS; 75 mg 1.136 and 20 mg MLS;

80 mg 1.136 and 1 mg MLS; 80 mg 1.136 and 2 mg MLS; 80 mg 1.136 and 3 mg MLS; 80 mg 1.136 and 4 mg MLS; 80 mg 1.136 and 5 mg MLS; 80 mg 1.136 and 6 mg MLS; 80 mg 1.136 and 7 mg MLS; 80 mg 1.136 and 8 mg MLS; 80 mg 1.136 and 9 mg MLS; 80 mg 1.136 and 10 mg MLS; 80 mg 1.136 and 11 mg MLS; 80 mg 1.136 and 12 mg MLS; 80 mg 1.136 and 13 mg MLS; 80 mg 1.136 and 14 mg MLS; 80 mg 1.136 and 15 mg MLS; 80 mg 1.136 and 16 mg MLS; 80 mg 1.136 and 17 mg MLS; 80 mg 1.136 and 18 mg MLS; 80 mg 1.136 and 19 mg MLS; 80 mg 1.136 and 20 mg MLS;

85 mg 1.136 and 1 mg MLS; 85 mg 1.136 and 2 mg MLS; 85 mg 1.136 and 3 mg MLS; 85 mg 1.136 and 4 mg MLS; 85 mg 1.136 and 5 mg MLS; 85 mg 1.136 and 6 mg MLS; 85 mg 1.136 and 7 mg MLS; 85 mg 1.136 and 8 mg MLS; 85 mg 1.136 and 9 mg MLS; 85 mg 1.136 and 10 mg MLS; 85 mg 1.136 and 11 mg MLS; 85 mg 1.136 and 12 mg MLS; 85 mg 1.136 and 13 mg MLS; 85 mg 1.136 and 14 mg MLS; 85 mg 1.136 and 15 mg MLS; 85 mg 1.136 and 16 mg MLS; 85 mg 1.136 and 17 mg MLS; 85 mg 1.136 and 18 mg MLS; 85 mg 1.136 and 19 mg MLS; 85 mg 1.136 and 20 mg MLS;

90 mg 1.136 and 1 mg MLS; 90 mg 1.136 and 2 mg MLS; 90 mg 1.136 and 3 mg MLS; 90 mg 1.136 and 4 mg MLS; 90 mg 1.136 and 5 mg MLS; 90 mg 1.136 and 6 mg MLS; 90 mg 1.136 and 7 mg MLS; 90 mg 1.136 and 8 mg MLS; 90 mg 1.136 and 9 mg MLS; 90 mg 1.136 and 10 mg MLS; 90 mg 1.136 and 11 mg MLS; 90 mg 1.136 and 12 mg MLS; 90 mg 1.136 and 13 mg MLS; 90 mg 1.136 and 14 mg MLS; 90 mg 1.136 and 15 mg MLS; 90 mg 1.136 and 16 mg MLS; 90 mg 1.136 and 17 mg MLS; 90 mg 1.136 and 18 mg MLS; 90 mg 1.136 and 19 mg MLS; 90 mg 1.136 and 20 mg MLS;

95 mg 1.136 and 1 mg MLS; 95 mg 1.136 and 2 mg MLS; 95 mg 1.136 and 3 mg MLS; 95 mg 1.136 and 4 mg MLS; 95 mg 1.136 and 5 mg MLS; 95 mg 1.136 and 6 mg MLS; 95 mg 1.136 and 7 mg MLS; 95 mg 1.136 and 8 mg MLS; 95 mg 1.136 and 9 mg MLS; 95 mg 1.136 and 10 mg MLS; 95 mg 1.136 and 11 mg MLS; 95 mg 1.136 and 12 mg MLS; 95 mg 1.136 and 13 mg MLS; 95 mg 1.136 and 14 mg MLS; 95 mg 1.136 and 15 mg MLS; 95 mg 1.136 and 16 mg MLS; 95 mg 1.136 and 17 mg MLS; 95 mg 1.136 and 18 mg MLS; 95 mg 1.136 and 19 mg MLS; 95 mg 1.136 and 20 mg MLS;

100 mg 1.136 and 1 mg MLS; 100 mg 1.136 and 2 mg MLS; 100 mg 1.136 and 3 mg MLS; 100 mg 1.136 and 4 mg MLS; 100 mg 1.136 and 5 mg MLS; 100 mg 1.136 and 6 mg MLS; 100 mg 1.136 and 7 mg MLS; 100 mg 1.136 and 8 mg MLS; 100 mg 1.136 and 9 mg MLS; 100 mg 1.136 and 10 mg MLS; 100 mg 1.136 and 11 mg MLS; 100 mg 1.136 and 12 mg MLS; 100 mg 1.136 and 13 mg MLS; 100 mg 1.136 and 14 mg MLS; 100 mg 1.136 and 15 mg MLS; 100 mg 1.136 and 16 mg MLS; 100 mg 1.136 and 17 mg MLS; 100 mg 1.136 and 18 mg MLS; 100 mg 1.136 and 19 mg MLS; 100 mg 1.136 and 20 mg MLS;

105 mg 1.136 and 1 mg MLS; 105 mg 1.136 and 2 mg MLS; 105 mg 1.136 and 3 mg MLS; 105 mg 1.136 and 4 mg MLS; 105 mg 1.136 and 5 mg MLS; 105 mg 1.136 and 6 mg MLS; 105 mg 1.136 and 7 mg MLS; 105 mg 1.136 and 8 mg MLS; 105 mg 1.136 and 9 mg MLS; 105 mg 1.136 and 10 mg MLS; 105 mg 1.136 and 11 mg MLS; 105 mg 1.136 and 12 mg MLS; 105 mg 1.136 and 13 mg MLS; 105 mg 1.136 and 14 mg MLS; 105 mg 1.136 and 15 mg MLS; 105 mg 1.136 and 16 mg MLS; 105 mg 1.136 and 17 mg MLS; 105 mg 1.136 and 18 mg MLS; 105 mg 1.136 and 19 mg MLS; 105 mg 1.136 and 20 mg MLS;

110 mg 1.136 and 1 mg MLS; 110 mg 1.136 and 2 mg MLS; 110 mg 1.136 and 3 mg MLS; 110 mg 1.136 and 4 mg MLS; 110 mg 1.136 and 5 mg MLS; 110 mg 1.136 and 6 mg MLS; 110 mg 1.136 and 7 mg MLS; 110 mg 1.136 and 8 mg MLS; 110 mg 1.136 and 9 mg MLS; 110 mg 1.136 and 10 mg MLS; 110 mg 1.136 and 11 mg MLS; 110 mg 1.136 and 12 mg MLS; 110 mg 1.136 and 13 mg MLS; 110 mg 1.136 and 14 mg MLS; 110 mg 1.136 and 15 mg MLS; 110 mg 1.136 and 16 mg MLS; 110 mg 1.136 and 17 mg MLS; 110 mg 1.136 and 18 mg MLS; 110 mg 1.136 and 19 mg MLS; 110 mg 1.136 and 20 mg MLS;

115 mg 1.136 and 1 mg MLS; 115 mg 1.136 and 2 mg MLS; 115 mg 1.136 and 3 mg MLS; 115 mg 1.136 and 4 mg MLS; 115 mg 1.136 and 5 mg MLS; 115 mg 1.136 and 6 mg MLS; 115 mg 1.136 and 7 mg MLS; 115 mg 1.136 and 8 mg MLS; 115 mg 1.136 and 9 mg MLS; 115 mg 1.136 and 10 mg MLS; 115 mg 1.136 and 11 mg MLS; 115 mg 1.136 and 12 mg MLS; 115 mg 1.136 and 13 mg MLS; 115 mg 1.136 and 14 mg MLS; 115 mg 1.136 and 15 mg MLS; 115 mg 1.136 and 16 mg MLS; 115 mg 1.136 and 17 mg MLS; 115 mg 1.136 and 18 mg MLS; 115 mg 1.136 and 19 mg MLS; 115 mg 1.136 and 20 mg MLS;

120 mg 1.136 and 1 mg MLS; 120 mg 1.136 and 2 mg MLS; 120 mg 1.136 and 3 mg MLS; 120 mg 1.136 and 4 mg MLS; 120 mg 1.136 and 5 mg MLS; 120 mg 1.136 and 6 mg MLS; 120 mg 1.136 and 7 mg MLS; 120 mg 1.136 and 8 mg MLS; 120 mg 1.136 and 9 mg MLS; 120 mg 1.136 and 10 mg MLS; 120 mg 1.136 and 11 mg MLS; 120 mg 1.136 and 12 mg MLS; 120 mg 1.136 and 13 mg MLS; 120 mg 1.136 and 14 mg MLS; 120 mg 1.136 and 15 mg MLS; 120 mg 1.136 and 16 mg MLS; 120 mg 1.136 and 17 mg MLS; 120 mg 1.136 and 18 mg MLS; 120 mg 1.136 and 19 mg MLS; 120 mg 1.136 and 20 mg MLS;

125 mg 1.136 and 1 mg MLS; 125 mg 1.136 and 2 mg MLS; 125 mg 1.136 and 3 mg MLS; 125 mg 1.136 and 4 mg MLS; 125 mg 1.136 and 5 mg MLS; 125 mg 1.136 and 6 mg MLS; 125 mg 1.136 and 7 mg MLS; 125 mg 1.136 and 8 mg MLS; 125 mg 1.136 and 9 mg MLS; 125 mg 1.136 and 10 mg MLS; 125 mg 1.136 and 11 mg MLS; 125 mg 1.136 and 12 mg MLS; 125 mg 1.136 and 13 mg MLS; 125 mg 1.136 and 14 mg MLS; 125 mg 1.136 and 15 mg MLS; 125 mg 1.136 and 16 mg MLS; 125 mg 1.136 and 17 mg MLS; 125 mg 1.136 and 18 mg MLS; 125 mg 1.136 and 19 mg MLS; 125 mg 1.136 and 20 mg MLS;

130 mg 1.136 and 1 mg MLS; 130 mg 1.136 and 2 mg MLS; 130 mg 1.136 and 3 mg MLS; 130 mg 1.136 and 4 mg MLS; 130 mg 1.136 and 5 mg MLS; 130 mg 1.136 and 6 mg MLS; 130 mg 1.136 and 7 mg MLS; 130 mg 1.136 and 8 mg MLS; 130 mg 1.136 and 9 mg MLS; 130 mg 1.136 and 10 mg MLS; 130 mg 1.136 and 11 mg MLS; 130 mg 1.136 and 12 mg MLS; 130 mg 1.136 and 13 mg MLS; 130 mg 1.136 and 14 mg MLS; 130 mg 1.136 and 15 mg MLS; 130 mg 1.136 and 16 mg MLS; 130 mg 1.136 and 17 mg MLS; 130 mg 1.136 and 18 mg MLS; 130 mg 1.136 and 19 mg MLS; 130 mg 1.136 and 20 mg MLS;

135 mg 1.136 and 1 mg MLS; 135 mg 1.136 and 2 mg MLS; 135 mg 1.136 and 3 mg MLS; 135 mg 1.136 and 4 mg MLS; 135 mg 1.136 and 5 mg MLS; 135 mg 1.136 and 6 mg MLS; 135 mg 1.136 and 7 mg MLS; 135 mg 1.136 and 8 mg MLS; 135 mg 1.136 and 9 mg MLS; 135 mg 1.136 and 10 mg MLS; 135 mg 1.136 and 11 mg MLS; 135 mg 1.136 and 12 mg MLS; 135 mg 1.136 and 13 mg MLS; 135 mg 1.136 and 14 mg MLS; 135 mg 1.136 and 15 mg MLS; 135 mg 1.136 and 16 mg MLS; 135 mg 1.136 and 17 mg MLS; 135 mg 1.136 and 18 mg MLS; 135 mg 1.136 and 19 mg MLS; 135 mg 1.136 and 20 mg MLS;

140 mg 1.136 and 1 mg MLS; 140 mg 1.136 and 2 mg MLS; 140 mg 1.136 and 3 mg MLS; 140 mg 1.136 and 4 mg MLS; 140 mg 1.136 and 5 mg MLS; 140 mg 1.136 and 6 mg MLS; 140 mg 1.136 and 7 mg MLS; 140 mg 1.136 and 8 mg MLS; 140 mg 1.136 and 9 mg MLS; 140 mg 1.136 and 10 mg MLS; 140 mg 1.136 and 11 mg MLS; 140 mg 1.136 and 12 mg MLS; 140 mg 1.136 and 13 mg MLS; 140 mg 1.136 and 14 mg MLS; 140 mg 1.136 and 15 mg MLS; 140 mg 1.136 and 16 mg MLS; 140 mg 1.136 and 17 mg MLS; 140 mg 1.136 and 18 mg MLS; 140 mg 1.136 and 19 mg MLS; 140 mg 1.136 and 20 mg MLS;

145 mg 1.136 and 1 mg MLS; 145 mg 1.136 and 2 mg MLS; 145 mg 1.136 and 3 mg MLS; 145 mg 1.136 and 4 mg MLS; 145 mg 1.136 and 5 mg MLS; 145 mg 1.136 and 6 mg MLS; 145 mg 1.136 and 7 mg MLS; 145 mg 1.136 and 8 mg MLS; 145 mg 1.136 and 9 mg MLS; 145 mg 1.136 and 10 mg MLS; 145 mg 1.136 and 11 mg MLS; 145 mg 1.136 and 12 mg MLS; 145 mg 1.136 and 13 mg MLS; 145 mg 1.136 and 14 mg MLS; 145 mg 1.136 and 15 mg MLS; 145 mg 1.136 and 16 mg MLS; 145 mg 1.136 and 17 mg MLS; 145 mg 1.136 and 18 mg MLS; 145 mg 1.136 and 19 mg MLS; 145 mg 1.136 and 20 mg MLS;

150 mg 1.136 and 1 mg MLS; 150 mg 1.136 and 2 mg MLS; 150 mg 1.136 and 3 mg MLS; 150 mg 1.136 and 4 mg MLS; 150 mg 1.136 and 5 mg MLS; 150 mg 1.136 and 6 mg MLS; 150 mg 1.136 and 7 mg MLS; 150 mg 1.136 and 8 mg MLS; 150 mg 1.136 and 9 mg MLS; 150 mg 1.136 and 10 mg MLS; 150 mg 1.136 and 11 mg MLS; 150 mg 1.136 and 12 mg MLS; 150 mg 1.136 and 13 mg MLS; 150 mg 1.136 and 14 mg MLS; 150 mg 1.136 and 15 mg MLS; 150 mg 1.136 and 16 mg MLS; 150 mg 1.136 and 17 mg MLS; 150 mg 1.136 and 18 mg MLS; 150 mg 1.136 and 19 mg MLS; 150 mg 1.136 and 20 mg MLS;

155 mg 1.136 and 1 mg MLS; 155 mg 1.136 and 2 mg MLS; 155 mg 1.136 and 3 mg MLS; 155 mg 1.136 and 4 mg MLS; 155 mg 1.136 and 5 mg MLS; 155 mg 1.136 and 6 mg MLS; 155 mg 1.136 and 7 mg MLS; 155 mg 1.136 and 8 mg MLS; 155 mg 1.136 and 9 mg MLS; 155 mg 1.136 and 10 mg MLS; 155 mg 1.136 and 11 mg MLS; 155 mg 1.136 and 12 mg MLS; 155 mg 1.136 and 13 mg MLS; 155 mg 1.136 and 14 mg MLS; 155 mg 1.136 and 15 mg MLS; 155 mg 1.136 and 16 mg MLS; 155 mg 1.136 and 17 mg MLS; 155 mg 1.136 and 18 mg MLS; 155 mg 1.136 and 19 mg MLS; 155 mg 1.136 and 20 mg MLS;

160 mg 1.136 and 1 mg MLS; 160 mg 1.136 and 2 mg MLS; 160 mg 1.136 and 3 mg MLS; 160 mg 1.136 and 4 mg MLS; 160 mg 1.136 and 5 mg MLS; 160 mg 1.136 and 6 mg MLS; 160 mg 1.136 and 7 mg MLS; 160 mg 1.136 and 8 mg MLS; 160 mg 1.136 and 9 mg MLS; 160 mg 1.136 and 10 mg MLS; 160 mg 1.136 and 11 mg MLS; 160 mg 1.136 and 12 mg MLS; 160 mg 1.136 and 13 mg MLS; 160 mg 1.136 and 14 mg MLS; 160 mg 1.136 and 15 mg MLS; 160 mg 1.136 and 16 mg MLS; 160 mg 1.136 and 17 mg MLS; 160 mg 1.136 and 18 mg MLS; 160 mg 1.136 and 19 mg MLS; 160 mg 1.136 and 20 mg MLS;

165 mg 1.136 and 1 mg MLS; 165 mg 1.136 and 2 mg MLS; 165 mg 1.136 and 3 mg MLS; 165 mg 1.136 and 4 mg MLS; 165 mg 1.136 and 5 mg MLS; 165 mg 1.136 and 6 mg MLS; 165 mg 1.136 and 7 mg MLS; 165 mg 1.136 and 8 mg MLS; 165 mg 1.136 and 9 mg MLS; 165 mg 1.136 and 10 mg MLS; 165 mg 1.136 and 11 mg MLS; 165 mg 1.136 and 12 mg MLS; 165 mg 1.136 and 13 mg MLS; 165 mg 1.136 and 14 mg MLS; 165 mg 1.136 and 15 mg MLS; 165 mg 1.136 and 16 mg MLS; 165 mg 1.136 and 17 mg MLS; 165 mg 1.136 and 18 mg MLS; 165 mg 1.136 and 19 mg MLS; 165 mg 1.136 and 20 mg MLS;

170 mg 1.136 and 1 mg MLS; 170 mg 1.136 and 2 mg MLS; 170 mg 1.136 and 3 mg MLS; 170 mg 1.136 and 4 mg MLS; 170 mg 1.136 and 5 mg MLS; 170 mg 1.136 and 6 mg MLS; 170 mg 1.136 and 7 mg MLS; 170 mg 1.136 and 8 mg MLS; 170 mg 1.136 and 9 mg MLS; 170 mg 1.136 and 10 mg MLS; 170 mg 1.136 and 11 mg MLS; 170 mg 1.136 and 12 mg MLS; 170 mg 1.136 and 13 mg MLS; 170 mg 1.136 and 14 mg MLS; 170 mg 1.136 and 15 mg MLS; 170 mg 1.136 and 16 mg MLS; 170 mg 1.136 and 17 mg MLS; 170 mg 1.136 and 18 mg MLS; 170 mg 1.136 and 19 mg MLS; 170 mg 1.136 and 20 mg MLS;

175 mg 1.136 and 1 mg MLS; 175 mg 1.136 and 2 mg MLS; 175 mg 1.136 and 3 mg MLS; 175 mg 1.136 and 4 mg MLS; 175 mg 1.136 and 5 mg MLS; 175 mg 1.136 and 6 mg MLS; 175 mg 1.136 and 7 mg MLS; 175 mg 1.136 and 8 mg MLS; 175 mg 1.136 and 9 mg MLS; 175 mg 1.136 and 10 mg MLS; 175 mg 1.136 and 11 mg MLS; 175 mg 1.136 and 12 mg MLS; 175 mg 1.136 and 13 mg MLS; 175 mg 1.136 and 14 mg MLS; 175 mg 1.136 and 15 mg MLS; 175 mg 1.136 and 16 mg MLS; 175 mg 1.136 and 17 mg MLS; 175 mg 1.136 and 18 mg MLS; 175 mg 1.136 and 19 mg MLS; 175 mg 1.136 and 20 mg MLS;

180 mg 1.136 and 1 mg MLS; 180 mg 1.136 and 2 mg MLS; 180 mg 1.136 and 3 mg MLS; 180 mg 1.136 and 4 mg MLS; 180 mg 1.136 and 5 mg MLS; 180 mg 1.136 and 6 mg MLS; 180 mg 1.136 and 7 mg MLS; 180 mg 1.136 and 8 mg MLS; 180 mg 1.136 and 9 mg MLS; 180 mg 1.136 and 10 mg MLS; 180 mg 1.136 and 11 mg MLS; 180 mg 1.136 and 12 mg MLS; 180 mg 1.136 and 13 mg MLS; 180 mg 1.136 and 14 mg MLS; 180 mg 1.136 and 15 mg MLS; 180 mg 1.136 and 16 mg MLS; 180 mg 1.136 and 17 mg MLS; 180 mg 1.136 and 18 mg MLS; 180 mg 1.136 and 19 mg MLS; 180 mg 1.136 and 20 mg MLS;

185 mg 1.136 and 1 mg MLS; 185 mg 1.136 and 2 mg MLS; 185 mg 1.136 and 3 mg MLS; 185 mg 1.136 and 4 mg MLS; 185 mg 1.136 and 5 mg MLS; 185 mg 1.136 and 6 mg MLS; 185 mg 1.136 and 7 mg MLS; 185 mg 1.136 and 8 mg MLS; 185 mg 1.136 and 9 mg MLS; 185 mg 1.136 and 10 mg MLS; 185 mg 1.136 and 11 mg MLS; 185 mg 1.136 and 12 mg MLS; 185 mg 1.136 and 13 mg MLS; 185 mg 1.136 and 14 mg MLS; 185 mg 1.136 and 15 mg MLS; 185 mg 1.136 and 16 mg MLS; 185 mg 1.136 and 17 mg MLS; 185 mg 1.136 and 18 mg MLS; 185 mg 1.136 and 19 mg MLS; 185 mg 1.136 and 20 mg MLS;

190 mg 1.136 and 1 mg MLS; 190 mg 1.136 and 2 mg MLS; 190 mg 1.136 and 3 mg MLS; 190 mg 1.136 and 4 mg MLS; 190 mg 1.136 and 5 mg MLS; 190 mg 1.136 and 6 mg MLS; 190 mg 1.136 and 7 mg MLS; 190 mg 1.136 and 8 mg MLS; 190 mg 1.136 and 9 mg MLS; 190 mg 1.136 and 10 mg MLS; 190 mg 1.136 and 11 mg MLS; 190 mg 1.136 and 12 mg MLS; 190 mg 1.136 and 13 mg MLS; 190 mg 1.136 and 14 mg MLS; 190 mg 1.136 and 15 mg MLS; 190 mg 1.136 and 16 mg MLS; 190 mg 1.136 and 17 mg MLS; 190 mg 1.136 and 18 mg MLS; 190 mg 1.136 and 19 mg MLS; 190 mg 1.136 and 20 mg MLS;

195 mg 1.136 and 1 mg MLS; 195 mg 1.136 and 2 mg MLS; 195 mg 1.136 and 3 mg MLS; 195 mg 1.136 and 4 mg MLS; 195 mg 1.136 and 5 mg MLS; 195 mg 1.136 and 6 mg MLS; 195 mg 1.136 and 7 mg MLS; 195 mg 1.136 and 8 mg MLS; 195 mg 1.136 and 9 mg MLS; 195 mg 1.136 and 10 mg MLS; 195 mg 1.136 and 11 mg MLS; 195 mg 1.136 and 12 mg MLS; 195 mg 1.136 and 13 mg MLS; 195 mg 1.136 and 14 mg MLS; 195 mg 1.136 and 15 mg MLS; 195 mg 1.136 and 16 mg MLS; 195 mg 1.136 and 17 mg MLS; 195 mg 1.136 and 18 mg MLS; 195 mg 1.136 and 19 mg MLS; 195 mg 1.136 and 20 mg MLS;

200 mg 1.136 and 1 mg MLS; 200 mg 1.136 and 2 mg MLS; 200 mg 1.136 and 3 mg MLS; 200 mg 1.136 and 4 mg MLS; 200 mg 1.136 and 5 mg MLS; 200 mg 1.136 and 6 mg MLS; 200 mg 1.136 and 7 mg MLS; 200 mg 1.136 and 8 mg MLS; 200 mg 1.136 and 9 mg MLS; 200 mg 1.136 and 10 mg MLS; 200 mg 1.136 and 11 mg MLS; 200 mg 1.136 and 12 mg MLS; 200 mg 1.136 and 13 mg MLS; 200 mg 1.136 and 14 mg MLS; 200 mg 1.136 and 15 mg MLS; 200 mg 1.136 and 16 mg MLS; 200 mg 1.136 and 17 mg MLS; 200 mg 1.136 and 18 mg MLS; 200 mg 1.136 and 19 mg MLS; 200 mg 1.136 and 20 mg MLS;

205 mg 1.136 and 1 mg MLS; 205 mg 1.136 and 2 mg MLS; 205 mg 1.136 and 3 mg MLS; 205 mg 1.136 and 4 mg MLS; 205 mg 1.136 and 5 mg MLS; 205 mg 1.136 and 6 mg MLS; 205 mg 1.136 and 7 mg MLS; 205 mg 1.136 and 8 mg MLS; 205 mg 1.136 and 9 mg MLS; 205 mg 1.136 and 10 mg MLS; 205 mg 1.136 and 11 mg MLS; 205 mg 1.136 and 12 mg MLS; 205 mg 1.136 and 13 mg MLS; 205 mg 1.136 and 14 mg MLS; 205 mg 1.136 and 15 mg MLS; 205 mg 1.136 and 16 mg MLS; 205 mg 1.136 and 17 mg MLS; 205 mg 1.136 and 18 mg MLS; 205 mg 1.136 and 19 mg MLS; 205 mg 1.136 and 20 mg MLS;

210 mg 1.136 and 1 mg MLS; 210 mg 1.136 and 2 mg MLS; 210 mg 1.136 and 3 mg MLS; 210 mg 1.136 and 4 mg MLS; 210 mg 1.136 and 5 mg MLS; 210 mg 1.136 and 6 mg MLS; 210 mg 1.136 and 7 mg MLS; 210 mg 1.136 and 8 mg MLS; 210 mg 1.136 and 9 mg MLS; 210 mg 1.136 and 10 mg MLS; 210 mg 1.136 and 11 mg MLS; 210 mg 1.136 and 12 mg MLS; 210 mg 1.136 and 13 mg MLS; 210 mg 1.136 and 14 mg MLS; 210 mg 1.136 and 15 mg MLS; 210 mg 1.136 and 16 mg MLS; 210 mg 1.136 and 17 mg MLS; 210 mg 1.136 and 18 mg MLS; 210 mg 1.136 and 19 mg MLS; 210 mg 1.136 and 20 mg MLS;

215 mg 1.136 and 1 mg MLS; 215 mg 1.136 and 2 mg MLS; 215 mg 1.136 and 3 mg MLS; 215 mg 1.136 and 4 mg MLS; 215 mg 1.136 and 5 mg MLS; 215 mg 1.136 and 6 mg MLS; 215 mg 1.136 and 7 mg MLS; 215 mg 1.136 and 8 mg MLS; 215 mg 1.136 and 9 mg MLS; 215 mg 1.136 and 10 mg MLS; 215 mg 1.136 and 11 mg MLS; 215 mg 1.136 and 12 mg MLS; 215 mg 1.136 and 13 mg MLS; 215 mg 1.136 and 14 mg MLS; 215 mg 1.136 and 15 mg MLS; 215 mg 1.136 and 16 mg MLS; 215 mg 1.136 and 17 mg MLS; 215 mg 1.136 and 18 mg MLS; 215 mg 1.136 and 19 mg MLS; 215 mg 1.136 and 20 mg MLS;

220 mg 1.136 and 1 mg MLS; 220 mg 1.136 and 2 mg MLS; 220 mg 1.136 and 3 mg MLS; 220 mg 1.136 and 4 mg MLS; 220 mg 1.136 and 5 mg MLS; 220 mg 1.136 and 6 mg MLS; 220 mg 1.136 and 7 mg MLS; 220 mg 1.136 and 8 mg MLS; 220 mg 1.136 and 9 mg MLS; 220 mg 1.136 and 10 mg MLS; 220 mg 1.136 and 11 mg MLS; 220 mg 1.136 and 12 mg MLS; 220 mg 1.136 and 13 mg MLS; 220 mg 1.136 and 14 mg MLS; 220 mg 1.136 and 15 mg MLS; 220 mg 1.136 and 16 mg MLS; 220 mg 1.136 and 17 mg MLS; 220 mg 1.136 and 18 mg MLS; 220 mg 1.136 and 19 mg MLS; 220 mg 1.136 and 20 mg MLS;

225 mg 1.136 and 1 mg MLS; 225 mg 1.136 and 2 mg MLS; 225 mg 1.136 and 3 mg MLS; 225 mg 1.136 and 4 mg MLS; 225 mg 1.136 and 5 mg MLS; 225 mg 1.136 and 6 mg MLS; 225 mg 1.136 and 7 mg MLS; 225 mg 1.136 and 8 mg MLS; 225 mg 1.136 and 9 mg MLS; 225 mg 1.136 and 10 mg MLS; 225 mg 1.136 and 11 mg MLS; 225 mg 1.136 and 12 mg MLS; 225 mg 1.136 and 13 mg MLS; 225 mg 1.136 and 14 mg MLS; 225 mg 1.136 and 15 mg MLS; 225 mg 1.136 and 16 mg MLS; 225 mg 1.136 and 17 mg MLS; 225 mg 1.136 and 18 mg MLS; 225 mg 1.136 and 19 mg MLS; 225 mg 1.136 and 20 mg MLS;

230 mg 1.136 and 1 mg MLS; 230 mg 1.136 and 2 mg MLS; 230 mg 1.136 and 3 mg MLS; 230 mg 1.136 and 4 mg MLS; 230 mg 1.136 and 5 mg MLS; 230 mg 1.136 and 6 mg MLS; 230 mg 1.136 and 7 mg MLS; 230 mg 1.136 and 8 mg MLS; 230 mg 1.136 and 9 mg MLS; 230 mg 1.136 and 10 mg MLS; 230 mg 1.136 and 11 mg MLS; 230 mg 1.136 and 12 mg MLS; 230 mg 1.136 and 13 mg MLS; 230 mg 1.136 and 14 mg MLS; 230 mg 1.136 and 15 mg MLS; 230 mg 1.136 and 16 mg MLS; 230 mg 1.136 and 17 mg MLS; 230 mg 1.136 and 18 mg MLS; 230 mg 1.136 and 19 mg MLS; 230 mg 1.136 and 20 mg MLS;

235 mg 1.136 and 1 mg MLS; 235 mg 1.136 and 2 mg MLS; 235 mg 1.136 and 3 mg MLS; 235 mg 1.136 and 4 mg MLS; 235 mg 1.136 and 5 mg MLS; 235 mg 1.136 and 6 mg MLS; 235 mg 1.136 and 7 mg MLS; 235 mg 1.136 and 8 mg MLS; 235 mg 1.136 and 9 mg MLS; 235 mg 1.136 and 10 mg MLS; 235 mg 1.136 and 11 mg MLS; 235 mg 1.136 and 12 mg MLS; 235 mg 1.136 and 13 mg MLS; 235 mg 1.136 and 14 mg MLS; 235 mg 1.136 and 15 mg MLS; 235 mg 1.136 and 16 mg MLS; 235 mg 1.136 and 17 mg MLS; 235 mg 1.136 and 18 mg MLS; 235 mg 1.136 and 19 mg MLS; 235 mg 1.136 and 20 mg MLS;

240 mg 1.136 and 1 mg MLS; 240 mg 1.136 and 2 mg MLS; 240 mg 1.136 and 3 mg MLS; 240 mg 1.136 and 4 mg MLS; 240 mg 1.136 and 5 mg MLS; 240 mg 1.136 and 6 mg MLS; 240 mg 1.136 and 7 mg MLS; 240 mg 1.136 and 8 mg MLS; 240 mg 1.136 and 9 mg MLS; 240 mg 1.136 and 10 mg MLS; 240 mg 1.136 and 11 mg MLS; 240 mg 1.136 and 12 mg MLS; 240 mg 1.136 and 13 mg MLS; 240 mg 1.136 and 14 mg MLS; 240 mg 1.136 and 15 mg MLS; 240 mg 1.136 and 16 mg MLS; 240 mg 1.136 and 17 mg MLS; 240 mg 1.136 and 18 mg MLS; 240 mg 1.136 and 19 mg MLS; 240 mg 1.136 and 20 mg MLS;

245 mg 1.136 and 1 mg MLS; 245 mg 1.136 and 2 mg MLS; 245 mg 1.136 and 3 mg MLS; 245 mg 1.136 and 4 mg MLS; 245 mg 1.136 and 5 mg MLS; 245 mg 1.136 and 6 mg MLS; 245 mg 1.136 and 7 mg MLS; 245 mg 1.136 and 8 mg MLS; 245 mg 1.136 and 9 mg MLS; 245 mg 1.136 and 10 mg MLS; 245 mg 1.136 and 11 mg MLS; 245 mg 1.136 and 12 mg MLS; 245 mg 1.136 and 13 mg MLS; 245 mg 1.136 and 14 mg MLS; 245 mg 1.136 and 15 mg MLS; 245 mg 1.136 and 16 mg MLS; 245 mg 1.136 and 17 mg MLS; 245 mg 1.136 and 18 mg MLS; 245 mg 1.136 and 19 mg MLS; 245 mg 1.136 and 20 mg MLS;

250 mg 1.136 and 1 mg MLS; 250 mg 1.136 and 2 mg MLS; 250 mg 1.136 and 3 mg MLS; 250 mg 1.136 and 4 mg MLS; 250 mg 1.136 and 5 mg MLS; 250 mg 1.136 and 6 mg MLS; 250 mg 1.136 and 7 mg MLS; 250 mg 1.136 and 8 mg MLS; 250 mg 1.136 and 9 mg MLS; 250 mg 1.136 and 10 mg MLS; 250 mg 1.136 and 11 mg MLS; 250 mg 1.136 and 12 mg MLS; 250 mg 1.136 and 13 mg MLS; 250 mg 1.136 and 14 mg MLS; 250 mg 1.136 and 15 mg MLS; 250 mg 1.136 and 16 mg MLS; 250 mg 1.136 and 17 mg MLS; 250 mg 1.136 and 18 mg MLS; 250 mg 1.136 and 19 mg MLS; 250 mg 1.136 and 20 mg MLS;

255 mg 1.136 and 1 mg MLS; 255 mg 1.136 and 2 mg MLS; 255 mg 1.136 and 3 mg MLS; 255 mg 1.136 and 4 mg MLS; 255 mg 1.136 and 5 mg MLS; 255 mg 1.136 and 6 mg MLS; 255 mg 1.136 and 7 mg MLS; 255 mg 1.136 and 8 mg MLS; 255 mg 1.136 and 9 mg MLS; 255 mg 1.136 and 10 mg MLS; 255 mg 1.136 and 11 mg MLS; 255 mg 1.136 and 12 mg MLS; 255 mg 1.136 and 13 mg MLS; 255 mg 1.136 and 14 mg MLS; 255 mg 1.136 and 15 mg MLS; 255 mg 1.136 and 16 mg MLS; 255 mg 1.136 and 17 mg MLS; 255 mg 1.136 and 18 mg MLS; 255 mg 1.136 and 19 mg MLS; 255 mg 1.136 and 20 mg MLS;

260 mg 1.136 and 1 mg MLS; 260 mg 1.136 and 2 mg MLS; 260 mg 1.136 and 3 mg MLS; 260 mg 1.136 and 4 mg MLS; 260 mg 1.136 and 5 mg MLS; 260 mg 1.136 and 6 mg MLS; 260 mg 1.136 and 7 mg MLS; 260 mg 1.136 and 8 mg MLS; 260 mg 1.136 and 9 mg MLS; 260 mg 1.136 and 10 mg MLS; 260 mg 1.136 and 11 mg MLS; 260 mg 1.136 and 12 mg MLS; 260 mg 1.136 and 13 mg MLS; 260 mg 1.136 and 14 mg MLS; 260 mg 1.136 and 15 mg MLS; 260 mg 1.136 and 16 mg MLS; 260 mg 1.136 and 17 mg MLS; 260 mg 1.136 and 18 mg MLS; 260 mg 1.136 and 19 mg MLS; 260 mg 1.136 and 20 mg MLS;

265 mg 1.136 and 1 mg MLS; 265 mg 1.136 and 2 mg MLS; 265 mg 1.136 and 3 mg MLS; 265 mg 1.136 and 4 mg MLS; 265 mg 1.136 and 5 mg MLS; 265 mg 1.136 and 6 mg MLS; 265 mg 1.136 and 7 mg MLS; 265 mg 1.136 and 8 mg MLS; 265 mg 1.136 and 9 mg MLS; 265 mg 1.136 and 10 mg MLS; 265 mg 1.136 and 11 mg MLS; 265 mg 1.136 and 12 mg MLS; 265 mg 1.136 and 13 mg MLS; 265 mg 1.136 and 14 mg MLS; 265 mg 1.136 and 15 mg MLS; 265 mg 1.136 and 16 mg MLS; 265 mg 1.136 and 17 mg MLS; 265 mg 1.136 and 18 mg MLS; 265 mg 1.136 and 19 mg MLS; 265 mg 1.136 and 20 mg MLS;

270 mg 1.136 and 1 mg MLS; 270 mg 1.136 and 2 mg MLS; 270 mg 1.136 and 3 mg MLS; 270 mg 1.136 and 4 mg MLS; 270 mg 1.136 and 5 mg MLS; 270 mg 1.136 and 6 mg MLS; 270 mg 1.136 and 7 mg MLS; 270 mg 1.136 and 8 mg MLS; 270 mg 1.136 and 9 mg MLS; 270 mg 1.136 and 10 mg MLS; 270 mg 1.136 and 11 mg MLS; 270 mg 1.136 and 12 mg MLS; 270 mg 1.136 and 13 mg MLS; 270 mg 1.136 and 14 mg MLS; 270 mg 1.136 and 15 mg MLS; 270 mg 1.136 and 16 mg MLS; 270 mg 1.136 and 17 mg MLS; 270 mg 1.136 and 18 mg MLS; 270 mg 1.136 and 19 mg MLS; 270 mg 1.136 and 20 mg MLS;

275 mg 1.136 and 1 mg MLS; 275 mg 1.136 and 2 mg MLS; 275 mg 1.136 and 3 mg MLS; 275 mg 1.136 and 4 mg MLS; 275 mg 1.136 and 5 mg MLS; 275 mg 1.136 and 6 mg MLS; 275 mg 1.136 and 7 mg MLS; 275 mg 1.136 and 8 mg MLS; 275 mg 1.136 and 9 mg MLS; 275 mg 1.136 and 10 mg MLS; 275 mg 1.136 and 11 mg MLS; 275 mg 1.136 and 12 mg MLS; 275 mg 1.136 and 13 mg MLS; 275 mg 1.136 and 14 mg MLS; 275 mg 1.136 and 15 mg MLS; 275 mg 1.136 and 16 mg MLS; 275 mg 1.136 and 17 mg MLS; 275 mg 1.136 and 18 mg MLS; 275 mg 1.136 and 19 mg MLS; 275 mg 1.136 and 20 mg MLS;

280 mg 1.136 and 1 mg MLS; 280 mg 1.136 and 2 mg MLS; 280 mg 1.136 and 3 mg MLS; 280 mg 1.136 and 4 mg MLS; 280 mg 1.136 and 5 mg MLS; 280 mg 1.136 and 6 mg MLS; 280 mg 1.136 and 7 mg MLS; 280 mg 1.136 and 8 mg MLS; 280 mg 1.136 and 9 mg MLS; 280 mg 1.136 and 10 mg MLS; 280 mg 1.136 and 11 mg MLS; 280 mg 1.136 and 12 mg MLS; 280 mg 1.136 and 13 mg MLS; 280 mg 1.136 and 14 mg MLS; 280 mg 1.136 and 15 mg MLS; 280 mg 1.136 and 16 mg MLS; 280 mg 1.136 and 17 mg MLS; 280 mg 1.136 and 18 mg MLS; 280 mg 1.136 and 19 mg MLS; 280 mg 1.136 and 20 mg MLS;

285 mg 1.136 and 1 mg MLS; 285 mg 1.136 and 2 mg MLS; 285 mg 1.136 and 3 mg MLS; 285 mg 1.136 and 4 mg MLS; 285 mg 1.136 and 5 mg MLS; 285 mg 1.136 and 6 mg MLS; 285 mg 1.136 and 7 mg MLS; 285 mg 1.136 and 8 mg MLS; 285 mg 1.136 and 9 mg MLS; 285 mg 1.136 and 10 mg MLS; 285 mg 1.136 and 11 mg MLS; 285 mg 1.136 and 12 mg MLS; 285 mg 1.136 and 13 mg MLS; 285 mg 1.136 and 14 mg MLS; 285 mg 1.136 and 15 mg MLS; 285 mg 1.136 and 16 mg MLS; 285 mg 1.136 and 17 mg MLS; 285 mg 1.136 and 18 mg MLS; 285 mg 1.136 and 19 mg MLS; 285 mg 1.136 and 20 mg MLS;

290 mg 1.136 and 1 mg MLS; 290 mg 1.136 and 2 mg MLS; 290 mg 1.136 and 3 mg MLS; 290 mg 1.136 and 4 mg MLS; 290 mg 1.136 and 5 mg MLS; 290 mg 1.136 and 6 mg MLS; 290 mg 1.136 and 7 mg MLS; 290 mg 1.136 and 8 mg MLS; 290 mg 1.136 and 9 mg MLS; 290 mg 1.136 and 10 mg MLS; 290 mg 1.136 and 11 mg MLS; 290 mg 1.136 and 12 mg MLS; 290 mg 1.136 and 13 mg MLS; 290 mg 1.136 and 14 mg MLS; 290 mg 1.136 and 15 mg MLS; 290 mg 1.136 and 16 mg MLS; 290 mg 1.136 and 17 mg MLS; 290 mg 1.136 and 18 mg MLS; 290 mg 1.136 and 19 mg MLS; 290 mg 1.136 and 20 mg MLS;

295 mg 1.136 and 1 mg MLS; 295 mg 1.136 and 2 mg MLS; 295 mg 1.136 and 3 mg MLS; 295 mg 1.136 and 4 mg MLS; 295 mg 1.136 and 5 mg MLS; 295 mg 1.136 and 6 mg MLS; 295 mg 1.136 and 7 mg MLS; 295 mg 1.136 and 8 mg MLS; 295 mg 1.136 and 9 mg MLS; 295 mg 1.136 and 10 mg MLS; 295 mg 1.136 and 11 mg MLS; 295 mg 1.136 and 12 mg MLS; 295 mg 1.136 and 13 mg MLS; 295 mg 1.136 and 14 mg MLS; 295 mg 1.136 and 15 mg MLS; 295 mg 1.136 and 16 mg MLS; 295 mg 1.136 and 17 mg MLS; 295 mg 1.136 and 18 mg MLS; 295 mg 1.136 and 19 mg MLS; 295 mg 1.136 and 20 mg MLS;

300 mg 1.136 and 1 mg MLS; 300 mg 1.136 and 2 mg MLS; 300 mg 1.136 and 3 mg MLS; 300 mg 1.136 and 4 mg MLS; 300 mg 1.136 and 5 mg MLS; 300 mg 1.136 and 6 mg MLS; 300 mg 1.136 and 7 mg MLS; 300 mg 1.136 and 8 mg MLS; 300 mg 1.136 and 9 mg MLS; 300 mg 1.136 and 10 mg MLS; 300 mg 1.136 and 11 mg MLS; 300 mg 1.136 and 12 mg MLS; 300 mg 1.136 and 13 mg MLS; 300 mg 1.136 and 14 mg MLS; 300 mg 1.136 and 15 mg MLS; 300 mg 1.136 and 16 mg MLS; 300 mg 1.136 and 17 mg MLS; 300 mg 1.136 and 18 mg MLS; 300 mg 1.136 and 19 mg MLS; 300 mg 1.136 and 20 mg MLS.

305 mg 1.136 and 1 mg MLS; 305 mg 1.136 and 2 mg MLS; 305 mg 1.136 and 3 mg MLS; 305 mg 1.136 and 4 mg MLS; 305 mg 1.136 and 5 mg MLS; 305 mg 1.136 and 6 mg MLS; 305 mg 1.136 and 7 mg MLS; 305 mg 1.136 and 8 mg MLS; 305 mg 1.136 and 9 mg MLS; 305 mg 1.136 and 10 mg MLS; 305 mg 1.136 and 11 mg MLS; 305 mg 1.136 and 12 mg MLS; 305 mg 1.136 and 13 mg MLS; 305 mg 1.136 and 14 mg MLS; 305 mg 1.136 and 15 mg MLS; 305 mg 1.136 and 16 mg MLS; 305 mg 1.136 and 17 mg MLS; 305 mg 1.136 and 18 mg MLS; 305 mg 1.136 and 19 mg MLS; 305 mg 1.136 and 20 mg MLS.

310 mg 1.136 and 1 mg MLS; 310 mg 1.136 and 2 mg MLS; 310 mg 1.136 and 3 mg MLS; 310 mg 1.136 and 4 mg MLS; 310 mg 1.136 and 5 mg MLS; 310 mg 1.136 and 6 mg MLS; 310 mg 1.136 and 7 mg MLS; 310 mg 1.136 and 8 mg MLS; 310 mg 1.136 and 9 mg MLS; 310 mg 1.136 and 10 mg MLS; 310 mg 1.136 and 11 mg MLS; 310 mg 1.136 and 12 mg MLS; 310 mg 1.136 and 13 mg MLS; 310 mg 1.136 and 14 mg MLS; 310 mg 1.136 and 15 mg MLS; 310 mg 1.136 and 16 mg MLS; 310 mg 1.136 and 17 mg MLS; 310 mg 1.136 and 18 mg MLS; 310 mg 1.136 and 19 mg MLS; 310 mg 1.136 and 20 mg MLS.

315 mg 1.136 and 1 mg MLS; 315 mg 1.136 and 2 mg MLS; 315 mg 1.136 and 3 mg MLS; 315 mg 1.136 and 4 mg MLS; 315 mg 1.136 and 5 mg MLS; 315 mg 1.136 and 6 mg MLS; 315 mg 1.136 and 7 mg MLS; 315 mg 1.136 and 8 mg MLS; 315 mg 1.136 and 9 mg MLS; 315 mg 1.136 and 10 mg MLS; 315 mg 1.136 and 11 mg MLS; 315 mg 1.136 and 12 mg MLS; 315 mg 1.136 and 13 mg MLS; 315 mg 1.136 and 14 mg MLS; 315 mg 1.136 and 15 mg MLS; 315 mg 1.136 and 16 mg MLS; 315 mg 1.136 and 17 mg MLS; 315 mg 1.136 and 18 mg MLS; 315 mg 1.136 and 19 mg MLS; 315 mg 1.136 and 20 mg MLS.

320 mg 1.136 and 1 mg MLS; 320 mg 1.136 and 2 mg MLS; 320 mg 1.136 and 3 mg MLS; 320 mg 1.136 and 4 mg MLS; 320 mg 1.136 and 5 mg MLS; 320 mg 1.136 and 6 mg MLS; 320 mg 1.136 and 7 mg MLS; 320 mg 1.136 and 8 mg MLS; 320 mg 1.136 and 9 mg MLS; 320 mg 1.136 and 10 mg MLS; 320 mg 1.136 and 11 mg MLS; 320 mg 1.136 and 12 mg MLS; 320 mg 1.136 and 13 mg MLS; 320 mg 1.136 and 14 mg MLS; 320 mg 1.136 and 15 mg MLS; 320 mg 1.136 and 16 mg MLS; 320 mg 1.136 and 17 mg MLS; 320 mg 1.136 and 18 mg MLS; 320 mg 1.136 and 19 mg MLS; 320 mg 1.136 and 20 mg MLS.

325 mg 1.136 and 1 mg MLS; 325 mg 1.136 and 2 mg MLS; 325 mg 1.136 and 3 mg MLS; 325 mg 1.136 and 4 mg MLS; 325 mg 1.136 and 5 mg MLS; 325 mg 1.136 and 6 mg MLS; 325 mg 1.136 and 7 mg MLS; 325 mg 1.136 and 8 mg MLS; 325 mg 1.136 and 9 mg MLS; 325 mg 1.136 and 10 mg MLS; 325 mg 1.136 and 11 mg MLS; 325 mg 1.136 and 12 mg MLS; 325 mg 1.136 and 13 mg MLS; 325 mg 1.136 and 14 mg MLS; 325 mg 1.136 and 15 mg MLS; 325 mg 1.136 and 16 mg MLS; 325 mg 1.136 and 17 mg MLS; 325 mg 1.136 and 18 mg MLS; 325 mg 1.136 and 19 mg MLS; 325 mg 1.136 and 20 mg MLS.

330 mg 1.136 and 1 mg MLS; 330 mg 1.136 and 2 mg MLS; 330 mg 1.136 and 3 mg MLS; 330 mg 1.136 and 4 mg MLS; 330 mg 1.136 and 5 mg MLS; 330 mg 1.136 and 6 mg MLS; 330 mg 1.136 and 7 mg MLS; 330 mg 1.136 and 8 mg MLS; 330 mg 1.136 and 9 mg MLS; 330 mg 1.136 and 10 mg MLS; 330 mg 1.136 and 11 mg MLS; 330 mg 1.136 and 12 mg MLS; 330 mg 1.136 and 13 mg MLS; 330 mg 1.136 and 14 mg MLS; 330 mg 1.136 and 15 mg MLS; 330 mg 1.136 and 16 mg MLS; 330 mg 1.136 and 17 mg MLS; 330 mg 1.136 and 18 mg MLS; 330 mg 1.136 and 19 mg MLS; 330 mg 1.136 and 20 mg MLS.

335 mg 1.136 and 1 mg MLS; 335 mg 1.136 and 2 mg MLS; 335 mg 1.136 and 3 mg MLS; 335 mg 1.136 and 4 mg MLS; 335 mg 1.136 and 5 mg MLS; 335 mg 1.136 and 6 mg MLS; 335 mg 1.136 and 7 mg MLS; 335 mg 1.136 and 8 mg MLS; 335 mg 1.136 and 9 mg MLS; 335 mg 1.136 and 10 mg MLS; 335 mg 1.136 and 11 mg MLS; 335 mg 1.136 and 12 mg MLS; 335 mg 1.136 and 13 mg MLS; 335 mg 1.136 and 14 mg MLS; 335 mg 1.136 and 15 mg MLS; 335 mg 1.136 and 16 mg MLS; 335 mg 1.136 and 17 mg MLS; 335 mg 1.136 and 18 mg MLS; 335 mg 1.136 and 19 mg MLS; 335 mg 1.136 and 20 mg MLS.

340 mg 1.136 and 1 mg MLS; 340 mg 1.136 and 2 mg MLS; 340 mg 1.136 and 3 mg MLS; 340 mg 1.136 and 4 mg MLS; 340 mg 1.136 and 5 mg MLS; 340 mg 1.136 and 6 mg MLS; 340 mg 1.136 and 7 mg MLS; 340 mg 1.136 and 8 mg MLS; 340 mg 1.136 and 9 mg MLS; 340 mg 1.136 and 10 mg MLS; 340 mg 1.136 and 11 mg MLS; 340 mg 1.136 and 12 mg MLS; 340 mg 1.136 and 13 mg MLS; 340 mg 1.136 and 14 mg MLS; 340 mg 1.136 and 15 mg MLS; 340 mg 1.136 and 16 mg MLS; 340 mg 1.136 and 17 mg MLS; 340 mg 1.136 and 18 mg MLS; 340 mg 1.136 and 19 mg MLS; 340 mg 1.136 and 20 mg MLS.

345 mg 1.136 and 1 mg MLS; 345 mg 1.136 and 2 mg MLS; 345 mg 1.136 and 3 mg MLS; 345 mg 1.136 and 4 mg MLS; 345 mg 1.136 and 5 mg MLS; 345 mg 1.136 and 6 mg MLS; 345 mg 1.136 and 7 mg MLS; 345 mg 1.136 and 8 mg MLS; 345 mg 1.136 and 9 mg MLS; 345 mg 1.136 and 10 mg MLS; 345 mg 1.136 and 11 mg MLS; 345 mg 1.136 and 12 mg MLS; 345 mg 1.136 and 13 mg MLS; 345 mg 1.136 and 14 mg MLS; 345 mg 1.136 and 15 mg MLS; 345 mg 1.136 and 16 mg MLS; 345 mg 1.136 and 17 mg MLS; 345 mg 1.136 and 18 mg MLS; 345 mg 1.136 and 19 mg MLS; 345 mg 1.136 and 20 mg MLS.

350 mg 1.136 and 1 mg MLS; 350 mg 1.136 and 2 mg MLS; 350 mg 1.136 and 3 mg MLS; 350 mg 1.136 and 4 mg MLS; 350 mg 1.136 and 5 mg MLS; 350 mg 1.136 and 6 mg MLS; 350 mg 1.136 and 7 mg MLS; 350 mg 1.136 and 8 mg MLS; 350 mg 1.136 and 9 mg MLS; 350 mg 1.136 and 10 mg MLS; 350 mg 1.136 and 11 mg MLS; 350 mg 1.136 and 12 mg MLS; 350 mg 1.136 and 13 mg MLS; 350 mg 1.136 and 14 mg MLS; 350 mg 1.136 and 15 mg MLS; 350 mg 1.136 and 16 mg MLS; 350 mg 1.136 and 17 mg MLS; 350 mg 1.136 and 18 mg MLS; 350 mg 1.136 and 19 mg MLS; 350 mg 1.136 and 20 mg MLS.

355 mg 1.136 and 1 mg MLS; 355 mg 1.136 and 2 mg MLS; 355 mg 1.136 and 3 mg MLS; 355 mg 1.136 and 4 mg MLS; 355 mg 1.136 and 5 mg MLS; 355 mg 1.136 and 6 mg MLS; 355 mg 1.136 and 7 mg MLS; 355 mg 1.136 and 8 mg MLS; 355 mg 1.136 and 9 mg MLS; 355 mg 1.136 and 10 mg MLS; 355 mg 1.136 and 11 mg MLS; 355 mg 1.136 and 12 mg MLS; 355 mg 1.136 and 13 mg MLS; 355 mg 1.136 and 14 mg MLS; 355 mg 1.136 and 15 mg MLS; 355 mg 1.136 and 16 mg MLS; 355 mg 1.136 and 17 mg MLS; 355 mg 1.136 and 18 mg MLS; 355 mg 1.136 and 19 mg MLS; 355 mg 1.136 and 20 mg MLS.

360 mg 1.136 and 1 mg MLS; 360 mg 1.136 and 2 mg MLS; 360 mg 1.136 and 3 mg MLS; 360 mg 1.136 and 4 mg MLS; 360 mg 1.136 and 5 mg MLS; 360 mg 1.136 and 6 mg MLS; 360 mg 1.136 and 7 mg MLS; 360 mg 1.136 and 8 mg MLS; 360 mg 1.136 and 9 mg MLS; 360 mg 1.136 and 10 mg MLS; 360 mg 1.136 and 11 mg MLS; 360 mg 1.136 and 12 mg MLS; 360 mg 1.136 and 13 mg MLS; 360 mg 1.136 and 14 mg MLS; 360 mg 1.136 and 15 mg MLS; 360 mg 1.136 and 16 mg MLS; 360 mg 1.136 and 17 mg MLS; 360 mg 1.136 and 18 mg MLS; 360 mg 1.136 and 19 mg MLS; 360 mg 1.136 and 20 mg MLS.

365 mg 1.136 and 1 mg MLS; 365 mg 1.136 and 2 mg MLS; 365 mg 1.136 and 3 mg MLS; 365 mg 1.136 and 4 mg MLS; 365 mg 1.136 and 5 mg MLS; 365 mg 1.136 and 6 mg MLS; 365 mg 1.136 and 7 mg MLS; 365 mg 1.136 and 8 mg MLS; 365 mg 1.136 and 9 mg MLS; 365 mg 1.136 and 10 mg MLS; 365 mg 1.136 and 11 mg MLS; 365 mg 1.136 and 12 mg MLS; 365 mg 1.136 and 13 mg MLS; 365 mg 1.136 and 14 mg MLS; 365 mg 1.136 and 15 mg MLS; 365 mg 1.136 and 16 mg MLS; 365 mg 1.136 and 17 mg MLS; 365 mg 1.136 and 18 mg MLS; 365 mg 1.136 and 19 mg MLS; 365 mg 1.136 and 20 mg MLS.

370 mg 1.136 and 1 mg MLS; 370 mg 1.136 and 2 mg MLS; 370 mg 1.136 and 3 mg MLS; 370 mg 1.136 and 4 mg MLS; 370 mg 1.136 and 5 mg MLS; 370 mg 1.136 and 6 mg MLS; 370 mg 1.136 and 7 mg MLS; 370 mg 1.136 and 8 mg MLS; 370 mg 1.136 and 9 mg MLS; 370 mg 1.136 and 10 mg MLS; 370 mg 1.136 and 11 mg MLS; 370 mg 1.136 and 12 mg MLS; 370 mg 1.136 and 13 mg MLS; 370 mg 1.136 and 14 mg MLS; 370 mg 1.136 and 15 mg MLS; 370 mg 1.136 and 16 mg MLS; 370 mg 1.136 and 17 mg MLS; 370 mg 1.136 and 18 mg MLS; 370 mg 1.136 and 19 mg MLS; 370 mg 1.136 and 20 mg MLS.

375 mg 1.136 and 1 mg MLS; 375 mg 1.136 and 2 mg MLS; 375 mg 1.136 and 3 mg MLS; 375 mg 1.136 and 4 mg MLS; 375 mg 1.136 and 5 mg MLS; 375 mg 1.136 and 6 mg MLS; 375 mg 1.136 and 7 mg MLS; 375 mg 1.136 and 8 mg MLS; 375 mg 1.136 and 9 mg MLS; 375 mg 1.136 and 10 mg MLS; 375 mg 1.136 and 11 mg MLS; 375 mg 1.136 and 12 mg MLS; 375 mg 1.136 and 13 mg MLS; 375 mg 1.136 and 14 mg MLS; 375 mg 1.136 and 15 mg MLS; 375 mg 1.136 and 16 mg MLS; 375 mg 1.136 and 17 mg MLS; 375 mg 1.136 and 18 mg MLS; 375 mg 1.136 and 19 mg MLS; 375 mg 1.136 and 20 mg MLS.

380 mg 1.136 and 1 mg MLS; 380 mg 1.136 and 2 mg MLS; 380 mg 1.136 and 3 mg MLS; 380 mg 1.136 and 4 mg MLS; 380 mg 1.136 and 5 mg MLS; 380 mg 1.136 and 6 mg MLS; 380 mg 1.136 and 7 mg MLS; 380 mg 1.136 and 8 mg MLS; 380 mg 1.136 and 9 mg MLS; 380 mg 1.136 and 10 mg MLS; 380 mg 1.136 and 11 mg MLS; 380 mg 1.136 and 12 mg MLS; 380 mg 1.136 and 13 mg MLS; 380 mg 1.136 and 14 mg MLS; 380 mg 1.136 and 15 mg MLS; 380 mg 1.136 and 16 mg MLS; 380 mg 1.136 and 17 mg MLS; 380 mg 1.136 and 18 mg MLS; 380 mg 1.136 and 19 mg MLS; 380 mg 1.136 and 20 mg MLS.

385 mg 1.136 and 1 mg MLS; 385 mg 1.136 and 2 mg MLS; 385 mg 1.136 and 3 mg MLS; 385 mg 1.136 and 4 mg MLS; 385 mg 1.136 and 5 mg MLS; 385 mg 1.136 and 6 mg MLS; 385 mg 1.136 and 7 mg MLS; 385 mg 1.136 and 8 mg MLS; 385 mg 1.136 and 9 mg MLS; 385 mg 1.136 and 10 mg MLS; 385 mg 1.136 and 11 mg MLS; 385 mg 1.136 and 12 mg MLS; 385 mg 1.136 and 13 mg MLS; 385 mg 1.136 and 14 mg MLS; 385 mg 1.136 and 15 mg MLS; 385 mg 1.136 and 16 mg MLS; 385 mg 1.136 and 17 mg MLS; 385 mg 1.136 and 18 mg MLS; 385 mg 1.136 and 19 mg MLS; 385 mg 1.136 and 20 mg MLS.

390 mg 1.136 and 1 mg MLS; 390 mg 1.136 and 2 mg MLS; 390 mg 1.136 and 3 mg MLS; 390 mg 1.136 and 4 mg MLS; 390 mg 1.136 and 5 mg MLS; 390 mg 1.136 and 6 mg MLS; 390 mg 1.136 and 7 mg MLS; 390 mg 1.136 and 8 mg MLS; 390 mg 1.136 and 9 mg MLS; 390 mg 1.136 and 10 mg MLS; 390 mg 1.136 and 11 mg MLS; 390 mg 1.136 and 12 mg MLS; 390 mg 1.136 and 13 mg MLS; 390 mg 1.136 and 14 mg MLS; 390 mg 1.136 and 15 mg MLS; 390 mg 1.136 and 16 mg MLS; 390 mg 1.136 and 17 mg MLS; 390 mg 1.136 and 18 mg MLS; 390 mg 1.136 and 19 mg MLS; 390 mg 1.136 and 20 mg MLS.

395 mg 1.136 and 1 mg MLS; 395 mg 1.136 and 2 mg MLS; 395 mg 1.136 and 3 mg MLS; 395 mg 1.136 and 4 mg MLS; 395 mg 1.136 and 5 mg MLS; 395 mg 1.136 and 6 mg MLS; 395 mg 1.136 and 7 mg MLS; 395 mg 1.136 and 8 mg MLS; 395 mg 1.136 and 9 mg MLS; 395 mg 1.136 and 10 mg MLS; 395 mg 1.136 and 11 mg MLS; 395 mg 1.136 and 12 mg MLS; 395 mg 1.136 and 13 mg MLS; 395 mg 1.136 and 14 mg MLS; 395 mg 1.136 and 15 mg MLS; 395 mg 1.136 and 16 mg MLS; 395 mg 1.136 and 17 mg MLS; 395 mg 1.136 and 18 mg MLS; 395 mg 1.136 and 19 mg MLS; 395 mg 1.136 and 20 mg MLS.

400 mg 1.136 and 1 mg MLS; 400 mg 1.136 and 2 mg MLS; 400 mg 1.136 and 3 mg MLS; 400 mg 1.136 and 4 mg MLS; 400 mg 1.136 and 5 mg MLS; 400 mg 1.136 and 6 mg MLS; 400 mg 1.136 and 7 mg MLS; 400 mg 1.136 and 8 mg MLS; 400 mg 1.136 and 9 mg MLS; 400 mg 1.136 and 10 mg MLS; 400 mg 1.136 and 11 mg MLS; 400 mg 1.136 and 12 mg MLS; 400 mg 1.136 and 13 mg MLS; 400 mg 1.136 and 14 mg MLS; 400 mg 1.136 and 15 mg MLS; 400 mg 1.136 and 16 mg MLS; 400 mg 1.136 and 17 mg MLS; 400 mg 1.136 and 18 mg MLS; 400 mg 1.136 and 19 mg MLS; 400 mg 1.136 and 20 mg MLS.

Based on the data listed herein unit dose forms containing the following amounts of 1.136 and zafirlukast (ZL) are explicitly encompassed by the present invention:

10 mg 1.136 and 1 mg ZL; 10 mg 1.136 and 2 mg ZL; 10 mg 1.136 and 3 mg ZL; 10 mg 1.136 and 4 mg ZL; 10 mg 1.136 and 5 mg ZL; 10 mg 1.136 and 6 mg ZL; 10 mg 1.136 and 7 mg ZL; 10 mg 1.136 and 8 mg ZL; 10 mg 1.136 and 9 mg ZL; 10 mg 1.136 and 10 mg ZL; 10 mg 1.136 and 11 mg ZL; 10 mg 1.136 and 12 mg ZL; 10 mg 1.136 and 13 mg ZL; 10 mg 1.136 and 14 mg ZL; 10 mg 1.136 and 15 mg ZL; 10 mg 1.136 and 16 mg ZL; 10 mg 1.136 and 17 mg ZL; 10 mg 1.136 and 18 mg ZL; 10 mg 1.136 and 19 mg ZL; 10 mg 1.136 and 20 mg ZL;

15 mg 1.136 and 1 mg ZL; 15 mg 1.136 and 2 mg ZL; 15 mg 1.136 and 3 mg ZL; 15 mg 1.136 and 4 mg ZL; 15 mg 1.136 and 5 mg ZL; 15 mg 1.136 and 6 mg ZL; 15 mg 1.136 and 7 mg ZL; 15 mg 1.136 and 8 mg ZL; 15 mg 1.136 and 9 mg ZL; 15 mg 1.136 and 10 mg ZL; 15 mg 1.136 and 11 mg ZL; 15 mg 1.136 and 12 mg ZL; 15 mg 1.136 and 13 mg ZL; 15 mg 1.136 and 14 mg ZL; 15 mg 1.136 and 15 mg ZL; 15 mg 1.136 and 16 mg ZL; 15 mg 1.136 and 17 mg ZL; 15 mg 1.136 and 18 mg ZL; 15 mg 1.136 and 19 mg ZL; 15 mg 1.136 and 20 mg ZL;

20 mg 1.136 and 1 mg ZL; 20 mg 1.136 and 2 mg ZL; 20 mg 1.136 and 3 mg ZL; 20 mg 1.136 and 4 mg ZL; 20 mg 1.136 and 5 mg ZL; 20 mg 1.136 and 6 mg ZL; 20 mg 1.136 and 7 mg ZL; 20 mg 1.136 and 8 mg ZL; 20 mg 1.136 and 9 mg ZL; 20 mg 1.136 and 10 mg ZL; 20 mg 1.136 and 11 mg ZL; 20 mg 1.136 and 12 mg ZL; 20 mg 1.136 and 13 mg ZL; 20 mg 1.136 and 14 mg ZL; 20 mg 1.136 and 15 mg ZL; 20 mg 1.136 and 16 mg ZL; 20 mg 1.136 and 17 mg ZL; 20 mg 1.136 and 18 mg ZL; 20 mg 1.136 and 19 mg ZL; 20 mg 1.136 and 20 mg ZL;

25 mg 1.136 and 1 mg ZL; 25 mg 1.136 and 2 mg ZL; 25 mg 1.136 and 3 mg ZL; 25 mg 1.136 and 4 mg ZL; 25 mg 1.136 and 5 mg ZL; 25 mg 1.136 and 6 mg ZL; 25 mg 1.136 and 7 mg ZL; 25 mg 1.136 and 8 mg ZL; 25 mg 1.136 and 9 mg ZL; 25 mg 1.136 and 10 mg ZL; 25 mg 1.136 and 11 mg ZL; 25 mg 1.136 and 12 mg ZL; 25 mg 1.136 and 13 mg ZL; 25 mg 1.136 and 14 mg ZL; 25 mg 1.136 and 15 mg ZL; 25 mg 1.136 and 16 mg ZL; 25 mg 1.136 and 17 mg ZL; 25 mg 1.136 and 18 mg ZL; 25 mg 1.136 and 19 mg ZL; 25 mg 1.136 and 20 mg ZL;

30 mg 1.136 and 1 mg ZL; 30 mg 1.136 and 2 mg ZL; 30 mg 1.136 and 3 mg ZL; 30 mg 1.136 and 4 mg ZL; 30 mg 1.136 and 5 mg ZL; 30 mg 1.136 and 6 mg ZL; 30 mg 1.136 and 7 mg ZL; 30 mg 1.136 and 8 mg ZL; 30 mg 1.136 and 9 mg ZL; 30 mg 1.136 and 10 mg ZL; 30 mg 1.136 and 11 mg ZL; 30 mg 1.136 and 12 mg ZL; 30 mg 1.136 and 13 mg ZL; 30 mg 1.136 and 14 mg ZL; 30 mg 1.136 and 15 mg ZL; 30 mg 1.136 and 16 mg ZL; 30 mg 1.136 and 17 mg ZL; 30 mg 1.136 and 18 mg ZL; 30 mg 1.136 and 19 mg ZL; 30 mg 1.136 and 20 mg ZL;

35 mg 1.136 and 1 mg ZL; 35 mg 1.136 and 2 mg ZL; 35 mg 1.136 and 3 mg ZL; 35 mg 1.136 and 4 mg ZL; 35 mg 1.136 and 5 mg ZL; 35 mg 1.136 and 6 mg ZL; 35 mg 1.136 and 7 mg ZL; 35 mg 1.136 and 8 mg ZL; 35 mg 1.136 and 9 mg ZL; 35 mg 1.136 and 10 mg ZL; 35 mg 1.136 and 11 mg ZL; 35 mg 1.136 and 12 mg ZL; 35 mg 1.136 and 13 mg ZL; 35 mg 1.136 and 14 mg ZL; 35 mg 1.136 and 15 mg ZL; 35 mg 1.136 and 16 mg ZL; 35 mg 1.136 and 17 mg ZL; 35 mg 1.136 and 18 mg ZL; 35 mg 1.136 and 19 mg ZL; 35 mg 1.136 and 20 mg ZL;

40 mg 1.136 and 1 mg ZL; 40 mg 1.136 and 2 mg ZL; 40 mg 1.136 and 3 mg ZL; 40 mg 1.136 and 4 mg ZL; 40 mg 1.136 and 5 mg ZL; 40 mg 1.136 and 6 mg ZL; 40 mg 1.136 and 7 mg ZL; 40 mg 1.136 and 8 mg ZL; 40 mg 1.136 and 9 mg ZL; 40 mg 1.136 and 10 mg ZL; 40 mg 1.136 and 11 mg ZL; 40 mg 1.136 and 12 mg ZL; 40 mg 1.136 and 13 mg ZL; 40 mg 1.136 and 14 mg ZL; 40 mg 1.136 and 15 mg ZL; 40 mg 1.136 and 16 mg ZL; 40 mg 1.136 and 17 mg ZL; 40 mg 1.136 and 18 mg ZL; 40 mg 1.136 and 19 mg ZL; 40 mg 1.136 and 20 mg ZL;

45 mg 1.136 and 1 mg ZL; 45 mg 1.136 and 2 mg ZL; 45 mg 1.136 and 3 mg ZL; 45 mg 1.136 and 4 mg ZL; 45 mg 1.136 and 5 mg ZL; 45 mg 1.136 and 6 mg ZL; 45 mg 1.136 and 7 mg ZL; 45 mg 1.136 and 8 mg ZL; 45 mg 1.136 and 9 mg ZL; 45 mg 1.136 and 10 mg ZL; 45 mg 1.136 and 11 mg ZL; 45 mg 1.136 and 12 mg ZL; 45 mg 1.136 and 13 mg ZL; 45 mg 1.136 and 14 mg ZL; 45 mg 1.136 and 15 mg ZL; 45 mg 1.136 and 16 mg ZL; 45 mg 1.136 and 17 mg ZL; 45 mg 1.136 and 18 mg ZL; 45 mg 1.136 and 19 mg ZL; 45 mg 1.136 and 20 mg ZL;

50 mg 1.136 and 1 mg ZL; 50 mg 1.136 and 2 mg ZL; 50 mg 1.136 and 3 mg ZL; 50 mg 1.136 and 4 mg ZL; 50 mg 1.136 and 5 mg ZL; 50 mg 1.136 and 6 mg ZL; 50 mg 1.136 and 7 mg ZL; 50 mg 1.136 and 8 mg ZL; 50 mg 1.136 and 9 mg ZL; 50 mg 1.136 and 10 mg ZL; 50 mg 1.136 and 11 mg ZL; 50 mg 1.136 and 12 mg ZL; 50 mg 1.136 and 13 mg ZL; 50 mg 1.136 and 14 mg ZL; 50 mg 1.136 and 15 mg ZL; 50 mg 1.136 and 16 mg ZL; 50 mg 1.136 and 17 mg ZL; 50 mg 1.136 and 18 mg ZL; 50 mg 1.136 and 19 mg ZL; 50 mg 1.136 and 20 mg ZL;

55 mg 1.136 and 1 mg ZL; 55 mg 1.136 and 2 mg ZL; 55 mg 1.136 and 3 mg ZL; 55 mg 1.136 and 4 mg ZL; 55 mg 1.136 and 5 mg ZL; 55 mg 1.136 and 6 mg ZL; 55 mg 1.136 and 7 mg ZL; 55 mg 1.136 and 8 mg ZL; 55 mg 1.136 and 9 mg ZL; 55 mg 1.136 and 10 mg ZL; 55 mg 1.136 and 11 mg ZL; 55 mg 1.136 and 12 mg ZL; 55 mg 1.136 and 13 mg ZL; 55 mg 1.136 and 14 mg ZL; 55 mg 1.136 and 15 mg ZL; 55 mg 1.136 and 16 mg ZL; 55 mg 1.136 and 17 mg ZL; 55 mg 1.136 and 18 mg ZL; 55 mg 1.136 and 19 mg ZL; 55 mg 1.136 and 20 mg ZL;

60 mg 1.136 and 1 mg ZL; 60 mg 1.136 and 2 mg ZL; 60 mg 1.136 and 3 mg ZL; 60 mg 1.136 and 4 mg ZL; 60 mg 1.136 and 5 mg ZL; 60 mg 1.136 and 6 mg ZL; 60 mg 1.136 and 7 mg ZL; 60 mg 1.136 and 8 mg ZL; 60 mg 1.136 and 9 mg ZL; 60 mg 1.136 and 10 mg ZL; 60 mg 1.136 and 11 mg ZL; 60 mg 1.136 and 12 mg ZL; 60 mg 1.136 and 13 mg ZL; 60 mg 1.136 and 14 mg ZL; 60 mg 1.136 and 15 mg ZL; 60 mg 1.136 and 16 mg ZL; 60 mg 1.136 and 17 mg ZL; 60 mg 1.136 and 18 mg ZL; 60 mg 1.136 and 19 mg ZL; 60 mg 1.136 and 20 mg ZL;

65 mg 1.136 and 1 mg ZL; 65 mg 1.136 and 2 mg ZL; 65 mg 1.136 and 3 mg ZL; 65 mg 1.136 and 4 mg ZL; 65 mg 1.136 and 5 mg ZL; 65 mg 1.136 and 6 mg ZL; 65 mg 1.136 and 7 mg ZL; 65 mg 1.136 and 8 mg ZL; 65 mg 1.136 and 9 mg ZL; 65 mg 1.136 and 10 mg ZL; 65 mg 1.136 and 11 mg ZL; 65 mg 1.136 and 12 mg ZL; 65 mg 1.136 and 13 mg ZL; 65 mg 1.136 and 14 mg ZL; 65 mg 1.136 and 15 mg ZL; 65 mg 1.136 and 16 mg ZL; 65 mg 1.136 and 17 mg ZL; 65 mg 1.136 and 18 mg ZL; 65 mg 1.136 and 19 mg ZL; 65 mg 1.136 and 20 mg ZL;

70 mg 1.136 and 1 mg ZL; 70 mg 1.136 and 2 mg ZL; 70 mg 1.136 and 3 mg ZL; 70 mg 1.136 and 4 mg ZL; 70 mg 1.136 and 5 mg ZL; 70 mg 1.136 and 6 mg ZL; 70 mg 1.136 and 7 mg ZL; 70 mg 1.136 and 8 mg ZL; 70 mg 1.136 and 9 mg ZL; 70 mg 1.136 and 10 mg ZL; 70 mg 1.136 and 11 mg ZL; 70 mg 1.136 and 12 mg ZL; 70 mg 1.136 and 13 mg ZL; 70 mg 1.136 and 14 mg ZL; 70 mg 1.136 and 15 mg ZL; 70 mg 1.136 and 16 mg ZL; 70 mg 1.136 and 17 mg ZL; 70 mg 1.136 and 18 mg ZL; 70 mg 1.136 and 19 mg ZL; 70 mg 1.136 and 20 mg ZL;

75 mg 1.136 and 1 mg ZL; 75 mg 1.136 and 2 mg ZL; 75 mg 1.136 and 3 mg ZL; 75 mg 1.136 and 4 mg ZL; 75 mg 1.136 and 5 mg ZL; 75 mg 1.136 and 6 mg ZL; 75 mg 1.136 and 7 mg ZL; 75 mg 1.136 and 8 mg ZL; 75 mg 1.136 and 9 mg ZL; 75 mg 1.136 and 10 mg ZL; 75 mg 1.136 and 11 mg ZL; 75 mg 1.136 and 12 mg ZL; 75 mg 1.136 and 13 mg ZL; 75 mg 1.136 and 14 mg ZL; 75 mg 1.136 and 15 mg ZL; 75 mg 1.136 and 16 mg ZL; 75 mg 1.136 and 17 mg ZL; 75 mg 1.136 and 18 mg ZL; 75 mg 1.136 and 19 mg ZL; 75 mg 1.136 and 20 mg ZL;

80 mg 1.136 and 1 mg ZL; 80 mg 1.136 and 2 mg ZL; 80 mg 1.136 and 3 mg ZL; 80 mg 1.136 and 4 mg ZL; 80 mg 1.136 and 5 mg ZL; 80 mg 1.136 and 6 mg ZL; 80 mg 1.136 and 7 mg ZL; 80 mg 1.136 and 8 mg ZL; 80 mg 1.136 and 9 mg ZL; 80 mg 1.136 and 10 mg ZL; 80 mg 1.136 and 11 mg ZL; 80 mg 1.136 and 12 mg ZL; 80 mg 1.136 and 13 mg ZL; 80 mg 1.136 and 14 mg ZL; 80 mg 1.136 and 15 mg ZL; 80 mg 1.136 and 16 mg ZL; 80 mg 1.136 and 17 mg ZL; 80 mg 1.136 and 18 mg ZL; 80 mg 1.136 and 19 mg ZL; 80 mg 1.136 and 20 mg ZL;

85 mg 1.136 and 1 mg ZL; 85 mg 1.136 and 2 mg ZL; 85 mg 1.136 and 3 mg ZL; 85 mg 1.136 and 4 mg ZL; 85 mg 1.136 and 5 mg ZL; 85 mg 1.136 and 6 mg ZL; 85 mg 1.136 and 7 mg ZL; 85 mg 1.136 and 8 mg ZL; 85 mg 1.136 and 9 mg ZL; 85 mg 1.136 and 10 mg ZL; 85 mg 1.136 and 11 mg ZL; 85 mg 1.136 and 12 mg ZL; 85 mg 1.136 and 13 mg ZL; 85 mg 1.136 and 14 mg ZL; 85 mg 1.136 and 15 mg ZL; 85 mg 1.136 and 16 mg ZL; 85 mg 1.136 and 17 mg ZL; 85 mg 1.136 and 18 mg ZL; 85 mg 1.136 and 19 mg ZL; 85 mg 1.136 and 20 mg ZL;

90 mg 1.136 and 1 mg ZL; 90 mg 1.136 and 2 mg ZL; 90 mg 1.136 and 3 mg ZL; 90 mg 1.136 and 4 mg ZL; 90 mg 1.136 and 5 mg ZL; 90 mg 1.136 and 6 mg ZL; 90 mg 1.136 and 7 mg ZL; 90 mg 1.136 and 8 mg ZL; 90 mg 1.136 and 9 mg ZL; 90 mg 1.136 and 10 mg ZL; 90 mg 1.136 and 11 mg ZL; 90 mg 1.136 and 12 mg ZL; 90 mg 1.136 and 13 mg ZL; 90 mg 1.136 and 14 mg ZL; 90 mg 1.136 and 15 mg ZL; 90 mg 1.136 and 16 mg ZL; 90 mg 1.136 and 17 mg ZL; 90 mg 1.136 and 18 mg ZL; 90 mg 1.136 and 19 mg ZL; 90 mg 1.136 and 20 mg ZL;

95 mg 1.136 and 1 mg ZL; 95 mg 1.136 and 2 mg ZL; 95 mg 1.136 and 3 mg ZL; 95 mg 1.136 and 4 mg ZL; 95 mg 1.136 and 5 mg ZL; 95 mg 1.136 and 6 mg ZL; 95 mg 1.136 and 7 mg ZL; 95 mg 1.136 and 8 mg ZL; 95 mg 1.136 and 9 mg ZL; 95 mg 1.136 and 10 mg ZL; 95 mg 1.136 and 11 mg ZL; 95 mg 1.136 and 12 mg ZL; 95 mg 1.136 and 13 mg ZL; 95 mg 1.136 and 14 mg ZL; 95 mg 1.136 and 15 mg ZL; 95 mg 1.136 and 16 mg ZL; 95 mg 1.136 and 17 mg ZL; 95 mg 1.136 and 18 mg ZL; 95 mg 1.136 and 19 mg ZL; 95 mg 1.136 and 20 mg ZL;

100 mg 1.136 and 1 mg ZL; 100 mg 1.136 and 2 mg ZL; 100 mg 1.136 and 3 mg ZL; 100 mg 1.136 and 4 mg ZL; 100 mg 1.136 and 5 mg ZL; 100 mg 1.136 and 6 mg ZL; 100 mg 1.136 and 7 mg ZL; 100 mg 1.136 and 8 mg ZL; 100 mg 1.136 and 9 mg ZL; 100 mg 1.136 and 10 mg ZL; 100 mg 1.136 and 11 mg ZL; 100 mg 1.136 and 12 mg ZL; 100 mg 1.136 and 13 mg ZL; 100 mg 1.136 and 14 mg ZL; 100 mg 1.136 and 15 mg ZL; 100 mg 1.136 and 16 mg ZL; 100 mg 1.136 and 17 mg ZL; 100 mg 1.136 and 18 mg ZL; 100 mg 1.136 and 19 mg ZL; 100 mg 1.136 and 20 mg ZL;

105 mg 1.136 and 1 mg ZL; 105 mg 1.136 and 2 mg ZL; 105 mg 1.136 and 3 mg ZL; 105 mg 1.136 and 4 mg ZL; 105 mg 1.136 and 5 mg ZL; 105 mg 1.136 and 6 mg ZL; 105 mg 1.136 and 7 mg ZL; 105 mg 1.136 and 8 mg ZL; 105 mg 1.136 and 9 mg ZL; 105 mg 1.136 and 10 mg ZL; 105 mg 1.136 and 11 mg ZL; 105 mg 1.136 and 12 mg ZL; 105 mg 1.136 and 13 mg ZL; 105 mg 1.136 and 14 mg ZL; 105 mg 1.136 and 15 mg ZL; 105 mg 1.136 and 16 mg ZL; 105 mg 1.136 and 17 mg ZL; 105 mg 1.136 and 18 mg ZL; 105 mg 1.136 and 19 mg ZL; 105 mg 1.136 and 20 mg ZL;

110 mg 1.136 and 1 mg ZL; 110 mg 1.136 and 2 mg ZL; 110 mg 1.136 and 3 mg ZL; 110 mg 1.136 and 4 mg ZL; 110 mg 1.136 and 5 mg ZL; 110 mg 1.136 and 6 mg ZL; 110 mg 1.136 and 7 mg ZL; 110 mg 1.136 and 8 mg ZL; 110 mg 1.136 and 9 mg ZL; 110 mg 1.136 and 10 mg ZL; 110 mg 1.136 and 11 mg ZL; 110 mg 1.136 and 12 mg ZL; 110 mg 1.136 and 13 mg ZL; 110 mg 1.136 and 14 mg ZL; 110 mg 1.136 and 15 mg ZL; 110 mg 1.136 and 16 mg ZL; 110 mg 1.136 and 17 mg ZL; 110 mg 1.136 and 18 mg ZL; 110 mg 1.136 and 19 mg ZL; 110 mg 1.136 and 20 mg ZL;

115 mg 1.136 and 1 mg ZL; 115 mg 1.136 and 2 mg ZL; 115 mg 1.136 and 3 mg ZL; 115 mg 1.136 and 4 mg ZL; 115 mg 1.136 and 5 mg ZL; 115 mg 1.136 and 6 mg ZL; 115 mg 1.136 and 7 mg ZL; 115 mg 1.136 and 8 mg ZL; 115 mg 1.136 and 9 mg ZL; 115 mg 1.136 and 10 mg ZL; 115 mg 1.136 and 11 mg ZL; 115 mg 1.136 and 12 mg ZL; 115 mg 1.136 and 13 mg ZL; 115 mg 1.136 and 14 mg ZL; 115 mg 1.136 and 15 mg ZL; 115 mg 1.136 and 16 mg ZL; 115 mg 1.136 and 17 mg ZL; 115 mg 1.136 and 18 mg ZL; 115 mg 1.136 and 19 mg ZL; 115 mg 1.136 and 20 mg ZL;

120 mg 1.136 and 1 mg ZL; 120 mg 1.136 and 2 mg ZL; 120 mg 1.136 and 3 mg ZL; 120 mg 1.136 and 4 mg ZL; 120 mg 1.136 and 5 mg ZL; 120 mg 1.136 and 6 mg ZL; 120 mg 1.136 and 7 mg ZL; 120 mg 1.136 and 8 mg ZL; 120 mg 1.136 and 9 mg ZL; 120 mg 1.136 and 10 mg ZL; 120 mg 1.136 and 11 mg ZL; 120 mg 1.136 and 12 mg ZL; 120 mg 1.136 and 13 mg ZL; 120 mg 1.136 and 14 mg ZL; 120 mg 1.136 and 15 mg ZL; 120 mg 1.136 and 16 mg ZL; 120 mg 1.136 and 17 mg ZL; 120 mg 1.136 and 18 mg ZL; 120 mg 1.136 and 19 mg ZL; 120 mg 1.136 and 20 mg ZL;

125 mg 1.136 and 1 mg ZL; 125 mg 1.136 and 2 mg ZL; 125 mg 1.136 and 3 mg ZL; 125 mg 1.136 and 4 mg ZL; 125 mg 1.136 and 5 mg ZL; 125 mg 1.136 and 6 mg ZL; 125 mg 1.136 and 7 mg ZL; 125 mg 1.136 and 8 mg ZL; 125 mg 1.136 and 9 mg ZL; 125 mg 1.136 and 10 mg ZL; 125 mg 1.136 and 11 mg ZL; 125 mg 1.136 and 12 mg ZL; 125 mg 1.136 and 13 mg ZL; 125 mg 1.136 and 14 mg ZL; 125 mg 1.136 and 15 mg ZL; 125 mg 1.136 and 16 mg ZL; 125 mg 1.136 and 17 mg ZL; 125 mg 1.136 and 18 mg ZL; 125 mg 1.136 and 19 mg ZL; 125 mg 1.136 and 20 mg ZL;

130 mg 1.136 and 1 mg ZL; 130 mg 1.136 and 2 mg ZL; 130 mg 1.136 and 3 mg ZL; 130 mg 1.136 and 4 mg ZL; 130 mg 1.136 and 5 mg ZL; 130 mg 1.136 and 6 mg ZL; 130 mg 1.136 and 7 mg ZL; 130 mg 1.136 and 8 mg ZL; 130 mg 1.136 and 9 mg ZL; 130 mg 1.136 and 10 mg ZL; 130 mg 1.136 and 11 mg ZL; 130 mg 1.136 and 12 mg ZL; 130 mg 1.136 and 13 mg ZL; 130 mg 1.136 and 14 mg ZL; 130 mg 1.136 and 15 mg ZL; 130 mg 1.136 and 16 mg ZL; 130 mg 1.136 and 17 mg ZL; 130 mg 1.136 and 18 mg ZL; 130 mg 1.136 and 19 mg ZL; 130 mg 1.136 and 20 mg ZL;

135 mg 1.136 and 1 mg ZL; 135 mg 1.136 and 2 mg ZL; 135 mg 1.136 and 3 mg ZL; 135 mg 1.136 and 4 mg ZL; 135 mg 1.136 and 5 mg ZL; 135 mg 1.136 and 6 mg ZL; 135 mg 1.136 and 7 mg ZL; 135 mg 1.136 and 8 mg ZL; 135 mg 1.136 and 9 mg ZL; 135 mg 1.136 and 10 mg ZL; 135 mg 1.136 and 11 mg ZL; 135 mg 1.136 and 12 mg ZL; 135 mg 1.136 and 13 mg ZL; 135 mg 1.136 and 14 mg ZL; 135 mg 1.136 and 15 mg ZL; 135 mg 1.136 and 16 mg ZL; 135 mg 1.136 and 17 mg ZL; 135 mg 1.136 and 18 mg ZL; 135 mg 1.136 and 19 mg ZL; 135 mg 1.136 and 20 mg ZL;

140 mg 1.136 and 1 mg ZL; 140 mg 1.136 and 2 mg ZL; 140 mg 1.136 and 3 mg ZL; 140 mg 1.136 and 4 mg ZL; 140 mg 1.136 and 5 mg ZL; 140 mg 1.136 and 6 mg ZL; 140 mg 1.136 and 7 mg ZL; 140 mg 1.136 and 8 mg ZL; 140 mg 1.136 and 9 mg ZL; 140 mg 1.136 and 10 mg ZL; 140 mg 1.136 and 11 mg ZL; 140 mg 1.136 and 12 mg ZL; 140 mg 1.136 and 13 mg ZL; 140 mg 1.136 and 14 mg ZL; 140 mg 1.136 and 15 mg ZL; 140 mg 1.136 and 16 mg ZL; 140 mg 1.136 and 17 mg ZL; 140 mg 1.136 and 18 mg ZL; 140 mg 1.136 and 19 mg ZL; 140 mg 1.136 and 20 mg ZL;

145 mg 1.136 and 1 mg ZL; 145 mg 1.136 and 2 mg ZL; 145 mg 1.136 and 3 mg ZL; 145 mg 1.136 and 4 mg ZL; 145 mg 1.136 and 5 mg ZL; 145 mg 1.136 and 6 mg ZL; 145 mg 1.136 and 7 mg ZL; 145 mg 1.136 and 8 mg ZL; 145 mg 1.136 and 9 mg ZL; 145 mg 1.136 and 10 mg ZL; 145 mg 1.136 and 11 mg ZL; 145 mg 1.136 and 12 mg ZL; 145 mg 1.136 and 13 mg ZL; 145 mg 1.136 and 14 mg ZL; 145 mg 1.136 and 15 mg ZL; 145 mg 1.136 and 16 mg ZL; 145 mg 1.136 and 17 mg ZL; 145 mg 1.136 and 18 mg ZL; 145 mg 1.136 and 19 mg ZL; 145 mg 1.136 and 20 mg ZL;

150 mg 1.136 and 1 mg ZL; 150 mg 1.136 and 2 mg ZL; 150 mg 1.136 and 3 mg ZL; 150 mg 1.136 and 4 mg ZL; 150 mg 1.136 and 5 mg ZL; 150 mg 1.136 and 6 mg ZL; 150 mg 1.136 and 7 mg ZL; 150 mg 1.136 and 8 mg ZL; 150 mg 1.136 and 9 mg ZL; 150 mg 1.136 and 10 mg ZL; 150 mg 1.136 and 11 mg ZL; 150 mg 1.136 and 12 mg ZL; 150 mg 1.136 and 13 mg ZL; 150 mg 1.136 and 14 mg ZL; 150 mg 1.136 and 15 mg ZL; 150 mg 1.136 and 16 mg ZL; 150 mg 1.136 and 17 mg ZL; 150 mg 1.136 and 18 mg ZL; 150 mg 1.136 and 19 mg ZL; 150 mg 1.136 and 20 mg ZL;

155 mg 1.136 and 1 mg ZL; 155 mg 1.136 and 2 mg ZL; 155 mg 1.136 and 3 mg ZL; 155 mg 1.136 and 4 mg ZL; 155 mg 1.136 and 5 mg ZL; 155 mg 1.136 and 6 mg ZL; 155 mg 1.136 and 7 mg ZL; 155 mg 1.136 and 8 mg ZL; 155 mg 1.136 and 9 mg ZL; 155 mg 1.136 and 10 mg ZL; 155 mg 1.136 and 11 mg ZL; 155 mg 1.136 and 12 mg ZL; 155 mg 1.136 and 13 mg ZL; 155 mg 1.136 and 14 mg ZL; 155 mg 1.136 and 15 mg ZL; 155 mg 1.136 and 16 mg ZL; 155 mg 1.136 and 17 mg ZL; 155 mg 1.136 and 18 mg ZL; 155 mg 1.136 and 19 mg ZL; 155 mg 1.136 and 20 mg ZL;

160 mg 1.136 and 1 mg ZL; 160 mg 1.136 and 2 mg ZL; 160 mg 1.136 and 3 mg ZL; 160 mg 1.136 and 4 mg ZL; 160 mg 1.136 and 5 mg ZL; 160 mg 1.136 and 6 mg ZL; 160 mg 1.136 and 7 mg ZL; 160 mg 1.136 and 8 mg ZL; 160 mg 1.136 and 9 mg ZL; 160 mg 1.136 and 10 mg ZL; 160 mg 1.136 and 11 mg ZL; 160 mg 1.136 and 12 mg ZL; 160 mg 1.136 and 13 mg ZL; 160 mg 1.136 and 14 mg ZL; 160 mg 1.136 and 15 mg ZL; 160 mg 1.136 and 16 mg ZL; 160 mg 1.136 and 17 mg ZL; 160 mg 1.136 and 18 mg ZL; 160 mg 1.136 and 19 mg ZL; 160 mg 1.136 and 20 mg ZL;

165 mg 1.136 and 1 mg ZL; 165 mg 1.136 and 2 mg ZL; 165 mg 1.136 and 3 mg ZL; 165 mg 1.136 and 4 mg ZL; 165 mg 1.136 and 5 mg ZL; 165 mg 1.136 and 6 mg ZL; 165 mg 1.136 and 7 mg ZL; 165 mg 1.136 and 8 mg ZL; 165 mg 1.136 and 9 mg ZL; 165 mg 1.136 and 10 mg ZL; 165 mg 1.136 and 11 mg ZL; 165 mg 1.136 and 12 mg ZL; 165 mg 1.136 and 13 mg ZL; 165 mg 1.136 and 14 mg ZL; 165 mg 1.136 and 15 mg ZL; 165 mg 1.136 and 16 mg ZL; 165 mg 1.136 and 17 mg ZL; 165 mg 1.136 and 18 mg ZL; 165 mg 1.136 and 19 mg ZL; 165 mg 1.136 and 20 mg ZL;

170 mg 1.136 and 1 mg ZL; 170 mg 1.136 and 2 mg ZL; 170 mg 1.136 and 3 mg ZL; 170 mg 1.136 and 4 mg ZL; 170 mg 1.136 and 5 mg ZL; 170 mg 1.136 and 6 mg ZL; 170 mg 1.136 and 7 mg ZL; 170 mg 1.136 and 8 mg ZL; 170 mg 1.136 and 9 mg ZL; 170 mg 1.136 and 10 mg ZL; 170 mg 1.136 and 11 mg ZL; 170 mg 1.136 and 12 mg ZL; 170 mg 1.136 and 13 mg ZL; 170 mg 1.136 and 14 mg ZL; 170 mg 1.136 and 15 mg ZL; 170 mg 1.136 and 16 mg ZL; 170 mg 1.136 and 17 mg ZL; 170 mg 1.136 and 18 mg ZL; 170 mg 1.136 and 19 mg ZL; 170 mg 1.136 and 20 mg ZL;

175 mg 1.136 and 1 mg ZL; 175 mg 1.136 and 2 mg ZL; 175 mg 1.136 and 3 mg ZL; 175 mg 1.136 and 4 mg ZL; 175 mg 1.136 and 5 mg ZL; 175 mg 1.136 and 6 mg ZL; 175 mg 1.136 and 7 mg ZL; 175 mg 1.136 and 8 mg ZL; 175 mg 1.136 and 9 mg ZL; 175 mg 1.136 and 10 mg ZL; 175 mg 1.136 and 11 mg ZL; 175 mg 1.136 and 12 mg ZL; 175 mg 1.136 and 13 mg ZL; 175 mg 1.136 and 14 mg ZL; 175 mg 1.136 and 15 mg ZL; 175 mg 1.136 and 16 mg ZL; 175 mg 1.136 and 17 mg ZL; 175 mg 1.136 and 18 mg ZL; 175 mg 1.136 and 19 mg ZL; 175 mg 1.136 and 20 mg ZL;

180 mg 1.136 and 1 mg ZL; 180 mg 1.136 and 2 mg ZL; 180 mg 1.136 and 3 mg ZL; 180 mg 1.136 and 4 mg ZL; 180 mg 1.136 and 5 mg ZL; 180 mg 1.136 and 6 mg ZL; 180 mg 1.136 and 7 mg ZL; 180 mg 1.136 and 8 mg ZL; 180 mg 1.136 and 9 mg ZL; 180 mg 1.136 and 10 mg ZL; 180 mg 1.136 and 11 mg ZL; 180 mg 1.136 and 12 mg ZL; 180 mg 1.136 and 13 mg ZL; 180 mg 1.136 and 14 mg ZL; 180 mg 1.136 and 15 mg ZL; 180 mg 1.136 and 16 mg ZL; 180 mg 1.136 and 17 mg ZL; 180 mg 1.136 and 18 mg ZL; 180 mg 1.136 and 19 mg ZL; 180 mg 1.136 and 20 mg ZL;

185 mg 1.136 and 1 mg ZL; 185 mg 1.136 and 2 mg ZL; 185 mg 1.136 and 3 mg ZL; 185 mg 1.136 and 4 mg ZL; 185 mg 1.136 and 5 mg ZL; 185 mg 1.136 and 6 mg ZL; 185 mg 1.136 and 7 mg ZL; 185 mg 1.136 and 8 mg ZL; 185 mg 1.136 and 9 mg ZL; 185 mg 1.136 and 10 mg ZL; 185 mg 1.136 and 11 mg ZL; 185 mg 1.136 and 12 mg ZL; 185 mg 1.136 and 13 mg ZL; 185 mg 1.136 and 14 mg ZL; 185 mg 1.136 and 15 mg ZL; 185 mg 1.136 and 16 mg ZL; 185 mg 1.136 and 17 mg ZL; 185 mg 1.136 and 18 mg ZL; 185 mg 1.136 and 19 mg ZL; 185 mg 1.136 and 20 mg ZL;

190 mg 1.136 and 1 mg ZL; 190 mg 1.136 and 2 mg ZL; 190 mg 1.136 and 3 mg ZL; 190 mg 1.136 and 4 mg ZL; 190 mg 1.136 and 5 mg ZL; 190 mg 1.136 and 6 mg ZL; 190 mg 1.136 and 7 mg ZL; 190 mg 1.136 and 8 mg ZL; 190 mg 1.136 and 9 mg ZL; 190 mg 1.136 and 10 mg ZL; 190 mg 1.136 and 11 mg ZL; 190 mg 1.136 and 12 mg ZL; 190 mg 1.136 and 13 mg ZL; 190 mg 1.136 and 14 mg ZL; 190 mg 1.136 and 15 mg ZL; 190 mg 1.136 and 16 mg ZL; 190 mg 1.136 and 17 mg ZL; 190 mg 1.136 and 18 mg ZL; 190 mg 1.136 and 19 mg ZL; 190 mg 1.136 and 20 mg ZL;

195 mg 1.136 and 1 mg ZL; 195 mg 1.136 and 2 mg ZL; 195 mg 1.136 and 3 mg ZL; 195 mg 1.136 and 4 mg ZL; 195 mg 1.136 and 5 mg ZL; 195 mg 1.136 and 6 mg ZL; 195 mg 1.136 and 7 mg ZL; 195 mg 1.136 and 8 mg ZL; 195 mg 1.136 and 9 mg ZL; 195 mg 1.136 and 10 mg ZL; 195 mg 1.136 and 11 mg ZL; 195 mg 1.136 and 12 mg ZL; 195 mg 1.136 and 13 mg ZL; 195 mg 1.136 and 14 mg ZL; 195 mg 1.136 and 15 mg ZL; 195 mg 1.136 and 16 mg ZL; 195 mg 1.136 and 17 mg ZL; 195 mg 1.136 and 18 mg ZL; 195 mg 1.136 and 19 mg ZL; 195 mg 1.136 and 20 mg ZL;

200 mg 1.136 and 1 mg ZL; 200 mg 1.136 and 2 mg ZL; 200 mg 1.136 and 3 mg ZL; 200 mg 1.136 and 4 mg ZL; 200 mg 1.136 and 5 mg ZL; 200 mg 1.136 and 6 mg ZL; 200 mg 1.136 and 7 mg ZL; 200 mg 1.136 and 8 mg ZL; 200 mg 1.136 and 9 mg ZL; 200 mg 1.136 and 10 mg ZL; 200 mg 1.136 and 11 mg ZL; 200 mg 1.136 and 12 mg ZL; 200 mg 1.136 and 13 mg ZL; 200 mg 1.136 and 14 mg ZL; 200 mg 1.136 and 15 mg ZL; 200 mg 1.136 and 16 mg ZL; 200 mg 1.136 and 17 mg ZL; 200 mg 1.136 and 18 mg ZL; 200 mg 1.136 and 19 mg ZL; 200 mg 1.136 and 20 mg ZL;

205 mg 1.136 and 1 mg ZL; 205 mg 1.136 and 2 mg ZL; 205 mg 1.136 and 3 mg ZL; 205 mg 1.136 and 4 mg ZL; 205 mg 1.136 and 5 mg ZL; 205 mg 1.136 and 6 mg ZL; 205 mg 1.136 and 7 mg ZL; 205 mg 1.136 and 8 mg ZL; 205 mg 1.136 and 9 mg ZL; 205 mg 1.136 and 10 mg ZL; 205 mg 1.136 and 11 mg ZL; 205 mg 1.136 and 12 mg ZL; 205 mg 1.136 and 13 mg ZL; 205 mg 1.136 and 14 mg ZL; 205 mg 1.136 and 15 mg ZL; 205 mg 1.136 and 16 mg ZL; 205 mg 1.136 and 17 mg ZL; 205 mg 1.136 and 18 mg ZL; 205 mg 1.136 and 19 mg ZL; 205 mg 1.136 and 20 mg ZL;

210 mg 1.136 and 1 mg ZL; 210 mg 1.136 and 2 mg ZL; 210 mg 1.136 and 3 mg ZL; 210 mg 1.136 and 4 mg ZL; 210 mg 1.136 and 5 mg ZL; 210 mg 1.136 and 6 mg ZL; 210 mg 1.136 and 7 mg ZL; 210 mg 1.136 and 8 mg ZL; 210 mg 1.136 and 9 mg ZL; 210 mg 1.136 and 10 mg ZL; 210 mg 1.136 and 11 mg ZL; 210 mg 1.136 and 12 mg ZL; 210 mg 1.136 and 13 mg ZL; 210 mg 1.136 and 14 mg ZL; 210 mg 1.136 and 15 mg ZL; 210 mg 1.136 and 16 mg ZL; 210 mg 1.136 and 17 mg ZL; 210 mg 1.136 and 18 mg ZL; 210 mg 1.136 and 19 mg ZL; 210 mg 1.136 and 20 mg ZL;

215 mg 1.136 and 1 mg ZL; 215 mg 1.136 and 2 mg ZL; 215 mg 1.136 and 3 mg ZL; 215 mg 1.136 and 4 mg ZL; 215 mg 1.136 and 5 mg ZL; 215 mg 1.136 and 6 mg ZL; 215 mg 1.136 and 7 mg ZL; 215 mg 1.136 and 8 mg ZL; 215 mg 1.136 and 9 mg ZL; 215 mg 1.136 and 10 mg ZL; 215 mg 1.136 and 11 mg ZL; 215 mg 1.136 and 12 mg ZL; 215 mg 1.136 and 13 mg ZL; 215 mg 1.136 and 14 mg ZL; 215 mg 1.136 and 15 mg ZL; 215 mg 1.136 and 16 mg ZL; 215 mg 1.136 and 17 mg ZL; 215 mg 1.136 and 18 mg ZL; 215 mg 1.136 and 19 mg ZL; 215 mg 1.136 and 20 mg ZL;

220 mg 1.136 and 1 mg ZL; 220 mg 1.136 and 2 mg ZL; 220 mg 1.136 and 3 mg ZL; 220 mg 1.136 and 4 mg ZL; 220 mg 1.136 and 5 mg ZL; 220 mg 1.136 and 6 mg ZL; 220 mg 1.136 and 7 mg ZL; 220 mg 1.136 and 8 mg ZL; 220 mg 1.136 and 9 mg ZL; 220 mg 1.136 and 10 mg ZL; 220 mg 1.136 and 11 mg ZL; 220 mg 1.136 and 12 mg ZL; 220 mg 1.136 and 13 mg ZL; 220 mg 1.136 and 14 mg ZL; 220 mg 1.136 and 15 mg ZL; 220 mg 1.136 and 16 mg ZL; 220 mg 1.136 and 17 mg ZL; 220 mg 1.136 and 18 mg ZL; 220 mg 1.136 and 19 mg ZL; 220 mg 1.136 and 20 mg ZL;

225 mg 1.136 and 1 mg ZL; 225 mg 1.136 and 2 mg ZL; 225 mg 1.136 and 3 mg ZL; 225 mg 1.136 and 4 mg ZL; 225 mg 1.136 and 5 mg ZL; 225 mg 1.136 and 6 mg ZL; 225 mg 1.136 and 7 mg ZL; 225 mg 1.136 and 8 mg ZL; 225 mg 1.136 and 9 mg ZL; 225 mg 1.136 and 10 mg ZL; 225 mg 1.136 and 11 mg ZL; 225 mg 1.136 and 12 mg ZL; 225 mg 1.136 and 13 mg ZL; 225 mg 1.136 and 14 mg ZL; 225 mg 1.136 and 15 mg ZL; 225 mg 1.136 and 16 mg ZL; 225 mg 1.136 and 17 mg ZL; 225 mg 1.136 and 18 mg ZL; 225 mg 1.136 and 19 mg ZL; 225 mg 1.136 and 20 mg ZL;

230 mg 1.136 and 1 mg ZL; 230 mg 1.136 and 2 mg ZL; 230 mg 1.136 and 3 mg ZL; 230 mg 1.136 and 4 mg ZL; 230 mg 1.136 and 5 mg ZL; 230 mg 1.136 and 6 mg ZL; 230 mg 1.136 and 7 mg ZL; 230 mg 1.136 and 8 mg ZL; 230 mg 1.136 and 9 mg ZL; 230 mg 1.136 and 10 mg ZL; 230 mg 1.136 and 11 mg ZL; 230 mg 1.136 and 12 mg ZL; 230 mg 1.136 and 13 mg ZL; 230 mg 1.136 and 14 mg ZL; 230 mg 1.136 and 15 mg ZL; 230 mg 1.136 and 16 mg ZL; 230 mg 1.136 and 17 mg ZL; 230 mg 1.136 and 18 mg ZL; 230 mg 1.136 and 19 mg ZL; 230 mg 1.136 and 20 mg ZL;

235 mg 1.136 and 1 mg ZL; 235 mg 1.136 and 2 mg ZL; 235 mg 1.136 and 3 mg ZL; 235 mg 1.136 and 4 mg ZL; 235 mg 1.136 and 5 mg ZL; 235 mg 1.136 and 6 mg ZL; 235 mg 1.136 and 7 mg ZL; 235 mg 1.136 and 8 mg ZL; 235 mg 1.136 and 9 mg ZL; 235 mg 1.136 and 10 mg ZL; 235 mg 1.136 and 11 mg ZL; 235 mg 1.136 and 12 mg ZL; 235 mg 1.136 and 13 mg ZL; 235 mg 1.136 and 14 mg ZL; 235 mg 1.136 and 15 mg ZL; 235 mg 1.136 and 16 mg ZL; 235 mg 1.136 and 17 mg ZL; 235 mg 1.136 and 18 mg ZL; 235 mg 1.136 and 19 mg ZL; 235 mg 1.136 and 20 mg ZL;

240 mg 1.136 and 1 mg ZL; 240 mg 1.136 and 2 mg ZL; 240 mg 1.136 and 3 mg ZL; 240 mg 1.136 and 4 mg ZL; 240 mg 1.136 and 5 mg ZL; 240 mg 1.136 and 6 mg ZL; 240 mg 1.136 and 7 mg ZL; 240 mg 1.136 and 8 mg ZL; 240 mg 1.136 and 9 mg ZL; 240 mg 1.136 and 10 mg ZL; 240 mg 1.136 and 11 mg ZL; 240 mg 1.136 and 12 mg ZL; 240 mg 1.136 and 13 mg ZL; 240 mg 1.136 and 14 mg ZL; 240 mg 1.136 and 15 mg ZL; 240 mg 1.136 and 16 mg ZL; 240 mg 1.136 and 17 mg ZL; 240 mg 1.136 and 18 mg ZL; 240 mg 1.136 and 19 mg ZL; 240 mg 1.136 and 20 mg ZL;

245 mg 1.136 and 1 mg ZL; 245 mg 1.136 and 2 mg ZL; 245 mg 1.136 and 3 mg ZL; 245 mg 1.136 and 4 mg ZL; 245 mg 1.136 and 5 mg ZL; 245 mg 1.136 and 6 mg ZL; 245 mg 1.136 and 7 mg ZL; 245 mg 1.136 and 8 mg ZL; 245 mg 1.136 and 9 mg ZL; 245 mg 1.136 and 10 mg ZL; 245 mg 1.136 and 11 mg ZL; 245 mg 1.136 and 12 mg ZL; 245 mg 1.136 and 13 mg ZL; 245 mg 1.136 and 14 mg ZL; 245 mg 1.136 and 15 mg ZL; 245 mg 1.136 and 16 mg ZL; 245 mg 1.136 and 17 mg ZL; 245 mg 1.136 and 18 mg ZL; 245 mg 1.136 and 19 mg ZL; 245 mg 1.136 and 20 mg ZL;

250 mg 1.136 and 1 mg ZL; 250 mg 1.136 and 2 mg ZL; 250 mg 1.136 and 3 mg ZL; 250 mg 1.136 and 4 mg ZL; 250 mg 1.136 and 5 mg ZL; 250 mg 1.136 and 6 mg ZL; 250 mg 1.136 and 7 mg ZL; 250 mg 1.136 and 8 mg ZL; 250 mg 1.136 and 9 mg ZL; 250 mg 1.136 and 10 mg ZL; 250 mg 1.136 and 11 mg ZL; 250 mg 1.136 and 12 mg ZL; 250 mg 1.136 and 13 mg ZL; 250 mg 1.136 and 14 mg ZL; 250 mg 1.136 and 15 mg ZL; 250 mg 1.136 and 16 mg ZL; 250 mg 1.136 and 17 mg ZL; 250 mg 1.136 and 18 mg ZL; 250 mg 1.136 and 19 mg ZL; 250 mg 1.136 and 20 mg ZL;

255 mg 1.136 and 1 mg ZL; 255 mg 1.136 and 2 mg ZL; 255 mg 1.136 and 3 mg ZL; 255 mg 1.136 and 4 mg ZL; 255 mg 1.136 and 5 mg ZL; 255 mg 1.136 and 6 mg ZL; 255 mg 1.136 and 7 mg ZL; 255 mg 1.136 and 8 mg ZL; 255 mg 1.136 and 9 mg ZL; 255 mg 1.136 and 10 mg ZL; 255 mg 1.136 and 11 mg ZL; 255 mg 1.136 and 12 mg ZL; 255 mg 1.136 and 13 mg ZL; 255 mg 1.136 and 14 mg ZL; 255 mg 1.136 and 15 mg ZL; 255 mg 1.136 and 16 mg ZL; 255 mg 1.136 and 17 mg ZL; 255 mg 1.136 and 18 mg ZL; 255 mg 1.136 and 19 mg ZL; 255 mg 1.136 and 20 mg ZL;

260 mg 1.136 and 1 mg ZL; 260 mg 1.136 and 2 mg ZL; 260 mg 1.136 and 3 mg ZL; 260 mg 1.136 and 4 mg ZL; 260 mg 1.136 and 5 mg ZL; 260 mg 1.136 and 6 mg ZL; 260 mg 1.136 and 7 mg ZL; 260 mg 1.136 and 8 mg ZL; 260 mg 1.136 and 9 mg ZL; 260 mg 1.136 and 10 mg ZL; 260 mg 1.136 and 11 mg ZL; 260 mg 1.136 and 12 mg ZL; 260 mg 1.136 and 13 mg ZL; 260 mg 1.136 and 14 mg ZL; 260 mg 1.136 and 15 mg ZL; 260 mg 1.136 and 16 mg ZL; 260 mg 1.136 and 17 mg ZL; 260 mg 1.136 and 18 mg ZL; 260 mg 1.136 and 19 mg ZL; 260 mg 1.136 and 20 mg ZL;

265 mg 1.136 and 1 mg ZL; 265 mg 1.136 and 2 mg ZL; 265 mg 1.136 and 3 mg ZL; 265 mg 1.136 and 4 mg ZL; 265 mg 1.136 and 5 mg ZL; 265 mg 1.136 and 6 mg ZL; 265 mg 1.136 and 7 mg ZL; 265 mg 1.136 and 8 mg ZL; 265 mg 1.136 and 9 mg ZL; 265 mg 1.136 and 10 mg ZL; 265 mg 1.136 and 11 mg ZL; 265 mg 1.136 and 12 mg ZL; 265 mg 1.136 and 13 mg ZL; 265 mg 1.136 and 14 mg ZL; 265 mg 1.136 and 15 mg ZL; 265 mg 1.136 and 16 mg ZL; 265 mg 1.136 and 17 mg ZL; 265 mg 1.136 and 18 mg ZL; 265 mg 1.136 and 19 mg ZL; 265 mg 1.136 and 20 mg ZL;

270 mg 1.136 and 1 mg ZL; 270 mg 1.136 and 2 mg ZL; 270 mg 1.136 and 3 mg ZL; 270 mg 1.136 and 4 mg ZL; 270 mg 1.136 and 5 mg ZL; 270 mg 1.136 and 6 mg ZL; 270 mg 1.136 and 7 mg ZL; 270 mg 1.136 and 8 mg ZL; 270 mg 1.136 and 9 mg ZL; 270 mg 1.136 and 10 mg ZL; 270 mg 1.136 and 11 mg ZL; 270 mg 1.136 and 12 mg ZL; 270 mg 1.136 and 13 mg ZL; 270 mg 1.136 and 14 mg ZL; 270 mg 1.136 and 15 mg ZL; 270 mg 1.136 and 16 mg ZL; 270 mg 1.136 and 17 mg ZL; 270 mg 1.136 and 18 mg ZL; 270 mg 1.136 and 19 mg ZL; 270 mg 1.136 and 20 mg ZL;

275 mg 1.136 and 1 mg ZL; 275 mg 1.136 and 2 mg ZL; 275 mg 1.136 and 3 mg ZL; 275 mg 1.136 and 4 mg ZL; 275 mg 1.136 and 5 mg ZL; 275 mg 1.136 and 6 mg ZL; 275 mg 1.136 and 7 mg ZL; 275 mg 1.136 and 8 mg ZL; 275 mg 1.136 and 9 mg ZL; 275 mg 1.136 and 10 mg ZL; 275 mg 1.136 and 11 mg ZL; 275 mg 1.136 and 12 mg ZL; 275 mg 1.136 and 13 mg ZL; 275 mg 1.136 and 14 mg ZL; 275 mg 1.136 and 15 mg ZL; 275 mg 1.136 and 16 mg ZL; 275 mg 1.136 and 17 mg ZL; 275 mg 1.136 and 18 mg ZL; 275 mg 1.136 and 19 mg ZL; 275 mg 1.136 and 20 mg ZL;

280 mg 1.136 and 1 mg ZL; 280 mg 1.136 and 2 mg ZL; 280 mg 1.136 and 3 mg ZL; 280 mg 1.136 and 4 mg ZL; 280 mg 1.136 and 5 mg ZL; 280 mg 1.136 and 6 mg ZL; 280 mg 1.136 and 7 mg ZL; 280 mg 1.136 and 8 mg ZL; 280 mg 1.136 and 9 mg ZL; 280 mg 1.136 and 10 mg ZL; 280 mg 1.136 and 11 mg ZL; 280 mg 1.136 and 12 mg ZL; 280 mg 1.136 and 13 mg ZL; 280 mg 1.136 and 14 mg ZL; 280 mg 1.136 and 15 mg ZL; 280 mg 1.136 and 16 mg ZL; 280 mg 1.136 and 17 mg ZL; 280 mg 1.136 and 18 mg ZL; 280 mg 1.136 and 19 mg ZL; 280 mg 1.136 and 20 mg ZL;

285 mg 1.136 and 1 mg ZL; 285 mg 1.136 and 2 mg ZL; 285 mg 1.136 and 3 mg ZL; 285 mg 1.136 and 4 mg ZL; 285 mg 1.136 and 5 mg ZL; 285 mg 1.136 and 6 mg ZL; 285 mg 1.136 and 7 mg ZL; 285 mg 1.136 and 8 mg ZL; 285 mg 1.136 and 9 mg ZL; 285 mg 1.136 and 10 mg ZL; 285 mg 1.136 and 11 mg ZL; 285 mg 1.136 and 12 mg ZL; 285 mg 1.136 and 13 mg ZL; 285 mg 1.136 and 14 mg ZL; 285 mg 1.136 and 15 mg ZL; 285 mg 1.136 and 16 mg ZL; 285 mg 1.136 and 17 mg ZL; 285 mg 1.136 and 18 mg ZL; 285 mg 1.136 and 19 mg ZL; 285 mg 1.136 and 20 mg ZL;

290 mg 1.136 and 1 mg ZL; 290 mg 1.136 and 2 mg ZL; 290 mg 1.136 and 3 mg ZL; 290 mg 1.136 and 4 mg ZL; 290 mg 1.136 and 5 mg ZL; 290 mg 1.136 and 6 mg ZL; 290 mg 1.136 and 7 mg ZL; 290 mg 1.136 and 8 mg ZL; 290 mg 1.136 and 9 mg ZL; 290 mg 1.136 and 10 mg ZL; 290 mg 1.136 and 11 mg ZL; 290 mg 1.136 and 12 mg ZL; 290 mg 1.136 and 13 mg ZL; 290 mg 1.136 and 14 mg ZL; 290 mg 1.136 and 15 mg ZL; 290 mg 1.136 and 16 mg ZL; 290 mg 1.136 and 17 mg ZL; 290 mg 1.136 and 18 mg ZL; 290 mg 1.136 and 19 mg ZL; 290 mg 1.136 and 20 mg ZL;

295 mg 1.136 and 1 mg ZL; 295 mg 1.136 and 2 mg ZL; 295 mg 1.136 and 3 mg ZL; 295 mg 1.136 and 4 mg ZL; 295 mg 1.136 and 5 mg ZL; 295 mg 1.136 and 6 mg ZL; 295 mg 1.136 and 7 mg ZL; 295 mg 1.136 and 8 mg ZL; 295 mg 1.136 and 9 mg ZL; 295 mg 1.136 and 10 mg ZL; 295 mg 1.136 and 11 mg ZL; 295 mg 1.136 and 12 mg ZL; 295 mg 1.136 and 13 mg ZL; 295 mg 1.136 and 14 mg ZL; 295 mg 1.136 and 15 mg ZL; 295 mg 1.136 and 16 mg ZL; 295 mg 1.136 and 17 mg ZL; 295 mg 1.136 and 18 mg ZL; 295 mg 1.136 and 19 mg ZL; 295 mg 1.136 and 20 mg ZL;

300 mg 1.136 and 1 mg ZL; 300 mg 1.136 and 2 mg ZL; 300 mg 1.136 and 3 mg ZL; 300 mg 1.136 and 4 mg ZL; 300 mg 1.136 and 5 mg ZL; 300 mg 1.136 and 6 mg ZL; 300 mg 1.136 and 7 mg ZL; 300 mg 1.136 and 8 mg ZL; 300 mg 1.136 and 9 mg ZL; 300 mg 1.136 and 10 mg ZL; 300 mg 1.136 and 11 mg ZL; 300 mg 1.136 and 12 mg ZL; 300 mg 1.136 and 13 mg ZL; 300 mg 1.136 and 14 mg ZL; 300 mg 1.136 and 15 mg ZL; 300 mg 1.136 and 16 mg ZL; 300 mg 1.136 and 17 mg ZL; 300 mg 1.136 and 18 mg ZL; 300 mg 1.136 and 19 mg ZL; 300 mg 1.136 and 20 mg ZL.

305 mg 1.136 and 1 mg ZL; 305 mg 1.136 and 2 mg ZL; 305 mg 1.136 and 3 mg ZL; 305 mg 1.136 and 4 mg ZL; 305 mg 1.136 and 5 mg ZL; 305 mg 1.136 and 6 mg ZL; 305 mg 1.136 and 7 mg ZL; 305 mg 1.136 and 8 mg ZL; 305 mg 1.136 and 9 mg ZL; 305 mg 1.136 and 10 mg ZL; 305 mg 1.136 and 11 mg ZL; 305 mg 1.136 and 12 mg ZL; 305 mg 1.136 and 13 mg ZL; 305 mg 1.136 and 14 mg ZL; 305 mg 1.136 and 15 mg ZL; 305 mg 1.136 and 16 mg ZL; 305 mg 1.136 and 17 mg ZL; 305 mg 1.136 and 18 mg ZL; 305 mg 1.136 and 19 mg ZL; 305 mg 1.136 and 20 mg ZL.

310 mg 1.136 and 1 mg ZL; 310 mg 1.136 and 2 mg ZL; 310 mg 1.136 and 3 mg ZL; 310 mg 1.136 and 4 mg ZL; 310 mg 1.136 and 5 mg ZL; 310 mg 1.136 and 6 mg ZL; 310 mg 1.136 and 7 mg ZL; 310 mg 1.136 and 8 mg ZL; 310 mg 1.136 and 9 mg ZL; 310 mg 1.136 and 10 mg ZL; 310 mg 1.136 and 11 mg ZL; 310 mg 1.136 and 12 mg ZL; 310 mg 1.136 and 13 mg ZL; 310 mg 1.136 and 14 mg ZL; 310 mg 1.136 and 15 mg ZL; 310 mg 1.136 and 16 mg ZL; 310 mg 1.136 and 17 mg ZL; 310 mg 1.136 and 18 mg ZL; 310 mg 1.136 and 19 mg ZL; 310 mg 1.136 and 20 mg ZL.

315 mg 1.136 and 1 mg ZL; 315 mg 1.136 and 2 mg ZL; 315 mg 1.136 and 3 mg ZL; 315 mg 1.136 and 4 mg ZL; 315 mg 1.136 and 5 mg ZL; 315 mg 1.136 and 6 mg ZL; 315 mg 1.136 and 7 mg ZL; 315 mg 1.136 and 8 mg ZL; 315 mg 1.136 and 9 mg ZL; 315 mg 1.136 and 10 mg ZL; 315 mg 1.136 and 11 mg ZL; 315 mg 1.136 and 12 mg ZL; 315 mg 1.136 and 13 mg ZL; 315 mg 1.136 and 14 mg ZL; 315 mg 1.136 and 15 mg ZL; 315 mg 1.136 and 16 mg ZL; 315 mg 1.136 and 17 mg ZL; 315 mg 1.136 and 18 mg ZL; 315 mg 1.136 and 19 mg ZL; 315 mg 1.136 and 20 mg ZL.

320 mg 1.136 and 1 mg ZL; 320 mg 1.136 and 2 mg ZL; 320 mg 1.136 and 3 mg ZL; 320 mg 1.136 and 4 mg ZL; 320 mg 1.136 and 5 mg ZL; 320 mg 1.136 and 6 mg ZL; 320 mg 1.136 and 7 mg ZL; 320 mg 1.136 and 8 mg ZL; 320 mg 1.136 and 9 mg ZL; 320 mg 1.136 and 10 mg ZL; 320 mg 1.136 and 11 mg ZL; 320 mg 1.136 and 12 mg ZL; 320 mg 1.136 and 13 mg ZL; 320 mg 1.136 and 14 mg ZL; 320 mg 1.136 and 15 mg ZL; 320 mg 1.136 and 16 mg ZL; 320 mg 1.136 and 17 mg ZL; 320 mg 1.136 and 18 mg ZL; 320 mg 1.136 and 19 mg ZL; 320 mg 1.136 and 20 mg ZL.

325 mg 1.136 and 1 mg ZL; 325 mg 1.136 and 2 mg ZL; 325 mg 1.136 and 3 mg ZL; 325 mg 1.136 and 4 mg ZL; 325 mg 1.136 and 5 mg ZL; 325 mg 1.136 and 6 mg ZL; 325 mg 1.136 and 7 mg ZL; 325 mg 1.136 and 8 mg ZL; 325 mg 1.136 and 9 mg ZL; 325 mg 1.136 and 10 mg ZL; 325 mg 1.136 and 11 mg ZL; 325 mg 1.136 and 12 mg ZL; 325 mg 1.136 and 13 mg ZL; 325 mg 1.136 and 14 mg ZL; 325 mg 1.136 and 15 mg ZL; 325 mg 1.136 and 16 mg ZL; 325 mg 1.136 and 17 mg ZL; 325 mg 1.136 and 18 mg ZL; 325 mg 1.136 and 19 mg ZL; 325 mg 1.136 and 20 mg ZL.

330 mg 1.136 and 1 mg ZL; 330 mg 1.136 and 2 mg ZL; 330 mg 1.136 and 3 mg ZL; 330 mg 1.136 and 4 mg ZL; 330 mg 1.136 and 5 mg ZL; 330 mg 1.136 and 6 mg ZL; 330 mg 1.136 and 7 mg ZL; 330 mg 1.136 and 8 mg ZL; 330 mg 1.136 and 9 mg ZL; 330 mg 1.136 and 10 mg ZL; 330 mg 1.136 and 11 mg ZL; 330 mg 1.136 and 12 mg ZL; 330 mg 1.136 and 13 mg ZL; 330 mg 1.136 and 14 mg ZL; 330 mg 1.136 and 15 mg ZL; 330 mg 1.136 and 16 mg ZL; 330 mg 1.136 and 17 mg ZL; 330 mg 1.136 and 18 mg ZL; 330 mg 1.136 and 19 mg ZL; 330 mg 1.136 and 20 mg ZL.

335 mg 1.136 and 1 mg ZL; 335 mg 1.136 and 2 mg ZL; 335 mg 1.136 and 3 mg ZL; 335 mg 1.136 and 4 mg ZL; 335 mg 1.136 and 5 mg ZL; 335 mg 1.136 and 6 mg ZL; 335 mg 1.136 and 7 mg ZL; 335 mg 1.136 and 8 mg ZL; 335 mg 1.136 and 9 mg ZL; 335 mg 1.136 and 10 mg ZL; 335 mg 1.136 and 11 mg ZL; 335 mg 1.136 and 12 mg ZL; 335 mg 1.136 and 13 mg ZL; 335 mg 1.136 and 14 mg ZL; 335 mg 1.136 and 15 mg ZL; 335 mg 1.136 and 16 mg ZL; 335 mg 1.136 and 17 mg ZL; 335 mg 1.136 and 18 mg ZL; 335 mg 1.136 and 19 mg ZL; 335 mg 1.136 and 20 mg ZL.

340 mg 1.136 and 1 mg ZL; 340 mg 1.136 and 2 mg ZL; 340 mg 1.136 and 3 mg ZL; 340 mg 1.136 and 4 mg ZL; 340 mg 1.136 and 5 mg ZL; 340 mg 1.136 and 6 mg ZL; 340 mg 1.136 and 7 mg ZL;

340 mg 1.136 and 8 mg ZL; 340 mg 1.136 and 9 mg ZL; 340 mg 1.136 and 10 mg ZL; 340 mg 1.136 and 11 mg ZL; 340 mg 1.136 and 12 mg ZL; 340 mg 1.136 and 13 mg ZL; 340 mg 1.136 and 14 mg ZL; 340 mg 1.136 and 15 mg ZL; 340 mg 1.136 and 16 mg ZL; 340 mg 1.136 and 17 mg ZL; 340 mg 1.136 and 18 mg ZL; 340 mg 1.136 and 19 mg ZL; 340 mg 1.136 and 20 mg ZL.

345 mg 1.136 and 1 mg ZL; 345 mg 1.136 and 2 mg ZL; 345 mg 1.136 and 3 mg ZL; 345 mg 1.136 and 4 mg ZL; 345 mg 1.136 and 5 mg ZL; 345 mg 1.136 and 6 mg ZL; 345 mg 1.136 and 7 mg ZL; 345 mg 1.136 and 8 mg ZL; 345 mg 1.136 and 9 mg ZL; 345 mg 1.136 and 10 mg ZL; 345 mg 1.136 and 11 mg ZL; 345 mg 1.136 and 12 mg ZL; 345 mg 1.136 and 13 mg ZL; 345 mg 1.136 and 14 mg ZL; 345 mg 1.136 and 15 mg ZL; 345 mg 1.136 and 16 mg ZL; 345 mg 1.136 and 17 mg ZL; 345 mg 1.136 and 18 mg ZL; 345 mg 1.136 and 19 mg ZL; 345 mg 1.136 and 20 mg ZL.

350 mg 1.136 and 1 mg ZL; 350 mg 1.136 and 2 mg ZL; 350 mg 1.136 and 3 mg ZL; 350 mg 1.136 and 4 mg ZL; 350 mg 1.136 and 5 mg ZL; 350 mg 1.136 and 6 mg ZL; 350 mg 1.136 and 7 mg ZL; 350 mg 1.136 and 8 mg ZL; 350 mg 1.136 and 9 mg ZL; 350 mg 1.136 and 10 mg ZL; 350 mg 1.136 and 11 mg ZL; 350 mg 1.136 and 12 mg ZL; 350 mg 1.136 and 13 mg ZL; 350 mg 1.136 and 14 mg ZL; 350 mg 1.136 and 15 mg ZL; 350 mg 1.136 and 16 mg ZL; 350 mg 1.136 and 17 mg ZL; 350 mg 1.136 and 18 mg ZL; 350 mg 1.136 and 19 mg ZL; 350 mg 1.136 and 20 mg ZL.

355 mg 1.136 and 1 mg ZL; 355 mg 1.136 and 2 mg ZL; 355 mg 1.136 and 3 mg ZL; 355 mg 1.136 and 4 mg ZL; 355 mg 1.136 and 5 mg ZL; 355 mg 1.136 and 6 mg ZL; 355 mg 1.136 and 7 mg ZL; 355 mg 1.136 and 8 mg ZL; 355 mg 1.136 and 9 mg ZL; 355 mg 1.136 and 10 mg ZL; 355 mg 1.136 and 11 mg ZL; 355 mg 1.136 and 12 mg ZL; 355 mg 1.136 and 13 mg ZL; 355 mg 1.136 and 14 mg ZL; 355 mg 1.136 and 15 mg ZL; 355 mg 1.136 and 16 mg ZL; 355 mg 1.136 and 17 mg ZL; 355 mg 1.136 and 18 mg ZL; 355 mg 1.136 and 19 mg ZL; 355 mg 1.136 and 20 mg ZL.

360 mg 1.136 and 1 mg ZL; 360 mg 1.136 and 2 mg ZL; 360 mg 1.136 and 3 mg ZL; 360 mg 1.136 and 4 mg ZL; 360 mg 1.136 and 5 mg ZL; 360 mg 1.136 and 6 mg ZL; 360 mg 1.136 and 7 mg ZL; 360 mg 1.136 and 8 mg ZL; 360 mg 1.136 and 9 mg ZL; 360 mg 1.136 and 10 mg ZL; 360 mg 1.136 and 11 mg ZL; 360 mg 1.136 and 12 mg ZL; 360 mg 1.136 and 13 mg ZL; 360 mg 1.136 and 14 mg ZL; 360 mg 1.136 and 15 mg ZL; 360 mg 1.136 and 16 mg ZL; 360 mg 1.136 and 17 mg ZL; 360 mg 1.136 and 18 mg ZL; 360 mg 1.136 and 19 mg ZL; 360 mg 1.136 and 20 mg ZL.

365 mg 1.136 and 1 mg ZL; 365 mg 1.136 and 2 mg ZL; 365 mg 1.136 and 3 mg ZL; 365 mg 1.136 and 4 mg ZL; 365 mg 1.136 and 5 mg ZL; 365 mg 1.136 and 6 mg ZL; 365 mg 1.136 and 7 mg ZL; 365 mg 1.136 and 8 mg ZL; 365 mg 1.136 and 9 mg ZL; 365 mg 1.136 and 10 mg ZL; 365 mg 1.136 and 11 mg ZL; 365 mg 1.136 and 12 mg ZL; 365 mg 1.136 and 13 mg ZL; 365 mg 1.136 and 14 mg ZL; 365 mg 1.136 and 15 mg ZL; 365 mg 1.136 and 16 mg ZL; 365 mg 1.136 and 17 mg ZL; 365 mg 1.136 and 18 mg ZL; 365 mg 1.136 and 19 mg ZL; 365 mg 1.136 and 20 mg ZL.

370 mg 1.136 and 1 mg ZL; 370 mg 1.136 and 2 mg ZL; 370 mg 1.136 and 3 mg ZL; 370 mg 1.136 and 4 mg ZL; 370 mg 1.136 and 5 mg ZL; 370 mg 1.136 and 6 mg ZL; 370 mg 1.136 and 7 mg ZL; 370 mg 1.136 and 8 mg ZL; 370 mg 1.136 and 9 mg ZL; 370 mg 1.136 and 10 mg ZL; 370 mg 1.136 and 11 mg ZL; 370 mg 1.136 and 12 mg ZL; 370 mg 1.136 and 13 mg ZL; 370 mg 1.136 and 14 mg ZL; 370 mg 1.136 and 15 mg ZL; 370 mg 1.136 and 16 mg ZL; 370 mg 1.136 and 17 mg ZL; 370 mg 1.136 and 18 mg ZL; 370 mg 1.136 and 19 mg ZL; 370 mg 1.136 and 20 mg ZL.

375 mg 1.136 and 1 mg ZL; 375 mg 1.136 and 2 mg ZL; 375 mg 1.136 and 3 mg ZL; 375 mg 1.136 and 4 mg ZL; 375 mg 1.136 and 5 mg ZL; 375 mg 1.136 and 6 mg ZL; 375 mg 1.136 and 7 mg ZL; 375 mg 1.136 and 8 mg ZL; 375 mg 1.136 and 9 mg ZL; 375 mg 1.136 and 10 mg ZL; 375 mg 1.136 and 11 mg ZL; 375 mg 1.136 and 12 mg ZL; 375 mg 1.136 and 13 mg ZL; 375 mg 1.136 and 14 mg ZL; 375 mg 1.136 and 15 mg ZL; 375 mg 1.136 and 16 mg ZL; 375 mg 1.136 and 17 mg ZL; 375 mg 1.136 and 18 mg ZL; 375 mg 1.136 and 19 mg ZL; 375 mg 1.136 and 20 mg ZL.

380 mg 1.136 and 1 mg ZL; 380 mg 1.136 and 2 mg ZL; 380 mg 1.136 and 3 mg ZL; 380 mg 1.136 and 4 mg ZL; 380 mg 1.136 and 5 mg ZL; 380 mg 1.136 and 6 mg ZL; 380 mg 1.136 and 7 mg ZL; 380 mg 1.136 and 8 mg ZL; 380 mg 1.136 and 9 mg ZL; 380 mg 1.136 and 10 mg ZL; 380 mg 1.136 and 11 mg ZL; 380 mg 1.136 and 12 mg ZL; 380 mg 1.136 and 13 mg ZL; 380 mg 1.136 and 14 mg ZL; 380 mg 1.136 and 15 mg ZL; 380 mg 1.136 and 16 mg ZL; 380 mg 1.136 and 17 mg ZL; 380 mg 1.136 and 18 mg ZL; 380 mg 1.136 and 19 mg ZL; 380 mg 1.136 and 20 mg ZL.

385 mg 1.136 and 1 mg ZL; 385 mg 1.136 and 2 mg ZL; 385 mg 1.136 and 3 mg ZL; 385 mg 1.136 and 4 mg ZL; 385 mg 1.136 and 5 mg ZL; 385 mg 1.136 and 6 mg ZL; 385 mg 1.136 and 7 mg ZL; 385 mg 1.136 and 8 mg ZL; 385 mg 1.136 and 9 mg ZL; 385 mg 1.136 and 10 mg ZL; 385 mg 1.136 and 11 mg ZL; 385 mg 1.136 and 12 mg ZL; 385 mg 1.136 and 13 mg ZL; 385 mg 1.136 and 14 mg ZL; 385 mg 1.136 and 15 mg ZL; 385 mg 1.136 and 16 mg ZL; 385 mg 1.136 and 17 mg ZL; 385 mg 1.136 and 18 mg ZL; 385 mg 1.136 and 19 mg ZL; 385 mg 1.136 and 20 mg ZL.

390 mg 1.136 and 1 mg ZL; 390 mg 1.136 and 2 mg ZL; 390 mg 1.136 and 3 mg ZL; 390 mg 1.136 and 4 mg ZL; 390 mg 1.136 and 5 mg ZL; 390 mg 1.136 and 6 mg ZL; 390 mg 1.136 and 7 mg ZL; 390 mg 1.136 and 8 mg ZL; 390 mg 1.136 and 9 mg ZL; 390 mg 1.136 and 10 mg ZL; 390 mg 1.136 and 11 mg ZL; 390 mg 1.136 and 12 mg ZL; 390 mg 1.136 and 13 mg ZL; 390 mg 1.136 and 14 mg ZL; 390 mg 1.136 and 15 mg ZL; 390 mg 1.136 and 16 mg ZL; 390 mg 1.136 and 17 mg ZL; 390 mg 1.136 and 18 mg ZL; 390 mg 1.136 and 19 mg ZL; 390 mg 1.136 and 20 mg ZL.

395 mg 1.136 and 1 mg ZL; 395 mg 1.136 and 2 mg ZL; 395 mg 1.136 and 3 mg ZL; 395 mg 1.136 and 4 mg ZL; 395 mg 1.136 and 5 mg ZL; 395 mg 1.136 and 6 mg ZL; 395 mg 1.136 and 7 mg ZL; 395 mg 1.136 and 8 mg ZL; 395 mg 1.136 and 9 mg ZL; 395 mg 1.136 and 10 mg ZL; 395 mg 1.136 and 11 mg ZL; 395 mg 1.136 and 12 mg ZL; 395 mg 1.136 and 13 mg ZL; 395 mg 1.136 and 14 mg ZL; 395 mg 1.136 and 15 mg ZL; 395 mg 1.136 and 16 mg ZL; 395 mg 1.136 and 17 mg ZL; 395 mg 1.136 and 18 mg ZL; 395 mg 1.136 and 19 mg ZL; 395 mg 1.136 and 20 mg ZL.

400 mg 1.136 and 1 mg ZL; 400 mg 1.136 and 2 mg ZL; 400 mg 1.136 and 3 mg ZL; 400 mg 1.136 and 4 mg ZL; 400 mg 1.136 and 5 mg ZL; 400 mg 1.136 and 6 mg ZL; 400 mg 1.136 and 7 mg ZL; 400 mg 1.136 and 8 mg ZL; 400 mg 1.136 and 9 mg ZL; 400 mg 1.136 and 10 mg ZL; 400 mg 1.136 and 11 mg ZL; 400 mg 1.136 and 12 mg ZL; 400 mg 1.136 and 13 mg ZL; 400 mg 1.136 and 14 mg ZL; 400 mg 1.136 and 15 mg ZL; 400 mg 1.136 and 16 mg ZL; 400 mg 1.136 and 17 mg ZL; 400 mg 1.136 and 18 mg ZL; 400 mg 1.136 and 19 mg ZL; 400 mg 1.136 and 20 mg ZL.

Based on the data listed herein unit dose forms containing the following amounts of 1.136 and pranlukast (PL) are explicitly encompassed by the present invention:

10 mg 1.136 and 1 mg PL; 10 mg 1.136 and 2 mg PL; 10 mg 1.136 and 3 mg PL; 10 mg 1.136 and 4 mg PL; 10 mg 1.136 and 5 mg PL; 10 mg 1.136 and 6 mg PL; 10 mg 1.136 and 7 mg PL; 10 mg 1.136 and 8 mg PL; 10 mg 1.136 and 9 mg PL; 10 mg 1.136 and 10 mg PL; 10 mg 1.136 and 11 mg PL; 10 mg 1.136 and 12 mg PL; 10 mg 1.136 and 13 mg PL; 10 mg 1.136 and 14 mg PL; 10 mg 1.136 and 15 mg PL; 10 mg 1.136 and 16 mg PL; 10 mg 1.136 and 17 mg PL; 10 mg 1.136 and 18 mg PL; 10 mg 1.136 and 19 mg PL; 10 mg 1.136 and 20 mg PL;

15 mg 1.136 and 1 mg PL; 15 mg 1.136 and 2 mg PL; 15 mg 1.136 and 3 mg PL; 15 mg 1.136 and 4 mg PL; 15 mg 1.136 and 5 mg PL; 15 mg 1.136 and 6 mg PL; 15 mg 1.136 and 7 mg PL; 15 mg 1.136 and 8 mg PL; 15 mg 1.136 and 9 mg PL; 15 mg 1.136 and 10 mg PL; 15 mg 1.136 and 11 mg PL; 15 mg 1.136 and 12 mg PL; 15 mg 1.136 and 13 mg PL; 15 mg 1.136 and 14 mg PL; 15 mg 1.136 and 15 mg PL; 15 mg 1.136 and 16 mg PL; 15 mg 1.136 and 17 mg PL; 15 mg 1.136 and 18 mg PL; 15 mg 1.136 and 19 mg PL; 15 mg 1.136 and 20 mg PL;

20 mg 1.136 and 1 mg PL; 20 mg 1.136 and 2 mg PL; 20 mg 1.136 and 3 mg PL; 20 mg 1.136 and 4 mg PL; 20 mg 1.136 and 5 mg PL; 20 mg 1.136 and 6 mg PL; 20 mg 1.136 and 7 mg PL; 20 mg 1.136 and 8 mg PL; 20 mg 1.136 and 9 mg PL; 20 mg 1.136 and 10 mg PL; 20 mg 1.136 and 11 mg PL; 20 mg 1.136 and 12 mg PL; 20 mg 1.136 and 13 mg PL; 20 mg 1.136 and 14 mg PL; 20 mg 1.136 and 15 mg PL; 20 mg 1.136 and 16 mg PL; 20 mg 1.136 and 17 mg PL; 20 mg 1.136 and 18 mg PL; 20 mg 1.136 and 19 mg PL; 20 mg 1.136 and 20 mg PL;

25 mg 1.136 and 1 mg PL; 25 mg 1.136 and 2 mg PL; 25 mg 1.136 and 3 mg PL; 25 mg 1.136 and 4 mg PL; 25 mg 1.136 and 5 mg PL; 25 mg 1.136 and 6 mg PL; 25 mg 1.136 and 7 mg PL; 25 mg 1.136 and 8 mg PL; 25 mg 1.136 and 9 mg PL; 25 mg 1.136 and 10 mg PL; 25 mg 1.136 and 11 mg PL; 25 mg 1.136 and 12 mg PL; 25 mg 1.136 and 13 mg PL; 25 mg 1.136 and 14 mg PL; 25 mg 1.136 and 15 mg PL; 25 mg 1.136 and 16 mg PL; 25 mg 1.136 and 17 mg PL; 25 mg 1.136 and 18 mg PL; 25 mg 1.136 and 19 mg PL; 25 mg 1.136 and 20 mg PL;

30 mg 1.136 and 1 mg PL; 30 mg 1.136 and 2 mg PL; 30 mg 1.136 and 3 mg PL; 30 mg 1.136 and 4 mg PL; 30 mg 1.136 and 5 mg PL; 30 mg 1.136 and 6 mg PL; 30 mg 1.136 and 7 mg PL; 30 mg 1.136 and 8 mg PL; 30 mg 1.136 and 9 mg PL; 30 mg 1.136 and 10 mg PL; 30 mg 1.136 and 11 mg PL; 30 mg 1.136 and 12 mg PL; 30 mg 1.136 and 13 mg PL; 30 mg 1.136 and 14 mg PL; 30 mg 1.136 and 15 mg PL; 30 mg 1.136 and 16 mg PL; 30 mg 1.136 and 17 mg PL; 30 mg 1.136 and 18 mg PL; 30 mg 1.136 and 19 mg PL; 30 mg 1.136 and 20 mg PL;

35 mg 1.136 and 1 mg PL; 35 mg 1.136 and 2 mg PL; 35 mg 1.136 and 3 mg PL; 35 mg 1.136 and 4 mg PL; 35 mg 1.136 and 5 mg PL; 35 mg 1.136 and 6 mg PL; 35 mg 1.136 and 7 mg PL; 35 mg 1.136 and 8 mg PL; 35 mg 1.136 and 9 mg PL; 35 mg 1.136 and 10 mg PL; 35 mg 1.136 and 11 mg PL; 35 mg 1.136 and 12 mg PL; 35 mg 1.136 and 13 mg PL; 35 mg 1.136 and 14 mg PL; 35 mg 1.136 and 15 mg PL; 35 mg 1.136 and 16 mg PL; 35 mg 1.136 and 17 mg PL; 35 mg 1.136 and 18 mg PL; 35 mg 1.136 and 19 mg PL; 35 mg 1.136 and 20 mg PL;

40 mg 1.136 and 1 mg PL; 40 mg 1.136 and 2 mg PL; 40 mg 1.136 and 3 mg PL; 40 mg 1.136 and 4 mg PL; 40 mg 1.136 and 5 mg PL; 40 mg 1.136 and 6 mg PL; 40 mg 1.136 and 7 mg PL; 40 mg 1.136 and 8 mg PL; 40 mg 1.136 and 9 mg PL; 40 mg 1.136 and 10 mg PL; 40 mg 1.136 and 11 mg PL; 40 mg 1.136 and 12 mg PL; 40 mg 1.136 and 13 mg PL; 40 mg 1.136 and 14 mg PL; 40 mg 1.136 and 15 mg PL; 40 mg 1.136 and 16 mg PL; 40 mg 1.136 and 17 mg PL; 40 mg 1.136 and 18 mg PL; 40 mg 1.136 and 19 mg PL; 40 mg 1.136 and 20 mg PL;

45 mg 1.136 and 1 mg PL; 45 mg 1.136 and 2 mg PL; 45 mg 1.136 and 3 mg PL; 45 mg 1.136 and 4 mg PL; 45 mg 1.136 and 5 mg PL; 45 mg 1.136 and 6 mg PL; 45 mg 1.136 and 7 mg PL; 45 mg 1.136 and 8 mg PL; 45 mg 1.136 and 9 mg PL; 45 mg 1.136 and 10 mg PL; 45 mg 1.136 and 11 mg PL; 45 mg 1.136 and 12 mg PL; 45 mg 1.136 and 13 mg PL; 45 mg 1.136 and 14 mg PL; 45 mg 1.136 and 15 mg PL; 45 mg 1.136 and 16 mg PL; 45 mg 1.136 and 17 mg PL; 45 mg 1.136 and 18 mg PL; 45 mg 1.136 and 19 mg PL; 45 mg 1.136 and 20 mg PL;

50 mg 1.136 and 1 mg PL; 50 mg 1.136 and 2 mg PL; 50 mg 1.136 and 3 mg PL; 50 mg 1.136 and 4 mg PL; 50 mg 1.136 and 5 mg PL; 50 mg 1.136 and 6 mg PL; 50 mg 1.136 and 7 mg PL; 50 mg 1.136 and 8 mg PL; 50 mg 1.136 and 9 mg PL; 50 mg 1.136 and 10 mg PL; 50 mg 1.136 and 11 mg PL; 50 mg 1.136 and 12 mg PL; 50 mg 1.136 and 13 mg PL; 50 mg 1.136 and 14 mg PL; 50 mg 1.136 and 15 mg PL; 50 mg 1.136 and 16 mg PL; 50 mg 1.136 and 17 mg PL; 50 mg 1.136 and 18 mg PL; 50 mg 1.136 and 19 mg PL; 50 mg 1.136 and 20 mg PL;

55 mg 1.136 and 1 mg PL; 55 mg 1.136 and 2 mg PL; 55 mg 1.136 and 3 mg PL; 55 mg 1.136 and 4 mg PL; 55 mg 1.136 and 5 mg PL; 55 mg 1.136 and 6 mg PL; 55 mg 1.136 and 7 mg PL; 55 mg 1.136 and 8 mg PL; 55 mg 1.136 and 9 mg PL; 55 mg 1.136 and 10 mg PL; 55 mg 1.136 and 11 mg PL; 55 mg 1.136 and 12 mg PL; 55 mg 1.136 and 13 mg PL; 55 mg 1.136 and 14 mg PL; 55 mg 1.136 and 15 mg PL; 55 mg 1.136 and 16 mg PL; 55 mg 1.136 and 17 mg PL; 55 mg 1.136 and 18 mg PL; 55 mg 1.136 and 19 mg PL; 55 mg 1.136 and 20 mg PL;

60 mg 1.136 and 1 mg PL; 60 mg 1.136 and 2 mg PL; 60 mg 1.136 and 3 mg PL; 60 mg 1.136 and 4 mg PL; 60 mg 1.136 and 5 mg PL; 60 mg 1.136 and 6 mg PL; 60 mg 1.136 and 7 mg PL; 60 mg 1.136 and 8 mg PL; 60 mg 1.136 and 9 mg PL; 60 mg 1.136 and 10 mg PL; 60 mg 1.136 and 11 mg PL; 60 mg 1.136 and 12 mg PL; 60 mg 1.136 and 13 mg PL; 60 mg 1.136 and 14 mg PL; 60 mg 1.136 and 15 mg PL; 60 mg 1.136 and 16 mg PL; 60 mg 1.136 and 17 mg PL; 60 mg 1.136 and 18 mg PL; 60 mg 1.136 and 19 mg PL; 60 mg 1.136 and 20 mg PL;

65 mg 1.136 and 1 mg PL; 65 mg 1.136 and 2 mg PL; 65 mg 1.136 and 3 mg PL; 65 mg 1.136 and 4 mg PL; 65 mg 1.136 and 5 mg PL; 65 mg 1.136 and 6 mg PL; 65 mg 1.136 and 7 mg PL; 65 mg 1.136 and 8 mg PL; 65 mg 1.136 and 9 mg PL; 65 mg 1.136 and 10 mg PL; 65 mg 1.136 and 11 mg PL; 65 mg 1.136 and 12 mg PL; 65 mg 1.136 and 13 mg PL; 65 mg 1.136 and 14 mg PL; 65 mg 1.136 and 15 mg PL; 65 mg 1.136 and 16 mg PL; 65 mg 1.136 and 17 mg PL; 65 mg 1.136 and 18 mg PL; 65 mg 1.136 and 19 mg PL; 65 mg 1.136 and 20 mg PL;

70 mg 1.136 and 1 mg PL; 70 mg 1.136 and 2 mg PL; 70 mg 1.136 and 3 mg PL; 70 mg 1.136 and 4 mg PL; 70 mg 1.136 and 5 mg PL; 70 mg 1.136 and 6 mg PL; 70 mg 1.136 and 7 mg PL; 70 mg 1.136 and 8 mg PL; 70 mg 1.136 and 9 mg PL; 70 mg 1.136 and 10 mg PL; 70 mg 1.136 and 11 mg PL; 70 mg 1.136 and 12 mg PL; 70 mg 1.136 and 13 mg PL; 70 mg 1.136 and 14 mg PL; 70 mg 1.136 and 15 mg PL; 70 mg 1.136 and 16 mg PL; 70 mg 1.136 and 17 mg PL; 70 mg 1.136 and 18 mg PL; 70 mg 1.136 and 19 mg PL; 70 mg 1.136 and 20 mg PL;

75 mg 1.136 and 1 mg PL; 75 mg 1.136 and 2 mg PL; 75 mg 1.136 and 3 mg PL; 75 mg 1.136 and 4 mg PL; 75 mg 1.136 and 5 mg PL; 75 mg 1.136 and 6 mg PL; 75 mg 1.136 and 7 mg PL; 75 mg 1.136 and 8 mg PL; 75 mg 1.136 and 9 mg PL; 75 mg 1.136 and 10 mg PL; 75 mg 1.136 and 11 mg PL; 75 mg 1.136 and 12 mg PL; 75 mg 1.136 and 13 mg PL; 75 mg 1.136 and 14 mg PL; 75 mg 1.136 and 15 mg PL; 75 mg 1.136 and 16 mg PL; 75 mg 1.136 and 17 mg PL; 75 mg 1.136 and 18 mg PL; 75 mg 1.136 and 19 mg PL; 75 mg 1.136 and 20 mg PL;

80 mg 1.136 and 1 mg PL; 80 mg 1.136 and 2 mg PL; 80 mg 1.136 and 3 mg PL; 80 mg 1.136 and 4 mg PL; 80 mg 1.136 and 5 mg PL; 80 mg 1.136 and 6 mg PL; 80 mg 1.136 and 7 mg PL; 80 mg 1.136 and 8 mg PL; 80 mg 1.136 and 9 mg PL; 80 mg 1.136 and 10 mg PL; 80 mg 1.136 and 11 mg PL; 80 mg 1.136 and 12 mg PL; 80 mg 1.136 and 13 mg PL; 80 mg 1.136 and 14 mg PL; 80 mg 1.136 and 15 mg PL; 80 mg 1.136 and 16 mg PL; 80 mg 1.136 and 17 mg PL; 80 mg 1.136 and 18 mg PL; 80 mg 1.136 and 19 mg PL; 80 mg 1.136 and 20 mg PL;

85 mg 1.136 and 1 mg PL; 85 mg 1.136 and 2 mg PL; 85 mg 1.136 and 3 mg PL; 85 mg 1.136 and 4 mg PL; 85 mg 1.136 and 5 mg PL; 85 mg 1.136 and 6 mg PL; 85 mg 1.136 and 7 mg PL; 85 mg 1.136 and 8 mg PL; 85 mg 1.136 and 9 mg PL; 85 mg 1.136 and 10 mg PL; 85 mg 1.136 and 11 mg PL; 85 mg 1.136 and 12 mg PL; 85 mg 1.136 and 13 mg PL; 85 mg 1.136 and 14 mg PL; 85 mg 1.136 and 15 mg PL; 85 mg 1.136 and 16 mg PL; 85 mg 1.136 and 17 mg PL; 85 mg 1.136 and 18 mg PL; 85 mg 1.136 and 19 mg PL; 85 mg 1.136 and 20 mg PL;

90 mg 1.136 and 1 mg PL; 90 mg 1.136 and 2 mg PL; 90 mg 1.136 and 3 mg PL; 90 mg 1.136 and 4 mg PL; 90 mg 1.136 and 5 mg PL; 90 mg 1.136 and 6 mg PL; 90 mg 1.136 and 7 mg PL; 90 mg 1.136 and 8 mg PL; 90 mg 1.136 and 9 mg PL; 90 mg 1.136 and 10 mg PL; 90 mg 1.136 and 11 mg PL; 90 mg 1.136 and 12 mg PL; 90 mg 1.136 and 13 mg PL; 90 mg 1.136 and 14 mg PL; 90 mg 1.136 and 15 mg PL; 90 mg 1.136 and 16 mg PL; 90 mg 1.136 and 17 mg PL; 90 mg 1.136 and 18 mg PL; 90 mg 1.136 and 19 mg PL; 90 mg 1.136 and 20 mg PL;

95 mg 1.136 and 1 mg PL; 95 mg 1.136 and 2 mg PL; 95 mg 1.136 and 3 mg PL; 95 mg 1.136 and 4 mg PL; 95 mg 1.136 and 5 mg PL; 95 mg 1.136 and 6 mg PL; 95 mg 1.136 and 7 mg PL; 95 mg 1.136 and 8 mg PL; 95 mg 1.136 and 9 mg PL; 95 mg 1.136 and 10 mg PL; 95 mg 1.136 and 11 mg PL; 95 mg 1.136 and 12 mg PL; 95 mg 1.136 and 13 mg PL; 95 mg 1.136 and 14 mg PL; 95 mg 1.136 and 15 mg PL; 95 mg 1.136 and 16 mg PL; 95 mg 1.136 and 17 mg PL; 95 mg 1.136 and 18 mg PL; 95 mg 1.136 and 19 mg PL; 95 mg 1.136 and 20 mg PL;

100 mg 1.136 and 1 mg PL; 100 mg 1.136 and 2 mg PL; 100 mg 1.136 and 3 mg PL; 100 mg 1.136 and 4 mg PL; 100 mg 1.136 and 5 mg PL; 100 mg 1.136 and 6 mg PL; 100 mg 1.136 and 7 mg PL; 100 mg 1.136 and 8 mg PL; 100 mg 1.136 and 9 mg PL; 100 mg 1.136 and 10 mg PL; 100 mg 1.136 and 11 mg PL; 100 mg 1.136 and 12 mg PL; 100 mg 1.136 and 13 mg PL; 100 mg 1.136 and 14 mg PL; 100 mg 1.136 and 15 mg PL; 100 mg 1.136 and 16 mg PL; 100 mg 1.136 and 17 mg PL; 100 mg 1.136 and 18 mg PL; 100 mg 1.136 and 19 mg PL; 100 mg 1.136 and 20 mg PL;

105 mg 1.136 and 1 mg PL; 105 mg 1.136 and 2 mg PL; 105 mg 1.136 and 3 mg PL; 105 mg 1.136 and 4 mg PL; 105 mg 1.136 and 5 mg PL; 105 mg 1.136 and 6 mg PL; 105 mg 1.136 and 7 mg PL; 105 mg 1.136 and 8 mg PL; 105 mg 1.136 and 9 mg PL; 105 mg 1.136 and 10 mg PL; 105 mg 1.136 and 11 mg PL; 105 mg 1.136 and 12 mg PL; 105 mg 1.136 and 13 mg PL; 105 mg 1.136 and 14 mg PL; 105 mg 1.136 and 15 mg PL; 105 mg 1.136 and 16 mg PL; 105 mg 1.136 and 17 mg PL; 105 mg 1.136 and 18 mg PL; 105 mg 1.136 and 19 mg PL; 105 mg 1.136 and 20 mg PL;

110 mg 1.136 and 1 mg PL; 110 mg 1.136 and 2 mg PL; 110 mg 1.136 and 3 mg PL; 110 mg 1.136 and 4 mg PL; 110 mg 1.136 and 5 mg PL; 110 mg 1.136 and 6 mg PL; 110 mg 1.136 and 7 mg PL; 110 mg 1.136 and 8 mg PL; 110 mg 1.136 and 9 mg PL; 110 mg 1.136 and 10 mg PL; 110 mg 1.136 and 11 mg PL; 110 mg 1.136 and 12 mg PL; 110 mg 1.136 and 13 mg PL; 110 mg 1.136 and 14 mg PL; 110 mg 1.136 and 15 mg PL; 110 mg 1.136 and 16 mg PL; 110 mg 1.136 and 17 mg PL; 110 mg 1.136 and 18 mg PL; 110 mg 1.136 and 19 mg PL; 110 mg 1.136 and 20 mg PL;

115 mg 1.136 and 1 mg PL; 115 mg 1.136 and 2 mg PL; 115 mg 1.136 and 3 mg PL; 115 mg 1.136 and 4 mg PL; 115 mg 1.136 and 5 mg PL; 115 mg 1.136 and 6 mg PL; 115 mg 1.136 and 7 mg PL; 115 mg 1.136 and 8 mg PL; 115 mg 1.136 and 9 mg PL; 115 mg 1.136 and 10 mg PL; 115 mg 1.136 and 11 mg PL; 115 mg 1.136 and 12 mg PL; 115 mg 1.136 and 13 mg PL; 115 mg 1.136 and 14 mg PL; 115 mg 1.136 and 15 mg PL; 115 mg 1.136 and 16 mg PL; 115 mg 1.136 and 17 mg PL; 115 mg 1.136 and 18 mg PL; 115 mg 1.136 and 19 mg PL; 115 mg 1.136 and 20 mg PL;

120 mg 1.136 and 1 mg PL; 120 mg 1.136 and 2 mg PL; 120 mg 1.136 and 3 mg PL; 120 mg 1.136 and 4 mg PL; 120 mg 1.136 and 5 mg PL; 120 mg 1.136 and 6 mg PL; 120 mg 1.136 and 7 mg PL; 120 mg 1.136 and 8 mg PL; 120 mg 1.136 and 9 mg PL; 120 mg 1.136 and 10 mg PL; 120 mg 1.136 and 11 mg PL; 120 mg 1.136 and 12 mg PL; 120 mg 1.136 and 13 mg PL; 120 mg 1.136 and 14 mg PL; 120 mg 1.136 and 15 mg PL; 120 mg 1.136 and 16 mg PL; 120 mg 1.136 and 17 mg PL; 120 mg 1.136 and 18 mg PL; 120 mg 1.136 and 19 mg PL; 120 mg 1.136 and 20 mg PL;

125 mg 1.136 and 1 mg PL; 125 mg 1.136 and 2 mg PL; 125 mg 1.136 and 3 mg PL; 125 mg 1.136 and 4 mg PL; 125 mg 1.136 and 5 mg PL; 125 mg 1.136 and 6 mg PL; 125 mg 1.136 and 7 mg PL; 125 mg 1.136 and 8 mg PL; 125 mg 1.136 and 9 mg PL; 125 mg 1.136 and 10 mg PL; 125 mg 1.136 and 11 mg PL; 125 mg 1.136 and 12 mg PL; 125 mg 1.136 and 13 mg PL; 125 mg 1.136 and 14 mg PL; 125 mg 1.136 and 15 mg PL; 125 mg 1.136 and 16 mg PL; 125 mg 1.136 and 17 mg PL; 125 mg 1.136 and 18 mg PL; 125 mg 1.136 and 19 mg PL; 125 mg 1.136 and 20 mg PL;

130 mg 1.136 and 1 mg PL; 130 mg 1.136 and 2 mg PL; 130 mg 1.136 and 3 mg PL; 130 mg 1.136 and 4 mg PL; 130 mg 1.136 and 5 mg PL; 130 mg 1.136 and 6 mg PL; 130 mg 1.136 and 7 mg PL; 130 mg 1.136 and 8 mg PL; 130 mg 1.136 and 9 mg PL; 130 mg 1.136 and 10 mg PL; 130 mg 1.136 and 11 mg PL; 130 mg 1.136 and 12 mg PL; 130 mg 1.136 and 13 mg PL; 130 mg 1.136 and 14 mg PL; 130 mg 1.136 and 15 mg PL; 130 mg 1.136 and 16 mg PL; 130 mg 1.136 and 17 mg PL; 130 mg 1.136 and 18 mg PL; 130 mg 1.136 and 19 mg PL; 130 mg 1.136 and 20 mg PL;

135 mg 1.136 and 1 mg PL; 135 mg 1.136 and 2 mg PL; 135 mg 1.136 and 3 mg PL; 135 mg 1.136 and 4 mg PL; 135 mg 1.136 and 5 mg PL; 135 mg 1.136 and 6 mg PL; 135 mg 1.136 and 7 mg PL; 135 mg 1.136 and 8 mg PL; 135 mg 1.136 and 9 mg PL; 135 mg 1.136 and 10 mg PL; 135 mg 1.136 and 11 mg PL; 135 mg 1.136 and 12 mg PL; 135 mg 1.136 and 13 mg PL; 135 mg 1.136 and 14 mg PL; 135 mg 1.136 and 15 mg PL; 135 mg 1.136 and 16 mg PL; 135 mg 1.136 and 17 mg PL; 135 mg 1.136 and 18 mg PL; 135 mg 1.136 and 19 mg PL; 135 mg 1.136 and 20 mg PL;

140 mg 1.136 and 1 mg PL; 140 mg 1.136 and 2 mg PL; 140 mg 1.136 and 3 mg PL; 140 mg 1.136 and 4 mg PL; 140 mg 1.136 and 5 mg PL; 140 mg 1.136 and 6 mg PL; 140 mg 1.136 and 7 mg PL; 140 mg 1.136 and 8 mg PL; 140 mg 1.136 and 9 mg PL; 140 mg 1.136 and 10 mg PL; 140 mg 1.136 and 11 mg PL; 140 mg 1.136 and 12 mg PL; 140 mg 1.136 and 13 mg PL; 140 mg 1.136 and 14 mg PL; 140 mg 1.136 and 15 mg PL; 140 mg 1.136 and 16 mg PL; 140 mg 1.136 and 17 mg PL; 140 mg 1.136 and 18 mg PL; 140 mg 1.136 and 19 mg PL; 140 mg 1.136 and 20 mg PL;

145 mg 1.136 and 1 mg PL; 145 mg 1.136 and 2 mg PL; 145 mg 1.136 and 3 mg PL; 145 mg 1.136 and 4 mg PL; 145 mg 1.136 and 5 mg PL; 145 mg 1.136 and 6 mg PL; 145 mg 1.136 and 7 mg PL; 145 mg 1.136 and 8 mg PL; 145 mg 1.136 and 9 mg PL; 145 mg 1.136 and 10 mg PL; 145 mg 1.136 and 11 mg PL; 145 mg 1.136 and 12 mg PL; 145 mg 1.136 and 13 mg PL; 145 mg 1.136 and 14 mg PL; 145 mg 1.136 and 15 mg PL; 145 mg 1.136 and 16 mg PL; 145 mg 1.136 and 17 mg PL; 145 mg 1.136 and 18 mg PL; 145 mg 1.136 and 19 mg PL; 145 mg 1.136 and 20 mg PL;

150 mg 1.136 and 1 mg PL; 150 mg 1.136 and 2 mg PL; 150 mg 1.136 and 3 mg PL; 150 mg 1.136 and 4 mg PL; 150 mg 1.136 and 5 mg PL; 150 mg 1.136 and 6 mg PL; 150 mg 1.136 and 7 mg PL; 150 mg 1.136 and 8 mg PL; 150 mg 1.136 and 9 mg PL; 150 mg 1.136 and 10 mg PL; 150 mg 1.136 and 11 mg PL; 150 mg 1.136 and 12 mg PL; 150 mg 1.136 and 13 mg PL; 150 mg 1.136 and 14 mg PL; 150 mg 1.136 and 15 mg PL; 150 mg 1.136 and 16 mg PL; 150 mg 1.136 and 17 mg PL; 150 mg 1.136 and 18 mg PL; 150 mg 1.136 and 19 mg PL; 150 mg 1.136 and 20 mg PL;

155 mg 1.136 and 1 mg PL; 155 mg 1.136 and 2 mg PL; 155 mg 1.136 and 3 mg PL; 155 mg 1.136 and 4 mg PL; 155 mg 1.136 and 5 mg PL; 155 mg 1.136 and 6 mg PL; 155 mg 1.136 and 7 mg PL; 155 mg 1.136 and 8 mg PL; 155 mg 1.136 and 9 mg PL; 155 mg 1.136 and 10 mg PL; 155 mg 1.136 and 11 mg PL; 155 mg 1.136 and 12 mg PL; 155 mg 1.136 and 13 mg PL; 155 mg 1.136 and 14 mg PL; 155 mg 1.136 and 15 mg PL; 155 mg 1.136 and 16 mg PL; 155 mg 1.136 and 17 mg PL; 155 mg 1.136 and 18 mg PL; 155 mg 1.136 and 19 mg PL; 155 mg 1.136 and 20 mg PL;

160 mg 1.136 and 1 mg PL; 160 mg 1.136 and 2 mg PL; 160 mg 1.136 and 3 mg PL; 160 mg 1.136 and 4 mg PL; 160 mg 1.136 and 5 mg PL; 160 mg 1.136 and 6 mg PL; 160 mg 1.136 and 7 mg PL; 160 mg 1.136 and 8 mg PL; 160 mg 1.136 and 9 mg PL; 160 mg 1.136 and 10 mg PL; 160 mg 1.136 and 11 mg PL; 160 mg 1.136 and 12 mg PL; 160 mg 1.136 and 13 mg PL; 160 mg 1.136 and 14 mg PL; 160 mg 1.136 and 15 mg PL; 160 mg 1.136 and 16 mg PL; 160 mg 1.136 and 17 mg PL; 160 mg 1.136 and 18 mg PL; 160 mg 1.136 and 19 mg PL; 160 mg 1.136 and 20 mg PL;

165 mg 1.136 and 1 mg PL; 165 mg 1.136 and 2 mg PL; 165 mg 1.136 and 3 mg PL; 165 mg 1.136 and 4 mg PL; 165 mg 1.136 and 5 mg PL; 165 mg 1.136 and 6 mg PL; 165 mg 1.136 and 7 mg PL; 165 mg 1.136 and 8 mg PL; 165 mg 1.136 and 9 mg PL; 165 mg 1.136 and 10 mg PL; 165 mg 1.136 and 11 mg PL; 165 mg 1.136 and 12 mg PL; 165 mg 1.136 and 13 mg PL; 165 mg 1.136 and 14 mg PL; 165 mg 1.136 and 15 mg PL; 165 mg 1.136 and 16 mg PL; 165 mg 1.136 and 17 mg PL; 165 mg 1.136 and 18 mg PL; 165 mg 1.136 and 19 mg PL; 165 mg 1.136 and 20 mg PL;

170 mg 1.136 and 1 mg PL; 170 mg 1.136 and 2 mg PL; 170 mg 1.136 and 3 mg PL; 170 mg 1.136 and 4 mg PL; 170 mg 1.136 and 5 mg PL; 170 mg 1.136 and 6 mg PL; 170 mg 1.136 and 7 mg PL; 170 mg 1.136 and 8 mg PL; 170 mg 1.136 and 9 mg PL; 170 mg 1.136 and 10 mg PL; 170 mg 1.136 and 11 mg PL; 170 mg 1.136 and 12 mg PL; 170 mg 1.136 and 13 mg PL; 170 mg 1.136 and 14 mg PL; 170 mg 1.136 and 15 mg PL; 170 mg 1.136 and 16 mg PL; 170 mg 1.136 and 17 mg PL; 170 mg 1.136 and 18 mg PL; 170 mg 1.136 and 19 mg PL; 170 mg 1.136 and 20 mg PL;

175 mg 1.136 and 1 mg PL; 175 mg 1.136 and 2 mg PL; 175 mg 1.136 and 3 mg PL; 175 mg 1.136 and 4 mg PL; 175 mg 1.136 and 5 mg PL; 175 mg 1.136 and 6 mg PL; 175 mg 1.136 and 7 mg PL; 175 mg 1.136 and 8 mg PL; 175 mg 1.136 and 9 mg PL; 175 mg 1.136 and 10 mg PL; 175 mg 1.136 and 11 mg PL; 175 mg 1.136 and 12 mg PL; 175 mg 1.136 and 13 mg PL; 175 mg 1.136 and 14 mg PL; 175 mg 1.136 and 15 mg PL; 175 mg 1.136 and 16 mg PL; 175 mg 1.136 and 17 mg PL; 175 mg 1.136 and 18 mg PL; 175 mg 1.136 and 19 mg PL; 175 mg 1.136 and 20 mg PL;

180 mg 1.136 and 1 mg PL; 180 mg 1.136 and 2 mg PL; 180 mg 1.136 and 3 mg PL; 180 mg 1.136 and 4 mg PL; 180 mg 1.136 and 5 mg PL; 180 mg 1.136 and 6 mg PL; 180 mg 1.136 and 7 mg PL; 180 mg 1.136 and 8 mg PL; 180 mg 1.136 and 9 mg PL; 180 mg 1.136 and 10 mg PL; 180 mg 1.136 and 11 mg PL; 180 mg 1.136 and 12 mg PL; 180 mg 1.136 and 13 mg PL; 180 mg 1.136 and 14 mg PL; 180 mg 1.136 and 15 mg PL; 180 mg 1.136 and 16 mg PL; 180 mg 1.136 and 17 mg PL; 180 mg 1.136 and 18 mg PL; 180 mg 1.136 and 19 mg PL; 180 mg 1.136 and 20 mg PL;

185 mg 1.136 and 1 mg PL; 185 mg 1.136 and 2 mg PL; 185 mg 1.136 and 3 mg PL; 185 mg 1.136 and 4 mg PL; 185 mg 1.136 and 5 mg PL; 185 mg 1.136 and 6 mg PL; 185 mg 1.136 and 7 mg PL; 185 mg 1.136 and 8 mg PL; 185 mg 1.136 and 9 mg PL; 185 mg 1.136 and 10 mg PL; 185 mg 1.136 and 11 mg PL; 185 mg 1.136 and 12 mg PL; 185 mg 1.136 and 13 mg PL; 185 mg 1.136 and 14 mg PL; 185 mg 1.136 and 15 mg PL; 185 mg 1.136 and 16 mg PL; 185 mg 1.136 and 17 mg PL; 185 mg 1.136 and 18 mg PL; 185 mg 1.136 and 19 mg PL; 185 mg 1.136 and 20 mg PL;

190 mg 1.136 and 1 mg PL; 190 mg 1.136 and 2 mg PL; 190 mg 1.136 and 3 mg PL; 190 mg 1.136 and 4 mg PL; 190 mg 1.136 and 5 mg PL; 190 mg 1.136 and 6 mg PL; 190 mg 1.136 and 7 mg PL; 190 mg 1.136 and 8 mg PL; 190 mg 1.136 and 9 mg PL; 190 mg 1.136 and 10 mg PL; 190 mg 1.136 and 11 mg PL; 190 mg 1.136 and 12 mg PL; 190 mg 1.136 and 13 mg PL; 190 mg 1.136 and 14 mg PL; 190 mg 1.136 and 15 mg PL; 190 mg 1.136 and 16 mg PL; 190 mg 1.136 and 17 mg PL; 190 mg 1.136 and 18 mg PL; 190 mg 1.136 and 19 mg PL; 190 mg 1.136 and 20 mg PL;

195 mg 1.136 and 1 mg PL; 195 mg 1.136 and 2 mg PL; 195 mg 1.136 and 3 mg PL; 195 mg 1.136 and 4 mg PL; 195 mg 1.136 and 5 mg PL; 195 mg 1.136 and 6 mg PL; 195 mg 1.136 and 7 mg PL; 195 mg 1.136 and 8 mg PL; 195 mg 1.136 and 9 mg PL; 195 mg 1.136 and 10 mg PL; 195 mg 1.136 and 11 mg PL; 195 mg 1.136 and 12 mg PL; 195 mg 1.136 and 13 mg PL; 195 mg 1.136 and 14 mg PL; 195 mg 1.136 and 15 mg PL; 195 mg 1.136 and 16 mg PL; 195 mg 1.136 and 17 mg PL; 195 mg 1.136 and 18 mg PL; 195 mg 1.136 and 19 mg PL; 195 mg 1.136 and 20 mg PL;

200 mg 1.136 and 1 mg PL; 200 mg 1.136 and 2 mg PL; 200 mg 1.136 and 3 mg PL; 200 mg 1.136 and 4 mg PL; 200 mg 1.136 and 5 mg PL; 200 mg 1.136 and 6 mg PL; 200 mg 1.136 and 7 mg PL; 200 mg 1.136 and 8 mg PL; 200 mg 1.136 and 9 mg PL; 200 mg 1.136 and 10 mg PL; 200 mg 1.136 and 11 mg PL; 200 mg 1.136 and 12 mg PL; 200 mg 1.136 and 13 mg PL; 200 mg 1.136 and 14 mg PL; 200 mg 1.136 and 15 mg PL; 200 mg 1.136 and 16 mg PL; 200 mg 1.136 and 17 mg PL; 200 mg 1.136 and 18 mg PL; 200 mg 1.136 and 19 mg PL; 200 mg 1.136 and 20 mg PL;

205 mg 1.136 and 1 mg PL; 205 mg 1.136 and 2 mg PL; 205 mg 1.136 and 3 mg PL; 205 mg 1.136 and 4 mg PL; 205 mg 1.136 and 5 mg PL; 205 mg 1.136 and 6 mg PL; 205 mg 1.136 and 7 mg PL; 205 mg 1.136 and 8 mg PL; 205 mg 1.136 and 9 mg PL; 205 mg 1.136 and 10 mg PL; 205 mg 1.136 and 11 mg PL; 205 mg 1.136 and 12 mg PL; 205 mg 1.136 and 13 mg PL; 205 mg 1.136 and 14 mg PL; 205 mg 1.136 and 15 mg PL; 205 mg 1.136 and 16 mg PL; 205 mg 1.136 and 17 mg PL; 205 mg 1.136 and 18 mg PL; 205 mg 1.136 and 19 mg PL; 205 mg 1.136 and 20 mg PL;

210 mg 1.136 and 1 mg PL; 210 mg 1.136 and 2 mg PL; 210 mg 1.136 and 3 mg PL; 210 mg 1.136 and 4 mg PL; 210 mg 1.136 and 5 mg PL; 210 mg 1.136 and 6 mg PL; 210 mg 1.136 and 7 mg PL; 210 mg 1.136 and 8 mg PL; 210 mg 1.136 and 9 mg PL; 210 mg 1.136 and 10 mg PL; 210 mg 1.136 and 11 mg PL; 210 mg 1.136 and 12 mg PL; 210 mg 1.136 and 13 mg PL; 210 mg 1.136 and 14 mg PL; 210 mg 1.136 and 15 mg PL; 210 mg 1.136 and 16 mg PL; 210 mg 1.136 and 17 mg PL; 210 mg 1.136 and 18 mg PL; 210 mg 1.136 and 19 mg PL; 210 mg 1.136 and 20 mg PL;

215 mg 1.136 and 1 mg PL; 215 mg 1.136 and 2 mg PL; 215 mg 1.136 and 3 mg PL; 215 mg 1.136 and 4 mg PL; 215 mg 1.136 and 5 mg PL; 215 mg 1.136 and 6 mg PL; 215 mg 1.136 and 7 mg PL; 215 mg 1.136 and 8 mg PL; 215 mg 1.136 and 9 mg PL; 215 mg 1.136 and 10 mg PL; 215 mg 1.136 and 11 mg PL; 215 mg 1.136 and 12 mg PL; 215 mg 1.136 and 13 mg PL; 215 mg 1.136 and 14 mg PL; 215 mg 1.136 and 15 mg PL; 215 mg 1.136 and 16 mg PL; 215 mg 1.136 and 17 mg PL; 215 mg 1.136 and 18 mg PL; 215 mg 1.136 and 19 mg PL; 215 mg 1.136 and 20 mg PL;

220 mg 1.136 and 1 mg PL; 220 mg 1.136 and 2 mg PL; 220 mg 1.136 and 3 mg PL; 220 mg 1.136 and 4 mg PL; 220 mg 1.136 and 5 mg PL; 220 mg 1.136 and 6 mg PL; 220 mg 1.136 and 7 mg PL; 220 mg 1.136 and 8 mg PL; 220 mg 1.136 and 9 mg PL; 220 mg 1.136 and 10 mg PL; 220 mg 1.136 and 11 mg PL; 220 mg 1.136 and 12 mg PL; 220 mg 1.136 and 13 mg PL; 220 mg 1.136 and 14 mg PL; 220 mg 1.136 and 15 mg PL; 220 mg 1.136 and 16 mg PL; 220 mg 1.136 and 17 mg PL; 220 mg 1.136 and 18 mg PL; 220 mg 1.136 and 19 mg PL; 220 mg 1.136 and 20 mg PL;

225 mg 1.136 and 1 mg PL; 225 mg 1.136 and 2 mg PL; 225 mg 1.136 and 3 mg PL; 225 mg 1.136 and 4 mg PL; 225 mg 1.136 and 5 mg PL; 225 mg 1.136 and 6 mg PL; 225 mg 1.136 and 7 mg PL; 225 mg 1.136 and 8 mg PL; 225 mg 1.136 and 9 mg PL; 225 mg 1.136 and 10 mg PL; 225 mg 1.136 and 11 mg PL; 225 mg 1.136 and 12 mg PL; 225 mg 1.136 and 13 mg PL; 225 mg 1.136 and 14 mg PL; 225 mg 1.136 and 15 mg PL; 225 mg 1.136 and 16 mg PL; 225 mg 1.136 and 17 mg PL; 225 mg 1.136 and 18 mg PL; 225 mg 1.136 and 19 mg PL; 225 mg 1.136 and 20 mg PL;

230 mg 1.136 and 1 mg PL; 230 mg 1.136 and 2 mg PL; 230 mg 1.136 and 3 mg PL; 230 mg 1.136 and 4 mg PL; 230 mg 1.136 and 5 mg PL; 230 mg 1.136 and 6 mg PL; 230 mg 1.136 and 7 mg PL; 230 mg 1.136 and 8 mg PL; 230 mg 1.136 and 9 mg PL; 230 mg 1.136 and 10 mg PL; 230 mg 1.136 and 11 mg PL; 230 mg 1.136 and 12 mg PL; 230 mg 1.136 and 13 mg PL; 230 mg 1.136 and 14 mg PL; 230 mg 1.136 and 15 mg PL; 230 mg 1.136 and 16 mg PL; 230 mg 1.136 and 17 mg PL; 230 mg 1.136 and 18 mg PL; 230 mg 1.136 and 19 mg PL; 230 mg 1.136 and 20 mg PL;

235 mg 1.136 and 1 mg PL; 235 mg 1.136 and 2 mg PL; 235 mg 1.136 and 3 mg PL; 235 mg 1.136 and 4 mg PL; 235 mg 1.136 and 5 mg PL; 235 mg 1.136 and 6 mg PL; 235 mg 1.136 and 7 mg PL; 235 mg 1.136 and 8 mg PL; 235 mg 1.136 and 9 mg PL; 235 mg 1.136 and 10 mg PL; 235 mg 1.136 and 11 mg PL; 235 mg 1.136 and 12 mg PL; 235 mg 1.136 and 13 mg PL; 235 mg 1.136 and 14 mg PL; 235 mg 1.136 and 15 mg PL; 235 mg 1.136 and 16 mg PL; 235 mg 1.136 and 17 mg PL; 235 mg 1.136 and 18 mg PL; 235 mg 1.136 and 19 mg PL; 235 mg 1.136 and 20 mg PL;

240 mg 1.136 and 1 mg PL; 240 mg 1.136 and 2 mg PL; 240 mg 1.136 and 3 mg PL; 240 mg 1.136 and 4 mg PL; 240 mg 1.136 and 5 mg PL; 240 mg 1.136 and 6 mg PL; 240 mg 1.136 and 7 mg PL; 240 mg 1.136 and 8 mg PL; 240 mg 1.136 and 9 mg PL; 240 mg 1.136 and 10 mg PL; 240 mg 1.136 and 11 mg PL; 240 mg 1.136 and 12 mg PL; 240 mg 1.136 and 13 mg PL; 240 mg 1.136 and 14 mg PL; 240 mg 1.136 and 15 mg PL; 240 mg 1.136 and 16 mg PL; 240 mg 1.136 and 17 mg PL; 240 mg 1.136 and 18 mg PL; 240 mg 1.136 and 19 mg PL; 240 mg 1.136 and 20 mg PL;

245 mg 1.136 and 1 mg PL; 245 mg 1.136 and 2 mg PL; 245 mg 1.136 and 3 mg PL; 245 mg 1.136 and 4 mg PL; 245 mg 1.136 and 5 mg PL; 245 mg 1.136 and 6 mg PL; 245 mg 1.136 and 7 mg PL; 245 mg 1.136 and 8 mg PL; 245 mg 1.136 and 9 mg PL; 245 mg 1.136 and 10 mg PL; 245 mg 1.136 and 11 mg PL; 245 mg 1.136 and 12 mg PL; 245 mg 1.136 and 13 mg PL; 245 mg 1.136 and 14 mg PL; 245 mg 1.136 and 15 mg PL; 245 mg 1.136 and 16 mg PL; 245 mg 1.136 and 17 mg PL; 245 mg 1.136 and 18 mg PL; 245 mg 1.136 and 19 mg PL; 245 mg 1.136 and 20 mg PL;

250 mg 1.136 and 1 mg PL; 250 mg 1.136 and 2 mg PL; 250 mg 1.136 and 3 mg PL; 250 mg 1.136 and 4 mg PL; 250 mg 1.136 and 5 mg PL; 250 mg 1.136 and 6 mg PL; 250 mg 1.136 and 7 mg PL; 250 mg 1.136 and 8 mg PL; 250 mg 1.136 and 9 mg PL; 250 mg 1.136 and 10 mg PL; 250 mg 1.136 and 11 mg PL; 250 mg 1.136 and 12 mg PL; 250 mg 1.136 and 13 mg PL; 250 mg 1.136 and 14 mg PL; 250 mg 1.136 and 15 mg PL; 250 mg 1.136 and 16 mg PL; 250 mg 1.136 and 17 mg PL; 250 mg 1.136 and 18 mg PL; 250 mg 1.136 and 19 mg PL; 250 mg 1.136 and 20 mg PL;

255 mg 1.136 and 1 mg PL; 255 mg 1.136 and 2 mg PL; 255 mg 1.136 and 3 mg PL; 255 mg 1.136 and 4 mg PL; 255 mg 1.136 and 5 mg PL; 255 mg 1.136 and 6 mg PL; 255 mg 1.136 and 7 mg PL; 255 mg 1.136 and 8 mg PL; 255 mg 1.136 and 9 mg PL; 255 mg 1.136 and 10 mg PL; 255 mg 1.136 and 11 mg PL; 255 mg 1.136 and 12 mg PL; 255 mg 1.136 and 13 mg PL; 255 mg 1.136 and 14 mg PL; 255 mg 1.136 and 15 mg PL; 255 mg 1.136 and 16 mg PL; 255 mg 1.136 and 17 mg PL; 255 mg 1.136 and 18 mg PL; 255 mg 1.136 and 19 mg PL; 255 mg 1.136 and 20 mg PL;

260 mg 1.136 and 1 mg PL; 260 mg 1.136 and 2 mg PL; 260 mg 1.136 and 3 mg PL; 260 mg 1.136 and 4 mg PL; 260 mg 1.136 and 5 mg PL; 260 mg 1.136 and 6 mg PL; 260 mg 1.136 and 7 mg PL; 260 mg 1.136 and 8 mg PL; 260 mg 1.136 and 9 mg PL; 260 mg 1.136 and 10 mg PL; 260 mg 1.136 and 11 mg PL; 260 mg 1.136 and 12 mg PL; 260 mg 1.136 and 13 mg PL; 260 mg 1.136 and 14 mg PL; 260 mg 1.136 and 15 mg PL; 260 mg 1.136 and 16 mg PL; 260 mg 1.136 and 17 mg PL; 260 mg 1.136 and 18 mg PL; 260 mg 1.136 and 19 mg PL; 260 mg 1.136 and 20 mg PL;

265 mg 1.136 and 1 mg PL; 265 mg 1.136 and 2 mg PL; 265 mg 1.136 and 3 mg PL; 265 mg 1.136 and 4 mg PL; 265 mg 1.136 and 5 mg PL; 265 mg 1.136 and 6 mg PL; 265 mg 1.136 and 7 mg PL; 265 mg 1.136 and 8 mg PL; 265 mg 1.136 and 9 mg PL; 265 mg 1.136 and 10 mg PL; 265 mg 1.136 and 11 mg PL; 265 mg 1.136 and 12 mg PL; 265 mg 1.136 and 13 mg PL; 265 mg 1.136 and 14 mg PL; 265 mg 1.136 and 15 mg PL; 265 mg 1.136 and 16 mg PL; 265 mg 1.136 and 17 mg PL; 265 mg 1.136 and 18 mg PL; 265 mg 1.136 and 19 mg PL; 265 mg 1.136 and 20 mg PL;

270 mg 1.136 and 1 mg PL; 270 mg 1.136 and 2 mg PL; 270 mg 1.136 and 3 mg PL; 270 mg 1.136 and 4 mg PL; 270 mg 1.136 and 5 mg PL; 270 mg 1.136 and 6 mg PL; 270 mg 1.136 and 7 mg PL; 270 mg 1.136 and 8 mg PL; 270 mg 1.136 and 9 mg PL; 270 mg 1.136 and 10 mg PL; 270 mg 1.136 and 11 mg PL; 270 mg 1.136 and 12 mg PL; 270 mg 1.136 and 13 mg PL; 270 mg 1.136 and 14 mg PL; 270 mg 1.136 and 15 mg PL; 270 mg 1.136 and 16 mg PL; 270 mg 1.136 and 17 mg PL; 270 mg 1.136 and 18 mg PL; 270 mg 1.136 and 19 mg PL; 270 mg 1.136 and 20 mg PL;

275 mg 1.136 and 1 mg PL; 275 mg 1.136 and 2 mg PL; 275 mg 1.136 and 3 mg PL; 275 mg 1.136 and 4 mg PL; 275 mg 1.136 and 5 mg PL; 275 mg 1.136 and 6 mg PL; 275 mg 1.136 and 7 mg PL; 275 mg 1.136 and 8 mg PL; 275 mg 1.136 and 9 mg PL; 275 mg 1.136 and 10 mg PL; 275 mg 1.136 and 11 mg PL; 275 mg 1.136 and 12 mg PL; 275 mg 1.136 and 13 mg PL; 275 mg 1.136 and 14 mg PL; 275 mg 1.136 and 15 mg PL; 275 mg 1.136 and 16 mg PL; 275 mg 1.136 and 17 mg PL; 275 mg 1.136 and 18 mg PL; 275 mg 1.136 and 19 mg PL; 275 mg 1.136 and 20 mg PL;

280 mg 1.136 and 1 mg PL; 280 mg 1.136 and 2 mg PL; 280 mg 1.136 and 3 mg PL; 280 mg 1.136 and 4 mg PL; 280 mg 1.136 and 5 mg PL; 280 mg 1.136 and 6 mg PL; 280 mg 1.136 and 7 mg PL; 280 mg 1.136 and 8 mg PL; 280 mg 1.136 and 9 mg PL; 280 mg 1.136 and 10 mg PL; 280 mg 1.136 and 11 mg PL; 280 mg 1.136 and 12 mg PL; 280 mg 1.136 and 13 mg PL; 280 mg 1.136 and 14 mg PL; 280 mg 1.136 and 15 mg PL; 280 mg 1.136 and 16 mg PL; 280 mg 1.136 and 17 mg PL; 280 mg 1.136 and 18 mg PL; 280 mg 1.136 and 19 mg PL; 280 mg 1.136 and 20 mg PL;

285 mg 1.136 and 1 mg PL; 285 mg 1.136 and 2 mg PL; 285 mg 1.136 and 3 mg PL; 285 mg 1.136 and 4 mg PL; 285 mg 1.136 and 5 mg PL; 285 mg 1.136 and 6 mg PL; 285 mg 1.136 and 7 mg PL; 285 mg 1.136 and 8 mg PL; 285 mg 1.136 and 9 mg PL; 285 mg 1.136 and 10 mg PL; 285 mg 1.136 and 11 mg PL; 285 mg 1.136 and 12 mg PL; 285 mg 1.136 and 13 mg PL; 285 mg 1.136 and 14 mg PL; 285 mg 1.136 and 15 mg PL; 285 mg 1.136 and 16 mg PL; 285 mg 1.136 and 17 mg PL; 285 mg 1.136 and 18 mg PL; 285 mg 1.136 and 19 mg PL; 285 mg 1.136 and 20 mg PL;

290 mg 1.136 and 1 mg PL; 290 mg 1.136 and 2 mg PL; 290 mg 1.136 and 3 mg PL; 290 mg 1.136 and 4 mg PL; 290 mg 1.136 and 5 mg PL; 290 mg 1.136 and 6 mg PL; 290 mg 1.136 and 7 mg PL; 290 mg 1.136 and 8 mg PL; 290 mg 1.136 and 9 mg PL; 290 mg 1.136 and 10 mg PL; 290 mg 1.136 and 11 mg PL; 290 mg 1.136 and 12 mg PL; 290 mg 1.136 and 13 mg PL; 290 mg 1.136 and 14 mg PL; 290 mg 1.136 and 15 mg PL; 290 mg 1.136 and 16 mg PL; 290 mg 1.136 and 17 mg PL; 290 mg 1.136 and 18 mg PL; 290 mg 1.136 and 19 mg PL; 290 mg 1.136 and 20 mg PL;

295 mg 1.136 and 1 mg PL; 295 mg 1.136 and 2 mg PL; 295 mg 1.136 and 3 mg PL; 295 mg 1.136 and 4 mg PL; 295 mg 1.136 and 5 mg PL; 295 mg 1.136 and 6 mg PL; 295 mg 1.136 and 7 mg PL; 295 mg 1.136 and 8 mg PL; 295 mg 1.136 and 9 mg PL; 295 mg 1.136 and 10 mg PL; 295 mg 1.136 and 11 mg PL; 295 mg 1.136 and 12 mg PL; 295 mg 1.136 and 13 mg PL; 295 mg 1.136 and 14 mg PL; 295 mg 1.136 and 15 mg PL; 295 mg 1.136 and 16 mg PL; 295 mg 1.136 and 17 mg PL; 295 mg 1.136 and 18 mg PL; 295 mg 1.136 and 19 mg PL; 295 mg 1.136 and 20 mg PL;

300 mg 1.136 and 1 mg PL; 300 mg 1.136 and 2 mg PL; 300 mg 1.136 and 3 mg PL; 300 mg 1.136 and 4 mg PL; 300 mg 1.136 and 5 mg PL; 300 mg 1.136 and 6 mg PL; 300 mg 1.136 and 7 mg PL; 300 mg 1.136 and 8 mg PL; 300 mg 1.136 and 9 mg PL; 300 mg 1.136 and 10 mg PL; 300 mg 1.136 and 11 mg PL; 300 mg 1.136 and 12 mg PL; 300 mg 1.136 and 13 mg PL; 300 mg 1.136 and 14 mg PL; 300 mg 1.136 and 15 mg PL; 300 mg 1.136 and 16 mg PL; 300 mg 1.136 and 17 mg PL; 300 mg 1.136 and 18 mg PL; 300 mg 1.136 and 19 mg PL; 300 mg 1.136 and 20 mg PL.

305 mg 1.136 and 1 mg PL; 305 mg 1.136 and 2 mg PL; 305 mg 1.136 and 3 mg PL; 305 mg 1.136 and 4 mg PL; 305 mg 1.136 and 5 mg PL; 305 mg 1.136 and 6 mg PL; 305 mg 1.136 and 7 mg PL; 305 mg 1.136 and 8 mg PL; 305 mg 1.136 and 9 mg PL; 305 mg 1.136 and 10 mg PL; 305 mg 1.136 and 11 mg PL; 305 mg 1.136 and 12 mg PL; 305 mg 1.136 and 13 mg PL; 305 mg 1.136 and 14 mg PL; 305 mg 1.136 and 15 mg PL; 305 mg 1.136 and 16 mg PL; 305 mg 1.136 and 17 mg PL; 305 mg 1.136 and 18 mg PL; 305 mg 1.136 and 19 mg PL; 305 mg 1.136 and 20 mg PL.

310 mg 1.136 and 1 mg PL; 310 mg 1.136 and 2 mg PL; 310 mg 1.136 and 3 mg PL; 310 mg 1.136 and 4 mg PL; 310 mg 1.136 and 5 mg PL; 310 mg 1.136 and 6 mg PL; 310 mg 1.136 and 7 mg PL; 310 mg 1.136 and 8 mg PL; 310 mg 1.136 and 9 mg PL; 310 mg 1.136 and 10 mg PL; 310 mg 1.136 and 11 mg PL; 310 mg 1.136 and 12 mg PL; 310 mg 1.136 and 13 mg PL; 310 mg 1.136 and 14 mg PL; 310 mg 1.136 and 15 mg PL; 310 mg 1.136 and 16 mg PL; 310 mg 1.136 and 17 mg PL; 310 mg 1.136 and 18 mg PL; 310 mg 1.136 and 19 mg PL; 310 mg 1.136 and 20 mg PL.

315 mg 1.136 and 1 mg PL; 315 mg 1.136 and 2 mg PL; 315 mg 1.136 and 3 mg PL; 315 mg 1.136 and 4 mg PL; 315 mg 1.136 and 5 mg PL; 315 mg 1.136 and 6 mg PL; 315 mg 1.136 and 7 mg PL; 315 mg 1.136 and 8 mg PL; 315 mg 1.136 and 9 mg PL; 315 mg 1.136 and 10 mg PL; 315 mg 1.136 and 11 mg PL; 315 mg 1.136 and 12 mg PL; 315 mg 1.136 and 13 mg PL; 315 mg 1.136 and 14 mg PL; 315 mg 1.136 and 15 mg PL; 315 mg 1.136 and 16 mg PL; 315 mg 1.136 and 17 mg PL; 315 mg 1.136 and 18 mg PL; 315 mg 1.136 and 19 mg PL; 315 mg 1.136 and 20 mg PL.

320 mg 1.136 and 1 mg PL; 320 mg 1.136 and 2 mg PL; 320 mg 1.136 and 3 mg PL; 320 mg 1.136 and 4 mg PL; 320 mg 1.136 and 5 mg PL; 320 mg 1.136 and 6 mg PL; 320 mg 1.136 and 7 mg PL; 320 mg 1.136 and 8 mg PL; 320 mg 1.136 and 9 mg PL; 320 mg 1.136 and 10 mg PL; 320 mg 1.136 and 11 mg PL; 320 mg 1.136 and 12 mg PL; 320 mg 1.136 and 13 mg PL; 320 mg 1.136 and 14 mg PL; 320 mg 1.136 and 15 mg PL; 320 mg 1.136 and 16 mg PL; 320 mg 1.136 and 17 mg PL; 320 mg 1.136 and 18 mg PL; 320 mg 1.136 and 19 mg PL; 320 mg 1.136 and 20 mg PL.

325 mg 1.136 and 1 mg PL; 325 mg 1.136 and 2 mg PL; 325 mg 1.136 and 3 mg PL; 325 mg 1.136 and 4 mg PL; 325 mg 1.136 and 5 mg PL; 325 mg 1.136 and 6 mg PL; 325 mg 1.136 and 7 mg PL; 325 mg 1.136 and 8 mg PL; 325 mg 1.136 and 9 mg PL; 325 mg 1.136 and 10 mg PL; 325 mg 1.136 and 11 mg PL; 325 mg 1.136 and 12 mg PL; 325 mg 1.136 and 13 mg PL; 325 mg 1.136 and 14 mg PL; 325 mg 1.136 and 15 mg PL; 325 mg 1.136 and 16 mg PL; 325 mg 1.136 and 17 mg PL; 325 mg 1.136 and 18 mg PL; 325 mg 1.136 and 19 mg PL; 325 mg 1.136 and 20 mg PL.

330 mg 1.136 and 1 mg PL; 330 mg 1.136 and 2 mg PL; 330 mg 1.136 and 3 mg PL; 330 mg 1.136 and 4 mg PL; 330 mg 1.136 and 5 mg PL; 330 mg 1.136 and 6 mg PL; 330 mg 1.136 and 7 mg PL; 330 mg 1.136 and 8 mg PL; 330 mg 1.136 and 9 mg PL; 330 mg 1.136 and 10 mg PL; 330 mg 1.136 and 11 mg PL; 330 mg 1.136 and 12 mg PL; 330 mg 1.136 and 13 mg PL; 330 mg 1.136 and 14 mg PL; 330 mg 1.136 and 15 mg PL; 330 mg 1.136 and 16 mg PL; 330 mg 1.136 and 17 mg PL; 330 mg 1.136 and 18 mg PL; 330 mg 1.136 and 19 mg PL; 330 mg 1.136 and 20 mg PL.

335 mg 1.136 and 1 mg PL; 335 mg 1.136 and 2 mg PL; 335 mg 1.136 and 3 mg PL; 335 mg 1.136 and 4 mg PL; 335 mg 1.136 and 5 mg PL; 335 mg 1.136 and 6 mg PL; 335 mg 1.136 and 7 mg PL; 335 mg 1.136 and 8 mg PL; 335 mg 1.136 and 9 mg PL; 335 mg 1.136 and 10 mg PL; 335 mg 1.136 and 11 mg PL; 335 mg 1.136 and 12 mg PL; 335 mg 1.136 and 13 mg PL; 335 mg 1.136 and 14 mg PL; 335 mg 1.136 and 15 mg PL; 335 mg 1.136 and 16 mg PL; 335 mg 1.136 and 17 mg PL; 335 mg 1.136 and 18 mg PL; 335 mg 1.136 and 19 mg PL; 335 mg 1.136 and 20 mg PL.

340 mg 1.136 and 1 mg PL; 340 mg 1.136 and 2 mg PL; 340 mg 1.136 and 3 mg PL; 340 mg 1.136 and 4 mg PL; 340 mg 1.136 and 5 mg PL; 340 mg 1.136 and 6 mg PL; 340 mg 1.136 and 7 mg PL; 340 mg 1.136 and 8 mg PL; 340 mg 1.136 and 9 mg PL; 340 mg 1.136 and 10 mg PL; 340 mg 1.136 and 11 mg PL; 340 mg 1.136 and 12 mg PL; 340 mg 1.136 and 13 mg PL; 340 mg 1.136 and 14 mg PL; 340 mg 1.136 and 15 mg PL; 340 mg 1.136 and 16 mg PL; 340 mg 1.136 and 17 mg PL; 340 mg 1.136 and 18 mg PL; 340 mg 1.136 and 19 mg PL; 340 mg 1.136 and 20 mg PL.

345 mg 1.136 and 1 mg PL; 345 mg 1.136 and 2 mg PL; 345 mg 1.136 and 3 mg PL; 345 mg 1.136 and 4 mg PL; 345 mg 1.136 and 5 mg PL; 345 mg 1.136 and 6 mg PL; 345 mg 1.136 and 7 mg PL; 345 mg 1.136 and 8 mg PL; 345 mg 1.136 and 9 mg PL; 345 mg 1.136 and 10 mg PL; 345 mg 1.136 and 11 mg PL; 345 mg 1.136 and 12 mg PL; 345 mg 1.136 and 13 mg PL; 345 mg 1.136 and 14 mg PL; 345 mg 1.136 and 15 mg PL; 345 mg 1.136 and 16 mg PL; 345 mg 1.136 and 17 mg PL; 345 mg 1.136 and 18 mg PL; 345 mg 1.136 and 19 mg PL; 345 mg 1.136 and 20 mg PL.

350 mg 1.136 and 1 mg PL; 350 mg 1.136 and 2 mg PL; 350 mg 1.136 and 3 mg PL; 350 mg 1.136 and 4 mg PL; 350 mg 1.136 and 5 mg PL; 350 mg 1.136 and 6 mg PL; 350 mg 1.136 and 7 mg PL; 350 mg 1.136 and 8 mg PL; 350 mg 1.136 and 9 mg PL; 350 mg 1.136 and 10 mg PL; 350 mg 1.136 and 11 mg PL; 350 mg 1.136 and 12 mg PL; 350 mg 1.136 and 13 mg PL; 350 mg 1.136 and 14 mg PL; 350 mg 1.136 and 15 mg PL; 350 mg 1.136 and 16 mg PL; 350 mg 1.136 and 17 mg PL; 350 mg 1.136 and 18 mg PL; 350 mg 1.136 and 19 mg PL; 350 mg 1.136 and 20 mg PL.

355 mg 1.136 and 1 mg PL; 355 mg 1.136 and 2 mg PL; 355 mg 1.136 and 3 mg PL; 355 mg 1.136 and 4 mg PL; 355 mg 1.136 and 5 mg PL; 355 mg 1.136 and 6 mg PL; 355 mg 1.136 and 7 mg PL; 355 mg 1.136 and 8 mg PL; 355 mg 1.136 and 9 mg PL; 355 mg 1.136 and 10 mg PL; 355 mg 1.136 and 11 mg PL; 355 mg 1.136 and 12 mg PL; 355 mg 1.136 and 13 mg PL; 355 mg 1.136 and 14 mg PL; 355 mg 1.136 and 15 mg PL; 355 mg 1.136 and 16 mg PL; 355 mg 1.136 and 17 mg PL; 355 mg 1.136 and 18 mg PL; 355 mg 1.136 and 19 mg PL; 355 mg 1.136 and 20 mg PL.

360 mg 1.136 and 1 mg PL; 360 mg 1.136 and 2 mg PL; 360 mg 1.136 and 3 mg PL; 360 mg 1.136 and 4 mg PL; 360 mg 1.136 and 5 mg PL; 360 mg 1.136 and 6 mg PL; 360 mg 1.136 and 7 mg PL; 360 mg 1.136 and 8 mg PL; 360 mg 1.136 and 9 mg PL; 360 mg 1.136 and 10 mg PL; 360 mg 1.136 and 11 mg PL; 360 mg 1.136 and 12 mg PL; 360 mg 1.136 and 13 mg PL; 360 mg 1.136 and 14 mg PL; 360 mg 1.136 and 15 mg PL; 360 mg 1.136 and 16 mg PL; 360 mg 1.136 and 17 mg PL; 360 mg 1.136 and 18 mg PL; 360 mg 1.136 and 19 mg PL; 360 mg 1.136 and 20 mg PL.

365 mg 1.136 and 1 mg PL; 365 mg 1.136 and 2 mg PL; 365 mg 1.136 and 3 mg PL; 365 mg 1.136 and 4 mg PL; 365 mg 1.136 and 5 mg PL; 365 mg 1.136 and 6 mg PL; 365 mg 1.136 and 7 mg PL; 365 mg 1.136 and 8 mg PL; 365 mg 1.136 and 9 mg PL; 365 mg 1.136 and 10 mg PL; 365 mg 1.136 and 11 mg PL; 365 mg 1.136 and 12 mg PL; 365 mg 1.136 and 13 mg PL; 365 mg 1.136 and 14 mg PL; 365 mg 1.136 and 15 mg PL; 365 mg 1.136 and 16 mg PL; 365 mg 1.136 and 17 mg PL; 365 mg 1.136 and 18 mg PL; 365 mg 1.136 and 19 mg PL; 365 mg 1.136 and 20 mg PL.

370 mg 1.136 and 1 mg PL; 370 mg 1.136 and 2 mg PL; 370 mg 1.136 and 3 mg PL; 370 mg 1.136 and 4 mg PL; 370 mg 1.136 and 5 mg PL; 370 mg 1.136 and 6 mg PL; 370 mg 1.136 and 7 mg PL; 370 mg 1.136 and 8 mg PL; 370 mg 1.136 and 9 mg PL; 370 mg 1.136 and 10 mg PL; 370 mg 1.136 and 11 mg PL; 370 mg 1.136 and 12 mg PL; 370 mg 1.136 and 13 mg PL; 370 mg 1.136 and 14 mg PL; 370 mg 1.136 and 15 mg PL; 370 mg 1.136 and 16 mg PL; 370 mg 1.136 and 17 mg PL; 370 mg 1.136 and 18 mg PL; 370 mg 1.136 and 19 mg PL; 370 mg 1.136 and 20 mg PL.

375 mg 1.136 and 1 mg PL; 375 mg 1.136 and 2 mg PL; 375 mg 1.136 and 3 mg PL; 375 mg 1.136 and 4 mg PL; 375 mg 1.136 and 5 mg PL; 375 mg 1.136 and 6 mg PL; 375 mg 1.136 and 7 mg PL; 375 mg 1.136 and 8 mg PL; 375 mg 1.136 and 9 mg PL; 375 mg 1.136 and 10 mg PL; 375 mg 1.136 and 11 mg PL; 375 mg 1.136 and 12 mg PL; 375 mg 1.136 and 13 mg PL; 375 mg 1.136 and 14 mg PL; 375 mg 1.136 and 15 mg PL; 375 mg 1.136 and 16 mg PL; 375 mg 1.136 and 17 mg PL; 375 mg 1.136 and 18 mg PL; 375 mg 1.136 and 19 mg PL; 375 mg 1.136 and 20 mg PL.

380 mg 1.136 and 1 mg PL; 380 mg 1.136 and 2 mg PL; 380 mg 1.136 and 3 mg PL; 380 mg 1.136 and 4 mg PL; 380 mg 1.136 and 5 mg PL; 380 mg 1.136 and 6 mg PL; 380 mg 1.136 and 7 mg PL; 380 mg 1.136 and 8 mg PL; 380 mg 1.136 and 9 mg PL; 380 mg 1.136 and 10 mg PL; 380 mg 1.136 and 11 mg PL; 380 mg 1.136 and 12 mg PL; 380 mg 1.136 and 13 mg PL; 380 mg 1.136 and 14 mg PL; 380 mg 1.136 and 15 mg PL; 380 mg 1.136 and 16 mg PL; 380 mg 1.136 and 17 mg PL; 380 mg 1.136 and 18 mg PL; 380 mg 1.136 and 19 mg PL; 380 mg 1.136 and 20 mg PL.

385 mg 1.136 and 1 mg PL; 385 mg 1.136 and 2 mg PL; 385 mg 1.136 and 3 mg PL; 385 mg 1.136 and 4 mg PL; 385 mg 1.136 and 5 mg PL; 385 mg 1.136 and 6 mg PL; 385 mg 1.136 and 7 mg PL; 385 mg 1.136 and 8 mg PL; 385 mg 1.136 and 9 mg PL; 385 mg 1.136 and 10 mg PL; 385 mg 1.136 and 11 mg PL; 385 mg 1.136 and 12 mg PL; 385 mg 1.136 and 13 mg PL; 385 mg 1.136 and 14 mg PL; 385 mg 1.136 and 15 mg PL; 385 mg 1.136 and 16 mg PL; 385 mg 1.136 and 17 mg PL; 385 mg 1.136 and 18 mg PL; 385 mg 1.136 and 19 mg PL; 385 mg 1.136 and 20 mg PL.

390 mg 1.136 and 1 mg PL; 390 mg 1.136 and 2 mg PL; 390 mg 1.136 and 3 mg PL; 390 mg 1.136 and 4 mg PL; 390 mg 1.136 and 5 mg PL; 390 mg 1.136 and 6 mg PL; 390 mg 1.136 and 7 mg PL; 390 mg 1.136 and 8 mg PL; 390 mg 1.136 and 9 mg PL; 390 mg 1.136 and 10 mg PL; 390 mg 1.136 and 11 mg PL; 390 mg 1.136 and 12 mg PL; 390 mg 1.136 and 13 mg PL; 390 mg 1.136 and 14 mg PL; 390 mg 1.136 and 15 mg PL; 390 mg 1.136 and 16 mg PL; 390 mg 1.136 and 17 mg PL; 390 mg 1.136 and 18 mg PL; 390 mg 1.136 and 19 mg PL; 390 mg 1.136 and 20 mg PL.

395 mg 1.136 and 1 mg PL; 395 mg 1.136 and 2 mg PL; 395 mg 1.136 and 3 mg PL; 395 mg 1.136 and 4 mg PL; 395 mg 1.136 and 5 mg PL; 395 mg 1.136 and 6 mg PL; 395 mg 1.136 and 7 mg PL; 395 mg 1.136 and 8 mg PL; 395 mg 1.136 and 9 mg PL; 395 mg 1.136 and 10 mg PL; 395 mg 1.136 and 11 mg PL; 395 mg 1.136 and 12 mg PL; 395 mg 1.136 and 13 mg PL; 395 mg 1.136 and 14 mg PL; 395 mg 1.136 and 15 mg PL; 395 mg 1.136 and 16 mg PL; 395 mg 1.136 and 17 mg PL; 395 mg 1.136 and 18 mg PL; 395 mg 1.136 and 19 mg PL; 395 mg 1.136 and 20 mg PL.

400 mg 1.136 and 1 mg PL; 400 mg 1.136 and 2 mg PL; 400 mg 1.136 and 3 mg PL; 400 mg 1.136 and 4 mg PL; 400 mg 1.136 and 5 mg PL; 400 mg 1.136 and 6 mg PL; 400 mg 1.136 and 7 mg PL; 400 mg 1.136 and 8 mg PL; 400 mg 1.136 and 9 mg PL; 400 mg 1.136 and 10 mg PL; 400 mg 1.136 and 11 mg PL; 400 mg 1.136 and 12 mg PL; 400 mg 1.136 and 13 mg PL; 400 mg 1.136 and 14 mg PL; 400 mg 1.136 and 15 mg PL; 400 mg 1.136 and 16 mg PL; 400 mg 1.136 and 17 mg PL; 400 mg 1.136 and 18 mg PL; 400 mg 1.136 and 19 mg PL; 400 mg 1.136 and 20 mg PL.

### Medical Indications

The pyrimidine derivatives of the formula (I) show excellent CRTH2 antagonistic activity. They are, therefore, suitable especially for the prophylaxis and treatment of diseases associated with CRTH2 activity.

It has been found that the pharmaceutical compositions described herein have a beneficial effect in terms of bronchospasmolysis and reduction of inflammations in the airways, as well as allergic diseases of the skin or the eyes. This is particularly true for combinations of 1.136 with LTD4 antagonists 2-5, particularly with montelukast, or combinations of 1.136 with 5-LO inhibitors 2-17, particularly with Zileuton. This is due to the different modes of action of the CRTH2 antagonists 1 and the LTD4 antagonists 2-5 or the 5-LO inhibitors 2-17 in the inflammatory process induced by arachidonic acid metabolites. CRTH2 antagonists 1 are decreasing the activity of prostaglandins on one side of the arachadonic acid inflammation cascade, whereas LTD4 antagonists 2-5 or 5-LO inhibitors 2-17 are decreasing the activity of leukotrienes on the other side of the arachadonic acid inflammation cascade. The combination of a CRTH2 antagonist 1 and a leukotriene antagonist 2-5 or 5-LO inhibitor 2-17 is of particular interest because cyclooxygenase inhibitors, that prevent the production of prostaglandins, can induce asthmatic attacks and are not useful for treatment of inflammation in asthmatic patients, whereas inhibition of a receptor further down-stream is not expected to have such effects. The induction of asthmatic attacks by COX inhibitors is believed to be mediated by shunting of the arachidonic acid metabolites to the leukotriene cascade causing increased production of LTC4 and LTB4. LTC4 and LTB4 cause bronchoconstriction and inflammation. In contrast, CRTH2 antagonists 1 do not increase the production of leukotrienes.

In another aspect it has been found that the pharmaceutical compositions described herein have a beneficial effect in terms of reduction of inflammations in the airways, as well as allergic diseases of the skin or the eyes. This is particularly true for combinations of 1, preferably 1.136, with histamine receptor antagonists 2-11, particularly with cetirizine or other second and third generation antihistamines Prostaglandins and histamine are important inflammatory mediators. Mast cells are the major sources of histamine and prostaglandins during inflammation of the airways, skin or eyes. Mast cells express CRTH2 and activation of CRTH2 plays a central role in the release of inflammatory mediators and activation of TH2 cells [Gyles et al., Immunology. 2006, 119(3):362-8] and other inflammatory cell types like basophils and eosinophils. CRTH2 antagonists 1 are inhibiting one side of the inflammation cascade mediated by prostaglandins, whereas histamine receptor antagonists 2-11 are inhibiting the activity of histamines on the other side of the inflammation cascade. The combination of a CRTH2 antagonist 1 and a histamine receptor antagonist 2-11 is of particular interest because two important inflammation cascades can be inhibited. Moreover, a positive feedback of mast cell-released prostaglandins on mast cells can be inhibited.

It has been found that the pharmaceutical compositions described herein have a beneficial effect in terms of bronchospasmolysis and reduction of inflammations in the airways, as well as inflammatory diseases of the joints, and allergic diseases of the oro-naso pharynx, skin or the eyes.

In another aspect this is particularly true for combinations of 1, especially 1.136, with PDE4 inhibitors 2-4, particularly with Roflumilast. CRTH2 is mainly expressed on TH2 cells, eosinophils, basophils and mast cells and weakly in monocytes or macrophages. CRTH2 is not expressed in neutrophils whereas PDE4 is also expressed in monocytes, macrophages and neutrophils. The combination of a CRTH2 antagonist 1 and a PDE4 inhibitor 2-4 is of particular interest because of the different modes of action of a CRTH2 antagonist 1 and a PDE4 inhibitor 2-4 and the inhibition of the two important inflammation cascades. The combination of a CRTH2 antagonist 1 and a PDE4 inhibitor 2-4 is of particular interest in diseases where target cells for CRTH2 antagonists 1 and target cells for PDE4 inhibitors 2-4 contribute to the inflammation.

In another aspect this is particularly true for combinations of 1, especially 1.136, with CCR5 antagonists 2-26, particularly with Maraviroc. CRTH2 is mainly expressed on Th2 cells, eosinophils, basophils and mast cells and weakly in monocytes or macrophages. CRTH2 is not expressed in neutrophils whereas CCR5 is also expressed in monocytes, macrophages and neutrophils. The combination of a CRTH2 antagonist 1 and a CCR5 antagonist 2-26 is of particular interest because of the different modes of action of the CRTH2 antagonists 1 and the CCR5 antagonists 2-26 and the inhibition of two important inflammation cascades in the inflammatory process of the airways, joints, skin or the eyes. The combination of a CRTH2 antagonist 1 and a CCR5 antagonist 2-26 is of particular interest in diseases where target cells for CRTH2 antagonists 1 and target cells for CCR5 antagonist 2-26 contribute to the inflammation.

In another aspect it has been found that the pharmaceutical compositions described herein have a beneficial effect in terms of reduction of inflammation in inflammatory bowel disease. This is particularly true for combinations of 1, especially 1.136, with CCR9 antagonists 2-27, particularly with Trafficet and with Sulfonamides 2-53 or amino-salicylates 2-54 particularly with Mesalazine and Sulfasalazine. The combination of a CRTH2 antagonist 1 and a CCR9 antagonist 2-27 is of particular interest because of the different modes of action of the CRTH2 antagonists 1 and the CCR9 antagonists 2-27 in the inflammatory process of the gastrointestinal tract. Gastrointestinal inflammation is often accompanied by eosinophilic inflammation and elevated cytokine levels but also by intestine-specific homing of inflammatory cells. CRTH2 antagonists 1 are inhibiting the recruitment and activation of eosinophils and Th2 cells accompanied by reduced cytokine levels, whereas CCR9 antagonists 2-27 inhibit the intestine-specific homing of inflammatory cells at the side of the inflammation.

The combination of a CRTH2 antagonist 1 and a sulfonamide 2-53 or amino-salicylate 2-54 is of particular interest because of the different modes of action of the CRTH2 antagonists 1 and the Sulfonamide 2-53 or aminosalicylate 2-54 in the inflammatory process of the gastrointestinal tract. Gastrointestinal inflammation is often accompanied by eosinophilic inflammation and elevated cytokine levels but also other inflammatory cells contribute to the inflammation process. Inflammatory cells are recruited from the blood stream and compounds that are rapidly metabolized can not fully prevent recruitment and activation of blood-derived inflammatory cells. Sulfonamides 2-53/amino-salicylates 2-54 like Mesalazine and Sulfasalazine are bowel-specific drugs that are metabolized in the gut and have its predominant actions there, thereby having fewer systemic side effects but also low systemic exposure. Hence, CRTH2 antagonists 1, which have high systemic exposure and high metabolic stability are inhibiting inflammatory cells that are attracted to the site of inflammation from the blood stream whereas the Sulfonamide 2-53/aminosalicylates 2-54 inhibits inflammatory process in the gut.

One embodiment of the invention is a method of treating an indication selected from indications (A): prevention and treatment of diseases of the airways and lungs which are accompanied by increased or altered production of mucus and/or inflammatory and/or obstructive diseases of the airways such as acute bronchitis, chronic bronchitis, chronic obstructive bronchitis (COPD), cough, pulmonary emphysema,

allergic or non-allergic rhinitis or sinusitis, chronic sinusitis or rhinitis,
nasal polyposis, chronic rhinosinusitis, acute rhinosinusitis,
asthma, allergic bronchitis, alveolitis, Farmer's disease, hyperreactive airways,
bronchitis or pneumonitis caused by infection, e.g. by bacteria or viruses or helminthes or fungi or protozoons or other pathogens,
pediatric asthma, bronchiectasis,
pulmonary fibrosis,
adult respiratory distress syndrome, bronchial and pulmonary edema,
bronchitis or pneumonitis or interstitial pneumonitis caused by different origins, e.g. aspiration, inhalation of toxic gases, vapors,
bronchitis or pneumonitis or interstitial pneumonitis caused by heart failure, X-rays, radiation, chemotherapy,
bronchitis or pneumonitis or interstitial pneumonitis associated with collagenosis, e.g. lupus erythematodes, systemic scleroderma,
lung fibrosis, idiopathic pulmonary lung fibrosis (IPF), interstitial lung diseases or interstitial pneumonitis of different origin, including asbestosis, silicosis, M. Boeck or sarcoidosis, granulomatosis,
cystic fibrosis or mucoviscidosis, or α1.antitrypsin deficiency,
comprising administering a therapeutically effective amount of pharmaceutical composition according to the invention to a patient in need thereof.

One embodiment of the invention is a method wherein indication (A) is selected from chronic bronchitis, chronic obstructive bronchitis (COPD), chronic sinusitis, nasal polyposis, allergic rhinitis, chronic rhinosinusitis, acute rhinosinusitis, and asthma.

One embodiment of the invention is a method of treating an indication selected from indications (B): inflammatory diseases of the gastrointestinal tract of various origins such as inflammatory pseudopolyps, Crohn's disease, ulcerative colitis, inflammatory diseases of the joints, such as rheumatoid arthritis, or allergic inflammatory diseases of the oro-nasopharynx, skin or the eyes, comprising administering a therapeutically effective amount of a pharmaceutical composition according to the invention to a patient in need thereof. Preferred is a method according to the invention, wherein indication (B) is selected from allergic inflammatory diseases of the oro-nasopharynx, skine or the eyes, Crohn's disease or ulcerative colitis.

One embodiment of the invention is the use of a pharmaceutical composition according to the invention for the manufacture of a medicament for treating respiratory diseases and conditions, preferably an indication selected from indications (A):

prevention and treatment of diseases of the airways and lungs which are accompanied by increased or altered production of mucus and/or inflammatory and/or obstructive diseases of the airways such as acute bronchitis, chronic bronchitis, chronic obstructive bronchitis (COPD), cough, pulmonary emphysema,
allergic or non.allergic rhinitis or sinusitis, chronic sinusitis or rhinitis,
nasal polyposis, chronic rhinosinusitis, acute rhinosinusitis,
asthma, allergic bronchitis, alveolitis, Farmers disease, hyperreactive airways,
bronchitis or pneumonits caused by infection, e.g. by bacteria or viruses or helminthes or fungi or protozoons or other pathogens,
pediatric asthma, bronchiectasis,
pulmonary fibrosis,
adult respiratory distress syndrome, bronchial and pulmonary edema,
bronchitis or pneumonitis or interstitial pneumonitis caused by different origins, e.g. aspiration, inhalation of toxic gases, vapors,
bronchitis or pneumonitis or interstitial pneumonitis caused by heart failure, X.rays, radiation, chemotherapy,
bronchitis or pneumonitis or interstitial pneumonitis associated with collagenosis, e.g. lupus erythematodes, systemic scleroderma,
lung fibrosis, idiopathic pulmonary lung fibrosis (IPF), interstitial lung diseases or interstitial pneumonitis of different origin, including asbestosis, silicosis, M. Boeck or sarcoidosis, granulomatosis,
cystic fibrosis or mucoviscidosis, or α1.antitrypsin deficiency.

Preferred is the use according to the invention, wherein indication (A) is selected from chronic (obstructive) bronchitis (COPD), chronic sinusitis, nasal polyposis, chronic rhinosinusitis, acute rhinosinusitis, and asthma.

One embodiment of the invention is the use of a pharmaceutical composition according to the invention for the manufacture of a medicament for treating an indication selected from indications (B): inflammatory or hypersecretory diseases of the gastrointestinal tract of various origins such as inflammatory pseudopolyps, Crohn's disease, ulcerative colitis, or

inflammatory diseases of the joints, such as rheumatoid arthritis,
or inflammatory allergic diseases of the oro-nasopharynx, skin or the eyes,
comprising administering a therapeutically effective amount of a pharmaceutical composition according to the invention to a patient in need thereof.

Preferably for the use according to the invention, wherein indication B is treated, the CRTH2 antagonist is selected from compounds 1.1 to 1.187.

Preferably for the use according to the invention, indication (B) is selected from inflammatory allergic diseases of the oro-nasopharynx, skin or the eyes, Crohn's disease, or ulcerative colitis.

In this context the present invention is also related to a method for making a medicament for treating any of the aforementioned diseases and conditions by using a pharmaceutical composition comprising one or more CRTH2 antagonists 1 and one or more further active compounds 2 as described herein before.

In a preferred embodiment the present invention also relates to a method for making a medicament for treating asthma and allergic and non-allergic rhinitis by using a pharmaceutical composition comprising one or more CRTH2 antagonists 1, preferably 1.136, and one or more LTD4 antagonist 2-5. In a particularly preferred embodiment the present invention also relates to a method for making a medicament for treating asthma and allergic and non-allergic rhinitis by using a pharmaceutical composition comprising 1.136 and montelukast.

In a preferred embodiment the present invention also relates to a method for making a medicament for treating asthma and allergic and non-allergic rhinitis by using a pharmaceutical composition comprising one or more CRTH2 antagonists 1, preferably 1.136, and one or more PDE4 inhibitors 2-4.

In a preferred embodiment the present invention also relates to a method for making a medicament for treating asthma and allergic and non-allergic rhinitis by using a pharmaceutical composition comprising one or more CRTH2 antagonists 1, preferably 1.136, and one or more histamine receptor antagonists antagonist 2-11.

In a preferred embodiment the present invention also relates to a method for making a medicament for treating asthma and allergic and non-allergic rhinitis by using a pharmaceutical composition comprising one or more CRTH2 antagonists 1, preferably 1.136, and one or more 5-LO inhibitors 2-17.

In a preferred embodiment the present invention also relates to a method for making a medicament for treating asthma and allergic and non-allergic rhinitis by using a pharmaceutical composition comprising one or more CRTH2 antagonists 1, preferably 1.136, and one or more CCR5 antagonist 2-26.

In a preferred embodiment the present invention also relates to a method for making a medicament for treating asthma and allergic and non-allergic rhinitis by using a pharmaceutical composition comprising one or more CRTH2 antagonists 1, preferably 1.136, and one or more CCR9 antagonist 2-7.

In a preferred embodiment the present invention also relates to a method for making a medicament for treating asthma and allergic and non-allergic rhinitis by using a pharmaceutical composition comprising one or more CRTH2 antagonists 1, preferably 1.136, and one or more sulfonamines 2-53.

In a preferred embodiment the present invention also relates to a method for making a medicament for treating asthma and allergic and non-allergic rhinitis by using a pharmaceutical composition comprising one or more CRTH2 antagonists 1, preferably 1.136, and one or more amino salicylates 2-54.

### Pharmaceutical Formulations

The pharmaceutical composition according to the invention may be administered in the form of a preparation suitable for inhalative, oral, intravenous, topical, subcutaneous, intramuscular, intraperitoneal, intranasal, transdermal or rectal administration.

In the pharmaceutical combinations according to the invention the active substances may be combined in a single preparation, e.g. as a fixed dose combination comprising the active ingredients in one formulation together, or contained in two or more separate formulations, e.g. as a kit of parts adapted for simultaneous, separate or sequential administration. Pharmaceutical compositions containing the active substances 1 and 2 in a single preparation are preferred according to the invention.

The actives of the combinations according to the invention may be administered simultaneously, separately or sequentially. The preferred route of administration depends on the indication to be treated. In case of gastrointestinal indications, inflammatory joint, skin and eyes disorders both components 1 and 2 may be administered orally, intravenously or rectally, using suitable formulations known in the art, such as tablets, coated tablets, pills, granules or granular powder, syrups, emulsions, suspensions, solutions or suppositories, optionally together with inert and non.toxic pharmaceutically acceptable excipients or solvents. In case of inflammatory joint or skin disorders both components 1 and 2 also may be may be administered topically, using suitable formulations known in the art, such as ointments or transdermal patches. Furthermore, in case of inflammatory disorders of the eye both components 1 and 2 preferably are administered topically using suitable formulations such as ophthalmic solutions, eye drops or viscoelastic gels.

### A) Oral Formulations Containing the Combinations of Active Substances 1 and 2 According to the Invention:

It is particularly preferable if the compounds of formula I are administered orally, and it is also particularly preferable if they are administered preferably once or alternatively twice a day. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

### B) Inhalative Formulations:

One embodiment of the invention is a pharmaceutical composition in the form of a preparation suitable for inhalation.

One embodiment of the invention is a pharmaceutical composition in the form of a preparation selected from among the inhalable powders, propellant-containing metered-dose aerosols and propellant-free inhalable solutions.

One embodiment of the invention is a pharmaceutical composition in the form of an inhalable powder which contains 1 and 2 in admixture with suitable physiologically acceptable excipients selected from among the monosaccharides, disaccharides, oligo- and polysaccharides, polyalcohols, salts, or mixtures of these excipients with one another.

One embodiment of the invention is a pharmaceutical composition in the form of an inhalable powder wherein the excipient has a maximum average particle size of up to 250 μm, preferably between 10 and 150 μm.

One embodiment of the invention is a pharmaceutical composition in the form of an inhalable powder which contains only the active substances 1 and 2 as its ingredients.

One embodiment of the invention is a pharmaceutical composition in the form of a propellant-containing inhalable aerosol which contains 1 and 2 in dissolved or dispersed form.

One embodiment of the invention is a pharmaceutical composition in the form of a propellant-containing inhalable aerosol that contains, as propellant gas, hydrocarbons such as n.propane, n.butane or isobutane or halohydrocarbons such as chlorinated and/or fluorinated derivatives of methane, ethane, propane, butane, cyclopropane or cyclobutane.

One embodiment of the invention is a pharmaceutical composition in the form of a propellant gas is TG11, TG12, TG134a, TG227 or mixtures thereof, preferably TG134a, TG227 or a mixture thereof.

One embodiment of the invention is a pharmaceutical composition in the form of a propellant-free inhalable solution which contains water, ethanol or a mixture of water and ethanol as solvent.

One embodiment of the invention is a pharmaceutical composition in the form of an inhalable solution wherein it optionally contains other co-solvents and/or excipients.

One embodiment of the invention is a pharmaceutical composition in the form of an inhalable solution wherein it contains as co-solvents ingredients which contain hydroxyl groups or other polar groups, e.g. alcohols. particularly isopropyl alcohol, glycols. particularly propyleneglycol, polyethyleneglycol, polypropyleneglycol, glycolether, glycerol, polyoxyethylene alcohols and polyoxyethylene fatty acid esters.

One embodiment of the invention is a pharmaceutical composition in the form of an inhalable solution wherein it contains as excipients surfactants, stabilisers, complexing agents, antioxidants and/or preservatives, flavourings, pharmacologically acceptable salts and/or vitamins.

In case of respiratory indications and if administered separately or sequentially preferably at least one of components 1 and 2 is given by inhalative route. If component 1 is administered by inhalation component 2, administered separately, may be given for instance orally, intravenously, subcutaneously, by intramuscular injection, intraperitoneally, intranasally or transdermally, using suitable formulations known in the art, such as tablets, coated tablets, pills, granules or granular powder, aerosols, syrups, emulsions, suspensions, powders, solutions or transdermal patches, optionally together with inert and non.toxic pharmaceutically acceptable excipients or solvents. The same applies with respect to component 1, vice versa, if component 2 is administered by inhalation.

In case of respiratory indications both components 1 and 2 of the pharmaceutical combinations according to the invention preferably are administered by inhalation. Inhalable preparations according to the invention include inhalable powders, propellant-containing metered dose aerosols or propellant-free inhalable solutions. Inhalable powders according to the invention containing the combination of active substances 1 and 2 may consist of the active substances on their own or of a mixture of the active substances with physiologically acceptable excipients. Within the scope of the present invention, the term carrier may optionally be used instead of the term excipient. Within the scope of the present invention, the term propellant-free inhalable solutions also includes concentrates or sterile inhalable solutions ready for use. The preparations according to the invention may contain the combination of active substances 1 and 2 either together in one formulation or in two separate formulations. These formulations which may be used within the scope of the present invention are described in more detail in the next part of the specification.

Any aforementioned possible doses applicable for the combinations according to the invention are to be understood as referring to doses per single application. However, these examples are not be understood as excluding the possibility of administering the combinations according to the invention multiple times. Depending on the medical need patients may receive also multiple inhalative applications. As an example patients may receive the combinations according to the invention for instance two or three times (e.g. two or three puffs with a powder inhaler, an MDI etc.) in the morning of each treatment day. As the aforementioned dose examples are only to be understood as dose examples per single application (i.e. per puff) multiple application of the combinations according to the invention leads to multiple doses of the aforementioned examples. The application of the compositions according to the invention can be for instance once a day, or depending on the duration of action of the agents twice a day, or once every 2 or 3 days.

Moreover it is emphazised that the aforementioned dosages are to be understood as examples of metered doses only. In other terms, the aforementioned doses are not to be understood as the effective doses of the combinations according to the invention that do in fact reach the lung. It is clear for the person of ordinary skill in the art that the delivered dose to the lung is generally lower than the metered dose of the administered active ingredients.

### B1) Inhalable Powder Containing the Combinations of Active Substances 1 and 2 According to the Invention:

The inhalable powders according to the invention may contain 1 and 2 either on their own or in admixture with suitable physiologically acceptable excipients.

If the active substances 1 and 2 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose, trehalose), oligo. and polysaccharides (e.g. dextran), polyalcohols (e.g. sorbitol, mannitol, xylitol), cyclodextrines (e.g. α-cyclodextrine, β-cyclodextrine, χ-cyclodextrine, methyl-β-cyclodextrine, hydroxypropyl-β-cyclodextrine), salts (e.g. sodium chloride, calcium carbonate) or mixtures of these excipients with one another. Preferably, mono. or disaccharides are used, while the use of lactose, trehalose or glucose is preferred, particularly, but not exclusively, in the form of their hydrates.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipient mentioned above. These finer excipients are also selected from the group of possible excipients listed hereinbefore. Finally, in order to prepare the inhalable powders according to the invention, micronised active substance 1 and 2, preferably with an average particle size of 0.5 to 10 μm, more preferably from 1 to 6 μm, is added to the excipient mixture. Processes for producing the inhalable powders according to the invention by grinding and micronising and by finally mixing the ingredients together are known from the prior art. The inhalable powders according to the invention may be prepared and administered either in the form of a single powder mixture which contains both 1 and 2 or in the form of separate inhalable powders which contain only 1 or 2.

The inhalable powders according to the invention may be administered using inhalers known from the prior art Inhalable powders according to the invention which contain one or more physiologically acceptable excipients in addition to 1 and 2 may be administered, for example, by means of inhalers which deliver a single dose from a supply using a measuring chamber as described in U.S. Pat. No. 4,570,630A, or by other means as described in DE 36 25 685 A. The inhalable powders according to the invention which contain 1 and 2 optionally in conjunction with a physiologically acceptable excipient may be administered, for example, using the inhaler known by the name Turbuhaler® or using inhalers as disclosed for example in EP 237507A. Preferably, the inhalable powders according to the invention which contain physiologically acceptable excipient in addition to 1 and 2 are packed into capsules (to produce so.called inhalettes) which are used in inhalers as described, for example, in WO 94/28958.

A particularly preferred inhaler for using the pharmaceutical combination according to the invention in inhalettes is shown in FIG. 1.

This inhaler (Handyhaler) for inhaling powdered pharmaceutical compositions from capsules is characterised by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut, as well as airholes 13 for adjusting the flow resistance.

If the inhalable powders according to the invention are packed into capsules (inhalers) for the preferred use described above, the quantities packed into each capsule should be 1 to 30 mg per capsule. These capsules contain, according to the invention, either together or separately, the doses of 1 and 2 mentioned hereinbefore for each single dose.

### B2) Propellant Gas-Driven Inhalation Aerosols Containing the Combinations of Active Substances 1 and 2:

Inhalation aerosols containing propellant gas according to the invention may contain substances 1 and 2 dissolved in the propellant gas or in dispersed form. 1 and 2 may be present in separate formulations or in a single preparation, in which 1 and 2 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n.propane, n.butane or isobutane and halohydrocarbons such as fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG11, TG12, TG134a (1,1,1,2.tetrafluoroethane) and TG227 (1,1,1,2,3,3,3.heptafluoropropane) and mixtures thereof, of which the propellant gases TG134a, TG227 and mixtures thereof are preferred.

The propellant.driven inhalation aerosols according to the invention may also contain other ingredients such as co-solvents, stabilisers, surfactants, antioxidants, lubricants and pH adjusters. All these ingredients are known in the art.

The inhalation aerosols containing propellant gas according to the invention may contain up to 5 wt. % of active substance 1 and/or 2. Aerosols according to the invention contain, for example, 0.002 to 5 wt. %, 0.01 to 3 wt. %, 0.015 to 2 wt. %, 0.1 to 2 wt. %, 0.5 to 2 wt. % or 0.5 to 1 wt. % of active substance 1 and/or 2.

If the active substances 1 and/or 2 are present in dispersed form, the particles of active substance preferably have an average particle size of up to 10 μm, preferably from 0.1 to 6 μm, more preferably from 1 to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant.driven aerosols as hereinbefore described combined with one or more inhalers suitable for administering these aerosols. In addition, the present invention relates to inhalers which are characterised in that they contain the propellant gas-containing aerosols described above according to the invention. The present invention also relates to cartridges fitted with a suitable valve which can be used in a suitable inhaler and which contain one of the above.mentioned propellant gas-containing inhalation aerosols according to the invention. Suitable cartridges and methods of filling these cartridges with the inhalable aerosols containing propellant gas according to the invention are known from the prior art.

### B3) Propellant-Free Inhalable Solutions or Suspensions Containing the Combinations of Active Substances 1 and 2 According to the Invention:

Propellant-free inhalable solutions and suspensions according to the invention contain, for example, aqueous or alcoholic, preferably ethanolic solvents, optionally ethanolic solvents mixed with aqueous solvents. If aqueous/ethanolic solvent mixtures are used the relative proportion of ethanol compared with water is not limited but preferably the maximum is up to 70 percent by volume, more particularly up to 60 percent by volume of ethanol. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

According to the invention, the addition of editic acid (EDTA) or one of the known salts thereof, sodium editate, as stabiliser or complexing agent is unnecessary in the present formulation. Other embodiments may contain this compound or these compounds. In a preferred embodiment the content based on sodium editate is less than 100 mg/100 ml, preferably less than 50 mg/100 ml, more preferably less than 20 mg/100 ml. Generally, inhalable solutions in which the content of sodium editate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g. alcohols. particularly isopropyl alcohol, glycols. particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents. The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the combination of active substances 1 and 2, only benzalkonium chloride and sodium editate. In another preferred embodiment, no sodium editate is present.

The propellant-free inhalable solutions according to the invention are administered in particular using inhalers of the kind which are capable of nebulising a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 20 and 30 μL of active substance solution can be nebulised in preferably one spray action to form an aerosol with an average particle size of less than 20 μm, preferably less than 10 μm, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulisers (devices) described therein are known by the name Respimat®.

1. A pharmaceutical composition comprising one or more CRTH2 antagonists 1 and one or more further active compounds 2, wherein 1 is a compound according to formula (I) 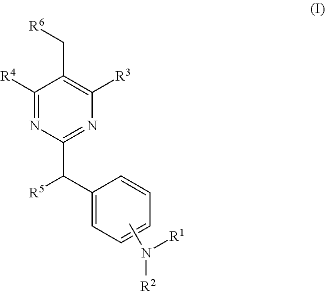
wherein
R¹ represents hydrogen, 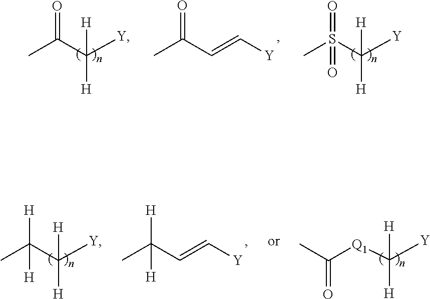
in which
n represents an integer of 0 to 6;
-Q₁- represents —NH—, —N(C₁₋₆ alkyl)-, or —O—;
Y represents hydrogen, C₃₋₈ cycloalkyl optionally substituted by C₁₋₆ alkyl, C₃₋₈ cycloalkyl fused by benzene, aryl or heteroaryl, wherein said aryl and heteroaryl are optionally substituted at a substitutable position with one or more substituents selected from the group consisting of cyano, halogen, nitro, guanidino, pyrrolyl, sulfamoyl, C₁₋₆ alkylaminosulfonyl, di(C₁₋₆ alkyl)aminosulfonyl, phenyloxy, phenyl, amino, C₁₋₆alkylamino, di(C₁₋₆)alkylamino, di(C₁₋₆)alkylamino, C₁₋₆ alkoxycarbonyl, C₁₋₆ alkanoyl, C₁₋₆ alkanoylamino, carbamoyl, C₁₋₆ alkylcarbamoyl, di-(C₁₋₆ alkyl)carbamoyl, C₁₋₆ alkylsulfonyl, C₁₋₆ alkyl optionally mono-, di-, or tri-substituted by halogen, C₁₋₆ alkoxy optionally mono-, di-, or tri-substituted by halogen and C₁₋₆ alkylthio optionally mono-, di-, or tri-substituted by halogen,
or aryl fused by 1,3-dioxolane;
R² represents hydrogen or C₁₋₆ alkyl;
R³ represents halogen, C₁₋₆ alkoxy optionally mono-, di-, or tri-substituted by halogen, 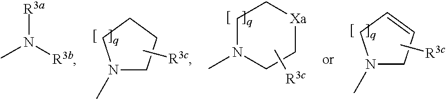
in which
R^{3a} and R^{3b} independently represent C₃₋₈ cycloalkyl, or C₁₋₆ alkyl, which C₁₋₆ alkyl is optionally substituted by hydroxy, carboxy, C₃₋₈ cycloalkyl, carbamoyl, C₁₋₆ alkylcarbamoyl, aryl-substituted C₁₋₆ alkylcarbamoyl, C₁₋₆ alkylcarbamoyl, di(C₁₋₆alkyl)carbamoyl, C₃₋₈ cycloalkylcarbamoyl, C₃₋₈ heterocyclocarbonyl, (C₁₋₆)alkylamino, di(C₁₋₆)alkylamino or C₁₋₆ alkoxy,
q represents an integer of 1 to 3;
R^{3c} represents hydrogen, hydroxy, carboxy, or C₁₋₆ alkyl optionally substituted by hydroxy, carboxy or (phenyl-substituted C₁₋₆ alkyl)carbamoyl;
Xa represents —O—, —S— or —N(R^{3d})—
in which
R^{3d} represents C₁₋₆ alkyl;
R⁴ represents hydrogen, halogen, C₁₋₆ alkoxy, di(C₁₋₆ alkyl)amino or C₁₋₆ alkyl optionally substituted by C₁₋₆ alkoxy, or mono-, di-, or tri-halogen;
R⁵ represents hydrogen, or C₁₋₆ alkyl; and
R⁶ represents carboxy, carboxamide, nitrile or tetrazolyl.
optionally in the form of an ester, an enantiomer, a racemate, a diastereomer, a tautomer or an addition salt with a pharmacologically acceptable acid or base,
and wherein 2 is selected from β2 adrenoceptor-agonists (short and long-acting beta mimetics), anti-cholinergics (short and long-acting), anti-inflammatory steroids (oral and topical corticosteroids), dissociated-glucocorticoidmimetics, PDE3 inhibitors, PDE4 inhibitors, PDE7 inhibitors, LTD4 antagonists, EGFR inhibitors, PAF antagonists, Lipoxin A4 derivatives, FPRL1 modulators, LTB4-receptor (BLT1, BLT2) antagonists, Histamine-receptor antagonists, PI3-kinase inhibitors, inhibitors of non-receptor tyrosine kinases as for example LYN, LCK, SYK, ZAP-70, FYN, BTK or ITK, inhibitors of MAP kinases as for example p38, ERK1, ERK2, JNK1, JNK2, JNK3 or SAP, inhibitors of the NF-κB signalling pathway as for example IKK2 kinase inhibitors, iNOS inhibitors, MRP4 inhibitors, leukotriene biosynthese inhibitors as for example 5-Lipoxygenase (5-LO) inhibitors, cPLA2 inhibitors, Leukotriene A4 Hydrolase inhibitors or FLAP inhibitors, Non-steroidale anti-inflammatory agents (NSAIDs), DP1-receptor modulators, Thromboxane receptor antagonists, CCR1 antagonists, CCR2 antagonists, CCR3 antagonists, CCR4 antagonists, CCR5 antagonists, CCR6 antagonists, CCR7 antagonists, CCR8 antagonists, CCR9 antagonists, CCR10 antagonists, CXCR1 antagonists, CXCR2 antagonists, CXCR3 antagonists, CXCR4 antagonists, CXCR5 antagonists, CXCR6 antagonists, CX3CR1 antagonists, Neurokinin (NK1, NK2) antagonists, Sphingosine 1-Phosphate receptor modulators, Sphingosine 1 phosphate lyase inhibitors, Adenosine receptor modulators as for example A2a-agonists, modulators of purinergic receptors as for example P2X7 inhibitors, Histone Deacetylase (HDAC) activators, Bradykinin (BK1, BK2) antagonists, TACE inhibitors, PPAR gamma modulators, Rho-kinase inhibitors, interleukin 1-beta converting enzyme (ICE) inhibitors, Toll-Like receptor (TLR) modulators, HMG-CoA reductase inhibitors, VLA-4 antagonists, ICAM-1 inhibitors, SHIP agonists, GABAa receptor antagonist, ENaC-inhibitors, Melanocortin receptor (MC1R, MC2R, MC3R, MC4R, MC5R) modulators, CGRP antagonists, Endothelin antagonists, mucoregulators, immunotherapeutic agents, compounds against swelling of the airways, compounds against cough, CB2 agonists, retinoids, immunosuppressants, mast cell stabilizers, methylxanthine, opioid receptor agonists, laxatives, anti-foaming agents, antispasmodic agents and 5-HT4 agonists.

2. The composition according to claim 1, wherein 1 is a compound wherein
R¹ represents 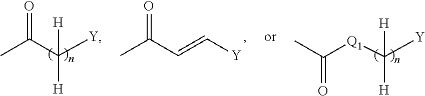
in which
n represents an integer of 0 to 2;
-Q₁- represents —NH—, —N(C₁₋₆ alkyl)-, or —O—;
Y represents C₁₋₆ alkyl, C₃₋₈ cycloalkyl optionally substituted by C₁₋₆ alkyl, C₃₋₈ cycloalkyl fused by benzene selected from the group consisting of indenyl, and tetrahydronaphthyl, aryl selected from the group consisting of phenyl and naphthyl, or heteroaryl selected from the group consisting of indolyl, quinolyl, benzofuranyl, furanyl, chromanyl, and pyridyl, wherein said aryl and heteroaryl are optionally substituted at a substitutable position with one or more substituents selected from the group consisting of cyano, halogen, nitro, pyrrolyl, sulfamoyl, C₁₋₆ alkylaminosulfonyl, di(C₁₋₆ alkyl)aminosulfonyl, phenyloxy, phenyl, C₁₋₆alkylamino, di(C₁₋₆)alkylamino, C₁₋₆ alkoxycarbonyl, C₁₋₆ alkanoylamino, carbamoyl, C₁₋₆ alkylcarbamoyl, di-(C₁₋₆ alkyl)carbamoyl, C₁₋₆ alkylsulfonyl, C₁₋₆ alkyl optionally mono-, di-, or tri-substituted by halogen, C₁₋₆ alkoxy optionally mono-, di-, or tri-substituted by halogen and C₁₋₆ alkylthio optionally mono-, di-, or tri-substituted by halogen; and
R² represents hydrogen.

3. The pharmaceutical composition according to claim 1, wherein 1 is a compound wherein
R³ represents C₁₋₆ alkoxy optionally mono-, di-, or tri-substituted by halogen, 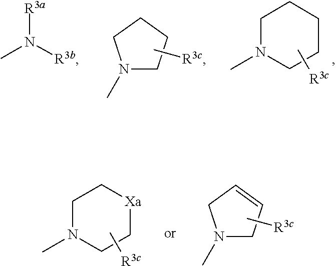
in which
R^{3a} and R^{3b} independently represent C₁₋₆ alkyl optionally substituted by hydroxy, carboxy, C₃₋₈ cycloalkyl, carbamoyl, C₁₋₆ alkylcarbamoyl, di(C₁₋₆ alkyl)carbamoyl, C₃₋₈ cycloalkylcarbamoyl, C₃₋₈ heterocyclocarbonyl, (C₁₋₆)alkylamino, di(C₁₋₆)alkylamino or C₁₋₆ alkoxy,
R^{3c} represents hydrogen, hydroxy, carboxy, or C₁₋₆ alkyl optionally substituted by hydroxy, carboxy or (phenyl-substituted C₁₋₆ alkyl)carbamoyl; and
Xa represents —O—, —S— or —N(R^{3d})—,
in which
R^{3d} represents C₁₋₆ alkyl.

4. The pharmaceutical composition according to claim 1, wherein 1 is a compound of formula (I-i) 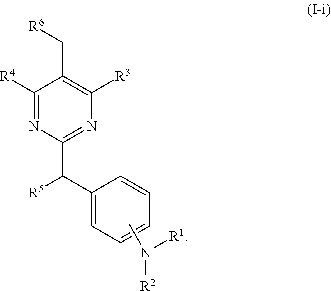
wherein
R¹ represents 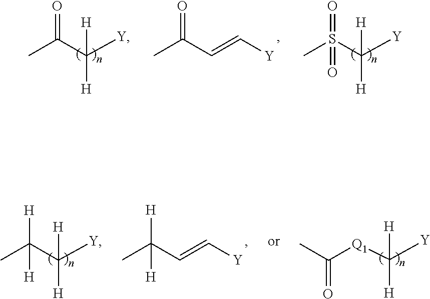
in which
n represents an integer of 0 to 2;
-Q₁- represents —NH—, —N(C₁₋₆ alkyl)-, or —O—;
Y represents phenyl, naphthyl, indolyl, quinolyl, benzofuranyl, furanyl or pyridyl, wherein said phenyl, naphthyl, indolyl, quinolyl, benzofuranyl, furanyl and pyridyl are optionally substituted at a substitutable position with one or two substituents selected from the group consisting of cyano, halogen, nitro, phenyloxy, phenyl, C₁₋₆ alkyl optionally mono-, di-, or tri-substituted by halogen, C₁₋₆ alkoxy optionally mono-, di-, or tri-substituted by halogen and C₁₋₆ alkylthio optionally mono-, di-, or tri-substituted by halogen;
R² represents hydrogen or C₁₋₆ alkyl;
R³ represents 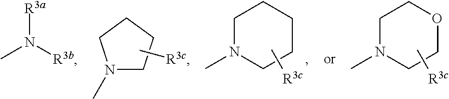
in which
R^{3a} and R^{3b} independently represent C₃₋₈ cycloalkyl, or C₁₋₆ alkyl optionally substituted by C₃₋₈ cycloalkyl, carbamoyl, C₁₋₆ alkylcarbamoyl, phenyl-substituted C₁₋₆ alkylcarbamoyl, C₁₋₆ alkylcarbamoyl, di(C₁₋₆ alkyl)carbamoyl, C₃₋₈ cycloalkylcarbamoyl, C₃₋₈heterocyclocarbonyl, (C₁₋₆)alkylamino, di(C₁₋₆)alkylamino or C₁₋₆ alkoxy,
R^{3c} represents hydrogen, hydroxy, carboxy, or C₁₋₆ alkyl optionally substituted by hydroxy, carboxy or (phenyl-substituted C₁₋₆ alkyl)-carbamoyl;
R⁴ represents hydrogen, chloro, bromo, C₁₋₆ alkoxy, di(C₁₋₆ alkyl)amino or C₁₋₆ alkyl optionally substituted by C₁₋₆ alkoxy;
R⁵ represents hydrogen, or methyl; and
R⁶ represents carboxy or tetrazolyl.

5. The pharmaceutical composition according to claim 1, wherein 1 is a compound selected from 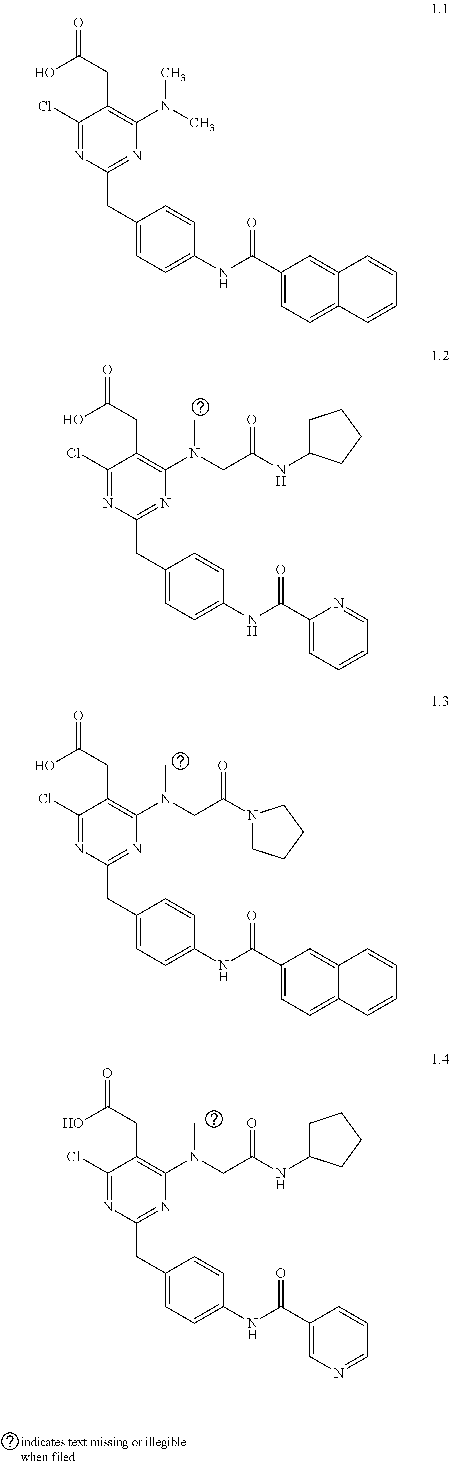 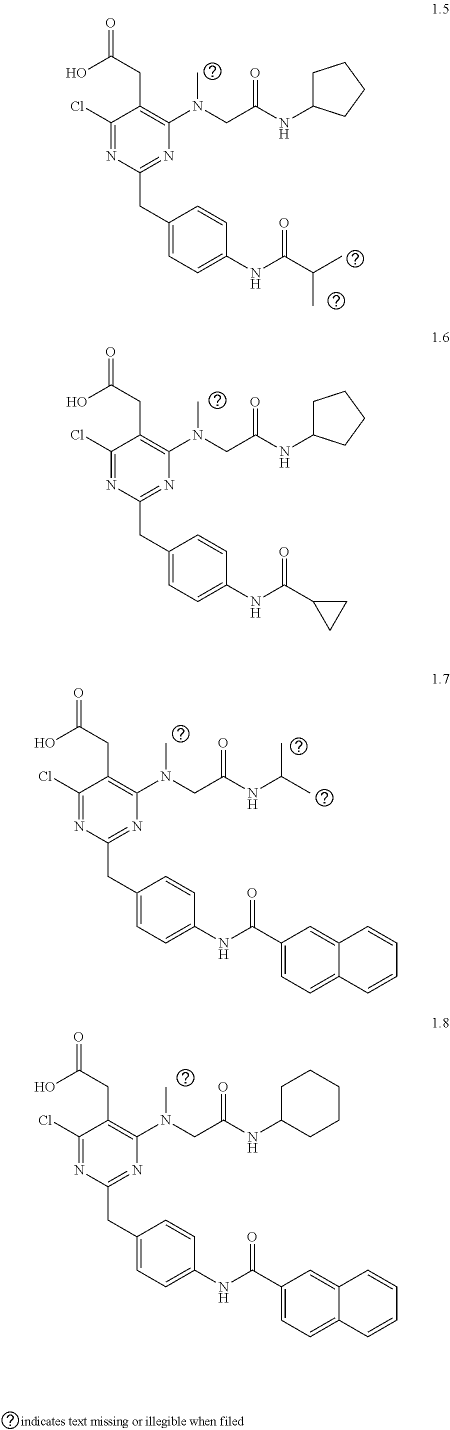 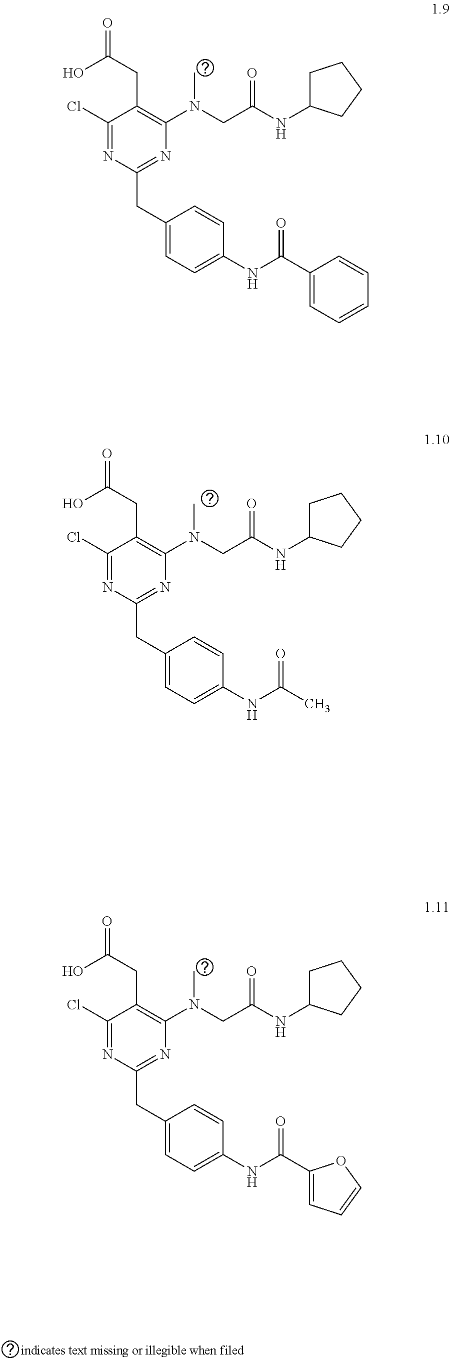 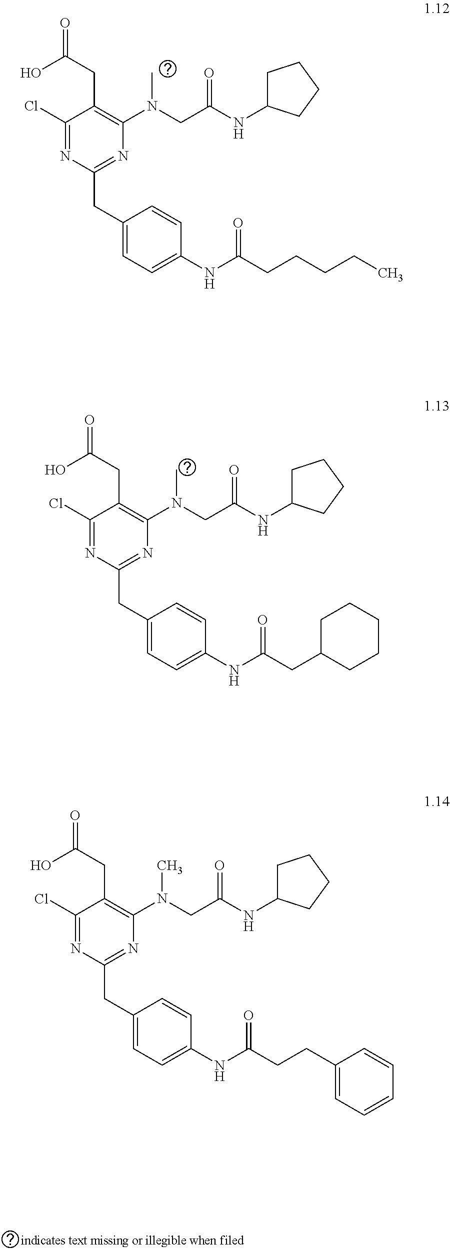 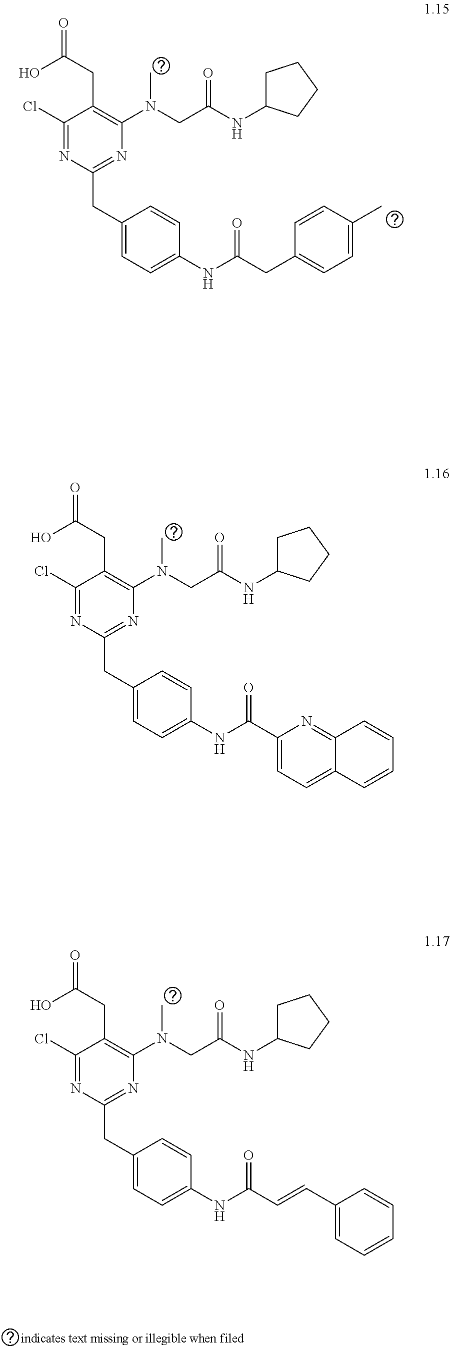 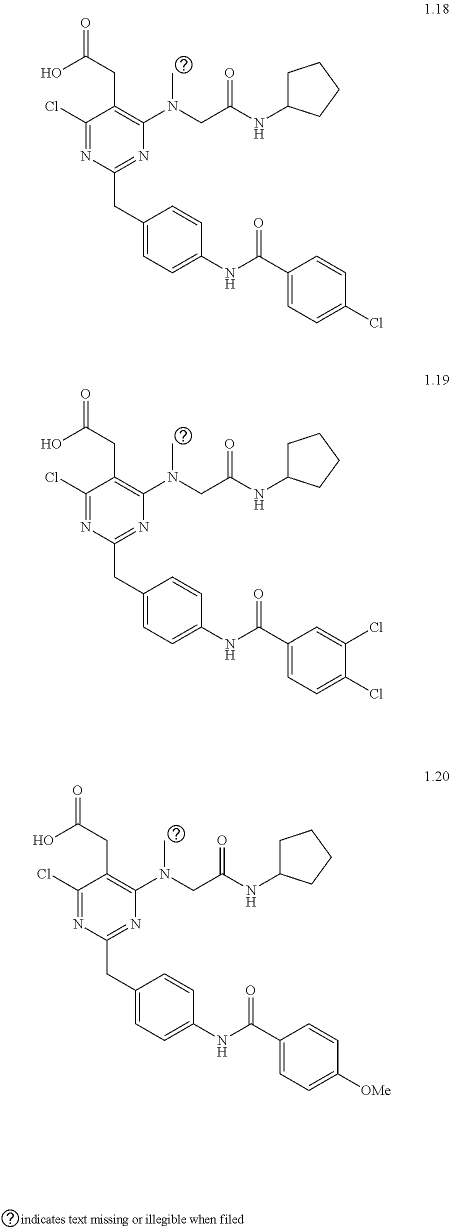 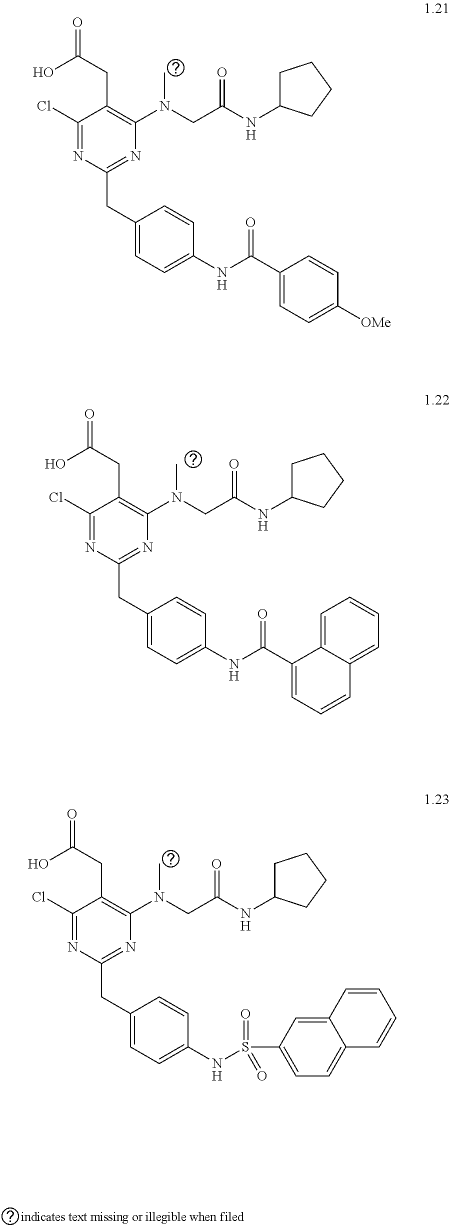 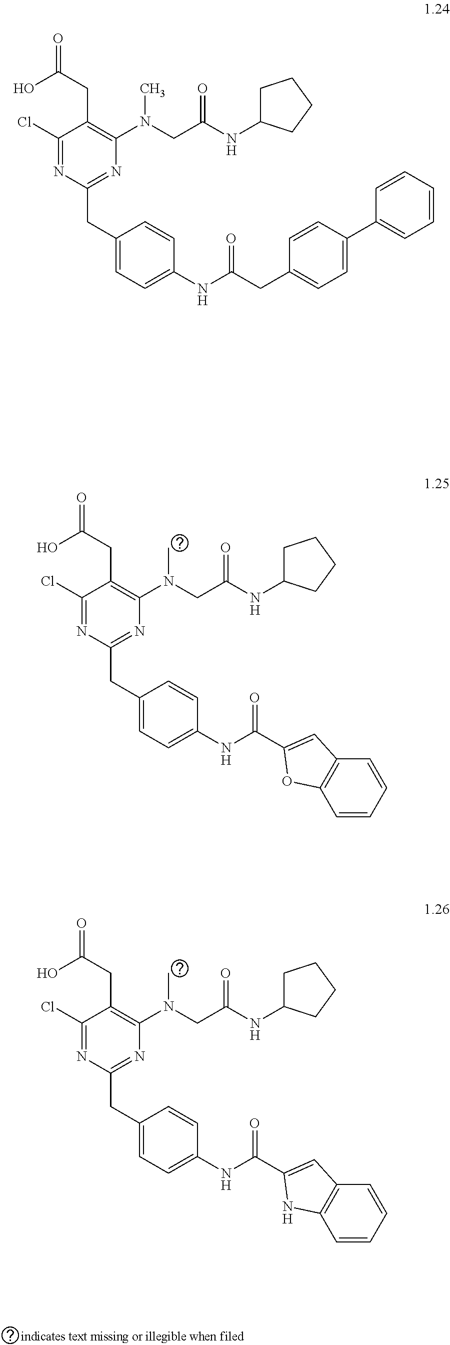 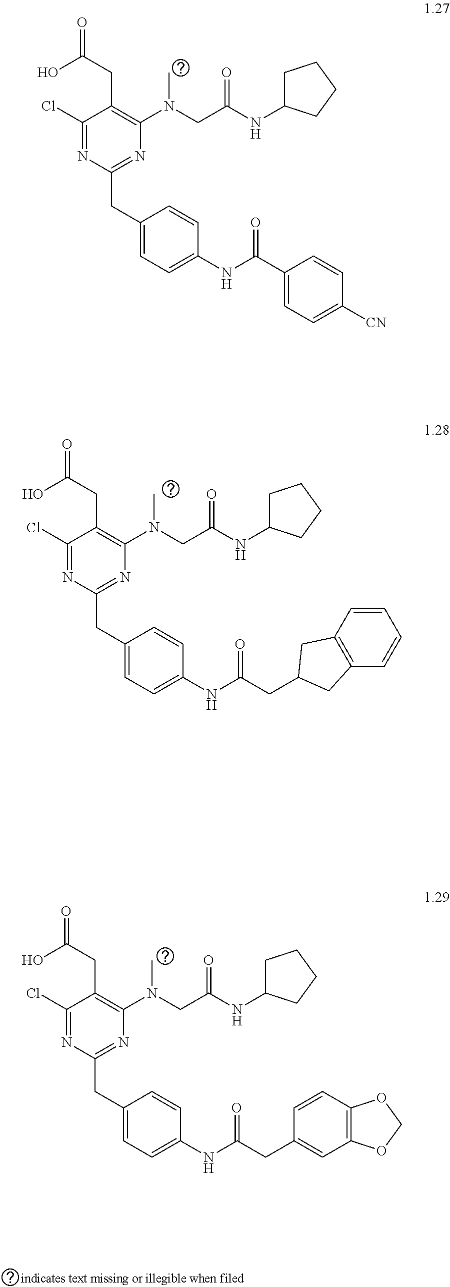 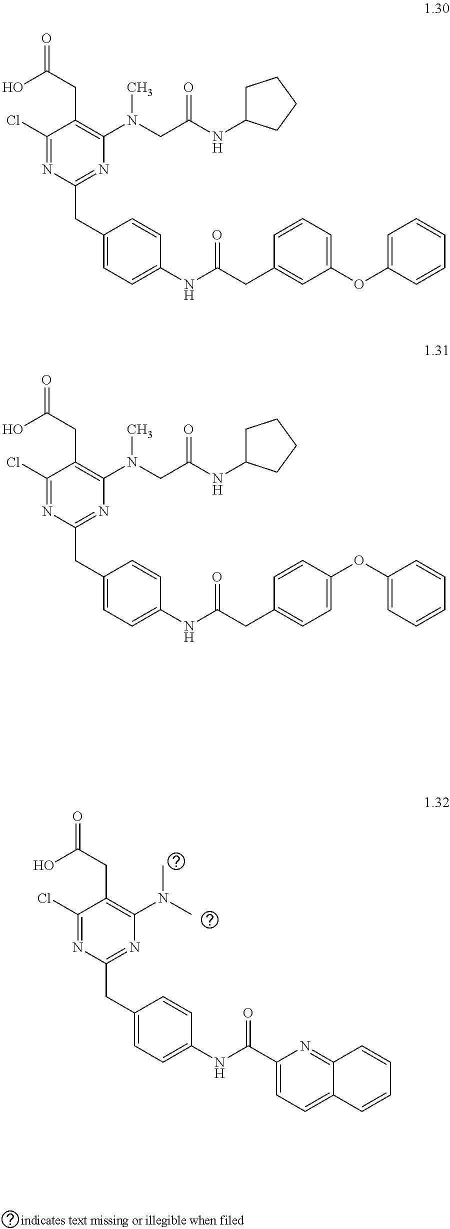 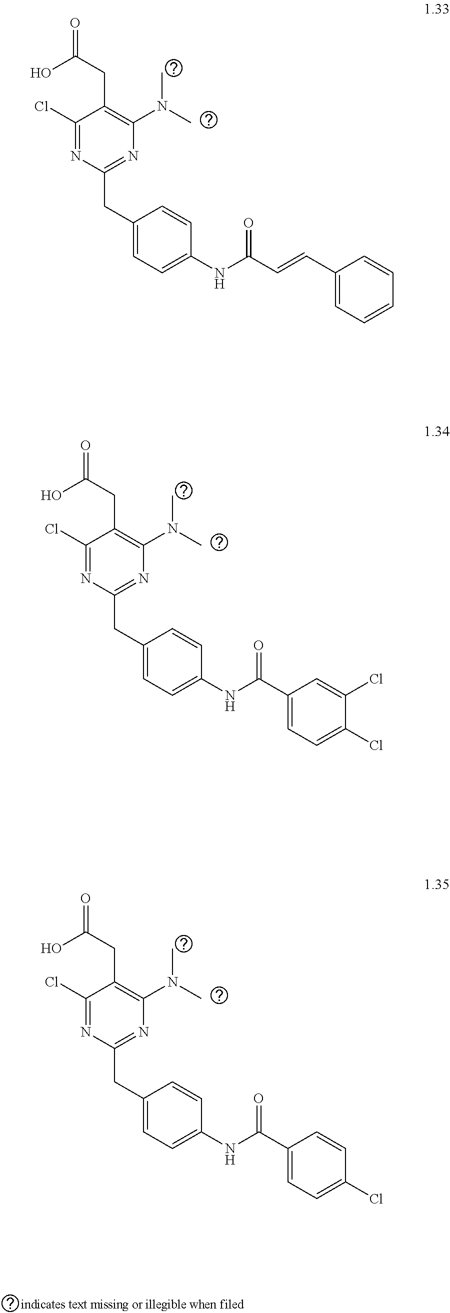 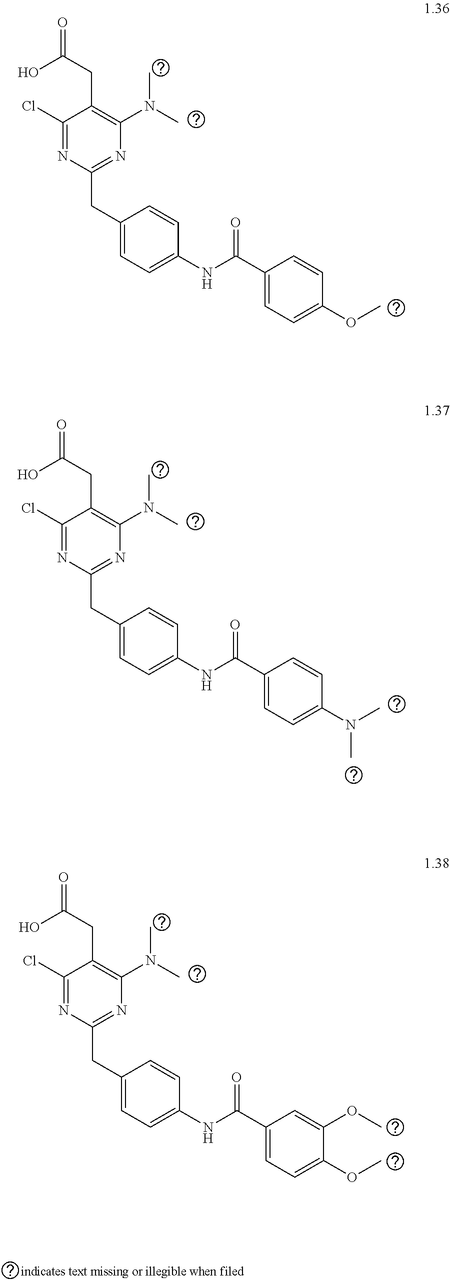 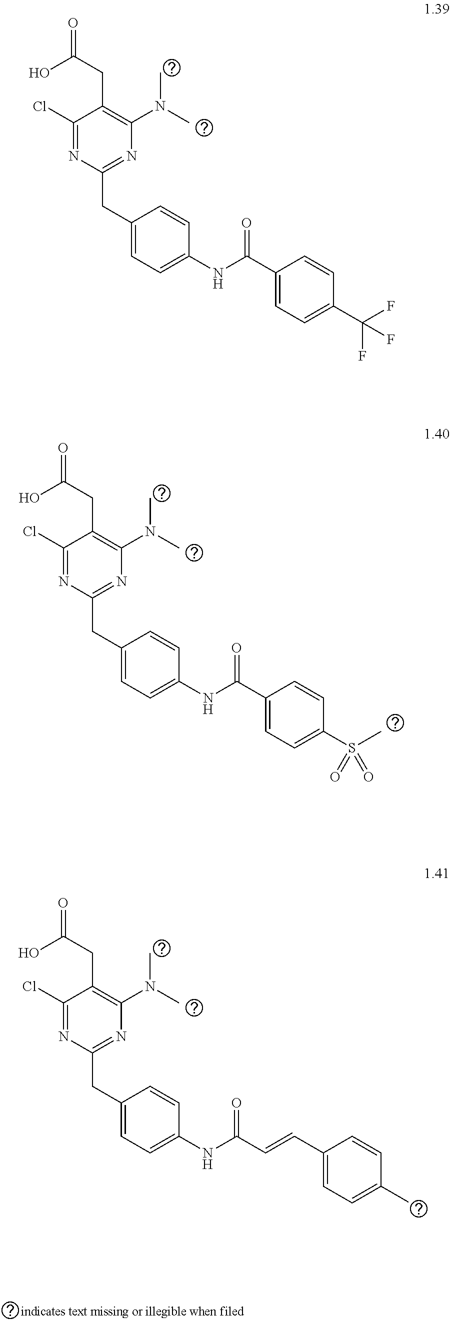 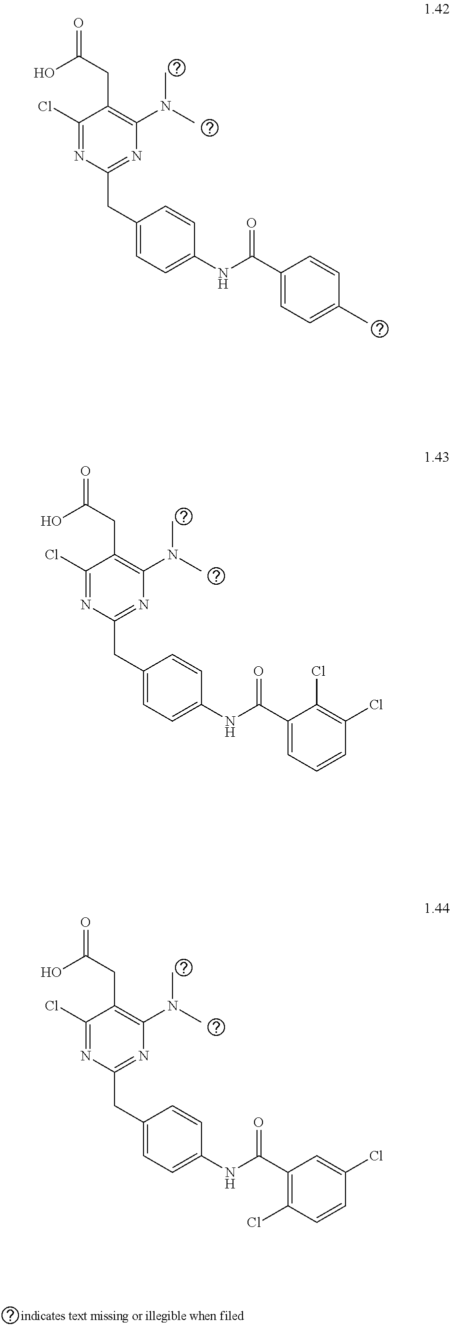 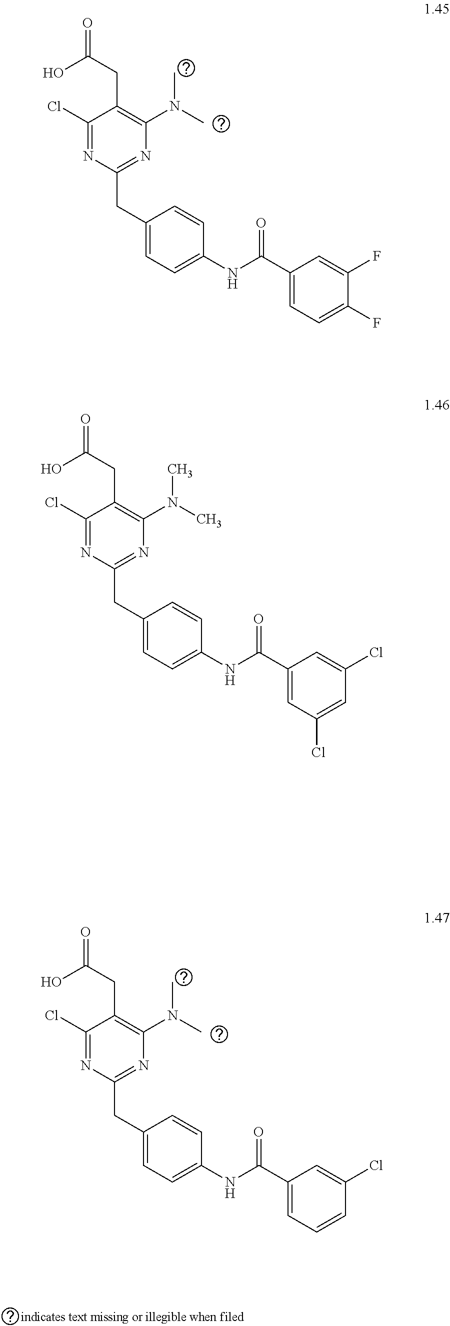 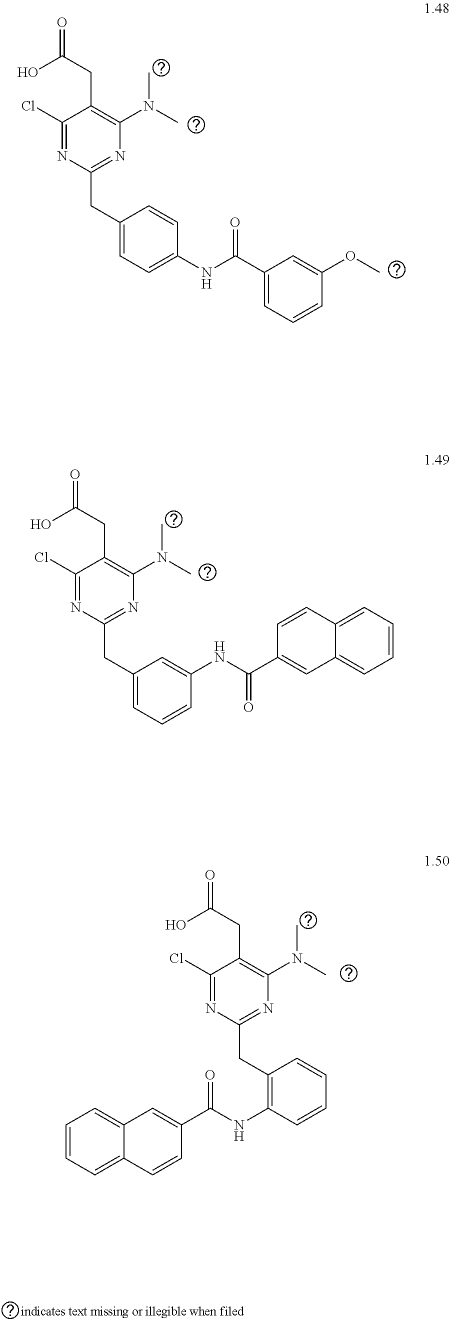 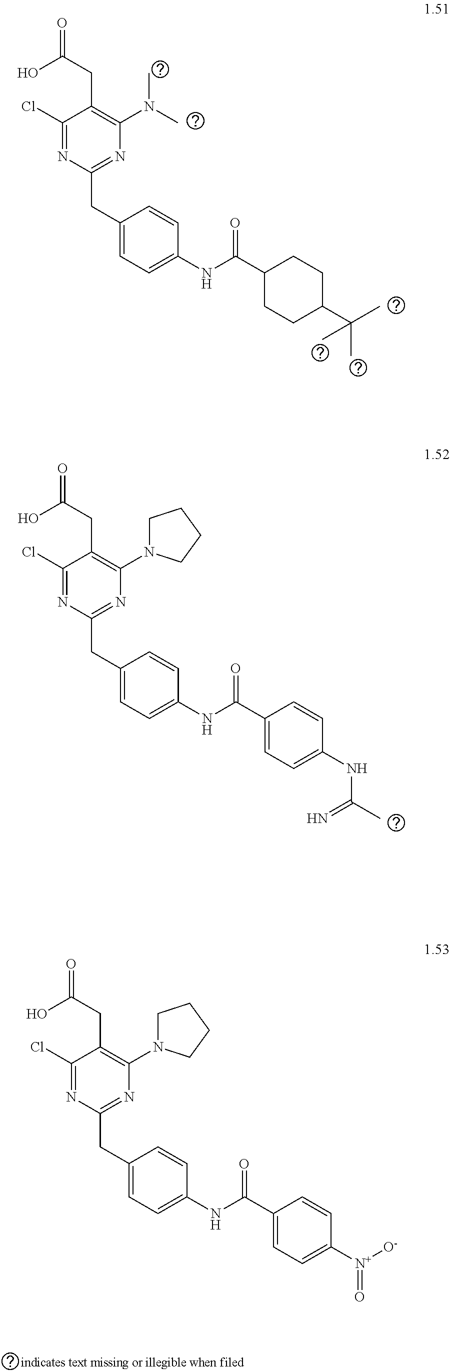 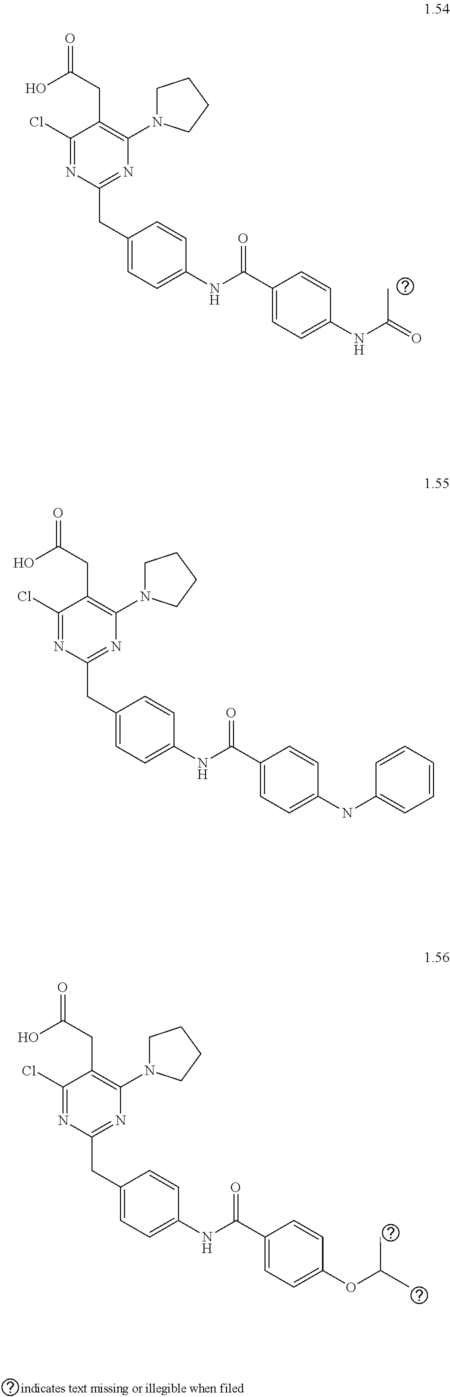 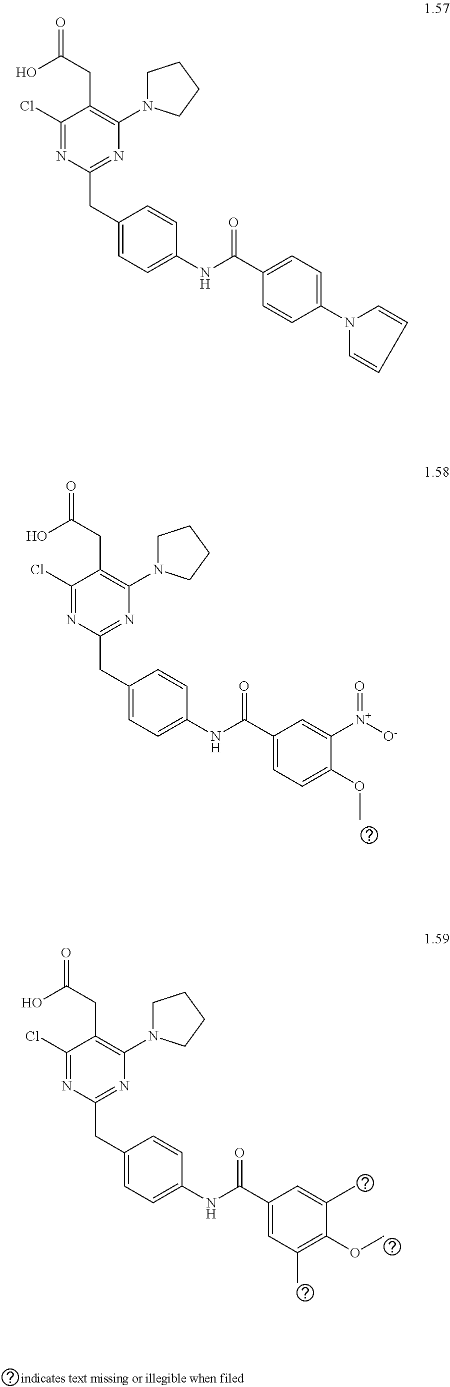 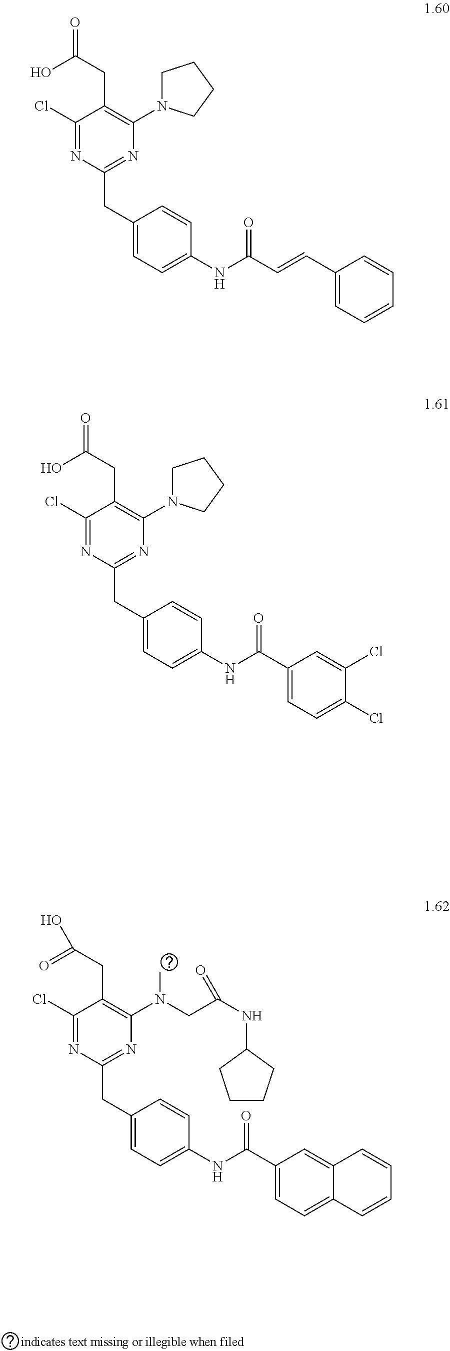 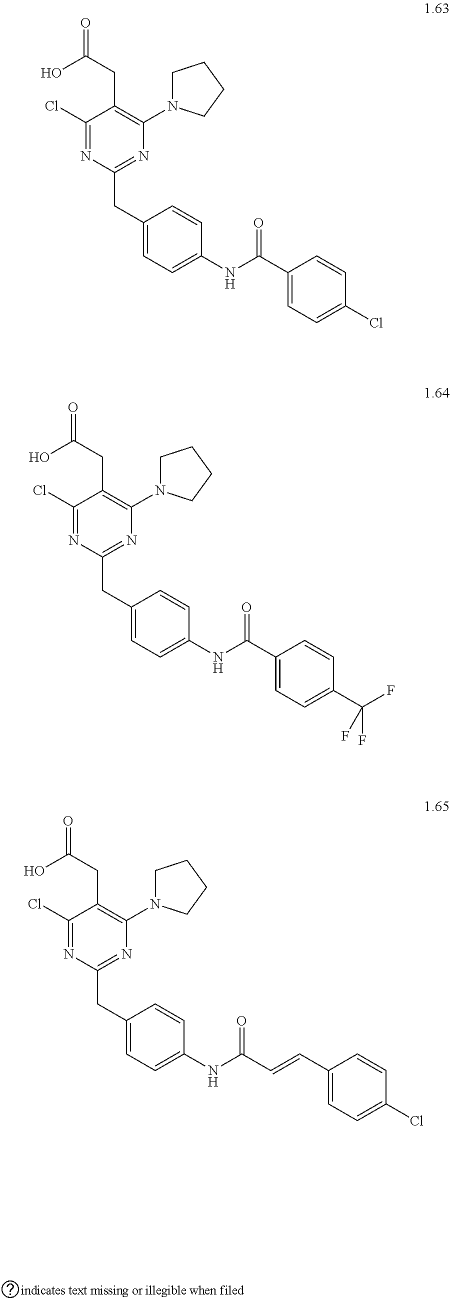 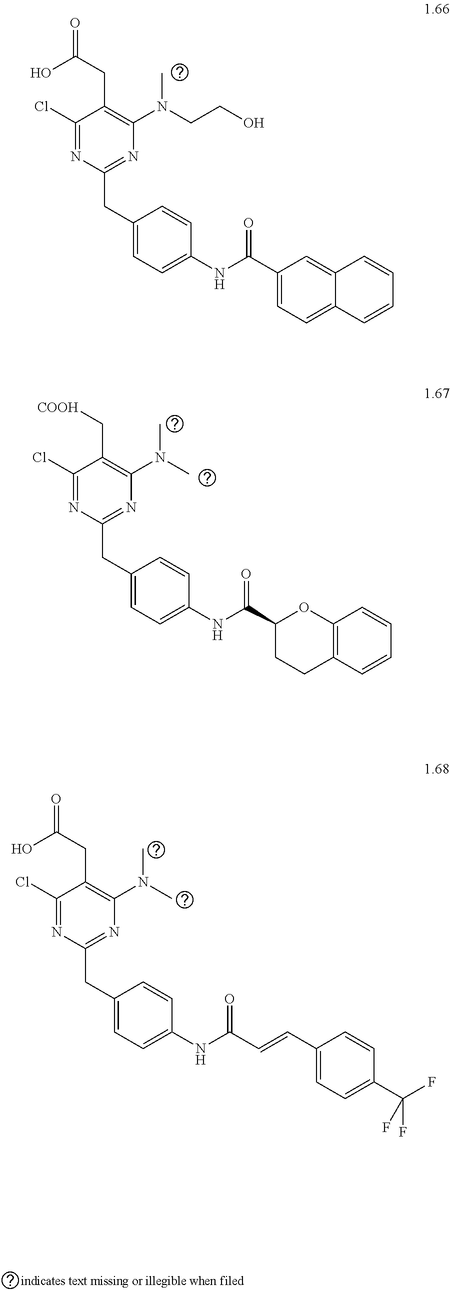 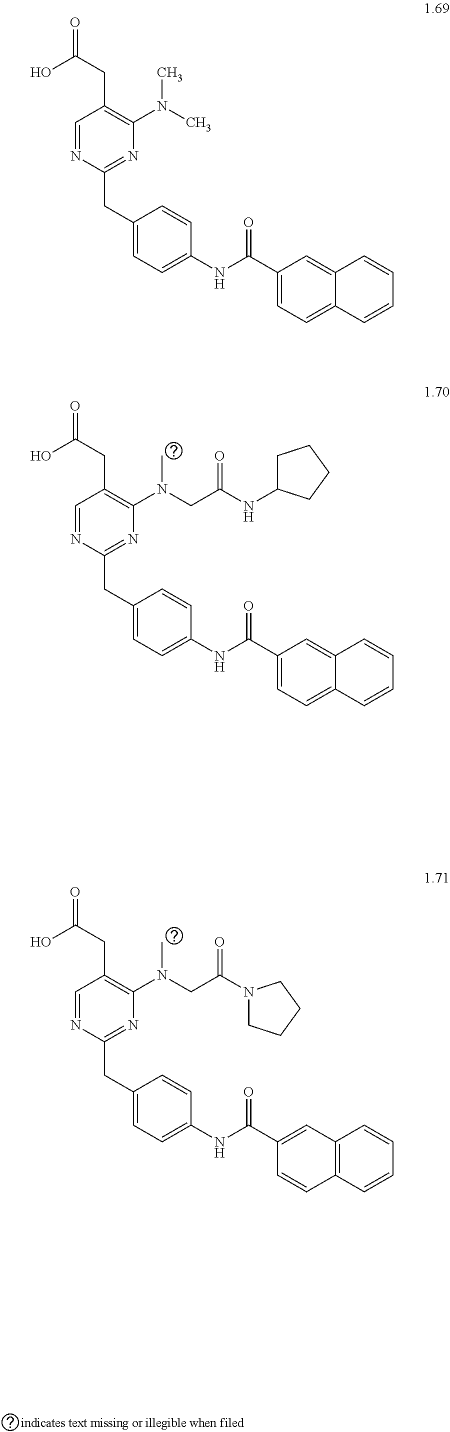 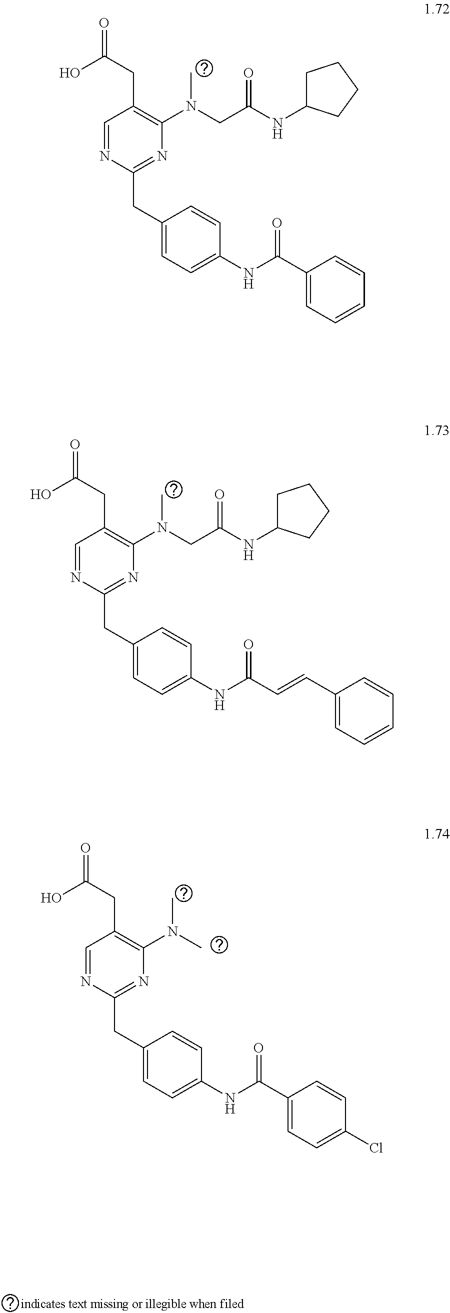 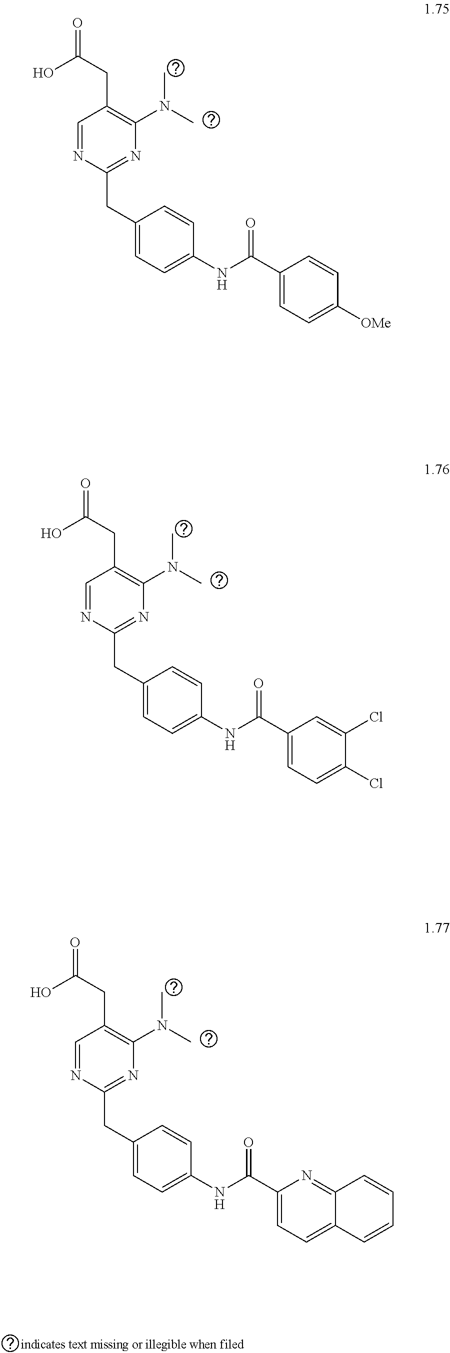 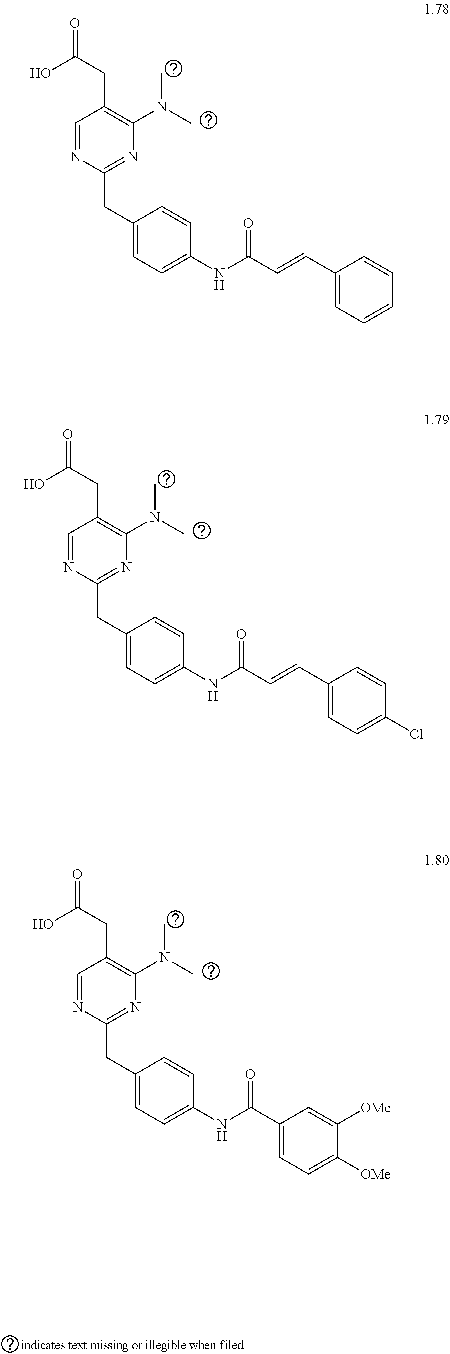 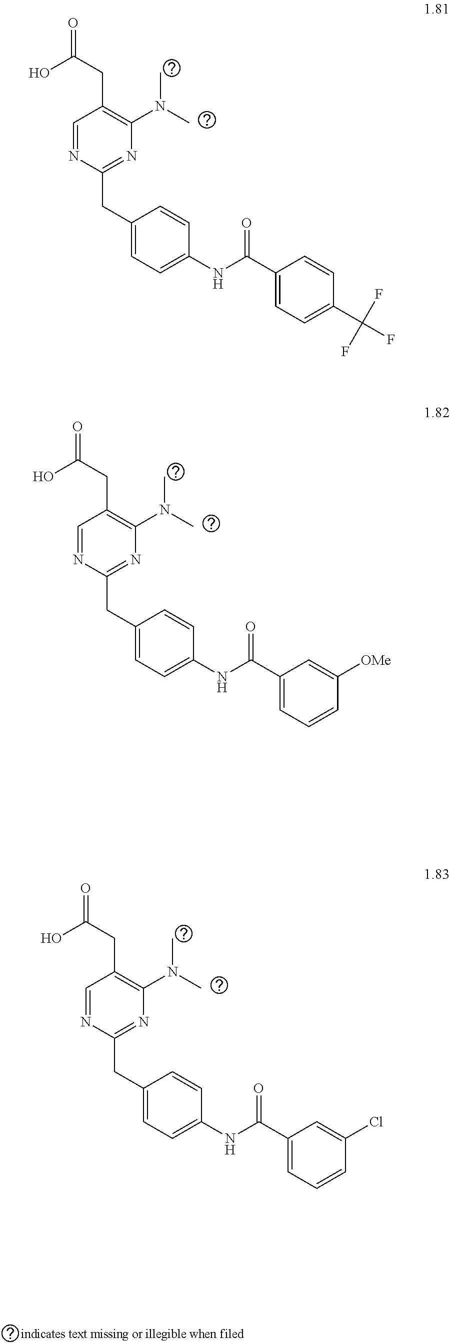 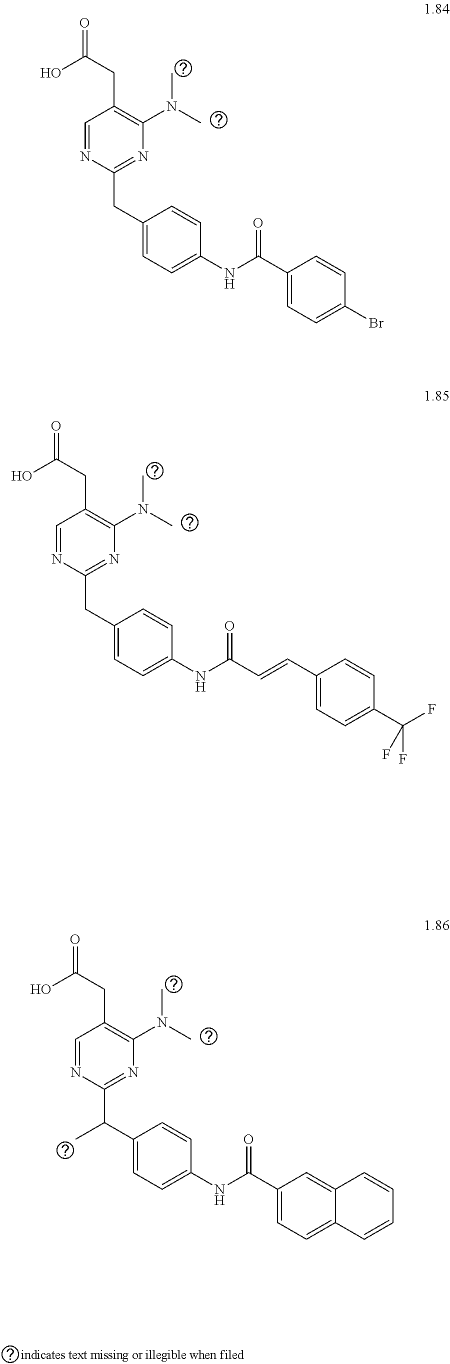 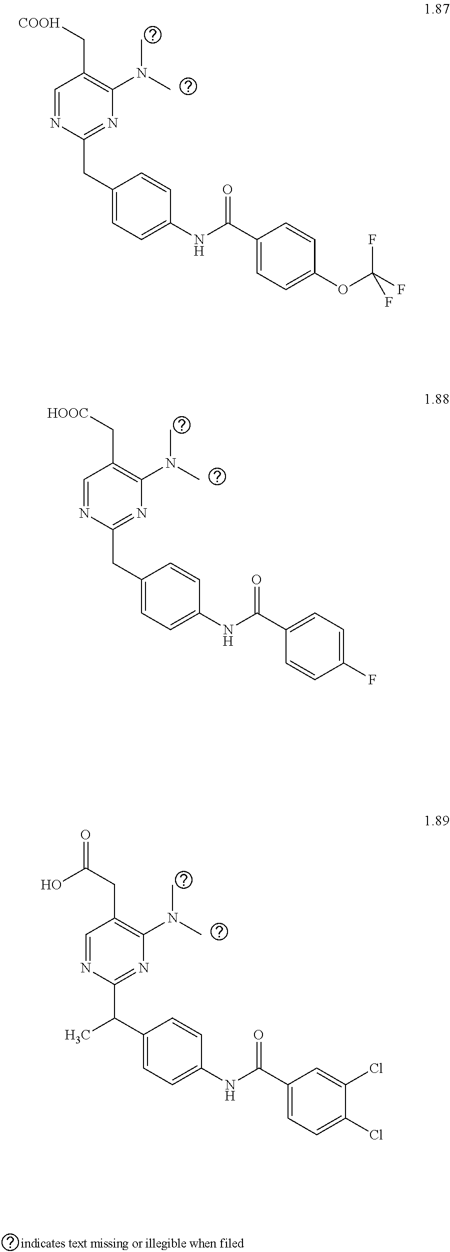 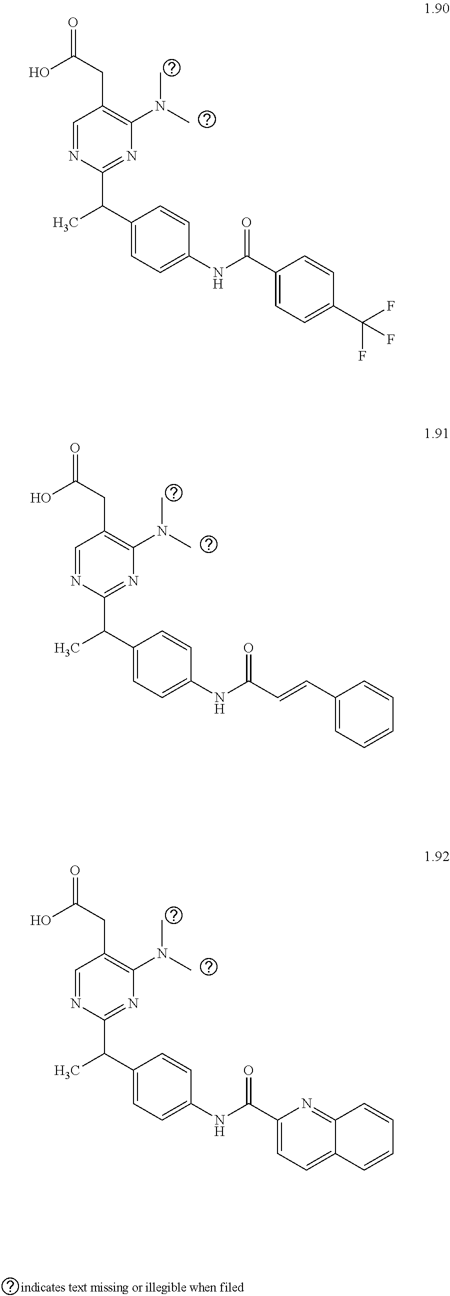 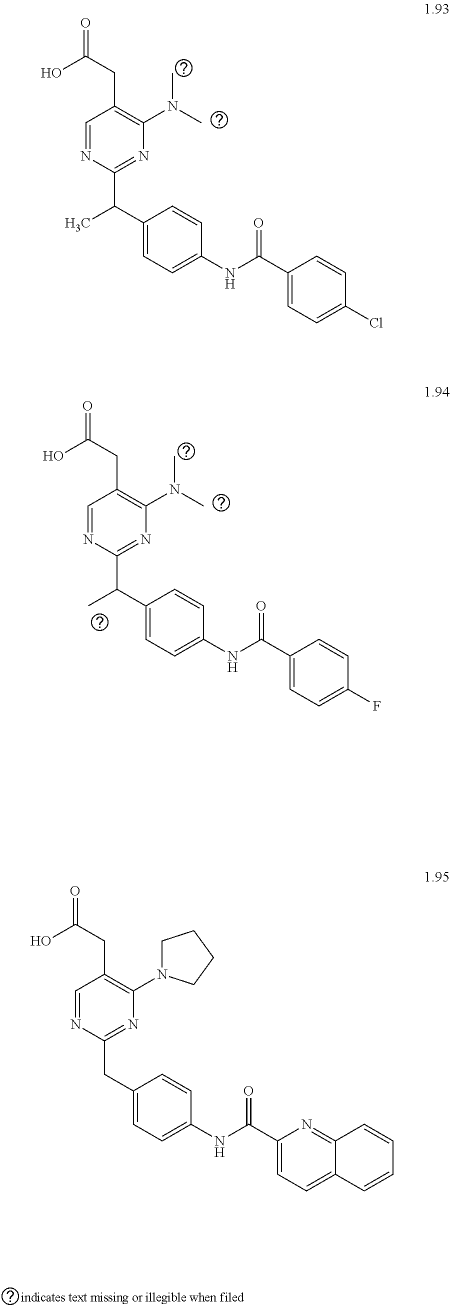 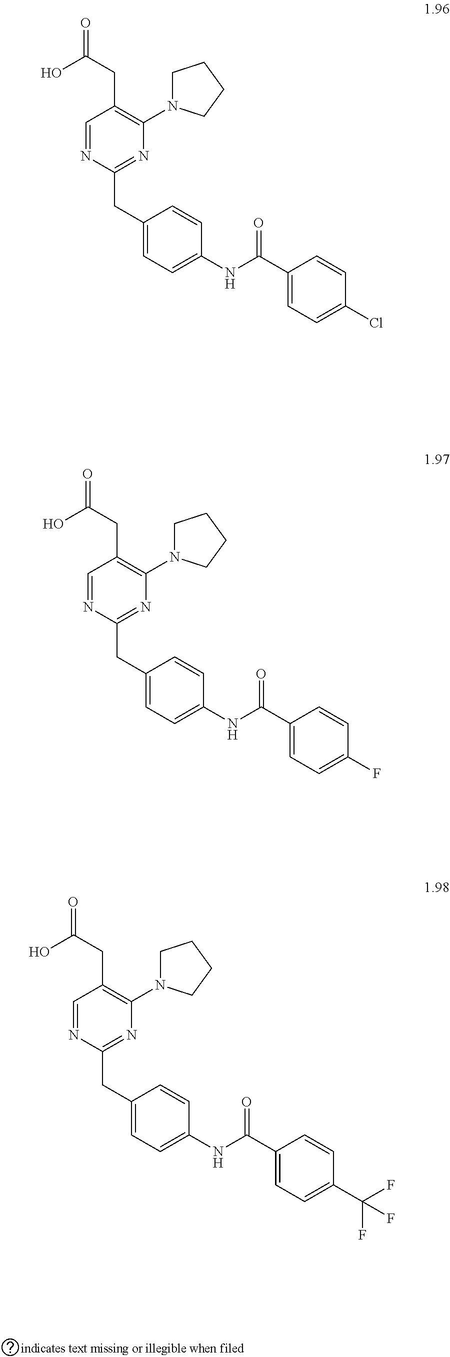 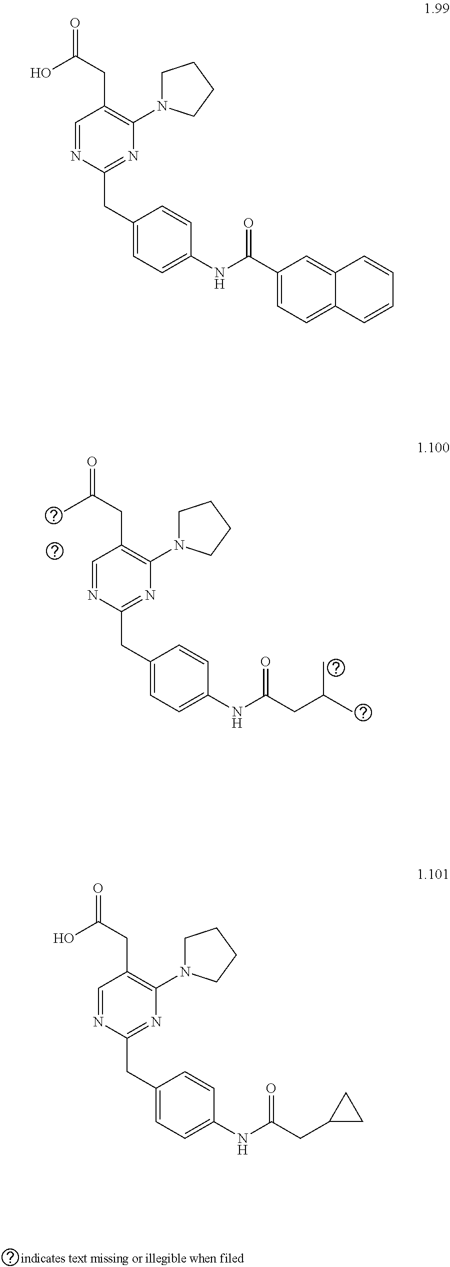 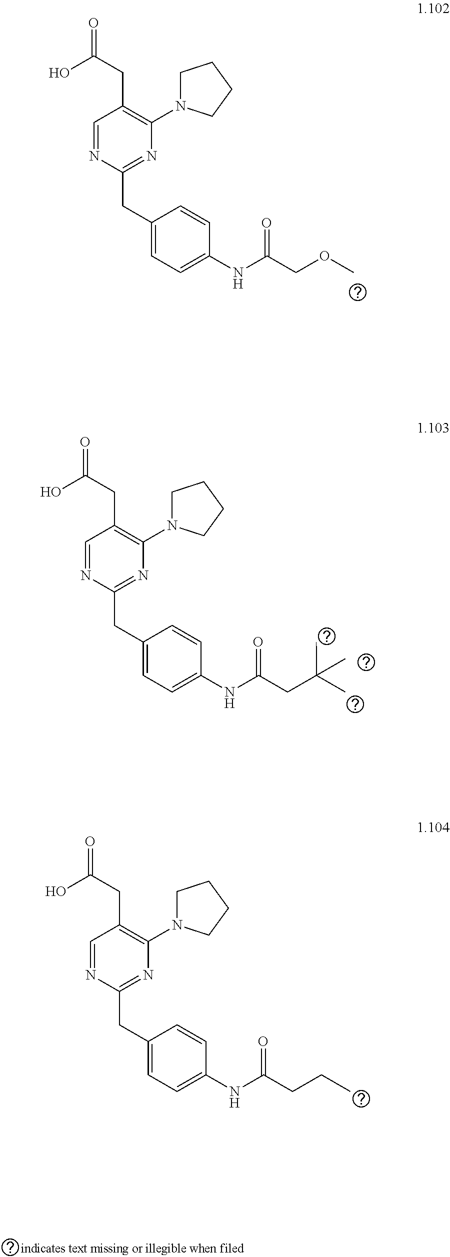 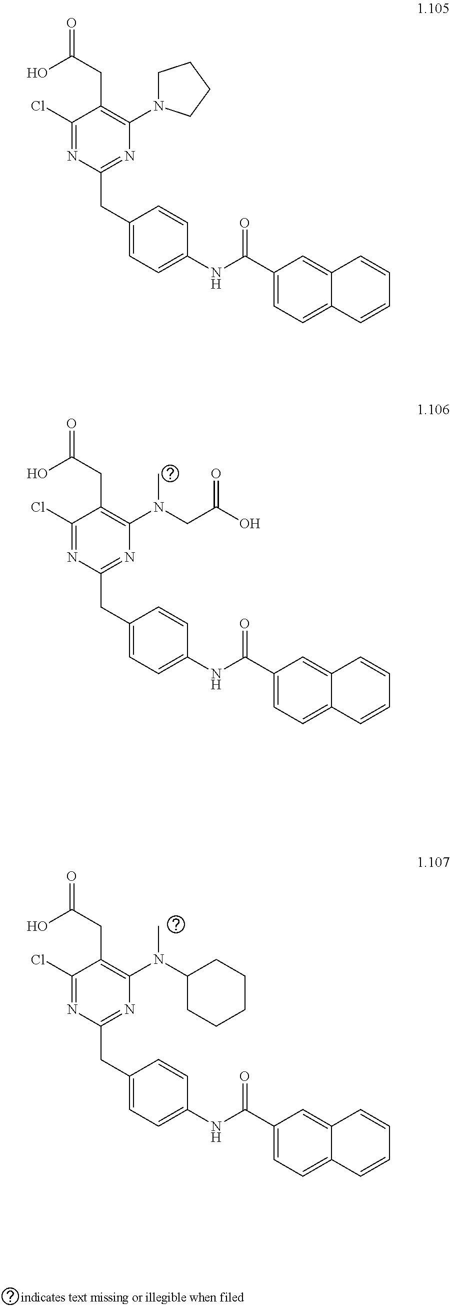 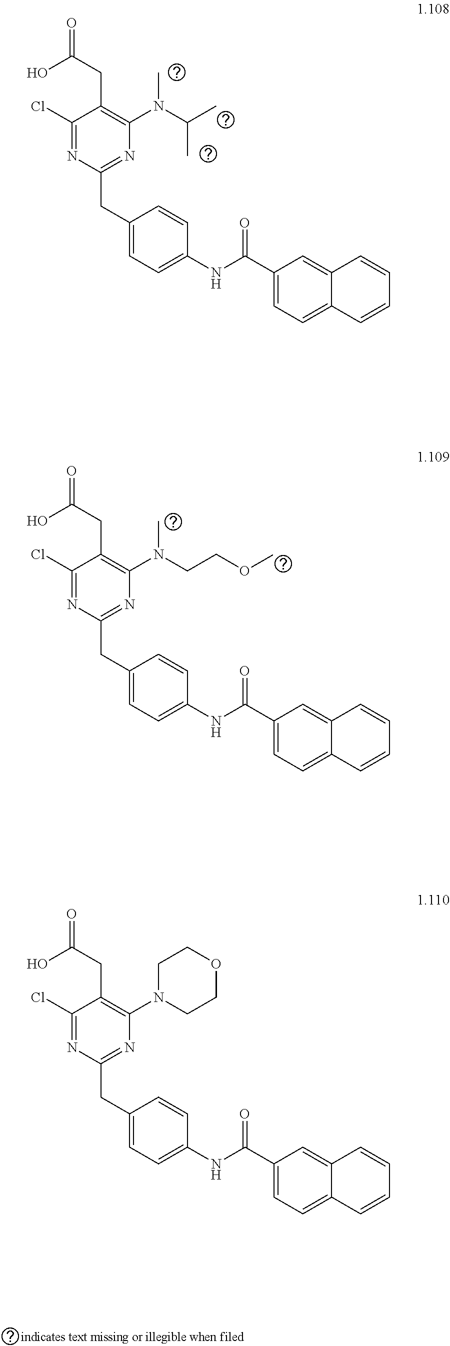 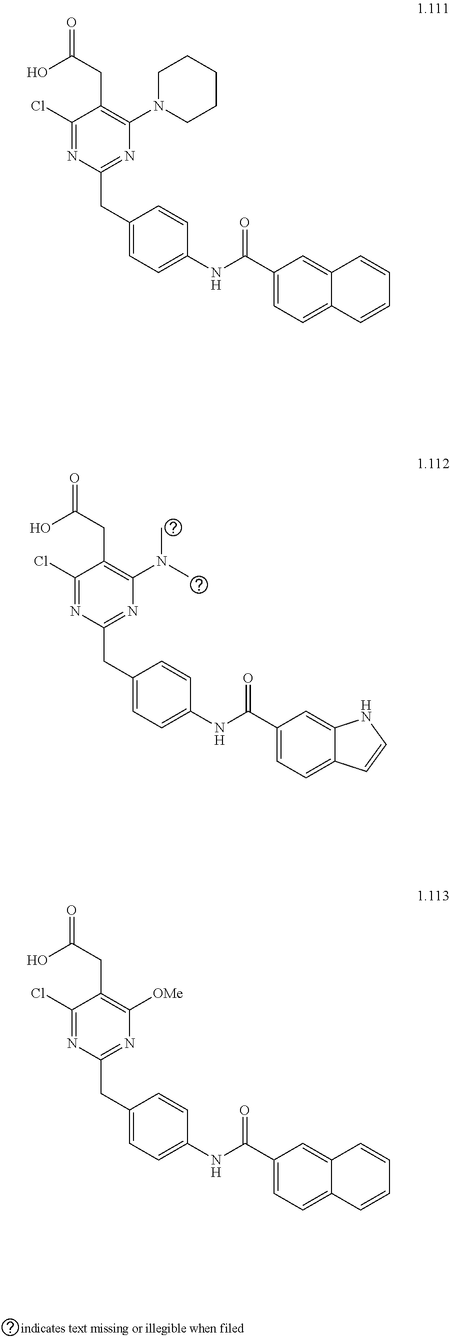 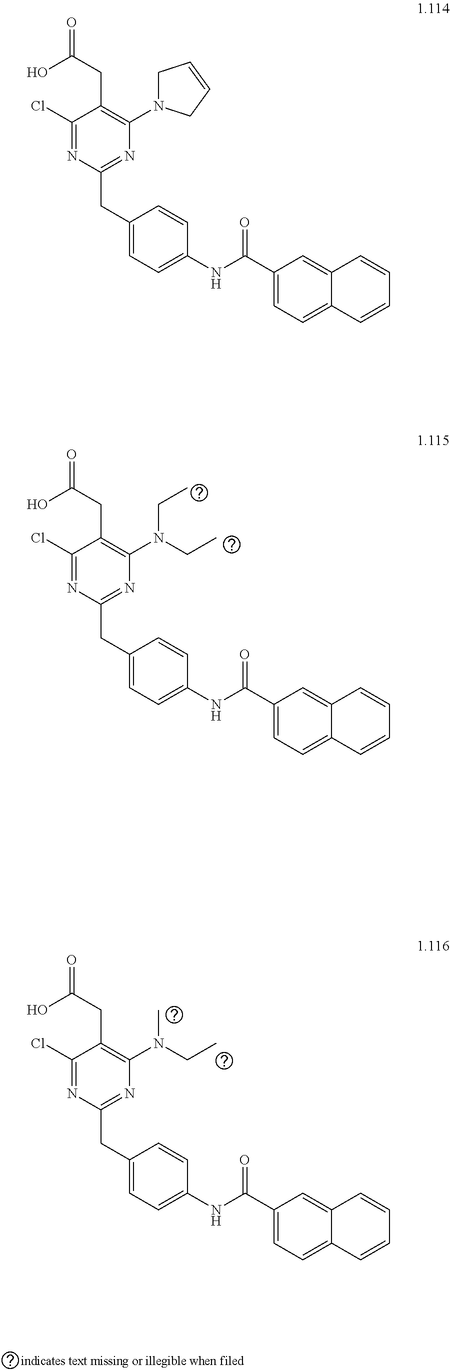 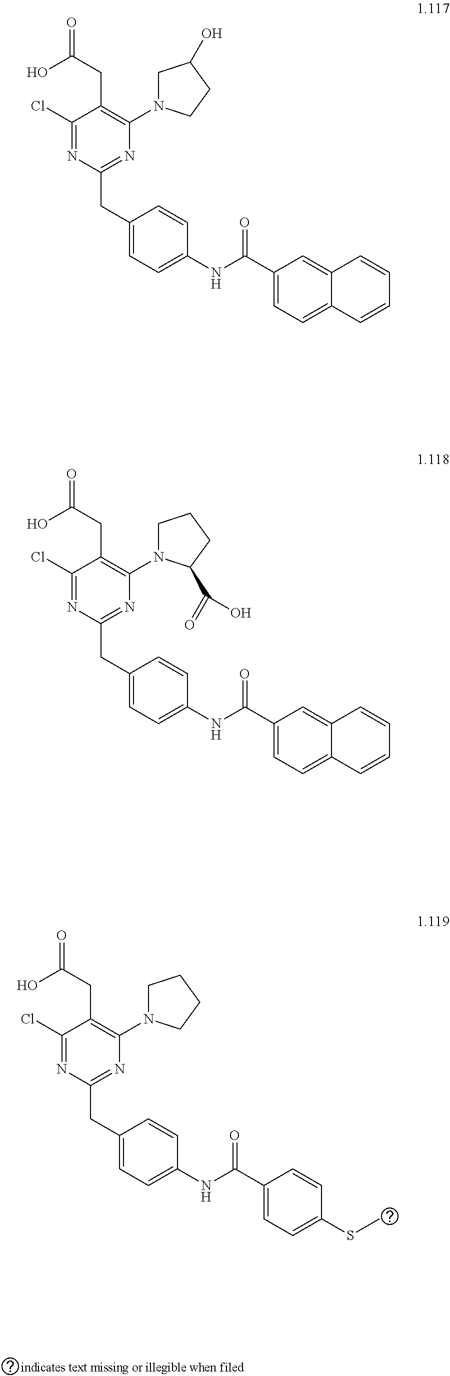 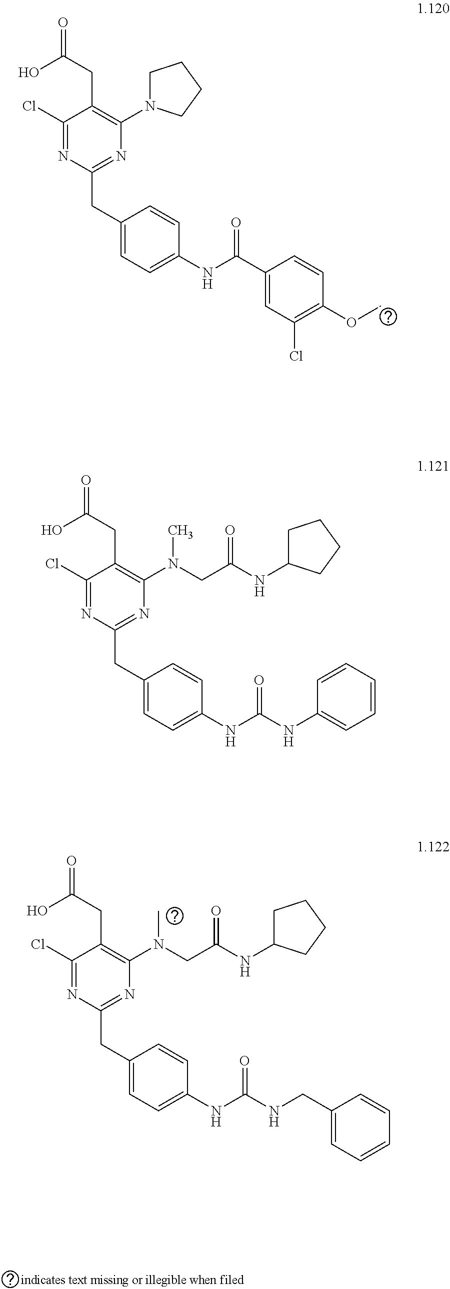 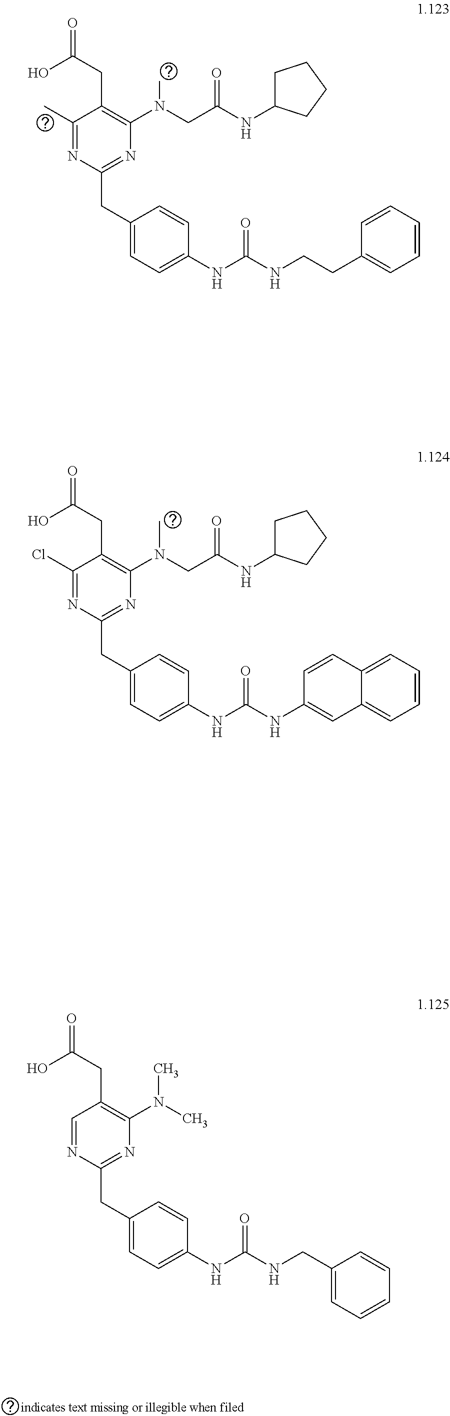 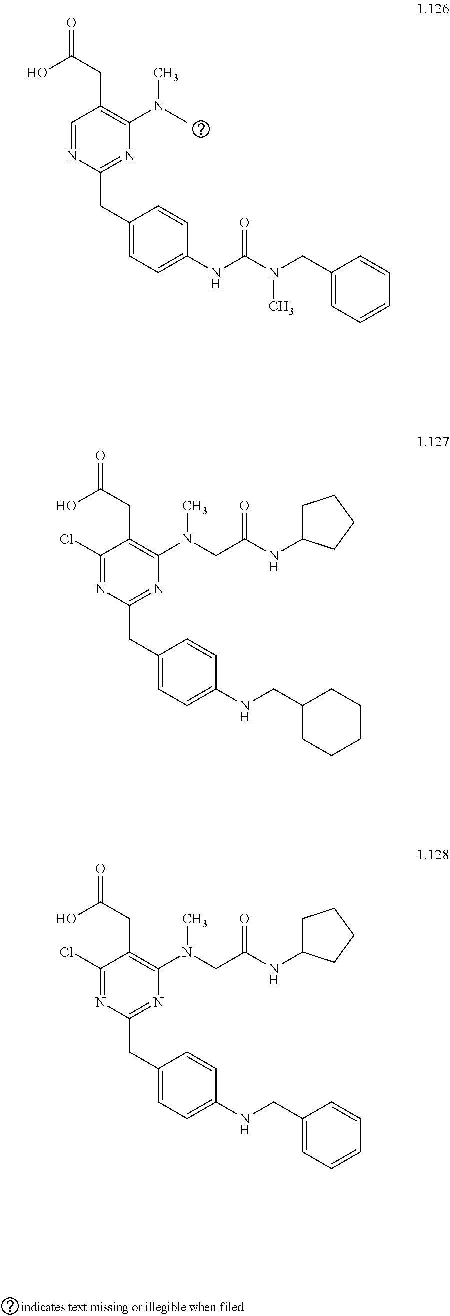 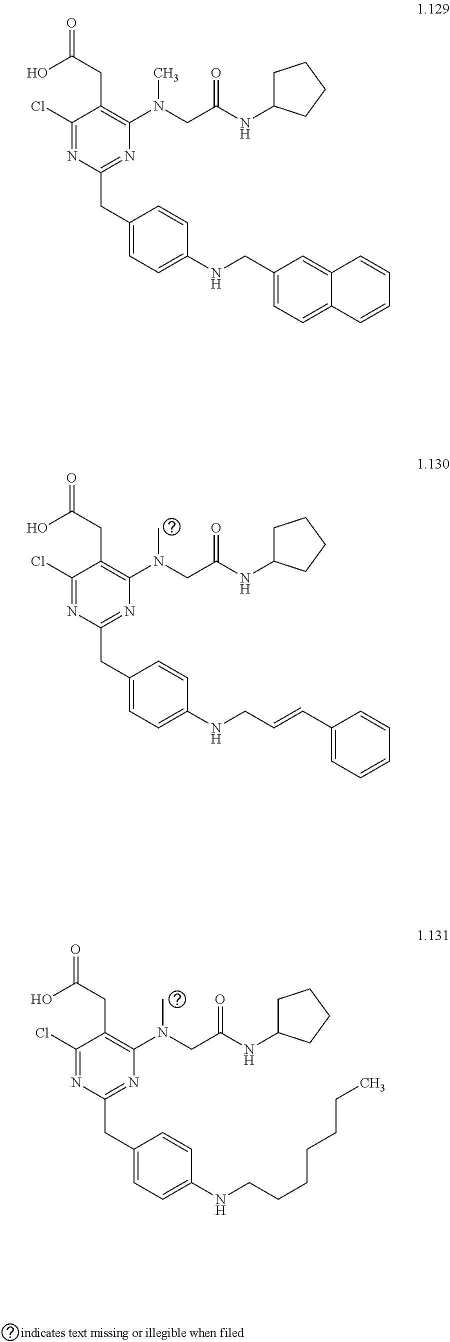 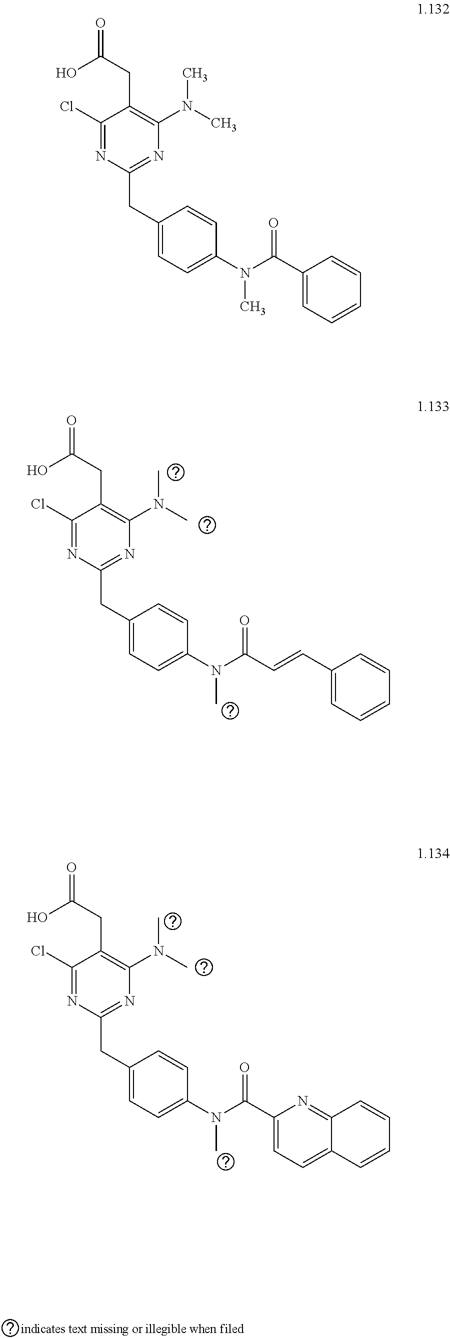 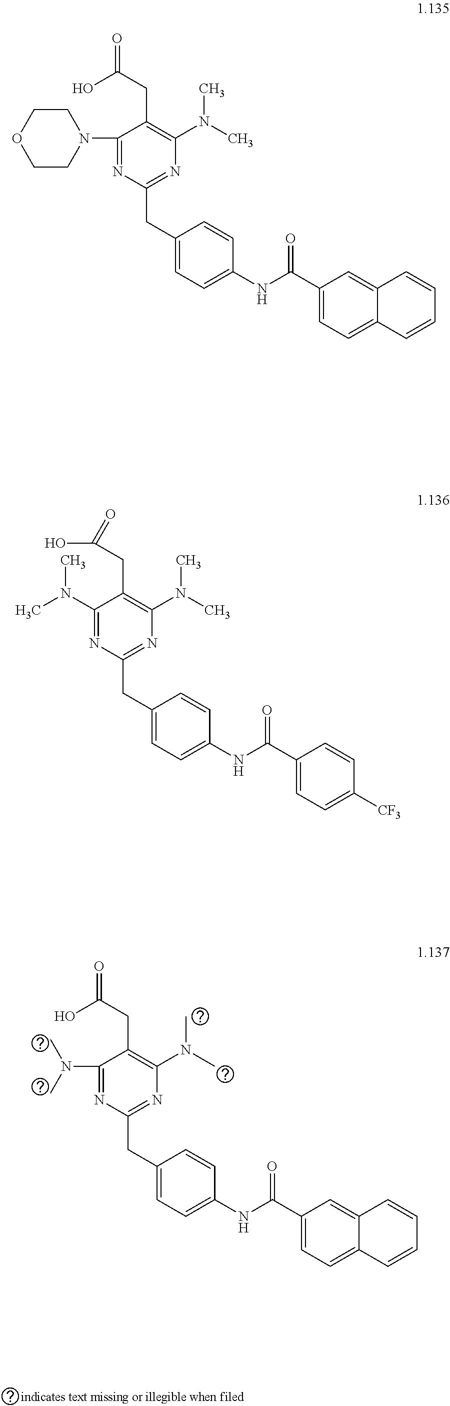 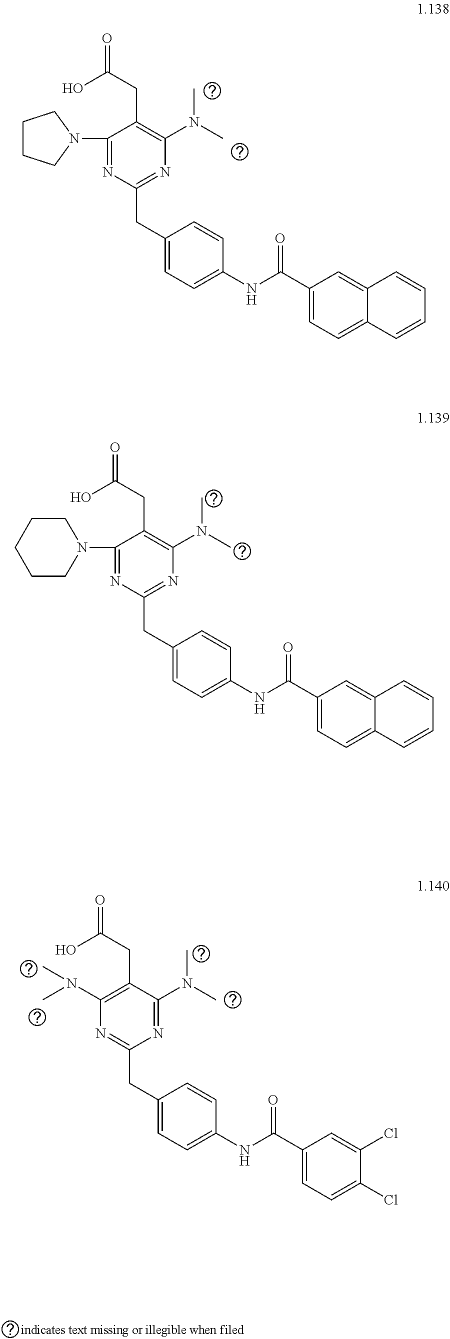 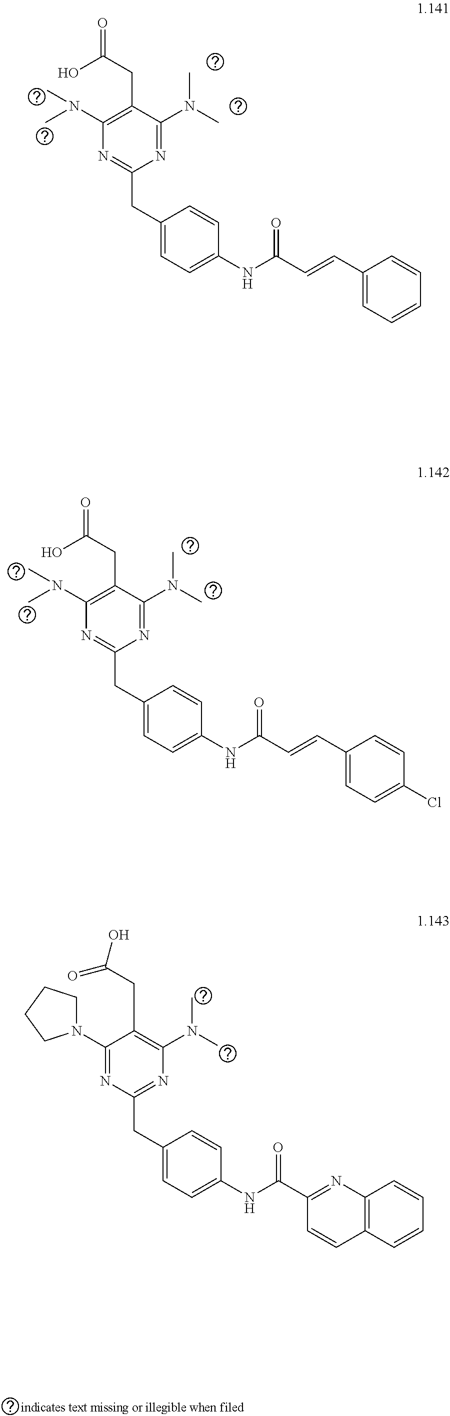 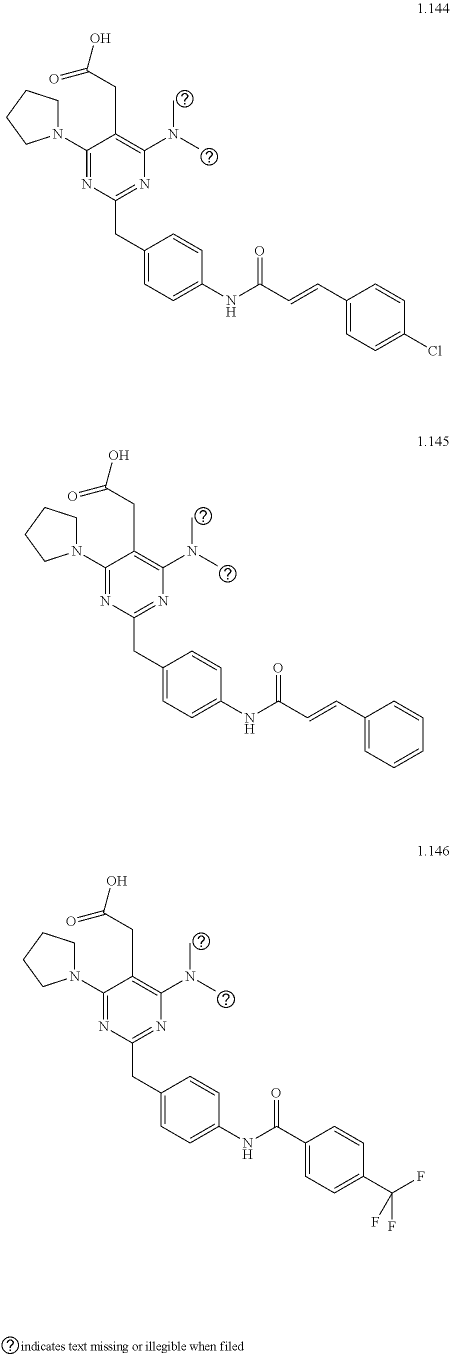 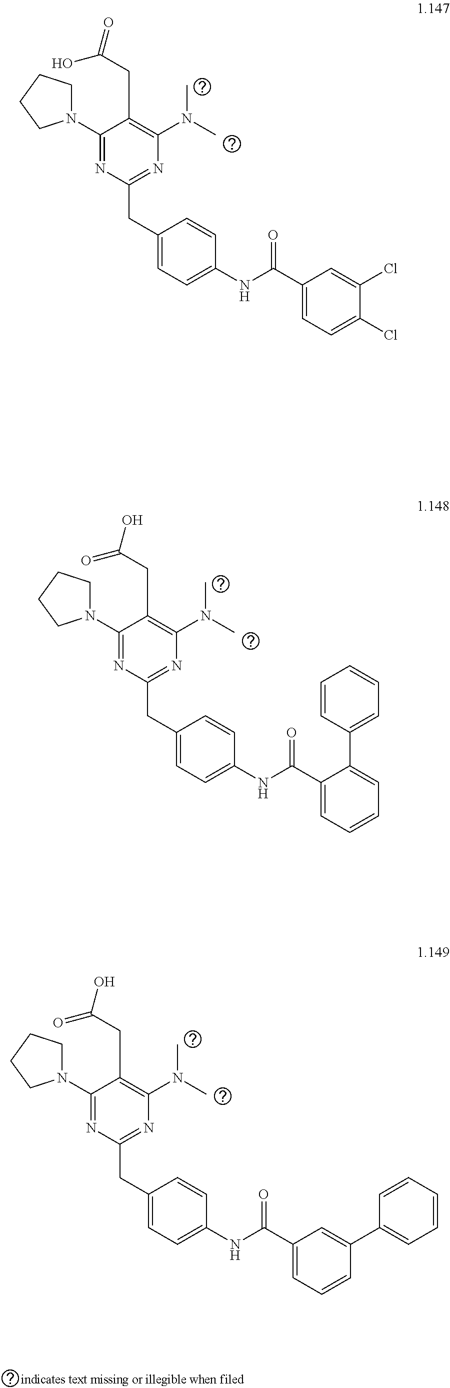 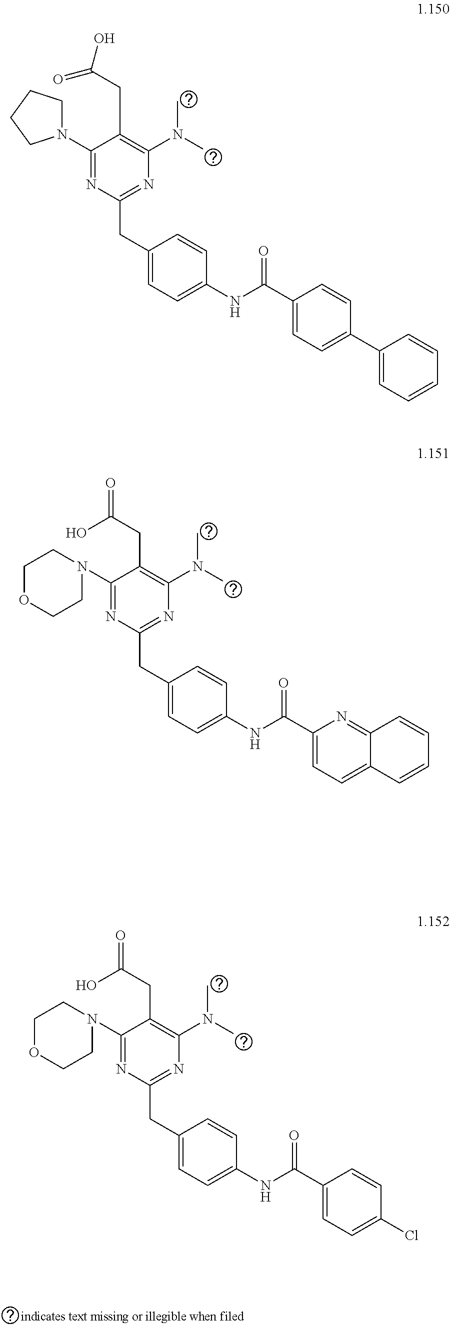 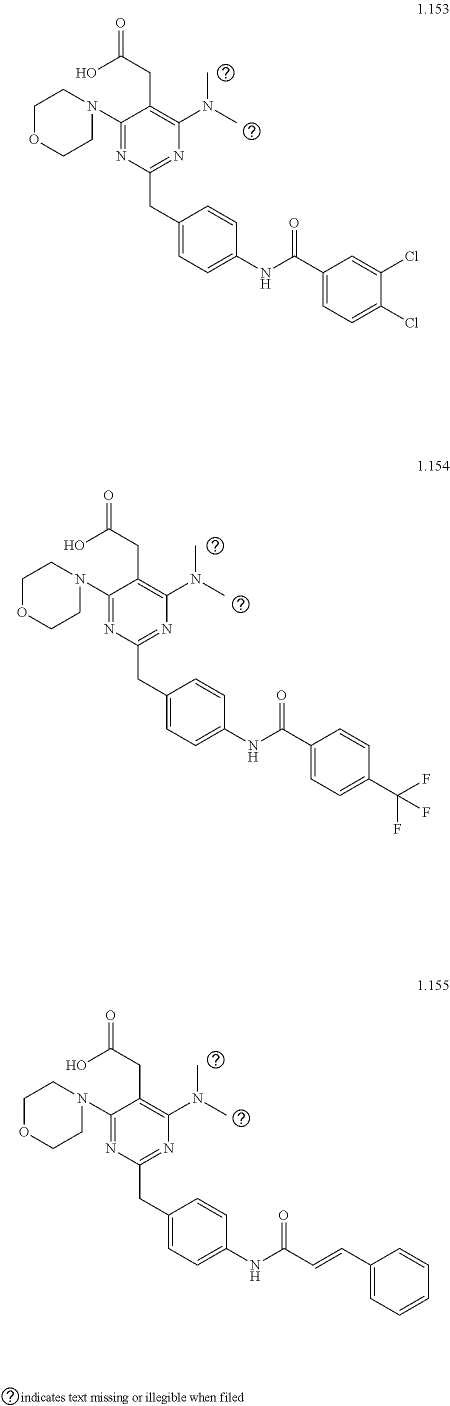 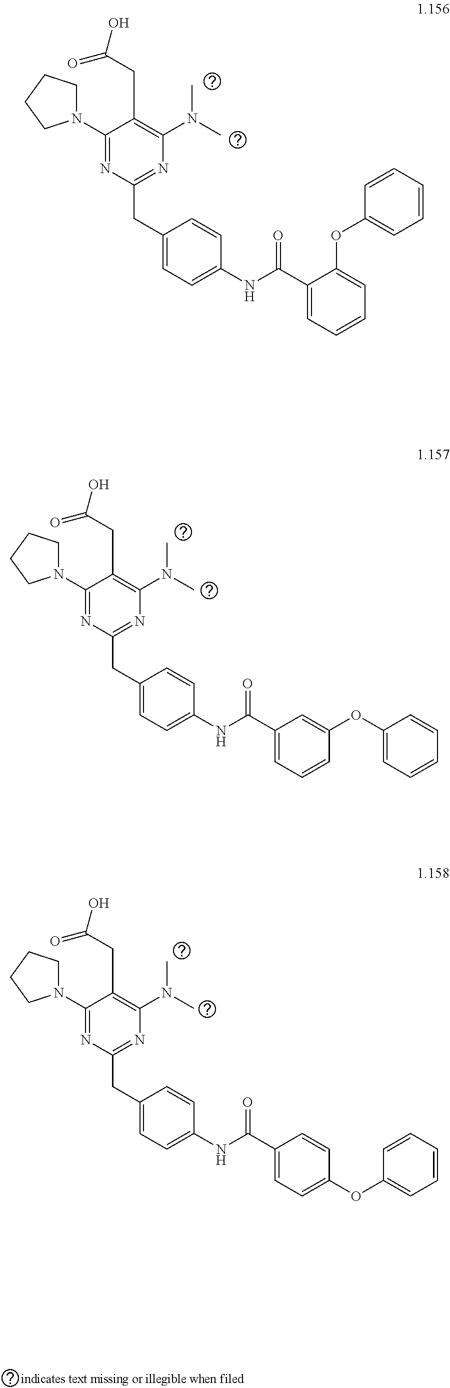 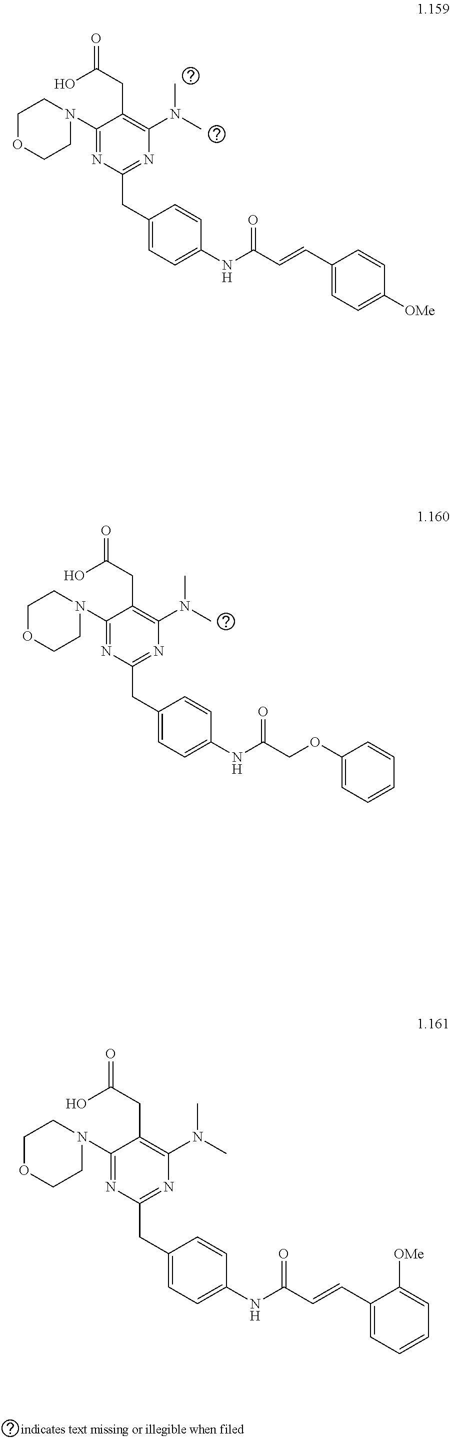 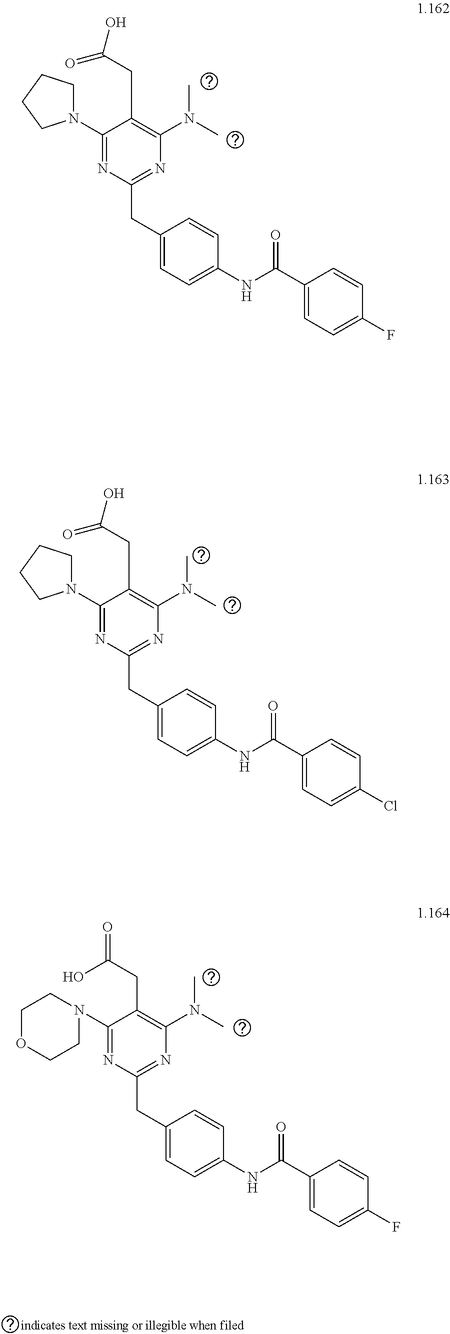 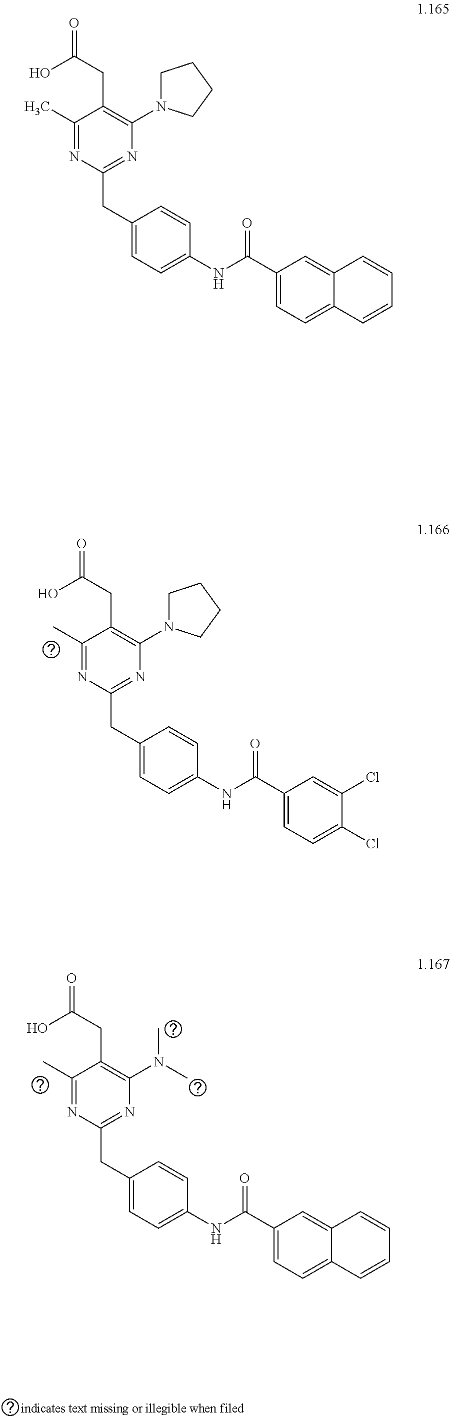 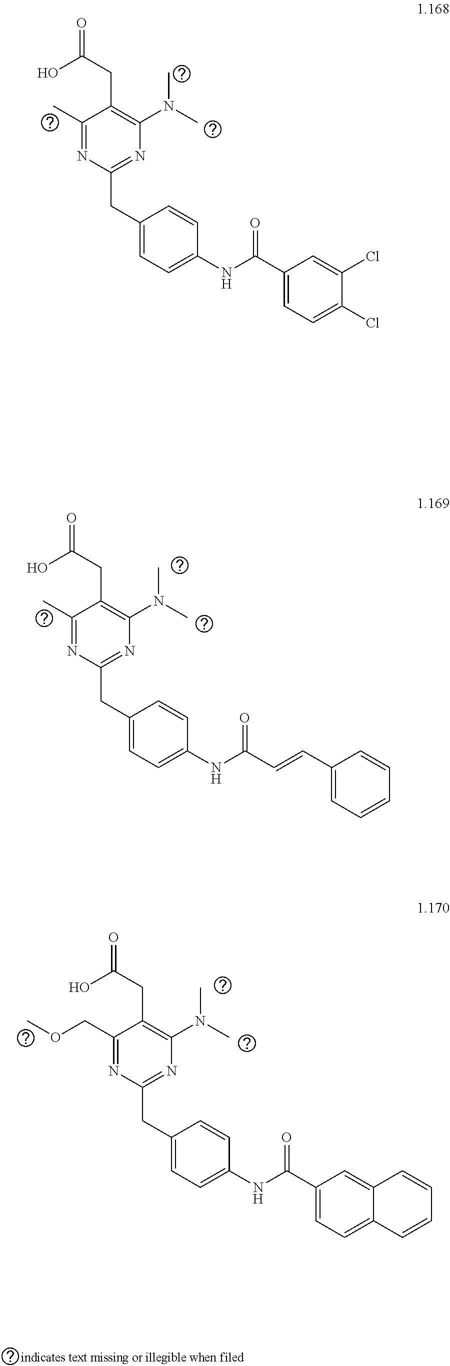 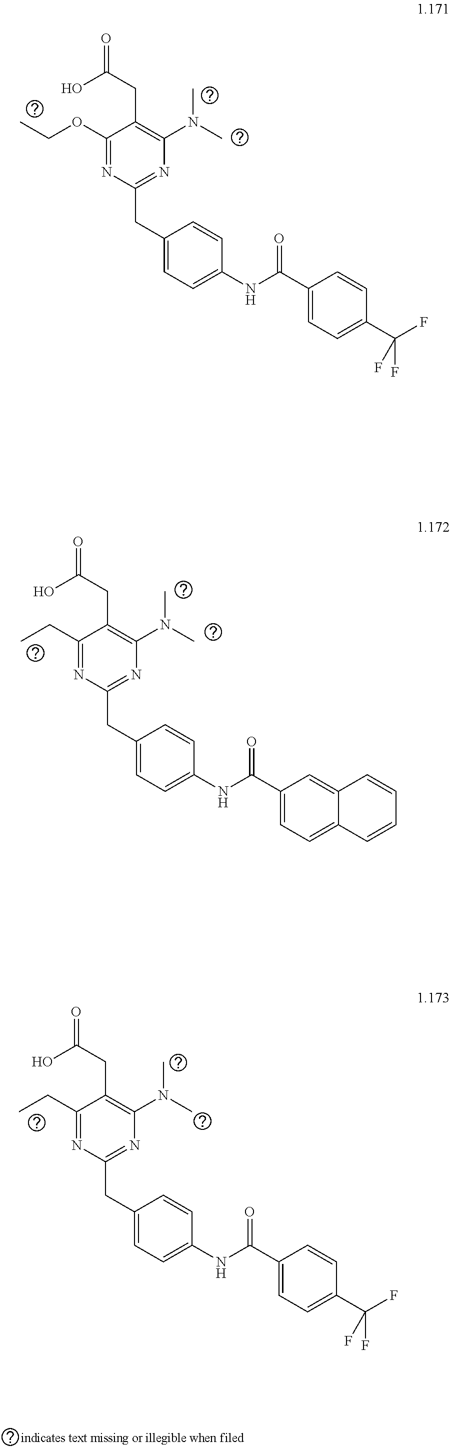 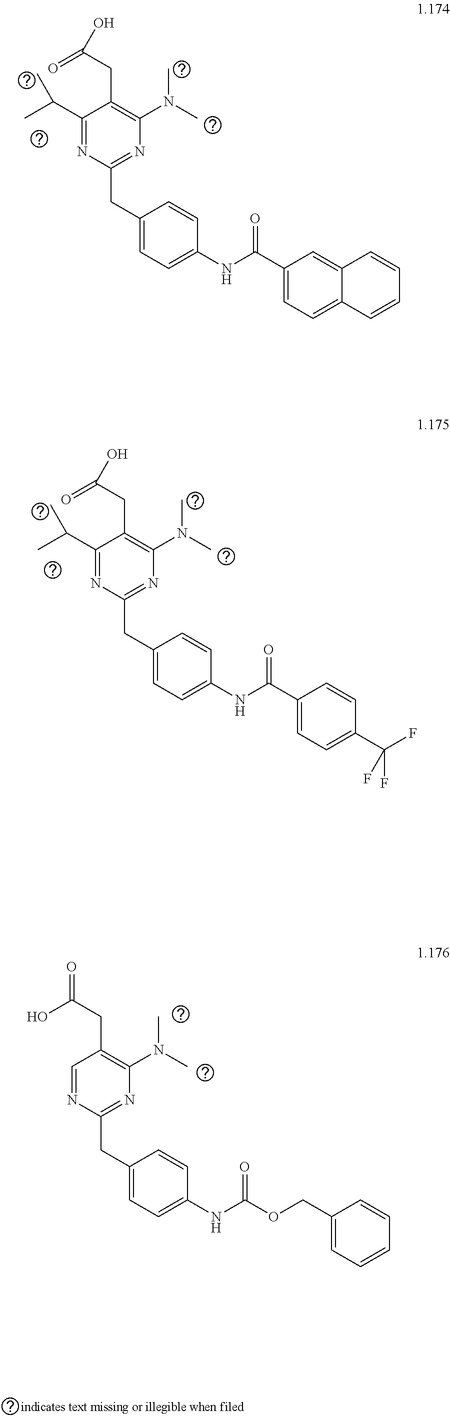 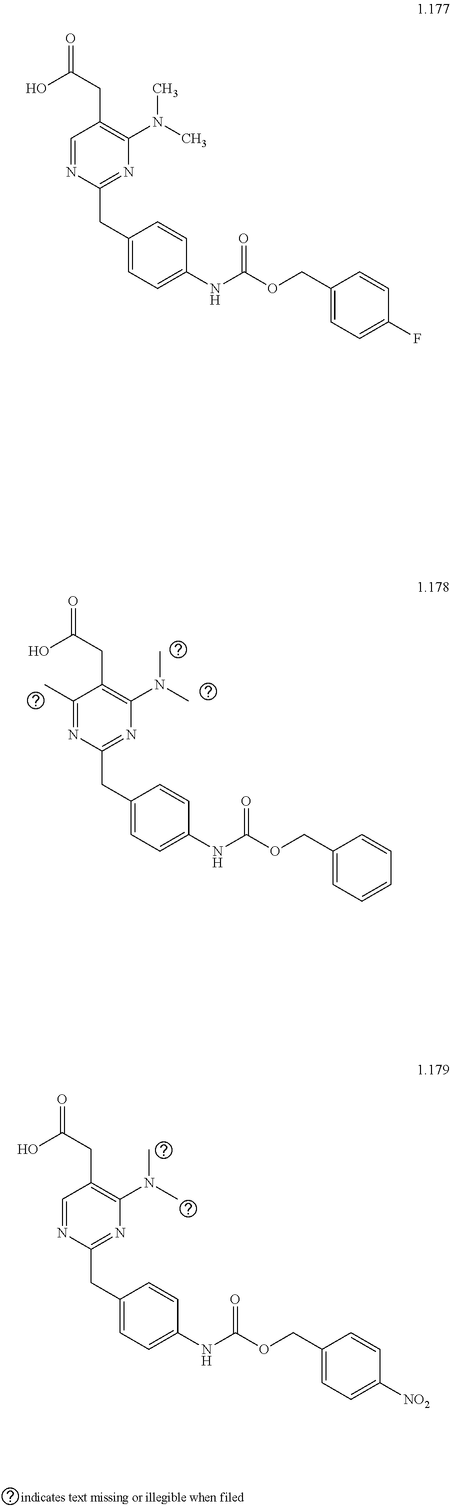 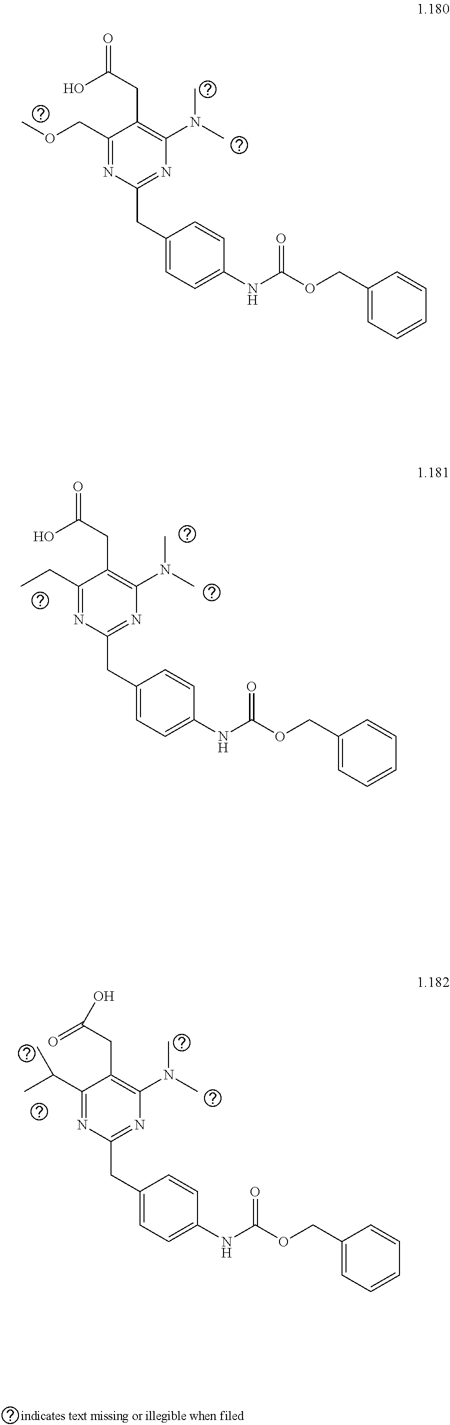 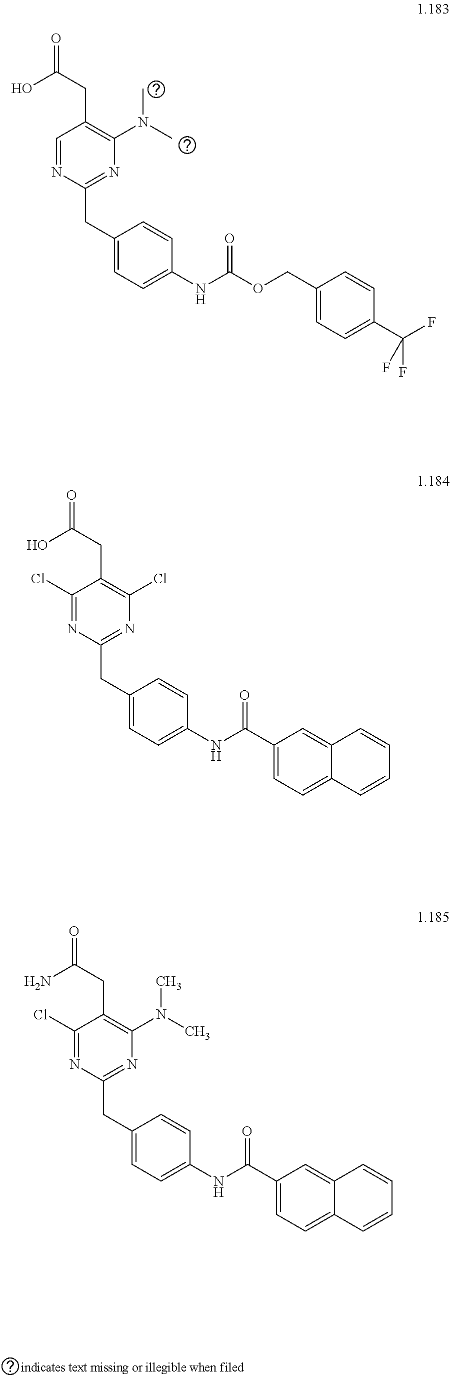 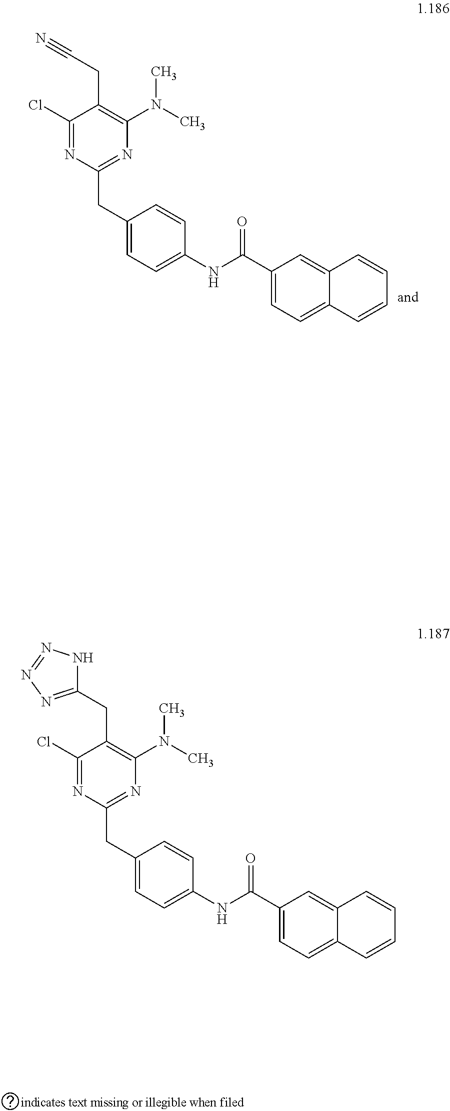
optionally in the form of an ester, an enantiomer, a racemate, a diastereomer, a tautomer, a solvate, a hydrate or an addition salt with a pharmacologically acceptable acid or base.

6. The pharmaceutical composition according to claim 1, wherein 1 is 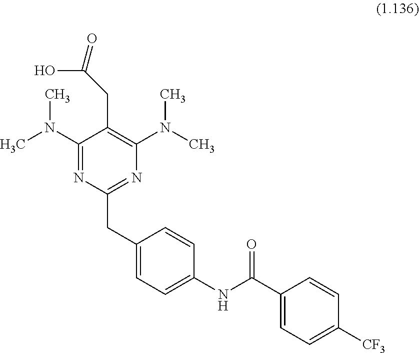
optionally in the form of an ester, an enantiomer, a racemate, a diastereomer, a tautomer or an addition salt with a pharmaceutically acceptable acid or base.

7. The pharmaceutical composition of claim 1, wherein the CRTH2 antagonists 1 are present as base addition salts, wherein the base is an amine selected from primary amines, including methylamine, ethylamine, ethanolamine, tris(hydroxymethyl)aminomethane, and ethylenediamine; secondary amines, including dimethylamine, diethylamine, diisopropylamine, dibutylamine, di-sec-butylamine, dicyclohexylamine, diethanolamine, meglumine, pyrrolidine, piperidine, piperazine, and benzathine; tertiary amines, including trimethylamine, triethylamine, triethanolamine, and 1-(2-hydroxyethyl)-pyrrolidine; quaternary ammoniums, including choline, tetra-methylammonium, and tetraethylammonium.

8. The pharmaceutical compositions according to claim 7, wherein the amine is selected from ethylenediamine and choline.

9. The pharmaceutical composition according to claim 1, wherein 2 is one or more LTD4 antagonists 2-5.

10. The pharmaceutical composition according to claim 1, wherein the LTD4 antagonist 2-5 is selected from montelukast, pranlukast and zafirlukast.

11. The pharmaceutical composition according to claim 10, wherein the LTD4 antagonist 2-5 is montelukast in the form of its monosodium salt.

12. The pharmaceutical composition according to claim 6, wherein 1.136 is present with a LTD4 antagonist at a weight ratio of from 2.5:1 to 40:1.

13. A unit dose form comprising the pharmaceutical composition according to claim 1, wherein said pharmaceutical composition in said pharmaceutical unit dose form comprises the CRTH2 antagonist 1 in an amount of from 1 mg to 1000 mg.

14. The unit dose form according to claim 13, wherein said pharmaceutical composition in said pharmaceutical unit dose form comprises the further active compounds 2 in an amount from 1 mg to 1000 mg.

15. The unit dose form according to claim 13, wherein said pharmaceutical composition in said pharmaceutical unit dose form comprises 1.136 as the CRTH2 antagonist 1 at an amount of from 25 mg to 400 mg and montelukast sodium as the LTD4 antagonist 2 at an amount of from 9 mg to 12 mg, preferably 10 mg.

16. (canceled)

17. (canceled)

18. A method for the treatment of respiratory disease or condition in a warm-blooded animal which comprises the step of administering to the animal a therapeutically effective amount of the pharmaceutical composition of claim 1.

19. The method according to claim 18 wherein the respiratory disease or condition is selected from among asthma, allergic rhinitis and non-allergic rhinitis.
